(19) United States
(12) Patent Application Publication
(10) Pub. No.: US 2008/0176862 A1
(43) Pub. Date: Jul. 24, 2008

(54) New Compounds 617

(75) Inventors: Stefan Berg, Sodertalje (SE); Jorg Holenz, Sodertalje (SE); Sofia Karlstrom, Sodertalje (SE); Jacob Kihlstrom, Sodertalje (SE); Johan Lindstrom, Sodertalje (SE); Laszlo Rakos, Sodertalje (SE)

Correspondence Address: PEPPER HAMILTON LLP, 400 BERWYN PARK, 899 CASSATT ROAD, BERWYN, PA, 19312-1183, US

(73) Assignees: ASTRAZENECA AB, Sodertalje (SE); ASTEX THERAPEUTICS LIMITED, Cambridge (GB)

(21) Appl. No.: 11/959,561

(22) Filed: Dec. 19, 2007

Related U.S. Application Data
(60) Provisional application No. 60/917,989, filed on May 15, 2007.
(60) Provisional application No. 60/870,936, filed on Dec. 20, 2006.

(51) Int. Cl. A61K 31/497; C07D 233/86; A61K 31/4166; A61P 25/28; A61P 25/00; C12N 9/99; C07D 403/10

(52) U.S. Cl. 514/255.05; 548/321.1; 514/391; 544/405; 435/184

(57) ABSTRACT

This invention relates to novel compounds having the structural formula I below:

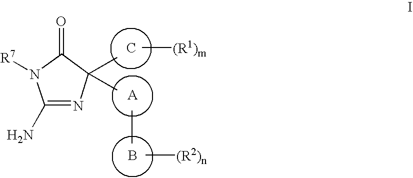

and to their pharmaceutically acceptable salt, compositions and methods of use. These novel compounds provide a treatment or prophylaxis of cognitive impairment, Alzheimer Disease, neurodegeneration and dementia.

The present invention relates to novel compounds and their pharmaceutical compositions. In addition, the present invention relates to therapeutic methods for the treatment and/or prevention of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class 1 transmembrane protein called APP, or amyloid precursor protein. Cleavage of APP by BACE generates the extracellular soluble APP□ fragment and the membrane bound CTF□ (C99) fragment that is subsequently cleaved by □-secretase to generate A□ peptide.

Alzheimer's disease (AD) is estimated to afflict more than 20 million people worldwide and is believed to be the most common form of dementia. Alzheimer's disease is a progressive dementia in which massive deposits of aggregated protein breakdown products
amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much higher risk of developing AD, and also of developing the disease at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to develop Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of Aβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041405, WO06/0065204, WO06/0065277, US2006287294, WO06/138265, US20050282826, US20050282825, US20060281729, WO06/138217, WO06/138230, WO06/138264, WO06/138265, WO06/138266, WO06/099379, WO06/076284, US20070004786, US20070004730, WO07/011833, WO07/011810, US20070099875, US20070099898, WO07/058,601, WO07/058,581, WO07/058,580, WO07/058,583, WO07/058,582, WO07/058,602, WO07/073,284, WO07/049,532, WO07/038,271, WO07/016,012, WO07/005,366, WO07/005,404, WO06/0009653).

The compounds of the present invention show beneficial properties compared to the potential inhibitors known in the art, e.g. improved hERG selectivity.

### DISCLOSURE OF THE INVENTION

Provided herein are novel compounds that are active BACE inhibitors. Thus, in one aspect of the invention, there is provided compounds according to formula I:

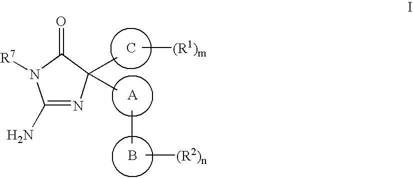

wherein
A is selected from phenyl and heteroaryl, wherein said phenyl or heteroaryl is optionally substituted with one or more R³;
B is selected from hydrogen, halo, cyano, phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl, and C₂₋₆alkenylC₃₋₆cycloalkyl, wherein said phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl or C₂₋₆alkenylC₃₋₆cycloalkyl, is optionally substituted with one or two R⁴;
C is selected from phenyl, heteroaryl and heterocyclyl, wherein said phenyl, heteroaryl or heterocyclyl is optionally substituted with one or two R⁵;

### R¹ and R² is OSO₂R⁶;

R³ is selected from halo and cyano;
R⁴ is selected from halo, C₁₋₆alkyl, methoxy, cyano, acetyl and trifluoromethoxy;
R⁵ is selected from halo, C₁₋₆alkyl, methoxy, trifluoromethyl, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂; said aryl being optionally fused with a 5 or 6 membered cyclyl or heterocyclyl group to form a bicyclic ring system; and said C₁₋₆alkyl, C₃₋₆cycloalkyl, aryl, heteroaryl, or bicyclic ring system is optionally substituted with one or more halogen, CN, NH₂, OH, COOH, OC₁₋₆alkyl, C₁₋₆alkylOH, SO₂H, C₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl or NC(O)(C₅₋₆heterocyclyl)₂;
R⁷ is C₁₋₆alkyl optionally substituted with halogen, CN, NH₂, OH, COOH, OC₁₋₆alkyl, SO₂H, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂;
m=0 or 1;
n=0 or 1;
wherein one of m or n is at least 1.

In another aspect of the invention, there is provided a compound according to formula I,

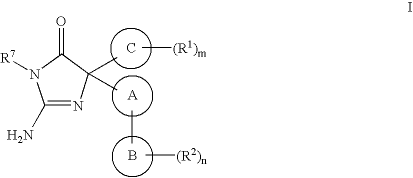

wherein

A is selected from phenyl and heteroaryl, wherein said phenyl or heteroaryl is optionally substituted with one or more R³;

B is selected from hydrogen, halo, cyano, phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl, and C₂₋₆alkenylC₃₋₆cycloalkyl, wherein said phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl or C₂₋₆alkenylC₃₋₆cycloalkyl, is optionally substituted with one or two R⁴;
C is selected from phenyl, heteroaryl and heterocyclyl, wherein said phenyl, heteroaryl or heterocyclyl is optionally substituted with one or two R⁵;

### R¹ and R² is OSO₂R⁶;

R³ is selected from halo and cyano;
R⁴ is selected from halo, C₁₋₆alkyl, methoxy, cyano, acetyl and trifluoromethoxy;
R⁵ is selected from halo, C₁₋₆alkyl, methoxy, trifluoromethyl, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂; said aryl being optionally fused with a 5 or 6 membered cyclyl or heterocyclyl group to form a bicyclic ring system; and said C₁₋₆alkyl, C₃₋₆cycloalkyl, aryl, heteroaryl, or bicyclic ring system is optionally substituted with one or more halogen, CN, NH₂, OH, COOH, OC₁₋₆alkyl, C₁₋₆alkylOH, SO₂H, C₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₁₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl or NC(O)(C₅₋₆heterocyclyl)₂;
R⁷ is C₁₋₆alkyl optionally substituted with halogen, CN, NH₂, OH, COOH, OC₁₋₆alkyl, SO₂H, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂;
m=0 or 1;
n=0 or 1;
wherein one of m or n is at least 1;
provided that the following compounds are excluded:
4-[2-Amino-4-(3′-methoxybiphenyl-3-yl)-1-methyl-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl trifluoromethanesulfonate;
3′-(2-Amino-1-methyl-5-oxo-4-phenyl-4,5-dihydro-1H-imidazol-4-yl)biphenyl-3-yl trifluoromethanesulfonate;
3′-(2-Amino-1-methyl-5-oxo-4-phenyl-4,5-dihydro-1H-imidazol-4-yl)-5-methoxybiphenyl-2-yl trifluoromethanesulfonate;
3-[2-Amino-4-(3′-methoxybiphenyl-3-yl)-1-methyl-5-oxo-4,5-dihydro-1H-imidazol-4-yl]phenyl trifluoromethanesulfonate;
3-[2-Amino-4-(3′-methoxybiphenyl-3-yl)-1-methyl-5-oxo-4,5-dihydro-1H-imidazol-4-yl]phenyl methanesulfonate;
4-[2-Amino-1-methyl-5-oxo-4-(3-pyridin-3-ylphenyl)-4,5-dihydro-1H-imidazol-4-yl]phenyl methanesulfonate;
4-[2-Amino-4-(5′-chloro-2′-methoxybiphenyl-3-yl)-1-methyl-5-oxo-4,5-dihydro-1H-imidazol-4-yl]phenyl methanesulfonate;
4-[2-Amino-4-(3′-cyanobiphenyl-3-yl)-1-methyl-5-oxo-4,5-dihydro-1H-imidazol-4-yl]phenyl methanesulfonate;
4-{2-Amino-1-methyl-5-oxo-4-[3′-(trifluoromethoxy)biphenyl-3-yl]-4,5-dihydro-1H-imidazol-4-yl}phenyl methanesulfonate;
4-{2-Amino-4-[3-(2-chloropyridin-4-yl)phenyl]-1-methyl-5-oxo-4,5-dihydro-1H-imidazol-4-yl}phenyl methanesulfonate;
4-[2-Amino-4-(3′-cyanobiphenyl-3-yl)-1-methyl-5-oxo-4,5-dihydro-1H-imidazol-4-yl]phenyl propane-1-sulfonate;
4-[2-Amino-4-(2′,5′-dimethoxybiphenyl-3-yl)-1-methyl-5-oxo-4,5-dihydro-1H-imidazol-4-yl]phenyl propane-1-sulfonate;
4-[2-Amino-4-(2′-fluoro-3′-methoxybiphenyl-3-yl)-1-methyl-5-oxo-4,5-dihydro-1H-imidazol-4-yl]phenyl propane-1-sulfonate;
4-[2-Amino-4-(2′-fluoro-5′-methoxybiphenyl-3-yl)-1-methyl-5-oxo-4,5-dihydro-1H-imidazol-4-yl]phenyl propane-1-sulfonate;
4-[2-Amino-4-(3′-cyano-4′-fluorobiphenyl-3-yl)-1-methyl-5-oxo-4,5-dihydro-1H-imidazol-4-yl]phenyl propane-1-sulfonate;
4-[2-Amino-4-(5′-cyano-2′-fluorobiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyridin-3-ylphenyl)-4,5-dihydro-1H-imidazol-4-yl]phenyl propane-1-sulfonate;
4-{2-Amino-4-[3-(2-fluoropyridin-3-yl)phenyl]-1-methyl-5-oxo-4,5-dihydro-1H-imidazol-4-yl}phenyl propane-1-sulfonate;
4-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
4-{2-Amino-4-[3-(6-fluoropyridin-3-yl)phenyl]-1-methyl-5-oxo-4,5-dihydro-1H-imidazol-4-yl}phenyl propane-1-sulfonate;
4-[2-Amino-1-methyl-5-oxo-4-(3-pyrimidin-5-ylphenyl)-4,5-dihydro-1H-imidazol-4-yl]phenyl propane-1-sulfonate;
4-{2-Amino-4-[3-(2-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-3-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-5-fluoropyridin-3-yl)phenyl]-1-methyl-5-oxo-4,5-dihydro-1H-imidazol-4-yl}phenyl propane-1-sulfonate;
4-{2-Amino-4-[3-(2,6-difluoropyridin-3-yl)phenyl]-1-methyl-5-oxo-4,5-dihydro-1H-imidazol-4-yl}phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl propane-1-sulfonate;
4-[2-Amino-4-(4′-fluoro-3′-methoxybiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyrazin-2-ylphenyl)-4,5-dihydro-1H-imidazol-4-yl]phenyl propane-1-sulfonate;
3′-(2-Amino-1-methyl-5-oxo-4-phenyl-4,5-dihydro-1H-imidazol-4-yl)-5-methoxybiphenyl-3-yl methanesulfonate;
4-{2-Amino-4-[3-(5-methoxypyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl trifluoromethanesulfonate;
3-[2-Amino-1-methyl-5-oxo-4-(3-pyrimidin-5-ylphenyl)-4,5-dihydro-1H-imidazol-4-yl]phenyl trifluoromethanesulfonate;
3-{2-Amino-4-[3-(5-methoxypyridin-3-yl)phenyl]-1-methyl-5-oxo-4,5-dihydro-1H-imidazol-4-yl}phenyl trifluoromethanesulfonate;
4-[2-Amino-4-(3′,5′-dichlorodiphenyl-3-yl)-1-methyl-5-oxo-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl methanesulfonate;
3-{2-Amino-4-[3-(6-fluoropyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl methanesulfonate;
3-{2-Amino-4-[3-(5-chloro-2-fluoropyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-5-ylphenyl)-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl propane-1-sulfonate;
3-{2-Amino-4-[3-(6-fluoropyridin-3-yl)phenyl]-1-methyl-5-oxo-4,5-dihydro-1H-imidazol-4-yl}phenyl propane-1-sulfonate;
3-{2-Amino-4-[3-(2-fluoropyridin-3-yl)phenyl]-1-methyl-5-oxo-4,5-dihydro-1H-imidazol-4-yl}phenyl propane-1-sulfonate;
3-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-5-ylphenyl)-4,5-dihydro-1H-imidazol-4-yl]phenyl propane-1-sulfonate;
3-[2-Amino-1-methyl-5-oxo-4-(3-pyridin-3-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl propane-2-sulfonate;
3-{2-Amino-4-[3-(2-fluoropyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-5-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl dimethylsulfamate;
3-{2-Amino-4-[3-(5-chloro-2-fluoropyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-5-ylphenyl)-4,5-dihydro-1H-imidazol-4-yl]phenyl dimethylsulfamate;
3-{2-Amino-4-[3-(5-methoxypyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-2-ylphenyl)-4,5-dihydro-1H-imidazol-4-yl]phenyl methanesulfonate;
3-{2-Amino-4-[3-(5-methoxypyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-2-ylphenyl)-4,5-dihydro-1H-imidazol-4-yl]phenyl propane-1-sulfonate;
3-{2-Amino-4-[3-(5-methoxypyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-2-ylphenyl)-4,5-dihydro-1H-imidazol-4-yl]phenyl propane-2-sulfonate;
3-{2-Amino-4-[3-(5-methoxypyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-2-ylphenyl)-4,5-dihydro-1H-imidazol-4-yl]phenyl dimethylsulfamate;
3-[2-Amino-4-(3′-methoxybiphenyl-3-yl)-1-methyl-5-oxo-4,5-dihydro-1H-imidazol-4-yl]phenyl propane-2-sulfonate;
3′-(2-Amino-1-methyl-5-oxo-4-phenyl-4,5-dihydro-1H-imidazol-4-yl)-5-methoxybiphenyl-3-yl trifluoromethanesulfonate;
4-{2-Amino-4-[3-(5-methoxypyridin-3-yl)phenyl]-1-methyl-5-oxo-4,5-dihydro-1H-imidazol-4-yl}phenyl propane-1-sulfonate;
4-[2-Amino-4-(3′-cyano-6-fluorobiphenyl-3-yl)-1-methyl-5-oxo-4,5-dihydro-1H-imidazol-4-yl]phenyl methanesulfonate;
4-[2-Amino-4-(3′-cyano-6-fluorobiphenyl-3-yl)-5-oxo-4,5-dihydro-1H-imidazol-4-yl]phenyl methanesulfonate;
4-[2-Amino-4-(2′,6-difluoro-3′-methoxybiphenyl-3-yl)-5-oxo-4,5-dihydro-1H-imidazol-4-yl]phenyl methanesulfonate;
4-[2-Amino-4-(2′,6-difluoro-5′-methoxybiphenyl-3-yl)-5-oxo-4,5-dihydro-1H-imidazol-4-yl]phenyl methanesulfonate;
4-[2-Amino-4-(3′-cyano-4′,6-difluorobiphenyl-3-yl)-5-oxo-4,5-dihydro-1H-imidazol-4-yl]phenyl methanesulfonate;
4-[2-Amino-4-(5′-cyano-2′,6-difluorobiphenyl-3-yl)-5-oxo-4,5-dihydro-1H-imidazol-4-yl]phenyl methanesulfonate;
4-[2-Amino-4-(4-fluoro-3-pyridin-3-ylphenyl)-5-oxo-4,5-dihydro-1H-imidazol-4-yl]phenyl methanesulfonate;
4-{2-Amino-4-[4-fluoro-3-(2-fluoropyridin-3-yl)phenyl]-5-oxo-4,5-dihydro-1H-imidazol-4-yl}phenyl methanesulfonate (racemate);
4-{2-Amino-4-[3-(5-chloro-2-fluoropyridin-3-yl)-4-fluorophenyl]-5-oxo-4,5-dihydro-1H-imidazol-4-yl}phenyl methanesulfonate;
4-{2-Amino-4-[4-fluoro-3-(6-fluoropyridin-3-yl)phenyl]-5-oxo-4,5-dihydro-1H-imidazol-4-yl}phenyl methanesulfonate;
4-{2-Amino-4-[4-fluoro-3-(2-fluoropyridin-4-yl)phenyl]-5-oxo-4,5-dihydro-1H-imidazol-4-yl}phenyl methanesulfonate;
4-{2-Amino-4-[3-(2-chloro-3-fluoropyridin-4-yl)-4-fluorophenyl]-5-oxo-4,5-dihydro-1H-imidazol-4-yl}phenyl methanesulfonate;
4-{2-Amino-4-[3-(2-chloro-5-fluoropyridin-3-yl)-4-fluorophenyl]-5-oxo-4,5-dihydro-1H-imidazol-4-yl}phenyl methanesulfonate;
4-{2-Amino-4-[3-(2,6-difluoropyridin-3-yl)-4-fluorophenyl]-5-oxo-4,5-dihydro-1H-imidazol-4-yl}phenyl methanesulfonate;
4-{2-Amino-4-[6-fluoro-3′-(trifluoromethoxy)biphenyl-3-yl]-5-oxo-4,5-dihydro-1H-imidazol-4-yl}phenyl methanesulfonate;
4-[2-Amino-4-(3′-chloro-6-fluorobiphenyl-3-yl)-5-oxo-4,5-dihydro-1H-imidazol-4-yl]phenyl methanesulfonate;
4-[2-Amino-4-(4-fluoro-3-pyridin-4-ylphenyl)-5-oxo-4,5-dihydro-1H-imidazol-4-yl]phenyl methanesulfonate;
4-[2-Amino-4-(4-fluoro-3-pyrimidin-5-ylphenyl)-5-oxo-4,5-dihydro-1H-imidazol-4-yl]phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-{2-Amino-4-[4-fluoro-3-(5-fluoropyridin-3-yl)phenyl]-5-oxo-4,5-dihydro-1H-imidazol-4-yl}phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl propane-1-sulfonate;
4-[2-Amino-4-(3′-methoxybiphenyl-3-yl)-1-methyl-5-oxo-4,5-dihydro-1H-imidazol-4-yl]phenyl propane-2-sulfonate;
4-[2-Amino-4-(3′-methoxybiphenyl-3-yl)-1-methyl-5-oxo-4,5-dihydro-1H-imidazol-4-yl]phenyl dimethylsulfamate;
4-(2-Amino-4-{3′-methoxy-5′-[(methylsulfonyl)oxy]biphenyl-3-yl}-1-methyl-5-oxo-4,5-dihydro-1H-imidazol-4-yl)phenyl propane-1-sulfonate;
3-[2-Amino-1-methyl-5-oxo-4-(3-pyridin-3-ylphenyl)-4,5-dihydro-1H-imidazol-4-yl]phenyl trifluoromethanesulfonate;
3-[2-Amino-1-methyl-5-oxo-4-(3-pyridin-3-ylphenyl)-4,5-dihydro-1H-imidazol-4-yl]phenyl dimethylsulfamate;
4-(2-Amino-4-{6-fluoro-3′-methoxy-5′-[(methylsulfonyl)oxy]biphenyl-3-yl}-5-oxo-4,5-dihydro-1H-imidazol-4-yl)phenyl methanesulfonate;
3-(2-Amino-1-methyl-5-oxo-4-phenyl-4,5-dihydro-1H-imidazol-4-yl)phenyl methanesulfonate;
3-(2-Amino-1-methyl-5-oxo-4-phenyl-4,5-dihydro-1H-imidazol-4-yl)phenyl trifluoromethanesulfonate;
3′-(2-Amino-1-methyl-5-oxo-4-pyridin-4-yl-4,5-dihydro-1H-imidazol-4-yl)-5-methoxybiphenyl-3-yl methanesulfonate;
3′-(2-Amino-1-methyl-5-oxo-4-pyridin-2-yl-4,5-dihydro-1H-imidazol-4-yl)-5-methoxybiphenyl-3-yl methanesulfonate;
3′-[2-Amino-4-(3-furyl)-1-methyl-5-oxo-4,5-dihydro-1H-imidazol-4-yl]-5-methoxybiphenyl-3-yl methanesulfonate;
3′-[2-Amino-1-methyl-5-oxo-4-(1,3-thiazol-5-yl)-4,5-dihydro-1H-imidazol-4-yl]-5-methoxybiphenyl-3-yl methanesulfonate;
3′-[2-Amino-1-methyl-5-oxo-4-(1,3-thiazol-4-yl)-4,5-dihydro-1H-imidazol-4-yl]-5-methoxybiphenyl-3-yl methanesulfonate;
4-[2-Amino-4-(5′-fluoro-2′-methylbiphenyl-3-yl)-1-methyl-5-oxo-4,5-dihydro-1H-imidazol-4-yl]phenyl methanesulfonate;
4-[2-Amino-4-(3′-cyanobiphenyl-3-yl)-1-methyl-5-oxo-4,5-dihydro-1H-imidazol-4-yl]phenyl 2-methoxyethanesulfonate;
4-[2-Amino-4-(2′,5′-dimethoxybiphenyl-3-yl)-1-methyl-5-oxo-4,5-dihydro-1H-imidazol-4-yl]phenyl 2-methoxyethanesulfonate;
4-[2-Amino-4-(2′-fluoro-3′-methoxybiphenyl-3-yl)-1-methyl-5-oxo-4,5-dihydro-1H-imidazol-4-yl]phenyl 2-methoxyethanesulfonate;
4-[2-Amino-4-(2′-fluoro-5′-methoxybiphenyl-3-yl)-1-methyl-5-oxo-4,5-dihydro-1H-imidazol-4-yl]phenyl 2-methoxyethanesulfonate;
4-[2-Amino-4-(3′-cyano-4′-fluorobiphenyl-3-yl)-1-methyl-5-oxo-4,5-dihydro-1H-imidazol-4-yl]phenyl 2-methoxyethanesulfonate;
4-[2-Amino-4-(5′-cyano-2′-fluorobiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyridin-3-ylphenyl)-4,5-dihydro-1H-imidazol-4-yl]phenyl 2-methoxyethanesulfonate;
4-{2-Amino-4-[3-(2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(6-fluoropyridin-3-yl)phenyl]-1-methyl-5-oxo-4,5-dihydro-1H-imidazol-4-yl}phenyl 2-methoxyethanesulfonate;
4-[2-Amino-1-methyl-5-oxo-4-(3-pyrimidin-5-ylphenyl)-4,5-dihydro-1H-imidazol-4-yl]phenyl 2-methoxyethanesulfonate;
4-{2-Amino-4-[3-(2-chloro-3-fluoropyridin-4-yl)phenyl]-1-methyl-5-oxo-4,5-dihydro-1H-imidazol-4-yl}phenyl 2-methoxyethanesulfonate;
4-{2-Amino-4-[3-(2-chloro-5-fluoropyridin-3-yl)phenyl]-1-methyl-5-oxo-4,5-dihydro-1H-imidazol-4-yl}phenyl 2-methoxyethanesulfonate;
4-{2-Amino-4-[3-(2,6-difluoropyridin-3-yl)phenyl]-1-methyl-5-oxo-4,5-dihydro-1H-imidazol-4-yl}phenyl 2-methoxyethanesulfonate;
4-{2-Amino-4-[3-(5-fluoropyridin-3-yl)phenyl]-1-methyl-5-oxo-4,5-dihydro-1H-imidazol-4-yl}phenyl 2-methoxyethanesulfonate;
4-[2-Amino-4-(4′-fluoro-3′-methoxybiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyrazin-2-ylphenyl)-4,5-dihydro-1H-imidazol-4-yl]phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl ethanesulfonate;
3-[2-Amino-1-methyl-5-oxo-4-(3-pyridin-3-ylphenyl)-4,5-dihydro-1H-imidazol-4-yl]phenyl 2-methoxyethanesulfonate;
3-{2-Amino-4-[3-(6-fluoropyridin-3-yl)phenyl]-1-methyl-5-oxo-4,5-dihydro-1H-imidazol-4-yl}phenyl 2-methoxyethanesulfonate;
3-{2-Amino-4-[3-(2-fluoropyridin-3-yl)phenyl]-1-methyl-5-oxo-4,5-dihydro-1H-imidazol-4-yl}phenyl 2-methoxyethanesulfonate;
3-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
3-[2-Amino-1-methyl-5-oxo-4-(3-pyrimidin-5-ylphenyl)-4,5-dihydro-1H-imidazol-4-yl]phenyl 2-methoxyethanesulfonate;
3-[2-Amino-1-methyl-5-oxo-4-(3-pyridin-3-ylphenyl)-4,5-dihydro-1H-imidazol-4-yl]phenyl cyclopropanesulfonate;
3-{2-Amino-4-[3-(6-fluoropyridin-3-yl)phenyl]-1-methyl-5-oxo-4,5-dihydro-1H-imidazol-4-yl}phenyl cyclopropanesulfonate;
3-{2-Amino-4-[3-(2-fluoropyridin-3-yl)phenyl]-1-methyl-5-oxo-4,5-dihydro-1H-imidazol-4-yl}phenyl cyclopropanesulfonate;
3-{2-Amino-4-[3-(5-chloro-2-fluoropyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-5-ylphenyl)-4,5-dihydro-1H-imidazol-4-yl]phenyl cyclopropanesulfonate;
3-{2-Amino-4-[3-(5-methoxypyridin-3-yl)phenyl]-1-methyl-5-oxo-4,5-dihydro-1H-imidazol-4-yl}phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-2-ylphenyl)-4,5-dihydro-1H-imidazol-4-yl]phenyl cyclopropanesulfonate;
3-[2-Amino-4-(4-methoxyphenyl)-1-methyl-5-oxo-4,5-dihydro-1H-imidazol-4-yl]phenyl 3-methoxypropane-1-sulfonate;
3-[2-Amino-4-(4-methoxyphenyl)-1-methyl-5-oxo-4,5-dihydro-1H-imidazol-4-yl]phenyl methanesulfonate;
3-[2-Amino-4-(4-methoxyphenyl)-1-methyl-5-oxo-4,5-dihydro-1H-imidazol-4-yl]phenyl 2-ethoxyethanesulfonate;
3-[2-Amino-4-(4-methoxyphenyl)-1-methyl-5-oxo-4,5-dihydro-1H-imidazol-4-yl]phenyl 2-methoxyethanesulfonate;
4-[2-Amino-4-(3′-methoxybiphenyl-3-yl)-1-methyl-5-oxo-4,5-dihydro-1H-imidazol-4-yl]phenyl 2-methoxyethanesulfonate; and
4-{2-Amino-4-[3-(1H-indol-5-yl)phenyl]-1-methyl-5-oxo-4,5-dihydro-1H-imidazol-4-yl}phenyl methanesulfonate; as a free base or a pharmaceutically acceptable salt, solvate or solvate of a salt thereof.

In another aspect of the invention, there is provided a compound according to formula I,

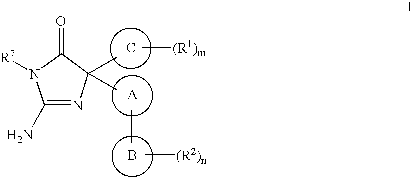

wherein
A is selected from phenyl and heteroaryl, wherein said phenyl or heteroaryl is optionally substituted with one or more R³;
B is selected from hydrogen, halo, cyano, phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl, and C₂₋₆alkenylC₃₋₆cycloalkyl, wherein said phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl or C₂₋₆alkenylC₃₋₆cycloalkyl, is optionally substituted with one or two R⁴;
C is selected from phenyl, heteroaryl and heterocyclyl, wherein said phenyl, heteroaryl or heterocyclyl is optionally substituted with one or two R⁵;

### R¹ and R² is OSO₂R⁶;

R³ is selected from halo and cyano;
R⁴ is selected from halo, C₁₋₆alkyl, methoxy, cyano and trifluoromethoxy;
R⁵ is selected from halo, C₁₋₆alkyl, methoxy, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂; said C₁₋₆alkyl, C₃₋₆cycloalkyl, aryl or heteroaryl is optionally substituted with halogen, CN, NH₂, OH, COOH, OC₁₋₆alkyl, C₁₋₆alkylOH, SO₂H, C₁₋₆alkyl, OC₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl or NC(O)(C₅₋₆heterocyclyl)₂;
R⁷ is C₁₋₆alkyl optionally substituted with halogen, CN, NH₂, OH, COOH, OC₁₋₆alkyl, SO₂H, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂;
m=0 or 1;
n=0 or 1;
wherein one of m or n is at least 1.

In another aspect of the invention, there is provided a compound according to formula I,

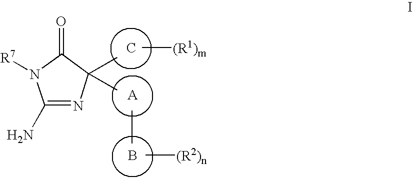

wherein
A is selected from phenyl and heteroaryl, wherein said phenyl or heteroaryl is optionally substituted with one or more R³;
B is selected from hydrogen, halo, cyano, phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl, and C₂₋₆alkenylC₃₋₆cycloalkyl, wherein said phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl or C₂₋₆alkenylC₃₋₆cycloalkyl, is optionally substituted with one or two R⁴;
C is selected from phenyl, heteroaryl and heterocyclyl, wherein said phenyl, heteroaryl or heterocyclyl is optionally substituted with one or two R⁵;

### R¹ and R² is OSO₂R⁶;

R³ is selected from halo and cyano;
R⁴ is selected from halo, C₁₋₆alkyl, methoxy, cyano and trifluoromethoxy;
R⁵ is selected from halo, C₁₋₆alkyl, methoxy, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂; said C₁₋₆alkyl, C₃₋₆cycloalkyl, aryl or heteroaryl is optionally substituted with halogen, CN, NH₂, OH, COOH, OC₁₋₆alkyl, C₁₋₆alkylOH, SO₂H, C₁₋₆alkyl, OC₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl or NC(O)(C₅₋₆heterocyclyl)₂;
R⁷ is C₁₋₆alkyl optionally substituted with halogen, CN, NH₂, OH, COOH, OC₁₋₆alkyl, SO₂H, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₁₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂;
m=0 or 1;
n=0 or 1;
wherein one of m or n is at least 1;
provided that said compound is not:
4-[2-Amino-4-(3′-methoxybiphenyl-3-yl)-1-methyl-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl trifluoromethanesulfonate;
3′-(2-Amino-1-methyl-5-oxo-4-phenyl-4,5-dihydro-1H-imidazol-4-yl)biphenyl-3-yl trifluoromethanesulfonate;
3′-(2-Amino-1-methyl-5-oxo-4-phenyl-4,5-dihydro-1H-imidazol-4-yl)-5-methoxybiphenyl-2-yl trifluoromethanesulfonate;
3-[2-Amino-4-(3′-methoxybiphenyl-3-yl)-1-methyl-5-oxo-4,5-dihydro-1H-imidazol-4-yl]phenyl trifluoromethanesulfonate;
3-[2-Amino-4-(3′-methoxybiphenyl-3-yl)-1-methyl-5-oxo-4,5-dihydro-1H-imidazol-4-yl]phenyl methanesulfonate;
4-[2-Amino-1-methyl-5-oxo-4-(3-pyridin-3-ylphenyl)-4,5-dihydro-1H-imidazol-4-yl]phenyl methanesulfonate;
4-[2-Amino-4-(5′-chloro-2′-methoxybiphenyl-3-yl)-1-methyl-5-oxo-4,5-dihydro-1H-imidazol-4-yl]phenyl methanesulfonate;
4-[2-Amino-4-(3′-cyanobiphenyl-3-yl)-1-methyl-5-oxo-4,5-dihydro-1H-imidazol-4-yl]phenyl methanesulfonate;
4-{2-Amino-1-methyl-5-oxo-4-[3′-(trifluoromethoxy)biphenyl-3-yl]-4,5-dihydro-1H-imidazol-4-yl}phenyl methanesulfonate;
4-{2-Amino-4-[3-(2-chloropyridin-4-yl)phenyl]-1-methyl-5-oxo-4,5-dihydro-1H-imidazol-4-yl}phenyl methanesulfonate;
4-[2-Amino-4-(3′-cyanobiphenyl-3-yl)-1-methyl-5-oxo-4,5-dihydro-1H-imidazol-4-yl]phenyl propane-1-sulfonate;
4-[2-Amino-4-(2′,5′-dimethoxybiphenyl-3-yl)-1-methyl-5-oxo-4,5-dihydro-1H-imidazol-4-yl]phenyl propane-1-sulfonate;
4-[2-Amino-4-(2′-fluoro-3′-methoxybiphenyl-3-yl)-1-methyl-5-oxo-4,5-dihydro-1H-imidazol-4-yl]phenyl propane-1-sulfonate;
4-[2-Amino-4-(2′-fluoro-5′-methoxybiphenyl-3-yl)-1-methyl-5-oxo-4,5-dihydro-1H-imidazol-4-yl]phenyl propane-1-sulfonate;
4-[2-Amino-4-(3′-cyano-4′-fluorobiphenyl-3-yl)-1-methyl-5-oxo-4,5-dihydro-1H-imidazol-4-yl]phenyl propane-1-sulfonate;
4-[2-Amino-4-(5′-cyano-2′-fluorobiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyridin-3-ylphenyl)-4,5-dihydro-1H-imidazol-4-yl]phenyl propane-1-sulfonate;
4-{2-Amino-4-[3-(2-fluoropyridin-3-yl)phenyl]-1-methyl-5-oxo-4,5-dihydro-1H-imidazol-4-yl}phenyl propane-1-sulfonate;
4-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
4-{2-Amino-4-[3-(6-fluoropyridin-3-yl)phenyl]-1-methyl-5-oxo-4,5-dihydro-1H-imidazol-4-yl}phenyl propane-1-sulfonate;
4-[2-Amino-1-methyl-5-oxo-4-(3-pyrimidin-5-ylphenyl)-4,5-dihydro-1H-imidazol-4-yl]phenyl propane-1-sulfonate;
4-{2-Amino-4-[3-(2-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-3-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-5-fluoropyridin-3-yl)phenyl]-1-methyl-5-oxo-4,5-dihydro-1H-imidazol-4-yl}phenyl propane-1-sulfonate;
4-{2-Amino-4-[3-(2,6-difluoropyridin-3-yl)phenyl]-1-methyl-5-oxo-4,5-dihydro-1H-imidazol-4-yl}phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl propane-1-sulfonate;
4-[2-Amino-4-(4′-fluoro-3′-methoxybiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyrazin-2-ylphenyl)-4,5-dihydro-1H-imidazol-4-yl]phenyl propane-1-sulfonate;
3′-(2-Amino-1-methyl-5-oxo-4-phenyl-4,5-dihydro-1H-imidazol-4-yl)-5-methoxybiphenyl-3-yl methanesulfonate;
4-{2-Amino-4-[3-(5-methoxypyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl trifluoromethanesulfonate;
3-[2-Amino-1-methyl-5-oxo-4-(3-pyrimidin-5-ylphenyl)-4,5-dihydro-1H-imidazol-4-yl]phenyl trifluoromethanesulfonate;
3-{2-Amino-4-[3-(5-methoxypyridin-3-yl)phenyl]-1-methyl-5-oxo-4,5-dihydro-1H-imidazol-4-yl}phenyl trifluoromethanesulfonate;
4-[2-Amino-4-(3′,5′-dichlorodiphenyl-3-yl)-1-methyl-5-oxo-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl methanesulfonate;
3-{2-Amino-4-[3-(6-fluoropyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl methanesulfonate;
3-{2-Amino-4-[3-(5-chloro-2-fluoropyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-5-ylphenyl)-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl propane-1-sulfonate;
3-{2-Amino-4-[3-(6-fluoropyridin-3-yl)phenyl]-1-methyl-5-oxo-4,5-dihydro-1H-imidazol-4-yl}phenyl propane-1-sulfonate;
3-{2-Amino-4-[3-(2-fluoropyridin-3-yl)phenyl]-1-methyl-5-oxo-4,5-dihydro-1H-imidazol-4-yl}phenyl propane-1-sulfonate;
3-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-5-ylphenyl)-4,5-dihydro-1H-imidazol-4-yl]phenyl propane-1-sulfonate;
3-[2-Amino-1-methyl-5-oxo-4-(3-pyridin-3-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl propane-2-sulfonate;
3-{2-Amino-4-[3-(2-fluoropyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-5-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl dimethylsulfamate;
3-{2-Amino-4-[3-(5-chloro-2-fluoropyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-5-ylphenyl)-4,5-dihydro-1H-imidazol-4-yl]phenyl dimethylsulfamate;
3-{2-Amino-4-[3-(5-methoxypyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-2-ylphenyl)-4,5-dihydro-1H-imidazol-4-yl]phenyl methanesulfonate;
3-{2-Amino-4-[3-(5-methoxypyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-2-ylphenyl)-4,5-dihydro-1H-imidazol-4-yl]phenyl propane-1-sulfonate;
3-{2-Amino-4-[3-(5-methoxypyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-2-ylphenyl)-4,5-dihydro-1H-imidazol-4-yl]phenyl propane-2-sulfonate;
3-{2-Amino-4-[3-(5-methoxypyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-2-ylphenyl)-4,5-dihydro-1H-imidazol-4-yl]phenyl dimethylsulfamate;
3-[2-Amino-4-(3′-methoxybiphenyl-3-yl)-1-methyl-5-oxo-4,5-dihydro-1H-imidazol-4-yl]phenyl propane-2-sulfonate;
3′-(2-Amino-1-methyl-5-oxo-4-phenyl-4,5-dihydro-1H-imidazol-4-yl)-5-methoxybiphenyl-3-yl trifluoromethanesulfonate;
4-{2-Amino-4-[3-(5-methoxypyridin-3-yl)phenyl]-1-methyl-5-oxo-4,5-dihydro-1H-imidazol-4-yl}phenyl propane-1-sulfonate;
4-[2-Amino-4-(3′-cyano-6-fluorobiphenyl-3-yl)-1-methyl-5-oxo-4,5-dihydro-1H-imidazol-4-yl]phenyl methanesulfonate;
4-[2-Amino-4-(3′-cyano-6-fluorobiphenyl-3-yl)-5-oxo-4,5-dihydro-1H-imidazol-4-yl]phenyl methanesulfonate;
4-[2-Amino-4-(2′,6-difluoro-3′-methoxybiphenyl-3-yl)-5-oxo-4,5-dihydro-1H-imidazol-4-yl]phenyl methanesulfonate;
4-[2-Amino-4-(2′,6-difluoro-5′-methoxybiphenyl-3-yl)-5-oxo-4,5-dihydro-1H-imidazol-4-yl]phenyl methanesulfonate;
4-[2-Amino-4-(3′-cyano-4′,6-difluorobiphenyl-3-yl)-5-oxo-4,5-dihydro-1H-imidazol-4-yl]phenyl methanesulfonate;
4-[2-Amino-4-(5′-cyano-2′,6-difluorobiphenyl-3-yl)-5-oxo-4,5-dihydro-1H-imidazol-4-yl]phenyl methanesulfonate;
4-[2-Amino-4-(4-fluoro-3-pyridin-3-ylphenyl)-5-oxo-4,5-dihydro-1H-imidazol-4-yl]phenyl methanesulfonate;
4-{2-Amino-4-[4-fluoro-3-(2-fluoropyridin-3-yl)phenyl]-5-oxo-4,5-dihydro-1H-imidazol-4-yl}phenyl methanesulfonate (racemate);
4-{2-Amino-4-[3-(5-chloro-2-fluoropyridin-3-yl)-4-fluorophenyl]-5-oxo-4,5-dihydro-1H-imidazol-4-yl}phenyl methanesulfonate;
4-{2-Amino-4-[4-fluoro-3-(6-fluoropyridin-3-yl)phenyl]-5-oxo-4,5-dihydro-1H-imidazol-4-yl}phenyl methanesulfonate;
4-{2-Amino-4-[4-fluoro-3-(2-fluoropyridin-4-yl)phenyl]-5-oxo-4,5-dihydro-1H-imidazol-4-yl}phenyl methanesulfonate;
4-{2-Amino-4-[3-(2-chloro-3-fluoropyridin-4-yl)-4-fluorophenyl]-5-oxo-4,5-dihydro-1H-imidazol-4-yl}phenyl methanesulfonate;
4-{2-Amino-4-[3-(2-chloro-5-fluoropyridin-3-yl)-4-fluorophenyl]-5-oxo-4,5-dihydro-1H-imidazol-4-yl}phenyl methanesulfonate;
4-{2-Amino-4-[3-(2,6-difluoropyridin-3-yl)-4-fluorophenyl]-5-oxo-4,5-dihydro-1H-imidazol-4-yl}phenyl methanesulfonate;
4-{2-Amino-4-[6-fluoro-3′-(trifluoromethoxy)biphenyl-3-yl]-5-oxo-4,5-dihydro-1H-imidazol-4-yl}phenyl methanesulfonate;
4-[2-Amino-4-(3′-chloro-6-fluorobiphenyl-3-yl)-5-oxo-4,5-dihydro-1H-imidazol-4-yl]phenyl methanesulfonate;
4-[2-Amino-4-(4-fluoro-3-pyridin-4-ylphenyl)-5-oxo-4,5-dihydro-1H-imidazol-4-yl]phenyl methanesulfonate;
4-[2-Amino-4-(4-fluoro-3-pyrimidin-5-ylphenyl)-5-oxo-4,5-dihydro-1H-imidazol-4-yl]phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-{2-Amino-4-[4-fluoro-3-(5-fluoropyridin-3-yl)phenyl]-5-oxo-4,5-dihydro-1H-imidazol-4-yl}phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl propane-1-sulfonate;
4-[2-Amino-4-(3′-methoxybiphenyl-3-yl)-1-methyl-5-oxo-4,5-dihydro-1H-imidazol-4-yl]phenyl propane-2-sulfonate;
4-[2-Amino-4-(3′-methoxybiphenyl-3-yl)-1-methyl-5-oxo-4,5-dihydro-1H-imidazol-4-yl]phenyl dimethylsulfamate;
4-(2-Amino-4-{3′-methoxy-5′-[(methylsulfonyl)oxy]biphenyl-3-yl}-1-methyl-5-oxo-4,5-dihydro-1H-imidazol-4-yl)phenyl propane-1-sulfonate;
3-[2-Amino-1-methyl-5-oxo-4-(3-pyridin-3-ylphenyl)-4,5-dihydro-1H-imidazol-4-yl]phenyl trifluoromethanesulfonate;
3-[2-Amino-1-methyl-5-oxo-4-(3-pyridin-3-ylphenyl)-4,5-dihydro-1H-imidazol-4-yl]phenyl dimethylsulfamate;
4-(2-Amino-4-{6-fluoro-3′-methoxy-5′-[(methylsulfonyl)oxy]biphenyl-3-yl}-5-oxo-4,5-dihydro-1H-imidazol-4-yl)phenyl methanesulfonate;
3-(2-Amino-1-methyl-5-oxo-4-phenyl-4,5-dihydro-1H-imidazol-4-yl)phenyl methanesulfonate;
3-(2-Amino-1-methyl-5-oxo-4-phenyl-4,5-dihydro-1H-imidazol-4-yl)phenyl trifluoromethanesulfonate;
3′-(2-Amino-1-methyl-5-oxo-4-pyridin-4-yl-4,5-dihydro-1H-imidazol-4-yl)-5-methoxybiphenyl-3-yl methanesulfonate;
3′-(2-Amino-1-methyl-5-oxo-4-pyridin-2-yl-4,5-dihydro-1H-imidazol-4-yl)-5-methoxybiphenyl-3-yl methanesulfonate;
3′-[2-Amino-4-(3-furyl)-1-methyl-5-oxo-4,5-dihydro-1H-imidazol-4-yl]-5-methoxybiphenyl-3-yl methanesulfonate;
3′-[2-Amino-1-methyl-5-oxo-4-(1,3-thiazol-5-yl)-4,5-dihydro-1H-imidazol-4-yl]-5-methoxybiphenyl-3-yl methanesulfonate;
3′-[2-Amino-1-methyl-5-oxo-4-(1,3-thiazol-4-yl)-4,5-dihydro-1H-imidazol-4-yl]-5-methoxybiphenyl-3-yl methanesulfonate;
4-[2-Amino-4-(5′-fluoro-2′-methylbiphenyl-3-yl)-1-methyl-5-oxo-4,5-dihydro-1H-imidazol-4-yl]phenyl methanesulfonate;
4-[2-Amino-4-(3′-cyanobiphenyl-3-yl)-1-methyl-5-oxo-4,5-dihydro-1H-imidazol-4-yl]phenyl 2-methoxyethanesulfonate;
4-[2-Amino-4-(2′,5′-dimethoxybiphenyl-3-yl)-1-methyl-5-oxo-4,5-dihydro-1H-imidazol-4-yl]phenyl 2-methoxyethanesulfonate;
4-[2-Amino-4-(2′-fluoro-3′-methoxybiphenyl-3-yl)-1-methyl-5-oxo-4,5-dihydro-1H-imidazol-4-yl]phenyl 2-methoxyethanesulfonate;
4-[2-Amino-4-(2′-fluoro-5′-methoxybiphenyl-3-yl)-1-methyl-5-oxo-4,5-dihydro-1H-imidazol-4-yl]phenyl 2-methoxyethanesulfonate;
4-[2-Amino-4-(3′-cyano-4′-fluorobiphenyl-3-yl)-1-methyl-5-oxo-4,5-dihydro-1H-imidazol-4-yl]phenyl 2-methoxyethanesulfonate;
4-[2-Amino-4-(5′-cyano-2′-fluorobiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyridin-3-ylphenyl)-4,5-dihydro-1H-imidazol-4-yl]phenyl 2-methoxyethanesulfonate;
4-{2-Amino-4-[3-(2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(6-fluoropyridin-3-yl)phenyl]-1-methyl-5-oxo-4,5-dihydro-1H-imidazol-4-yl}phenyl 2-methoxyethanesulfonate;
4-[2-Amino-1-methyl-5-oxo-4-(3-pyrimidin-5-ylphenyl)-4,5-dihydro-1H-imidazol-4-yl]phenyl 2-methoxyethanesulfonate;
4-{2-Amino-4-[3-(2-chloro-3-fluoropyridin-4-yl)phenyl]-1-methyl-5-oxo-4,5-dihydro-1H-imidazol-4-yl}phenyl 2-methoxyethanesulfonate;
4-{2-Amino-4-[3-(2-chloro-5-fluoropyridin-3-yl)phenyl]-1-methyl-5-oxo-4,5-dihydro-1H-imidazol-4-yl}phenyl 2-methoxyethanesulfonate;
4-{2-Amino-4-[3-(2,6-difluoropyridin-3-yl)phenyl]-1-methyl-5-oxo-4,5-dihydro-1H-imidazol-4-yl}phenyl 2-methoxyethanesulfonate;
4-{2-Amino-4-[3-(5-fluoropyridin-3-yl)phenyl]-1-methyl-5-oxo-4,5-dihydro-1H-imidazol-4-yl}phenyl 2-methoxyethanesulfonate;
4-[2-Amino-4-(4′-fluoro-3′-methoxybiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyrazin-2-ylphenyl)-4,5-dihydro-1H-imidazol-4-yl]phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl ethanesulfonate;
3-[2-Amino-1-methyl-5-oxo-4-(3-pyridin-3-ylphenyl)-4,5-dihydro-1H-imidazol-4-yl]phenyl 2-methoxyethanesulfonate;
3-{2-Amino-4-[3-(6-fluoropyridin-3-yl)phenyl]-1-methyl-5-oxo-4,5-dihydro-1H-imidazol-4-yl}phenyl 2-methoxyethanesulfonate;
3-{2-Amino-4-[3-(2-fluoropyridin-3-yl)phenyl]-1-methyl-5-oxo-4,5-dihydro-1H-imidazol-4-yl}phenyl 2-methoxyethanesulfonate;
3-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
3-[2-Amino-1-methyl-5-oxo-4-(3-pyrimidin-5-ylphenyl)-4,5-dihydro-1H-imidazol-4-yl]phenyl 2-methoxyethanesulfonate;
3-[2-Amino-1-methyl-5-oxo-4-(3-pyridin-3-ylphenyl)-4,5-dihydro-1H-imidazol-4-yl]phenyl cyclopropanesulfonate;
3-{2-Amino-4-[3-(6-fluoropyridin-3-yl)phenyl]-1-methyl-5-oxo-4,5-dihydro-1H-imidazol-4-yl}phenyl cyclopropanesulfonate;
3-{2-Amino-4-[3-(2-fluoropyridin-3-yl)phenyl]-1-methyl-5-oxo-4,5-dihydro-1H-imidazol-4-yl}phenyl cyclopropanesulfonate;
3-{2-Amino-4-[3-(5-chloro-2-fluoropyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-5-ylphenyl)-4,5-dihydro-1H-imidazol-4-yl]phenyl cyclopropanesulfonate;
3-{2-Amino-4-[3-(5-methoxypyridin-3-yl)phenyl]-1-methyl-5-oxo-4,5-dihydro-1H-imidazol-4-yl}phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-2-ylphenyl)-4,5-dihydro-1H-imidazol-4-yl]phenyl cyclopropanesulfonate;
3-[2-Amino-4-(4-methoxyphenyl)-1-methyl-5-oxo-4,5-dihydro-1H-imidazol-4-yl]phenyl 3-methoxypropane-1-sulfonate;
3-[2-Amino-4-(4-methoxyphenyl)-1-methyl-5-oxo-4,5-dihydro-1H-imidazol-4-yl]phenyl methanesulfonate;
3-[2-Amino-4-(4-methoxyphenyl)-1-methyl-5-oxo-4,5-dihydro-1H-imidazol-4-yl]phenyl 2-ethoxyethanesulfonate;
3-[2-Amino-4-(4-methoxyphenyl)-1-methyl-5-oxo-4,5-dihydro-1H-imidazol-4-yl]phenyl 2-methoxyethanesulfonate;
4-[2-Amino-4-(3′-methoxybiphenyl-3-yl)-1-methyl-5-oxo-4,5-dihydro-1H-imidazol-4-yl]phenyl 2-methoxyethanesulfonate; and
4-{2-Amino-4-[3-(1H-indol-5-yl)phenyl]-1-methyl-5-oxo-4,5-dihydro-1H-imidazol-4-yl}phenyl methanesulfonate; as a free base or a pharmaceutically acceptable salt, solvate or solvate of a salt thereof.

In another aspect of the invention, there is provided a compound according to formula I, wherein:

A is selected from phenyl or pyridine, wherein said phenyl or pyridine is optionally substituted with one or more R³;
B is selected from phenyl, pyridine, pyrimidine, pyrazine, CH₂CH₂cyclopropyl and CHCHcyclopropyl, wherein said phenyl, pyridine, pyrimidine or pyrazine is optionally substituted with one or two R⁴;
C is phenyl optionally substituted with one or two R⁵;

### R¹ and R² is OSO₂R⁶;

R³ is fluoro;
R⁴ is selected from fluoro, chloro, methoxy, cyano and trifluoromethoxy;
R⁵ is selected from methyl, methoxy, difluoromethoxy and trifluoromethoxy or; two R⁵ may together form a 2,3-dihydro-1,4-benzodioxine;
R⁶ is selected from methyl, trifluoromethyl, propyl, isopropyl, phenyl and N(CH₃)₂;
R⁷ is selected from methyl, ethyl or propyl;
m=0 or 1;
n=0 or 1;
wherein one of m or n is at least 1.

In another aspect of the invention, there is provided a compound according to formula I, wherein m is 1, n is 0, and R⁶ is selected from methyl, trifluoromethyl, propyl, isopropyl, phenyl and N(CH₃)₂.

In another aspect of the invention, there is provided a compound according to formula I, wherein m is 0 and n is 1, and R⁶ is methyl.

In another aspect of the invention, there is provided a compound according to formula I, wherein A is phenyl.

In another aspect of the invention, there is provided a compound according to formula I, wherein A is phenyl substituted with one R³ and R³ is fluoro.

In another aspect of the invention, there is provided a compound according to formula I, wherein A is pyridine.

In another aspect of the invention, there is provided a compound according to formula I, wherein R⁷ is methyl.

In another aspect of the invention, there is provided a compound according to formula I, wherein R⁷ is ethyl.

In another aspect of the invention, there is provided a compound according to formula I, wherein R⁷ is propyl.

In another aspect of the invention, there is provided a compound according to formula I, wherein C is phenyl.

In another aspect of the invention, there is provided a compound according to formula I, wherein C is phenyl substituted with one R⁵, said R⁵ being selected from methyl, methoxy, difluoromethoxy or trifluoromethoxy.

In another aspect of the invention, there is provided a compound according to formula I, wherein C is phenyl substituted with two R⁵, said two R⁵ being independently selected from methyl, methoxy and trifluoromethoxy or; two R⁵ together form a 2,3-dihydro-1,4-benzodioxine.

In another aspect of the invention, there is provided a compound according to formula I, wherein B is phenyl.

In another aspect of the invention, there is provided a compound according to formula I, wherein B is phenyl substituted with one R⁴, said R⁴ being selected from fluoro, chloro, methoxy and cyano.

In another aspect of the invention, there is provided a compound according to formula I, wherein B is phenyl substituted with two R⁴, said two R⁴ being independently selected from fluoro, chloro, methoxy, cyano and trifluoromethoxy.

In another aspect of the invention, there is provided a compound according to formula I, wherein B is pyridine.

In another aspect of the invention, there is provided a compound according to formula I, wherein B is pyridine substituted with one R⁴, said R⁴ being selected from fluoro, chloro, methoxy and cyano.

In another aspect of the invention, there is provided a compound according to formula I, wherein B is pyridine substituted with two R⁴, said two R⁴ being independently selected from fluoro and chloro.

In another aspect of the invention, there is provided a compound according to formula I, wherein B is pyrimidine.

In another aspect of the invention, there is provided a compound according to formula I, wherein B is pyrazine.

In another aspect of the invention, there is provided a compound according to formula I, wherein B is CH₂CH₂cyclopropyl.

In another aspect of the invention, there is provided a compound according to formula I, wherein B is CHCHcyclopropyl.

In another aspect of the invention, there is provided a compound according to formula I, wherein R⁴ is acetyl.

In another aspect of the invention, there is provided a compound according to formula I, wherein R⁶ is selected from C₁₋₆alkyl, C₃₋₆cycloalkyl, trifluoromethyl, aryl, heteroaryl and N(CH₃)₂; said aryl being optionally fused with a 5 or 6 membered cyclyl or heterocyclyl group to form a bicyclic ring system; and said aryl, heteroaryl, or bicyclic ring system is optionally substituted with one or more halogen, OC₁₋₆alkyl, C₁₋₆alkyl or heteroaryl.

In another aspect of the invention, there is provided a compound according to formula I, wherein:

A is selected from phenyl and heteroaryl, wherein said phenyl or heteroaryl is optionally substituted with one or more R³;
B is selected from hydrogen, halo, phenyl, heteroaryl, C₃₋₆cycloalkyl, C₃₋₆cycloalkenyl, C₀₋₆alkylC₃₋₆cycloalkyl, and C₂₋₆alkenylC₃₋₆cycloalkyl, wherein said phenyl is optionally substituted with one or two R⁴;
C is selected from phenyl, heteroaryl and heterocyclyl, wherein said phenyl or heteroaryl is optionally substituted with one or two R⁵;

### R¹ and R² is OSO₂R⁶;

R³ is halo;
R⁴ is selected from halo, methoxy, cyano, acetyl and trifluoromethoxy;
R⁵ is selected from halo, C₁₋₆alkyl, methoxy, trifluoromethyl, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂; said aryl being optionally fused with a 5 or 6 membered cyclyl or heterocyclyl group to form a bicyclic ring system; and said aryl, heteroaryl, or bicyclic ring system is optionally substituted with one or more halogen, OC₁₋₆alkyl, C₁₋₆alkyl or heteroaryl;
R⁷ is C₁₋₆alkyl;
m=0 or 1;
n=0 or 1;
wherein one of m or n is at least 1.

In another aspect of the invention, there is provided a compound according to formula I, wherein C is heteroaryl.

In another aspect of the invention, there is provided a compound according to formula I, wherein C is selected from thiazolyl, pyrimidinyl and pyridinyl.

In another aspect of the invention, there is provided a compound according to formula I, wherein C is heterocyclyl.

In another aspect of the invention, there is provided a compound according to formula I, wherein said C is cyclopentyl.

In another aspect of the invention, there is provided a compound according to formula I, selected from:
4-{2-Amino-4-[4-fluoro-3-(4-methoxypyrimidin-2-yl)phenyl]-1-methyl-5-oxo-4,5-dihydro-1H-imidazol-4-yl}phenyl methanesulfonate 0.25 acetate;
3-{2-Amino-4-[4-fluoro-3-(4-methoxypyrimidin-2-yl)phenyl]-1-methyl-5-oxo-4,5-dihydro-1H-imidazol-4-yl}phenyl methanesulfonate 0.5 acetate;
5-[2-Amino-4-(4-fluoro-3-pyrimidin-5-ylphenyl)-1-methyl-5-oxo-4,5-dihydro-1H-imidazol-4-yl]-2-methoxyphenyl trifluoromethanesulfonate 0.5 acetate;
4-{2-Amino-4-[5-(2-fluoro-5-methoxyphenyl)pyridin-3-yl]-1-methyl-5-oxo-4,5-dihydro-1H-imidazol-4-yl}phenyl methanesulfonate 0.5 acetate;
4-[2-amino-4-(2′-fluoro-3,3′-bipyridin-5-yl)-1-methyl-5-oxo-4,5-dihydro-1H-imidazol-4-yl]phenyl methanesulfonate 0.5 acetate;
4-[2-Amino-4-(2′-fluoro-2,3′-bipyridin-4-yl)-1-methyl-5-oxo-4,5-dihydro-1H-imidazol-4-yl]phenyl methanesulfonate 0.25 acetate;
4-{2-Amino-4-[2-(3-cyanophenyl)pyridin-4-yl]-1-methyl-5-oxo-4,5-dihydro-1H-imidazol-4-yl}phenyl methanesulfonate 0.25 acetate;
4-{2-Amino-4-[2-(3-methoxyphenyl)pyridin-4-yl]-1-methyl-5-oxo-4,5-dihydro-1H-imidazol-4-yl}phenyl methanesulfonate 0.25 acetate;
3′-[2-Amino-4-(4-methoxyphenyl)-1-methyl-5-oxo-4,5-dihydro-1H-imidazol-4-yl]-5-methoxybiphenyl-3-yl methanesulfonate;
3′-{2-Amino-1-methyl-5-oxo-4-[4-(trifluoromethoxy)phenyl]-4,5-dihydro-1H-imidazol-4-yl}-5-methoxybiphenyl-3-yl methanesulfonate;
3′-{2-Amino-1-methyl-5-oxo-4-[4-(trifluoromethoxy)phenyl]-4,5-dihydro-1H-imidazol-4-yl}-5-chlorobiphenyl-3-yl methanesulfonate;
3′-{2-Amino-4-[4-methoxy-3-(trifluoromethyl)phenyl]-1-methyl-5-oxo-4,5-dihydro-1H-imidazol-4-yl}-5-methoxybiphenyl-3-yl methanesulfonate;
3′-{2-Amino-4-[4-methoxy-3-(trifluoromethyl)phenyl]-1-methyl-5-oxo-4,5-dihydro-1H-imidazol-4-yl}-5-chlorobiphenyl-3-yl methanesulfonate;
3′-[2-Amino-4-(4-methoxy-3-methylphenyl)-1-methyl-5-oxo-4,5-dihydro-1H-imidazol-4-yl]-5-methoxybiphenyl-3-yl methanesulfonate 0.25 acetate;
3′-{2-Amino-4-[4-(difluoromethoxy)phenyl]-1-methyl-5-oxo-4,5-dihydro-1H-imidazol-4-yl}-5-methoxybiphenyl-3-yl methanesulfonate 0.25 acetate;
3′-{2-Amino-4-[4-(difluoromethoxy)phenyl]-1-methyl-5-oxo-4,5-dihydro-1H-imidazol-4-yl}-5-chlorobiphenyl-3-yl methanesulfonate;
5′-{2-Amino-4-[4-(difluoromethoxy)phenyl]-1-methyl-5-oxo-4,5-dihydro-1H-imidazol-4-yl}-2′-fluoro-5-methoxybiphenyl-3-yl methanesulfonate hydrochloride;
5′-{2-Amino-4-[4-(difluoromethoxy)phenyl]-1-methyl-5-oxo-4,5-dihydro-1H-imidazol-4-yl}-5-cyano-2′-fluorobiphenyl-3-yl methanesulfonate hydrochloride;
4-[2-Amino-4-(3′-cyano-6-fluoro-5′-methoxybiphenyl-3-yl)-1-methyl-5-oxo-4,5-dihydro-1H-imidazol-4-yl]phenyl methanesulfonate hydrochloride;
5-(5-{2-Amino-4-[4-(difluoromethoxy)phenyl]-1-methyl-5-oxo-4,5-dihydro-1H-imidazol-4-yl}-2-fluorophenyl)pyridin-3-yl methanesulfonate hydrochloride;
4-[2-Amino-4-(3-fluoro-4-pyrimidin-5-ylphenyl)-1-methyl-5-oxo-4,5-dihydro-1H-imidazol-4-yl]phenyl trifluoromethanesulfonate hydrochloride;
4-(2-Amino-4-{3-[(E)-2-cyclopropylvinyl]-4-fluorophenyl}-1-methyl-5-oxo-4,5-dihydro-1H-imidazol-4-yl)phenyl methanesulfonate;
4-{2-Amino-4-[3-(2-cyclopropylethyl)-4-fluorophenyl]-1-methyl-5-oxo-4,5-dihydro-1H-imidazol-4-yl}phenyl methanesulfonate;
5′-(2-Amino-1-methyl-5-oxo-4-phenyl-4,5-dihydro-1H-imidazol-4-yl)-2′-fluoro-5-methoxybiphenyl-3-yl methanesulfonate;
3-{2-Amino-4-[3-(5-chloro-2-fluoropyridin-3-yl)-4-fluorophenyl]-1-methyl-5-oxo-4,5-dihydro-1H-imidazol-4-yl}phenyl methanesulfonate;
3-{2-Amino-4-[4-fluoro-3-(5-fluoropyridin-3-yl)phenyl]-1-methyl-5-oxo-4,5-dihydro-1H-imidazol-4-yl}phenyl methanesulfonate;
3-{2-Amino-4-[4-fluoro-3-(2-fluoropyridin-3-yl)phenyl]-1-methyl-5-oxo-4,5-dihydro-1H-imidazol-4-yl}phenyl methanesulfonate;
3-[2-Amino-4-(3′,5′-dichloro-6-fluorobiphenyl-3-yl)-1-methyl-5-oxo-4,5-dihydro-1H-imidazol-4-yl]phenyl methanesulfonate;
3-[2-Amino-4-(4-fluoro-3-pyridin-3-ylphenyl)-1-methyl-5-oxo-4,5-dihydro-1H-imidazol-4-yl]phenyl methanesulfonate,
3-[2-Amino-4-(3′-chloro-6-fluorobiphenyl-3-yl)-1-methyl-5-oxo-4,5-dihydro-1H-imidazol-4-yl]phenyl methanesulfonate;
3-[2-Amino-4-(3′-chloro-6-fluoro-5′-methoxybiphenyl-3-yl)-1-methyl-5-oxo-4,5-dihydro-1H-imidazol-4-yl]phenyl methanesulfonate;
3-[2-Amino-4-(6-fluoro-3′-methoxybiphenyl-3-yl)-1-methyl-5-oxo-4,5-dihydro-1H-imidazol-4-yl]phenyl methanesulfonate;
3-[2-Amino-4-(4-fluoro-3-pyrimidin-5-ylphenyl)-1-methyl-5-oxo-4,5-dihydro-1H-imidazol-4-yl]phenyl methanesulfonate;
3-{2-Amino-4-[3-(5-chloro-2-fluoropyridin-3-yl)-4-fluorophenyl]-1-methyl-5-oxo-4,5-dihydro-1H-imidazol-4-yl}phenyl propane-1-sulfonate;
3-{2-Amino-4-[4-fluoro-3-(2-fluoropyridin-3-yl)phenyl]-1-methyl-5-oxo-4,5-dihydro-1H-imidazol-4-yl}phenyl propane-1-sulfonate;
3-[2-Amino-4-(4-fluoro-3-pyridin-3-ylphenyl)-1-methyl-5-oxo-4,5-dihydro-1H-imidazol-4-yl]phenyl propane-1-sulfonate;
3-[2-Amino-4-(3′-chloro-6-fluorobiphenyl-3-yl)-1-methyl-5-oxo-4,5-dihydro-1H-imidazol-4-yl]phenyl propane-1-sulfonate;
3-[2-Amino-4-(3′-chloro-6-fluoro-5′-methoxybiphenyl-3-yl)-1-methyl-5-oxo-4,5-dihydro-1H-imidazol-4-yl]phenyl propane-1-sulfonate;
3-[2-Amino-4-(3′,5′-dichloro-6-fluorobiphenyl-3-yl)-1-methyl-5-oxo-4,5-dihydro-1H-imidazol-4-yl]phenyl propane-1-sulfonate;
3-[2-Amino-4-(6-fluoro-3′-methoxybiphenyl-3-yl)-1-methyl-5-oxo-4,5-dihydro-1H-imidazol-4-yl]phenyl propane-1-sulfonate;
3-[2-Amino-4-(4-fluoro-3-pyrimidin-5-ylphenyl)-1-methyl-5-oxo-4,5-dihydro-1H-imidazol-4-yl]phenyl propane-1-sulfonate;
3-{2-Amino-4-[3-(5-chloro-2-fluoropyridin-3-yl)-4-fluorophenyl]-1-methyl-5-oxo-4,5-dihydro-1H-imidazol-4-yl}phenyl propane-2-sulfonate;
3-{2-Amino-4-[4-fluoro-3-(2-fluoropyridin-3-yl)phenyl]-1-methyl-5-oxo-4,5-dihydro-1H-imidazol-4-yl}phenyl propane-2-sulfonate;
3-[2-Amino-4-(4-fluoro-3-pyridin-3-ylphenyl)-1-methyl-5-oxo-4,5-dihydro-1H-imidazol-4-yl]phenyl propane-2-sulfonate;
3-[2-Amino-4-(3′-chloro-6-fluorobiphenyl-3-yl)-1-methyl-5-oxo-4,5-dihydro-1H-imidazol-4-yl]phenyl propane-2-sulfonate;
3-[2-Amino-4-(3′-chloro-6-fluoro-5′-methoxybiphenyl-3-yl)-1-methyl-5-oxo-4,5-dihydro-1H-imidazol-4-yl]phenyl propane-2-sulfonate;
3-[2-Amino-4-(6-fluoro-3′-methoxybiphenyl-3-yl)-1-methyl-5-oxo-4,5-dihydro-1H-imidazol-4-yl]phenyl propane-2-sulfonate;
3-[2-Amino-4-(3′,5′-dichloro-6-fluorobiphenyl-3-yl)-1-methyl-5-oxo-4,5-dihydro-1H-imidazol-4-yl]phenyl propane-2-sulfonate;
3-{2-Amino-4-[4-fluoro-3-(2-fluoropyridin-3-yl)phenyl]-1-methyl-5-oxo-4,5-dihydro-1H-imidazol-4-yl}phenyl dimethylsulfamate;
3-[2-Amino-4-(4-fluoro-3-pyridin-3-ylphenyl)-1-methyl-5-oxo-4,5-dihydro-1H-imidazol-4-yl]phenyl dimethylsulfamate;
3-[2-Amino-4-(6-fluoro-3′-methoxybiphenyl-3-yl)-1-methyl-5-oxo-4,5-dihydro-1H-imidazol-4-yl]phenyl dimethylsulfamate;
3-[2-Amino-4-(3′,5′-dichloro-6-fluorobiphenyl-3-yl)-1-methyl-5-oxo-4,5-dihydro-1H-imidazol-4-yl]phenyl dimethylsulfamate;
4-{2-Amino-4-[4-fluoro-3-(5-methoxypyridin-3-yl)phenyl]-1-methyl-5-oxo-4,5-dihydro-1H-imidazol-4-yl}phenyl propane-1-sulfonate hydrochloride;
3-{2-Amino-4-[4-fluoro-3-(5-methoxypyridin-3-yl)phenyl]-1-methyl-5-oxo-4,5-dihydro-1H-imidazol-4-yl}phenyl propane-1-sulfonate hydrochloride;
3-[2-Amino-4-(4-fluoro-3-pyrimidin-5-ylphenyl)-1-methyl-5-oxo-4,5-dihydro-1H-imidazol-4-yl]phenyl benzenesulfonate hydrochloride;
(S)-4-{2-Amino-4-[4-fluoro-3-(2-fluoropyridin-3-yl)phenyl]-5-oxo-4,5-dihydro-1H-imidazol-4-yl}phenyl methanesulfonate;
4-{2-Amino-4-[3-(5-chloropyridin-3-yl)-4-fluorophenyl]-1-methyl-5-oxo-4,5-dihydro-1H-imidazol-4-yl}phenyl trifluoromethanesulfonate 0.75 acetate;
4-[2-Amino-4-(4-fluoro-3-pyridin-3-ylphenyl)-1-methyl-5-oxo-4,5-dihydro-1H-imidazol-4-yl]phenyl trifluoromethanesulfonate 0.5 acetate;
4-{2-Amino-4-[4-fluoro-3-(2-fluoropyridin-3-yl)phenyl]-1-methyl-5-oxo-4,5-dihydro-1H-imidazol-4-yl}phenyl trifluoromethanesulfonate 0.75 acetate;
4-[2-Amino-4-(4-fluoro-3-pyrazin-2-ylphenyl)-1-methyl-5-oxo-4,5-dihydro-1H-imidazol-4-yl]phenyl trifluoromethanesulfonate 0.5 acetate;
4-{2-Amino-4-[3-(5-cyanopyridin-3-yl)-4-fluorophenyl]-1-methyl-5-oxo-4,5-dihydro-1H-imidazol-4-yl}phenyl trifluoromethanesulfonate 0.5 acetate;
4-{2-amino-4-[4-fluoro-3-(5-fluoropyridin-3-yl)phenyl]-1-methyl-5-oxo-4,5-dihydro-1H-imidazol-4-yl}phenyl trifluoromethanesulfonate 0.5 acetate;
4-{2-Amino-4-[4-fluoro-3-(5-methoxypyridin-3-yl)phenyl]-1-methyl-5-oxo-4,5-dihydro-1H-imidazol-4-yl}phenyl trifluoromethanesulfonate 0.5 acetate;
4-{2-Amino-4-[3-(4-chloropyridin-2-yl)-4-fluorophenyl]-1-methyl-5-oxo-4,5-dihydro-1H-imidazol-4-yl}phenyl trifluoromethanesulfonate 0.75 acetate;
4-[2-Amino-4-(3′,5′-dichloro-6-fluorobiphenyl-3-yl)-1-ethyl-5-oxo-4,5-dihydro-1H-imidazol-4-yl]phenyl methanesulfonate;
4-[2-Amino-4-(4-fluoro-3-pyrimidin-5-ylphenyl)-1-ethyl-5-oxo-4,5-dihydro-1H-imidazol-4-yl]phenyl methanesulfonate;
4-[2-Amino-4-(6-fluoro-3′-methoxybiphenyl-3-yl)-1-ethyl-5-oxo-4,5-dihydro-1H-imidazol-4-yl]phenyl methanesulfonate 0.25 acetate;
4-[2-Amino-4-(3′-chloro-6-fluorobiphenyl-3-yl)-1-ethyl-5-oxo-4,5-dihydro-1H-imidazol-4-yl]phenyl methanesulfonate;
4-[2-Amino-4-(4-fluoro-3-pyrimidin-5-ylphenyl)-1-propyl-5-oxo-4,5-dihydro-1H-imidazol-4-yl]phenyl methanesulfonate 0.5 acetate;
4-[2-Amino-4-(2′-cyano-6-fluoro-5′-methoxybiphenyl-3-yl)-1-methyl-5-oxo-4,5-dihydro-1H-imidazol-4-yl]phenyl methanesulfonate;
4-{2-Amino-4-[6-fluoro-5′-methoxy-2′-(trifluoromethoxy)biphenyl-3-yl]-1-methyl-5-oxo-4,5-dihydro-1H-imidazol-4-yl}phenyl methanesulfonate;
4-{2-amino-4-[3-(4-chloropyridin-3-yl)-4-fluorophenyl]-1-methyl-5-oxo-4,5-dihydro-1H-imidazol-4-yl}phenyl methanesulfonate hydrochloride;
3-{2-Amino-4-[3-(4-chloropyridin-3-yl)phenyl]-1-methyl-5-oxo-4,5-dihydro-1H-imidazol-4-yl}phenyl methanesulfonate hydrochloride;
3-[2-Amino-4-(2′-cyano-5′-methoxybiphenyl-3-yl)-1-methyl-5-oxo-4,5-dihydro-1H-imidazol-4-yl]phenyl methanesulfonate hydrochloride;
3-[2-Amino-4-(2′-cyano-6-fluoro-5′-methoxybiphenyl-3-yl)-1-methyl-5-oxo-4,5-dihydro-1H-imidazol-4-yl]phenyl methanesulfonate hydrochloride;
3′-[2-Amino-4-(2,3-dihydro-1,4-benzodioxin-6-yl)-1-methyl-5-oxo-4,5-dihydro-1H-imidazol-4-yl]-5-methoxybiphenyl-3-yl methanesulfonate;
4-[2-Amino-4-(4-fluoro-3-pyridin-3-ylphenyl)-1-methyl-5-oxo-4,5-dihydro-1H-imidazol-4-yl]phenyl 1-methyl-1H-imidazole-4-sulfonate;
4-{2-amino-4-[4-(3-methoxyphenyl)pyridin-2-yl]-1-methyl-5-oxo-4,5-dihydro-1H-imidazol-4-yl}phenyl methanesulfonate 0.25 acetate;
4-{2-amino-4-[4-(3-methoxyphenyl)pyridin-2-yl]-1-methyl-5-oxo-4,5-dihydro-1H-imidazol-4-yl}phenyl trifluoromethanesulfonate 0.5 acetate;
4-[2-amino-4-(3-cyclohex-1-en-1-yl-4-fluorophenyl)-1-methyl-5-oxo-4,5-dihydro-1H-imidazol-4-yl]phenyl methanesulfonate 0.5 acetate;
4-[2-amino-4-(3-cyclohexyl-4-fluorophenyl)-1-methyl-5-oxo-4,5-dihydro-1H-imidazol-4-yl]phenyl methanesulfonate 0.5 acetate; and
4-{2-amino-4-[4-fluoro-3-(4-methoxypyridin-2-yl)phenyl]-1-methyl-5-oxo-4,5-dihydro-1H-imidazol-4-yl}phenyl methanesulfonate 0.75 acetate; as a free base, alternative salt or a pharmaceutically acceptable salt, solvate or solvate of a salt thereof.

In another aspect of the invention, there is provided a compound according to formula I, selected from:
4-[2-Amino-4-(4-fluoro-3-pyrimidin-5-ylphenyl)-1-methyl-5-oxo-4,5-dihydro-1H-imidazol-4-yl]phenyl benzenesulfonate hydrochloride;
4-{2-Amino-4-[4-fluoro-3-(1,3-thiazol-5-yl)phenyl]-1-methyl-5-oxo-4,5-dihydro-1H-imidazol-4-yl}phenyl trifluoromethanesulfonate hydrochloride;
4-{2-Amino-4-[4-fluoro-3-(1,3-thiazol-5-yl)phenyl]-1-methyl-5-oxo-4,5-dihydro-1H-imidazol-4-yl}phenyl methanesulfonate hydrochloride;
5′-[2-Amino-1-methyl-5-oxo-4-(tetrahydro-2H-pyran-4-yl)-4,5-dihydro-1H-imidazol-4-yl]-2′-fluoro-5-methoxybiphenyl-3-yl methanesulfonate hydrochloride;
5′-[2-Amino-1-methyl-5-oxo-4-(tetrahydro-2H-pyran-4-yl)-4,5-dihydro-1H-imidazol-4-yl]-5-chloro-2′-fluorobiphenyl-3-yl methanesulfonate hydrochloride;
3-[2-Amino-4-(3-cyclopent-1-en-1-yl-4-fluorophenyl)-1-methyl-5-oxo-4,5-dihydro-1H-imidazol-4-yl]phenyl methanesulfonate hydrochloride;
3-[2-Amino-4-(3-cyclopent-1-en-1-yl-4-fluorophenyl)-1-methyl-5-oxo-4,5-dihydro-1H-imidazol-4-yl]phenyl methanesulfonate hydrochloride;
3-[2-Amino-4-(3-cyclopent-1-en-1-ylphenyl)-1-methyl-5-oxo-4,5-dihydro-1H-imidazol-4-yl]phenyl trifluoromethanesulfonate hydrochloride;
5′-[2-Amino-4-(2,6-diethylpyridin-4-yl)-1-methyl-5-oxo-4,5-dihydro-1H-imidazol-4-yl]-2′-fluoro-5-methoxybiphenyl-3-yl methanesulfonate hydrochloride;
5′-[2-Amino-4-(2,6-dimethylpyridin-4-yl)-1-methyl-5-oxo-4,5-dihydro-1H-imidazol-4-yl]-5-chloro-2′-fluorobiphenyl-3-yl methanesulfonate hydrochloride;
5′-[2-Amino-1-methyl-4-(2-methylpyridin-4-yl)-5-oxo-4,5-dihydro-1H-imidazol-4-yl]-2′-fluoro-5-methoxybiphenyl-3-yl methanesulfonate hydrochloride;
3-[2-Amino-4-(3-bromo-4-fluorophenyl)-1-methyl-5-oxo-4,5-dihydro-1H-imidazol-4-yl]phenyl 2,6-difluorobenzenesulfonate;
3-[2-Amino-4-(3-bromo-4-fluorophenyl)-1-methyl-5-oxo-4,5-dihydro-1H-imidazol-4-yl]phenyl pyridine-2-sulfonate;
3-[2-Amino-4-(3-bromo-4-fluorophenyl)-1-methyl-5-oxo-4,5-dihydro-1H-imidazol-4-yl]phenyl 4-(1H-pyrazol-1-yl)benzenesulfonate;
3-[2-Amino-4-(3-bromo-4-fluorophenyl)-1-methyl-5-oxo-4,5-dihydro-1H-imidazol-4-yl]phenyl indane-5-sulfonate;
3-[2-Amino-4-(3-bromo-4-fluorophenyl)-1-methyl-5-oxo-4,5-dihydro-1H-imidazol-4-yl]phenyl 3-methyl-2-oxo-2,3-dihydro-1,3-benzoxazole-6-sulfonate;
3-[2-Amino-4-(3-bromo-4-fluorophenyl)-1-methyl-5-oxo-4,5-dihydro-1H-imidazol-4-yl]phenyl 5-chloro-1,3-dimethyl-1H-pyrazole-4-sulfonate;
3-[2-Amino-4-(3-bromo-4-fluorophenyl)-1-methyl-5-oxo-4,5-dihydro-1H-imidazol-4-yl]phenyl 5-chlorothiophene-2-sulfonate;
3-[2-Amino-4-(3-bromo-4-fluorophenyl)-1-methyl-5-oxo-4,5-dihydro-1H-imidazol-4-yl]phenyl 1-methyl-1H-imidazole-4-sulfonate;
3-[2-Amino-4-(3-bromo-4-fluorophenyl)-1-methyl-5-oxo-4,5-dihydro-1H-imidazol-4-yl]phenyl 6-chloroimidazo[2,1-b][1,3]thiazole-5-sulfonate;
3-[2-Amino-4-(4-fluoro-3-pyrimidin-5-ylphenyl)-1-methyl-5-oxo-4,5-dihydro-1H-imidazol-4-yl]phenyl 4-(1,1-dimethylpropyl)benzenesulfonate;
3-[2-Amino-4-(4-fluoro-3-pyrimidin-5-ylphenyl)-1-methyl-5-oxo-4,5-dihydro-1H-imidazol-4-yl]phenyl 2,5-dimethoxybenzenesulfonate;
3-[2-Amino-4-(4-fluoro-3-pyrimidin-5-ylphenyl)-1-methyl-5-oxo-4,5-dihydro-1H-imidazol-4-yl]phenyl 2,6-difluorobenzenesulfonate;
3-[2-Amino-4-(4-fluoro-3-pyrimidin-5-ylphenyl)-1-methyl-5-oxo-4,5-dihydro-1H-imidazol-4-yl]phenyl pyridine-2-sulfonate;
3-[2-Amino-4-(4-fluoro-3-pyrimidin-5-ylphenyl)-1-methyl-5-oxo-4,5-dihydro-1H-imidazol-4-yl]phenyl 4-(1H-pyrazol-1-yl)benzenesulfonate;
3-[2-Amino-4-(4-fluoro-3-pyrimidin-5-ylphenyl)-1-methyl-5-oxo-4,5-dihydro-1H-imidazol-4-yl]phenyl indane-5-sulfonate;
3-[2-Amino-4-(4-fluoro-3-pyrimidin-5-ylphenyl)-1-methyl-5-oxo-4,5-dihydro-1H-imidazol-4-yl]phenyl 3-methyl-2-oxo-2,3-dihydro-1,3-benzoxazole-6-sulfonate;
3-[2-Amino-4-(4-fluoro-3-pyrimidin-5-ylphenyl)-1-methyl-5-oxo-4,5-dihydro-1H-imidazol-4-yl]phenyl 5-chloro-1,3-dimethyl-1H-pyrazole-4-sulfonate;
3-[2-Amino-4-(4-fluoro-3-pyrimidin-5-ylphenyl)-1-methyl-5-oxo-4,5-dihydro-1H-imidazol-4-yl]phenyl 5-chlorothiophene-2-sulfonate;
3-[2-Amino-4-(4-fluoro-3-pyrimidin-5-ylphenyl)-1-methyl-5-oxo-4,5-dihydro-1H-imidazol-4-yl]phenyl 1-methyl-1H-imidazole-4-sulfonate;
5-[2-Amino-4-(4-fluoro-3-pyrimidin-5-ylphenyl)-1-methyl-5-oxo-4,5-dihydro-1H-imidazol-4-yl]-2-ethylphenyl methanesulfonate 0.5 acetate;
4-[2-Amino-4-(4-fluoro-3-pyrimidin-5-ylphenyl)-1-methyl-5-oxo-4,5-dihydro-1H-imidazol-4-yl]phenyl 4-(1,1-dimethylpropyl)benzenesulfonate;
4-[2-Amino-4-(4-fluoro-3-pyrimidin-5-ylphenyl)-1-methyl-5-oxo-4,5-dihydro-1H-imidazol-4-yl]phenyl 2,5-dimethoxybenzenesulfonate;
4-[2-Amino-4-(4-fluoro-3-pyrimidin-5-ylphenyl)-1-methyl-5-oxo-4,5-dihydro-1H-imidazol-4-yl]phenyl 2,6-difluorobenzenesulfonate;
4-[2-amino-4-(4-fluoro-3-pyrimidin-5-ylphenyl)-1-methyl-5-oxo-4,5-dihydro-1H-imidazol-4-yl]phenyl 4-methoxybenzenesulfonate;
4-[2-amino-4-(4-fluoro-3-pyrimidin-5-ylphenyl)-1-methyl-5-oxo-4,5-dihydro-1H-imidazol-4-yl]phenyl 3,5-dimethylisoxazol-4-sulfonate;
4-[2-Amino-4-(4-fluoro-3-pyridin-3-ylphenyl)-1-methyl-5-oxo-4,5-dihydro-1H-imidazol-4-yl]phenyl 4-(1,1-dimethylpropyl)benzenesulfonate;
4-[2-Amino-4-(4-fluoro-3-pyridin-3-ylphenyl)-1-methyl-5-oxo-4,5-dihydro-1H-imidazol-4-yl]phenyl 2,5-dimethoxybenzenesulfonate 0.25 acetate;
3-[2-Amino-4-(4-fluoro-3-pyridin-3-ylphenyl)-1-methyl-5-oxo-4,5-dihydro-1H-imidazol-4-yl]phenyl indane-5-sulfonate 0.25 acetate;
4-[2-Amino-4-(4-fluoro-3-pyridin-3-ylphenyl)-1-methyl-5-oxo-4,5-dihydro-1H-imidazol-4-yl]phenyl 3-methyl-2-oxo-2,3-dihydro-1,3-benzoxazole-6-sulfonate 0.5 acetate;
4-[2-Amino-4-(4-fluoro-3-pyridin-3-ylphenyl)-1-methyl-5-oxo-4,5-dihydro-1H-imidazol-4-yl]phenyl 5-chloro-1,3-dimethyl-1H-pyrazole-4-sulfonate 0.25 acetate;
4-[2-Amino-4-(4-fluoro-3-pyridin-3-ylphenyl)-1-methyl-5-oxo-4,5-dihydro-1H-imidazol-4-yl]phenyl 6-chloroimidazo[2,1-b][1,3]thiazole-5-sulfonate 0.75 acetate;
4-[2-Amino-4-(4-fluorophenyl)-1-methyl-5-oxo-4,5-dihydro-1H-imidazol-4-yl]phenyl 3-methyl-2-oxo-2,3-dihydro-1,3-benzoxazole-6-sulfonate;
4-[4-(2′-Acetyl-6-fluoro-5′-methoxybiphenyl-3-yl)-2-amino-1-methyl-5-oxo-4,5-dihydro-1H-imidazol-4-yl]phenyl methanesulfonate;
4-[2-Amino-4-(4-fluoro-3-pyridin-3-ylphenyl)-1-methyl-5-oxo-4,5-dihydro-1H-imidazol-4-yl]phenyl 1-methyl-1H-imidazole-4-sulfonate;
4-{2-amino-4-[4-(3-methoxyphenyl)pyridin-2-yl]-1-methyl-5-oxo-4,5-dihydro-1H-imidazol-4-yl}phenyl methanesulfonate 0.25 acetate;
4-{2-amino-4-[4-(3-methoxyphenyl)pyridin-2-yl]-1-methyl-5-oxo-4,5-dihydro-1H-imidazol-4-yl}phenyl trifluoromethanesulfonate 0.5 acetate;
4-[2-amino-4-(3-cyclohex-1-en-1-yl-4-fluorophenyl)-1-methyl-5-oxo-4,5-dihydro-1H-imidazol-4-yl]phenyl methanesulfonate 0.5 acetate;
4-[2-amino-4-(3-cyclohexyl-4-fluorophenyl)-1-methyl-5-oxo-4,5-dihydro-1H-imidazol-4-yl]phenyl methanesulfonate 0.5 acetate; and
4-{2-amino-4-[4-fluoro-3-(4-methoxypyridin-2-yl)phenyl]-1-methyl-5-oxo-4,5-dihydro-1H-imidazol-4-yl}phenyl methanesulfonate 0.75 acetate
   as a free base, alternative salt or a pharmaceutically acceptable salt, solvate or solvate of a salt thereof.

In another aspect of the invention, there is provided a pharmaceutical composition comprising as active ingredient a therapeutically effective amount of a compound according formula I in association with pharmaceutically acceptable excipients, carriers or diluents.

In another aspect of the invention, there is provided a compound according to formula I, or a pharmaceutically acceptable salt thereof, for use as a medicament.

In another aspect of the invention, there is provided use of a compound according to formula I, as a medicament for treating or preventing an Aβ-related pathology.

In another aspect of the invention, there is provided use of a compound according to formula I, as a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer Disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of a compound according to formula I, as a medicament for treating or preventing Alzheimer Disease.

In another aspect of the invention, there is provided use of a compound according to formula I, in the manufacture of a medicament for treating or preventing an Aβ-related pathology.

In another aspect of the invention, there is provided use of a compound according to formula I, in the manufacture of a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of a compound according to formula I, in the manufacture of a medicament for treating or preventing Alzheimer's Disease.

In another aspect of the invention, there is provided a method of inhibiting activity of BACE comprising contacting said BACE with a compound according to formula I.

In another aspect of the invention, there is provided a method of treating or preventing an Aβ-related pathology in a mammal, comprising administering to said patient a therapeutically effective amount of a compound according to formula I.

In another aspect of the invention, there is provided a method of treating or preventing an Aβ-related pathology in a mammal, comprising administering to said patient a therapeutically effective amount of a compound according to formula I, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing Alzheimer's Disease, comprising administering to said patient a therapeutically effective amount of a compound according to formula I.

In another aspect of the invention, there is provided a method of treating or preventing Alzheimer's Disease, comprising administering to said patient a therapeutically effective amount of a compound according to formula I, wherein said mammal is a human.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor, wherein said Aβ-related pathology is Alzheimer Disease.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor, wherein said mammal is a human.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides use of compounds of formula I or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula I, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula I or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halo, CN, NH₂, OH, COOH, OC₁₋₆alkyl, C₁₋₆alkylOH, SO₂H, C₁₋₆alkyl, OC₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, the term “cycloalkenyl” is intended to include unsaturated ring groups, having the specified number of carbon atoms. These may include fused or bridged polycyclic systems. Preferred cycloalkenyls have from 3 to 10 carbon atoms in their ring structure, and more preferably have 3, 4, 5, and 6 carbons in the ring structure. For example, “C₃₋₆ cycloalkenyl” denotes such groups as cyclopropenyl, cyclobutenyl, cyclopentenyl, or cyclohexenyl.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, imidazothiazolyl and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom. As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, 77Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I. ³⁵S or will generally be most useful. For radio-imaging applications ¹¹C, ¹⁸F, ¹²⁵I, ¹²³I, ¹²⁴I, ¹³¹I, ⁷⁵Br, ⁷³Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula I or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comma 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or deposition of Aβ peptide. Therefore, it is believed that the compounds of the present invention may be used for the treatment of Alzheimer disease and disease associated with dementia Hence, compounds of the present invention and their salts are expected to be active against age-related diseases such as Alzheimer, as well as other Aβ related pathologies such as Downs syndrome and β-amyloid angiopathy. It is expected that the compounds of the present invention would most likely be used as single agents but could also be used in combination with a broad range of cognition deficit enhancement agents.

### Methods of Preparation

The present invention also relates to processes for preparing the compound of formula I as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in “Protective Groups in Organic Synthesis”, T. W. Greene, P. G. M Wutz, Wiley-Interscience, New York, 1999. It is understood that microwaves can be used for the heating of reaction mixtures.

### Preparation of Intermediates

The process, wherein R¹, R², R³, R⁴, R⁵, R⁶, R⁷, A, B and C unless otherwise specified, are as defined hereinbefore, comprises,

(i) conversion of a compound of formula II to obtain a compound of formula III, wherein R⁸ is as defined for A or C above,

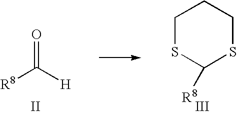

may be carried out by reaction with a suitable reagent such as 1,3-propanedithiol in the presence of an acid such as hydrochloric acid or p-toluenesulfonic acid, or a Lewis acid such as boron trifluoride or titanium tetrachloride or ruthenium(III) chloride. The reaction may be preformed in a suitable solvent such as dichloromethane, acetonitrile, chloroform, toluene or diethyl ether, at a temperature between −78° C. and reflux.
(ii) reaction between a compound of formula IV with a compound of formula III to obtain a compound of formula V, wherein R⁸ and R⁹ are as defined for A or C above,

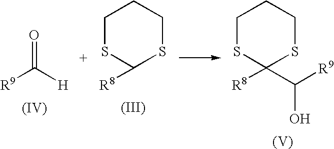

may be carried out by treating III with a suitable base such as an alkyllithium reagent e.g. n-butyl lithium, t-butyl lithium, lithium bis(trimethylsilyl)amide, or lithium diisopropylamide before the addition of II. The reaction may be carried out in a solvent such as tetrahydrofuran or diethyl ether, or a mixture of tetrahydrofuran or diethyl ether with hexane, at a temperature between −100° C. and 25° C. The reaction may be aided by the presence of reagents such as hexamethylphosphoric triamide or N,N,N,N-tetramethyl-1,2-ethanediamide.
(iii) oxidative deprotection and oxidation of a compound of formula V to obtain a compound of formula VI, wherein R⁸ and R⁹ are as defined for A or C above,

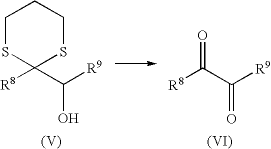

may be carried out by:
a) reaction with a suitable reagent such as 1,1,1-tris(acetyloxy)-1,1-dihydro-1,2-benziodoxol-3(1H)-one, bis(trifluoroacetoxy)iodobenzene, N-bromosuccinimide, or a mixture of trifluoroacetic acid with either sodium nitrite or formaldehyde, in a suitable solvent such as dichloromethane, acetonitrile, chloroform, acetone or water or a mixture thereof, between −5° C. to 40° C. The reaction may be aided by the presence of an alcohol such as t-butanol.
or,
b) hydrolysis by treatment with a suitable reagent or reagent combination such as N-chlorosuccinimide and silver nitrate, N-iodosuccinimide, 3-chloroperoxybenzoic acid, ammonium cerium(IV) nitrate, thallium(III) nitrate, mercury(II) chloride and calcium carbonate, or mercery(II) acetate, in a suitable solvent such as water, acetonitrile, methanol, acetone or diethyl ether or mixtures thereof, between −50° C. and 50° C., followed or preceded by, oxidation with a reagent such as 1,1,1-tris(acetyloxy)-1,1-dihydro-1,2-benziodoxol-3(1H)-one, manganese dioxide, hydrogen peroxide, potassium permanganate, pyridinium chlorochromate, copper sulfate or bromine, in a suitable solvent such as dichloromethane, water, acetonitrile, chloroform or dimethyl formamide, at between 0° C. and reflux.
(iv) conversion of a compound of formula VI to obtain a compound of formula VII, wherein R⁸ and R⁹ are as defined for A or C above,

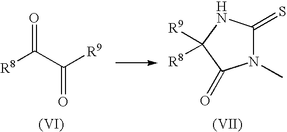

may be carried out by reaction with an appropriately N-substituted thiourea, such as N-methylthiourea, N-ethylthiourea, or N-propylthiourea, in the presence of a suitable base such as potassium hydroxide or sodium hydroxide in a suitable solvent such as water, dimethyl sulfoxide, ethanol or methanol or mixtures thereof, between 20° C. and reflux.
(v) conversion of a compound of formula VII to obtain a compound of formula VIII, wherein R⁸ and R⁹ are as defined for A or C above,

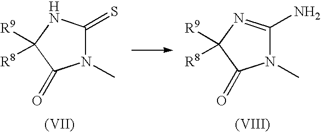

may be carried out by reaction with ammonia, or an ammonia equivalent, together with an alkylhydroperoxide such as t-butylhydroperoxide in a solvent such as ethanol, methanol or water, or a mixture thereof, at 0° C. to 50° C.
(vi) conversion of a compound of formula IX to obtain a compound of formula X, wherein R¹⁰ is as defined for A above and R¹¹ is as defined for R⁶ above,

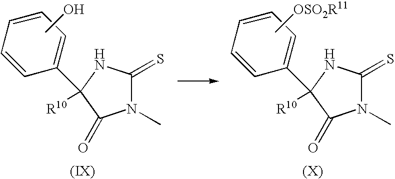

may be carried out by reaction with a suitable reagent such as an alkyl sulfonyl chloride e.g. methane sulfonyl chloride, an alkyl sulfonic anhydride, e.g. trifluoromethanesulfonic anhydride, or a sulfonamide e.g. N-phenyl-bis(trifluoromethanesulfonimide), in the presence of a suitable base such as an organic amine base such as pyridine, 2,6-lutidine, s-collidine, triethylamine, diisopropyl ethylamine, morpholine, N-methylmorpholine, diazabicyclo[5.4.0]undec-7-ene or tetramethylguanidine, or an alkali metal or an alkaline earth metal carbonate such as sodium carbonate, potassium carbonate or calcium carbonate, or potassium phosphate, in a suitable solvent such as dichloromethane, tetrahydrofuran, chloroform, toluene, dimethyl formamide or pyridine at a temperature of −78° C. to 120° C. 4-Dimethylaminopyridine may aid the reaction.
(vii) cross coupling of a compound of formula XI and a compound of formula XII to obtain a compound of formula XIII, wherein Halo is a halogen such as bromine, chlorine or iodine, R¹² and R¹³ are as defined for A and C above or hydrogen or trimethylsilyl,

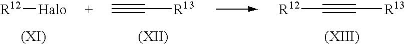

may be performed with a suitable arylhalide such as a compound of formula XI and an alkyne such as ethynyltrimethylsilane, an arylethyne or a heteroarylethyne, in the presence of copper(I) iodide and a suitable palladium catalyst such as dichlorobis(benzonitrile)palladium(II), bis(triphenylphosphine)palladium(II) dichloride, palladium(II) chloride, palladium(0) tetrakistriphenylphosphine with or without a suitable ligand such as tri-tert-butylphosphine or triphenylphosphine, and a suitable base, such as trietylamine, diisopropylamine or piperidine may be used. The reaction may be performed in a solvent such as tetrahydrofuran or N,N-dimethylformamide, at temperatures between 20° C. and 100° C.
(viii) oxidation of a compound of formula XIII to obtain a compound of formula XIV,

wherein R¹² and R¹³ are defined as A and C above,

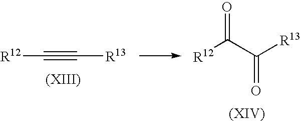

may be performed by reaction with a suitable reagent or mixture of reagents, such as sodium periodate and ruthenium dioxide, iodine and dimethyl sulfoxide, palladium chloride and dimethyl sulfoxide, oxone, hydrogen peroxide, oxygen, potassium permanganate, ruthenium tetroxide, or selenium dioxide, in a suitable solvent such as dimethyl sulfoxide, dichloromethane, acetonitrile, water, acetone, chloroform or carbon tetrachloride at a temperature between −78° C. and 150° C. The reaction may be aided by the presence of a catalyst such as ruthenium(III) chloride or iron(III) chloride.
(ix) reaction of a compound of formula XV wherein R¹⁴ is as defined for C above, to a compound of formula XVI,

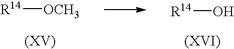

The reaction may be carried out by treating the methyl ether with a suitable Lewis acid such as boron tribromide, aluminum chloride or aluminum bromide in a suitable solvent such as dichloromethane, methanol or toluene at a temperature between −78° C. and reflux, or by treating the methyl ether with sodium methylmercaptide, in a suitable solvent such as 1-methyl-2-pyrrolidinone at a temperature between 20° C. and 120° C. The reaction may also be performed by treatment of XV with an acid, such as an aqueous solution of hydrogen bromide, at temperature between 20° C. and reflux, or by treating XV with a hydride reagent such as sodium hydride in the presence of ethanethiol in a solvent such as N,N-dimethylformamide at a temperature of 100° C.-reflux.

(x) cross coupling of a compound of formula XVII to obtain a compound of formula XIX, wherein Halo represents halogen such as chlorine, bromine or iodine, R¹⁵ is as defined for A above, R¹⁶ is as defined for B above and R¹⁷ may be a group outlined in Scheme I, wherein R¹⁸ and R¹⁹ are groups such as OH, C₁₋₆alkylO or C₂₋₃alkylO fused together to form a 5 or 6 membered boron containing heterocycle and the alkyl, cycloalkyl or aryl moieties may be optionally substituted,

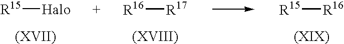

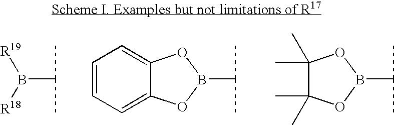

may be carried out by a de-halogen coupling with a suitable compound of formula XVIII. The reaction may be carried out by coupling of a compound of formula XVII with an appropriate aryl boronic acid or boronic ester of formula XVIII. The reaction may be carried out using a suitable palladium catalyst such as tetrakis(triphenylphosphine)palladium(0), palladium diphenylphosphinoferrocene dichloride or palladium acetate, together with, or without, a suitable ligand such as tri-tert-butylphosphine or 2-(dicyclohexylphosphino)biphenyl, or using a nickel catalyst such as nickel on charcoal or 1,2-Bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a temperature range between 20° C. and 160° C., in a suitable solvent such as toluene, tetrahydrofuran, dioxane, dimethoxyethane, water, ethanol or N,N-dimethylformamide, or mixtures thereof.
(xi) reaction of a compound of formula XX to obtain a compound of formula XXI, wherein Halo represents halogen such as chlorine, bromine or iodine and R²⁰ is as defined for A or C above,

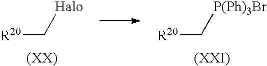

may be performed by treating an appropriate halide such as a compound of formula XX with triphenylphosphine. The reaction may be preformed in a suitable solvent such as toluene or benzene at a temperature between 20° C. and reflux.
(xii) reaction of a compound of formula XXI with a compound of formula XXII to obtain a compound of formula XXIII, wherein R¹⁹ and R²⁰ are as defined for A or C above,

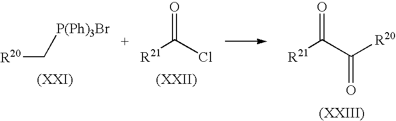

may be preformed by treating the appropriate phosphonium halide such as a compound of formula XXI with a suitable alkyllithium such as butyllithium followed by addition of an appropriate acyl chloride such as a compound of formula XXII. The reaction may be preformed in a suitable solvent such as toluene or benzene at a temperature between −78° C. and 25° C.
(xiii) borylation of a compound of formula XXIV to obtain a compound of formula XXV, wherein R²² is an optionally substituted phenyl or alkyne and R²³ may be a group outlined in Scheme II, wherein R¹⁸ and R¹⁹ are groups such as OH, C₁₋₆alkylO or C₂₋₃alkylO fused together to form a 5 or 6 membered boron containing heterocycle and the alkyl, cycloalkyl or aryl moieties may be optionally substituted,

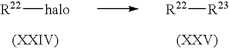

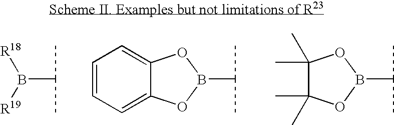

may be carried out by a reaction with:

a) an alkyllithium such as butyllithium, or magnesium, and a suitable boron compound such as trimethyl borate or triisopropyl borate. The reaction may be performed in a suitable solvent such as tetrahydrofuran, hexane or dichloromethane in a temperature range between −78° C. and 20° C.;

or,
b) a suitable boron species such as biscatecholatodiboron, bispinacolatodiboron or pinacolborane in the presence of a suitable palladium catalyst such as palladium(0) tetrakistriphenylphosphine, palladium diphenylphosphinoferrocene dichloride or palladium acetate, with or without a suitable ligand such as 2-(dicyclohexylphosphino)biphenyl, and a suitable base, such as a tertiary amine, such as trietylamine or diisopropylethylamine, or potassium acetate may be used. The reaction may be performed in a solvent such as dioxane, toluene, acetonitrile, water, ethanol or 1,2-dimethoxyethane, or mixtures thereof, at temperatures between 20° C. and 160° C.
(xiv) reduction of a compound of formula XXVI to a compound of formula XXVII, wherein R²⁴ is cyclopropyl and R²⁵ is a substituted phenyl,

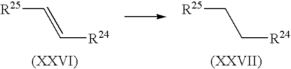

may be preformed using a suitable catalyst such as palladium-on-charcoal, Raney nickel or Wilkinson's catalyst, and a hydrogen source such as hydrogen gas. The reaction may be preformed in a suitable solvent such as ethyl acetate, methanol or ethanol, at temperatures between 20° C. and reflux.
(xv) conversion of a compound of formula XXVIII to obtain a compound of formula XXIX, wherein R²⁷ is as defined for C above an R²⁶ is as defined for A above,

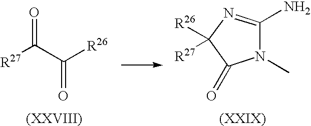

may be carried out by reaction with an appropriately N-substituted guanidine such as N-methylguanidine, N-ethylguanidine, or N-propylguanidine, in the presence of a suitable base such as potassium hydroxide, sodium hydroxide or sodium carbonate in a suitable solvent such as water, dimethyl sulfoxide, dioxane, ethanol or methanol or mixtures thereof, between 20° C. and reflux.
(xvi) cross coupling of a compound of formula XVII to obtain a compound of formula XIX, wherein Halo represents halogen such as chlorine, bromine or iodine, R¹⁵ is as defined for A above, may be carried out by a de-halogen coupling with a suitable compound of formula XXX, wherein R¹⁶ is as defined for B above and R²⁸ may be a trialkyltin such as a trimethyltin or tributyltin.

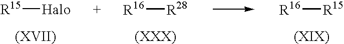

The reaction may be carried out using a suitable palladium catalyst such as, dichlorobis(triphenylphosphine)palladium(II), tetrakis(triphenylphosphine)palladium(0) or bis(dibenzylideneacetone) palladium (0), together with, or without, a suitable ligand such as triphenylarsine and/or a suitable additive such as silver(I) oxide in a solvent such as N,N-dimethylformamide, tetrahydrofuran, toluene, 1-methyl-2-pyrrolidinone or dioxane at a temperature between 20° C. and 150° C. using conventional or microwave assisted heating.

(xvii) silylation of a compound of formula XXXI to obtain a compound of formula XXXII, wherein R²⁹ is an optionally substituted aryl and R³⁰ is alkyl or aryl,

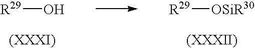

may be carried out with a suitable reagent such as a trialkyl and/or aryl silyl chloride such as t-butyldiphenylsilyl chloride, trimethylsilyl chloride or triisopropyl silylchloride, or a trialkylsilyltrifluoromethane sulfonate such as triethylsilyltrifluoromethane sulfonate, in the presence of a suitable base such as an organic amine base such as imidazole, pyridine, 2,6-lutidine, collidine, 4-dimethylaminopyridine, triethylamine, morpholine, N-methylmorpholine, diazabicyclo[5.4.0]undec-7-ene, tetramethylguanidine or an alkali metal hydride such as sodium hydride, in a suitable solvent such as dichloromethane, tetrahydrofuran, dimethylformamide at a temperature between 0° C. and 100° C. The reaction may be aided by 4-dimethylaminopyridine.
(xviii) borylation of a compound of formula XXXIII to obtain a compound of formula XXXIV, wherein 1 is an optionally substituted alkyl or cycloalkyl and R³² may be a group outlined in Scheme III, wherein R¹⁸ and R¹⁹ are groups such as OH, C₁₋₆alkylO or C₂₋₃alkylO fused together to form a 5 or 6 membered boron containing heterocycle and the alkyl, cycloalkyl or aryl moieties may be optionally substituted,

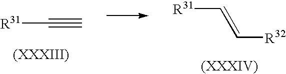

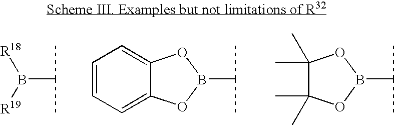

may be carried out by a reaction with a suitable boron species such as catecholborane, dibromoborane dimethyl sulfide complex, dichloroborane dioxane complex or pinacolborane. The reaction may be performed in a solvent such as dioxane, tetrahydrofuran or dichloromethane at temperatures between 0° C. and reflux.
(xix) reduction of a compound of formula XXXV to obtain a compound of formula XXXVI, wherein R³³ is as defined for C above,

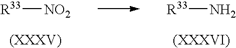

may be carried out by a reaction with:
a) a suitable catalyst such as platinum(IV) oxide, palladium- or platinum-on-carbon, Raney nickel or zinc metal, and a hydrogen source such as hydrogen gas. The reaction may be performed in a solvent such as methanol, ethanol, tetrahydrofuran or ethyl acetate or mixtures thereof at temperatures between 20° C. and reflux; or by a reaction with,
b) tin(II) chloride and aqueous hydrogen chloride or ammonium acetate in a solvent such as methanol or ethanol at temperatures between −10° C. and reflux.
(xx) conversion of a compound of formula XXXVI to obtain a compound of formula XXXVII wherein R³³ is as defined for C above,

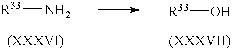

may be carried out in an acidic media such as an aqueous solution of hydrogen chloride or sulfuric acid, in the presence of a nitrite source such as sodium nitrite, tert-butyl nitrite or isoamyl nitrite. The reaction may be performed in a solvent such as water, methanol, dioxane, acetonitrile or N,N-dimethylformamide or mixtures thereof. at temperatures between 0° C. and reflux.
(xxi) nitration of a compound of formula XXXVIII to obtain a compound of formula XXXIX wherein R³⁴ is an optionally substituted heteroaryl,

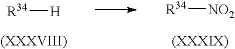

may be carried out in an acidic media such as sulfuric acid or acetic acid in the presence of a reagent such as nitric acid, sodium nitrite, silver nitrate or cerium(IV) ammonium nitrate. The reaction may be performed with or without a solvent such as acetonitrile or methanol at temperatures between 0° C. and reflux.
(xxii) bromination of a compound of formula XXXIX to obtain a compound of formula XL wherein R³⁴ is an optionally substituted heteroaryl,

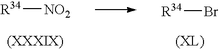

may be carried out with a reagent such as hydrogen bromide or acetyl bromide in acetic acid at temperatures between 0° C. and reflux.
(xxiii) reduction of a compound of formula XLI to obtain a compound of formula XLII wherein R³⁵ is independently chosen from alkyl or halogen and n is a number between 1 and 3,

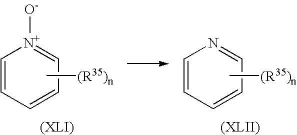

may be carried out with:
a) a suitable catalyst such as Raney nickel, palladium-on-charcoal, indium in combination with titanium(IV) chloride, or ruthenium trichloride possibly in combination with sodium hypophosphite hydrate, with or without a hydrogen source such as hydrogen gas. The reaction may be performed in a solvent such as acetonitrile, acetic acid, methanol or tetrahydrofuran at temperatures between 0° C. and reflux;
or,
b) a reagent such as phosphorus tribromide or phosphorus trichloride in a solvent such as chloroform at temperatures between 20° C. and reflux.
(xxiv) conversion of a compound of formula XLIII to obtain a compound of formula XLIV wherein R³⁶ is as defined in A or C above,

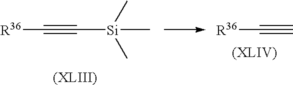

may be carried out with a suitable reagent such as tetrabutylammonium fluoride, potassium carbonate or potassium hydroxide, in a solvent such as chloroform, tetrahydrofuran, methanol or diethylether at temperatures between 0° C. and reflux.
(xxv) conversion of a compound of formula XLV to obtain a compound of formula XLVI wherein R³⁷ is a substituted aryl,

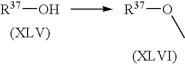

may be carried out with:
a) an alkylating agent such as dimethyl sulfate or methyl iodide together with a suitable base such as sodium- or potassium hydroxide, potassium carbonate or sodium hydride, with or without tetrabutylammonium sulfate. The reaction may be performed in a solvent such as dichloromethane, acetone, methanol, N,N-dimethylformamide or tetrahydrofuran or mixtures thereof, at a temperature between 0° C. and reflux;

### Methods of Preparation of End Products

Another object of the invention are processes a, b, c, d or e for the preparation of compounds of general formula I, wherein R¹, R², R³, R⁴, R⁵, R⁶, R⁷, A, B and C unless otherwise specified, are defined as hereinbefore, and salts thereof. When it is desired to obtain the acid salt, the free base may be treated with an acid such as a hydrogen halide such as hydrogen chloride, sulphuric acid, a sulphonic acid such as methane sulphonic acid or a carboxylic acid such as acetic or citric acid in a suitable solvent such as tetrahydrofuran, diethyl ether, methanol, ethanol, chloroform or dichloromethane or mixtures thereof, the reaction may occur between −30° C. to 50° C.

These processes comprise;
(a) conversion of a compound of formula XLVII to a compound of formula I, wherein halo represents halogen such as chlorine, bromine or iodine, R¹, R², A, B and C are as defined above, and R³⁸ may be a group outlined in Scheme II above,

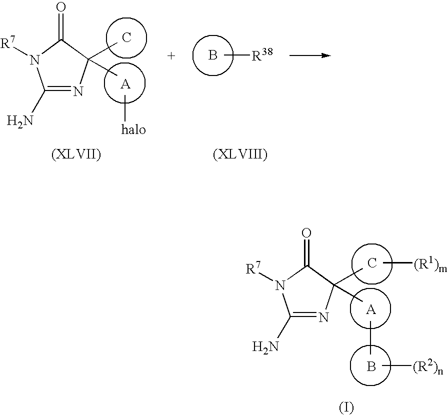

may be carried out by a de-halogen coupling with a suitable compound of formula XLVIII. The reaction may be carried out by coupling of a compound of formula XLVII with an appropriate aryl boronic acid or boronic ester of formula XLVIII. The reaction may be carried out using a suitable palladium catalyst such as tetrakis(triphenylphosphine)palladium(0), palladium diphenylphosphinoferrocene dichloride or palladium acetate, together with, or without, a suitable ligand such as tri-tert-butylphosphine or 2-(dicyclohexylphosphino)biphenyl, or using a nickel catalyst such as nickel on charcoal or 1,2-Bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a temperature range between 20° C. and 160° C., in a suitable solvent such as toluene, tetrahydrofuran, dioxane, 1,2-dimethoxyethane, water, ethanol or N,N-dimethylformamide, or mixtures thereof
(b) conversion of a compound of formula XLIX to a compound of formula I, wherein halo represents halogen such as chlorine, bromine or iodine, R¹, R², A, B and C are as defined above.

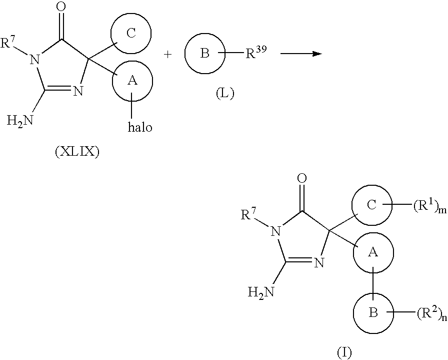

The reaction of process (b) may be carried out by coupling of a suitable compound such as a compound of formula XLIX with an appropriate stannane of formula L wherein R³⁹ is a trialkyltin such as trimethyltin or tributyltin. The reaction may be carried out using a suitable palladium catalyst such as, dichlorobis(triphenylphosphine)palladium(II), tetrakis(triphenylphosphine)palladium(0) or bis(dibenzylideneacetone) palladium (0), together with, or without, a suitable ligand such as triphenylarsine and/or a suitable additive such as silver(I) oxide, in a solvent such as N,N-dimethylformamide, tetrahydrofuran, toluene, 1-methyl-2-pyrrolidinone or dioxane at a temperature between 20° C. and 150° C. using conventional or microwave assisted heating.

(c) reduction of a compound of formula LI to a compound of formula I, wherein R¹, R², A, and C are as defined above and B is CH₂CH₂cyclopropyl.

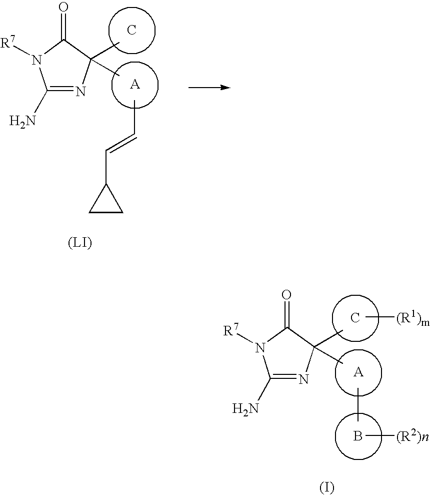

may be performed using a suitable catalyst such as palladium-on-charcoal, Raney nickel or Wilkinson's catalyst, and hydrogen. The reaction may be performed in a suitable solvent such as ethyl acetate, methanol or ethanol, at temperatures between 20° C. and reflux.
(d) conversion of a compound of formula LII to obtain a compound of formula I, wherein R¹, R², m, n, A, B and C are as defined above,

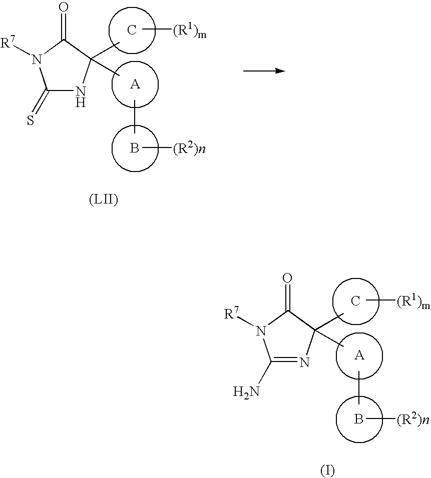

may be carried out by reaction with ammonia, or an ammonia equivalent, together with an alkylhydroperoxide such as t-butylhydroperoxide in a solvent such as ethanol, methanol or water, or a mixture thereof, at 0° C. to 50° C.
(e) conversion of a compound of formula LIII to obtain a compound of formula I, wherein R¹, R², m, n, A, B and C are as defined above,

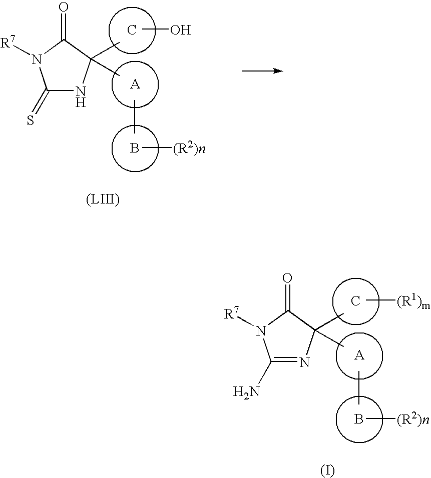

may be carried out by reaction with a suitable reagent such as an alkyl sulfonyl chloride e.g. methane sulfonyl chloride, an alkyl sulfonic anhydride, e.g. trifluoromethanesulfonic anhydride, or a sulfonimide e.g. N-phenyl-bis(trifluoromethanesulfonimide), in the presence of a suitable base such as an organic amine base such as pyridine, 2,6-lutidine, s-collidine, triethylamine, diisopropyl ethylamine, morpholine, N-methylmorpholine, diazabicyclo[5.4.0]undec-7-ene or tetramethylguanidine, or an alkali metal or an alkaline earth metal carbonate such as sodium carbonate, potassium carbonate or calcium carbonate, or potassium phosphate, in a suitable solvent such as dichloromethane, tetrahydrofuran, chloroform, toluene, dimethyl formamide or pyridine at a temperature of −78° C. to 120° C. 4-Dimethylaminopyridine may aid the reaction.

### General Methods

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at 400 MHz. The 400 MHz spectra were obtained unless stated otherwise, using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR spectrometer equipped with a 4-nucleus probehead with Z-gradients. Chemical shifts are given in ppm down- and upfield from TMS. Resonance multiplicities are denoted s, d, t, q, m and br for singlet, doublet, triplet, quartet, multiplet, and broad respectively.

LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm. Or LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was configured with an atmospheric pressure chemical ionisation (APCI) ion source which was further equipped with atmospheric pressure photo ionisation (APPI) device. The mass spectrometer scanned in the positive mode, switching between APCI and APPI mode. The mass range was set to m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode. Separation was performed using a Gemini column C18, 3.0 mm×50 mm, 3 μm, (Phenomenex) and run at a flow rate of 1 ml/min. A linear gradient was used starting at 100% A (A: 10 mM ammonium acetate in 5% methanol) and ending at 100% B (methanol). The column oven temperature was set to 40° C.

Mass spectra (MS) were run using an automated system with atmospheric pressure chemical (APCI or CT) or electrospray (+ESI) ionization. Generally, only spectra where parent masses are observed are reported. The lowest mass major ion is reported for molecules where isotope splitting results in multiple mass spectral peaks (for example when chlorine is present).

GC-MS analyses were performed on a Agilent 6890N GC equipped with a Chrompack CP-Sil 5CB column (25 m×0.25 mm i.d. df=0.25)), coupled to an Agilent 5973 Mass Selective Detector operating in a chemical ionization (CI) mode and the MS was scanned between m/z 50-500.

Accurate mass analyses were performed on a QTOF micro (Waters). The mass spectrometer was equipped with an electrospray ionsource that uses two probes, a sample probe and a lock mass probe, respectively. The lock mass solution was Leucine Enkephaline (0.5 ng/uL in MilliQ water) infused at flow rate of 0.1 mL/min. The reference scan frequency was set to 5.5 s. Before the analysis, the mass spectrometer was calibrated in the positive mode between 90-1000 Da using a solution of NaFormate. The mass spectrometer scanned in the centroid mode between m/z 100-1000 with a scan time of 1.0 s. The capillary voltage was set to 3.3 kV and the ES cone voltage was set to 28 V. The source temperature and desolvation temperature were set to 110° C. and 350° C., respectively. The collision energy was set to 6.0 V. The QTOF micro was equipped with an LC(HP1100 Agilent, Degasser, Binary pump, ALS and a column compartment). The column used was a Gemini C18, 3.0×50 mm, 3 u run at a flowrate of 1.0 mL/min. A linear gradient was applied starting at 100% A (A: 10 mM ammonium acetate) and ending at 100% B (B: acetonitrile) after 4 min. The column oven temperature was set to 40° C. The flow was splitted 1:4 prior to the ion source. 3 uL of the sample was injected on the column.

HPLC assays were performed using an Agilent HP1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min. Mobile phase A: 10 mM ammonium acetate in water/acetonitrile (95:5), mobile phase B: acetonitrile.

Preparative HPLC was performed on a Waters Auto purification HPLC-UV system with a diode array detector using a Waters XTerra MS C₈ column (19×300 mm, 7 μm) and a linear gradient of mobile phase B was applied. Mobile phase A: 0.1 M ammonium acetate in water/acetonitrile (95:5) and mobile phase B: acetonitrile. Flow rate: 20 mL/min. Thin layer chromatography (TLC) was performed on Merch TLC-plates (Silica gel 60 F₂₅₄) and spots were UV visualized. Flash chromatography was performed using Merck Silica gel 60 (0.040-0.063 mm), or employing a Combi Flash® Companion™ system using RediSep normal-phase flash columns.

Compounds have been named using ACD/Name, version 9.0, software from Advanced Chemistry Development, Inc. (ACD/Labs), Toronto ON, Canada, www.acdlabs.com, 2004.

### EXAMPLES

Below follows a number of non-limiting examples of compounds of the invention.

### Example 1

### (3-Bromo-4-fluorophenyl) [2-(4-{[tert-butyl(diphenyl)silyl]oxy}phenyl)-1,3-dithian-2-yl]methanol

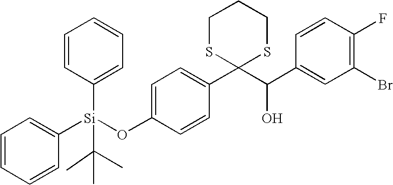

To a solution of tert-butyl[4-(1,3-dithian-2-yl)phenoxy]diphenylsilane (2.0 g, 4.44 mmol; described in: Philip C. Bulman Page et al, Tetrahedron, 1992, 48, 7265-7274) in tetrahydrofuran (50 mL) was added a solution of n-butyllithium in hexane (3.05 mL, 4.88 mmol, 1.6 M) at −78° C. and the mixture was stirred at −78° C. for 2 h. 3-Bromo-4-fluorobenzaldehyde (0.95 g, 4.66 mmol) was added at −78° C. and the reaction was allowed to reach room temperature overnight. The reaction mixture was poured into a saturated ammonium chloride solution and the aqueous layer was extracted with dichloromethane. The combined organic layers were dried over magnesium sulfate and the solvent was evaporated. The residue was purified by column chromatography, using heptane/ethyl/acetate (7:1 to 5:1) as the eluent, to give 1.95 g (67% yield) of the title compound: ¹H NMR (CDCl₃) δ 7.74 (dd, J=6.8, 1.3 Hz, 4H), 7.43-7.48 (m, 2H), 7.37-7.43 (m, 4H), 7.31-7.36 (m, 2H), 7.10 (dd, J=6.7, 2.1 Hz, 1H), 6.68-6.74 (m, 3H), 6.54-6.61 (m, 1H), 4.84 (s, 1H), 2.61-2.76 (m, 4H), 1.86-1.95 (m, 2H), 1.09-1.17 (m, 9H). HR MS (ES) m/z 635.0881, 637.0852 [M-18]⁺.

### Example 2

### 1-(3-Bromo-4-fluorophenyl)-2-(4-{[tert-butyl(diphenyl)silyl]oxy}phenyl)ethane-1,2-dione

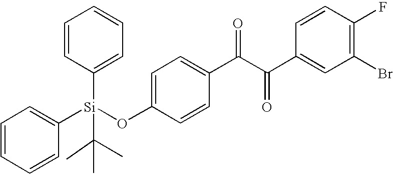

Dess-Martin periodinane (3.1 g, 7.23 mmol) was added to a solution of (3-bromo-4-fluorophenyl)[2-(4-{[tert-butyl(diphenyl)silyl]oxy}phenyl)-1,3-dithian-2-yl]methanol (1.90 g, 2.91 mmol) and tert-butanol (0.93 mL, 10.2 mmol) in dichloromethane (40 mL) under an argon atmosphere and the reaction mixture was stirred overnight. Sodium thiosulfate (2.5 g) dissolved in saturated aqueous sodium hydrogencarbonate (40 mL) was added and the resulting mixture was stirred for 30 min. Dichloromethane was added and the organic phase separated. The aqueous phase was extracted with dichloromethane, the combined organic phases were dried over magnesium sulfate and the solvent was evaporated. The residue was purified by column chromatography, using heptane/ethyl/acetate (9:1 to 8:1) as the eluent, to give 1.54 g (94% yield) of the title compound: HR MS (ES) m/z 561.0924, 563.0913 [M+H]⁺.

### Example 3

### 5-(3-Bromo-4-fluorophenyl)-5-(4-hydroxyphenyl)-3-methyl-2-thioxoimidazolidin-4-one

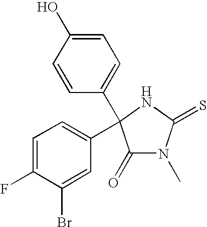

Aqueous potassium hydroxide (1.2 M, 4.45 mL, 5.34 mmol) was added to a solution of 1-(3-bromo-4-fluorophenyl)-2-(4-{[tert-butyl(diphenyl)silyl]oxy}phenyl)ethane-1,2-dione (1.50 g, 2.67 mmol) and methyl-2-thiourea (0.48 g, 5.34 mmol) in dimethyl sulfoxide (10 mL) at 100° C., stirred for 1 h and the reaction mixture was allowed to cool down to room temperature. The reaction mixture was diluted with water and dichloromethane. The pH was adjusted to 3-4 by addition of aqueous hydrochloric acid (2 M). The organic phase was separated and the aqueous phase was washed with dichloromethane. The combined organic phases were dried over magnesium sulfate and the solvent was evaporated. The residue was purified by column chromatography, using heptane/ethyl acetate (3:1 to 2:1) as the eluent, to give 0.73 g (69% yield) of the title compound: ¹H NMR (CDCl₃) δ 8.75 (s, 1H), 7.57 (dd, J=6.2, 2.1 Hz, 1H), 7.22-7.39 (m, 1H), 7.01-7.17 (m, 3H), 6.78 (d, J=8.6 Hz, 2H), 3.33 (s, 3H): MS (ES) m/z 392.97, 394.94 [M−H]⁻.

### Example 4

### 4-[4-(3-Bromo-4-fluorophenyl)-1-methyl-5-oxo-2-thioxoimidazolidin-4-yl]phenyl methanesulfonate

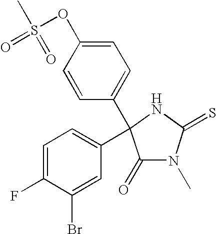

Methanesulfonyl chloride (0.96 mL, 12.5 mmol) was added to a cooled (0° C.) solution of 5-(3-bromo-4-fluorophenyl)-5-(4-hydroxyphenyl)-3-methyl-2-thioxoimidazolidin-4-one (3.98 g, 10.1 mmol) and triethylamine (1.74 mL, 12.6 mmol) in dichloromethane (100 mL) and the reaction was kept at 8° C. overnight. The reaction mixture was washed with water and brine. The organic phase was dried over magnesium sulfate and the solvent was evaporated to give 4.77 g (97% yield) of the title compound. MS (ES) m/z 470.95, 472.95 [M−H]⁻.

### Example 5

### 4-[2-Amino-4-(3-bromo-4-fluorophenyl)-1-methyl-5-oxo-4,5-dihydro-1H-imidazol-4-yl]phenyl methanesulfonate

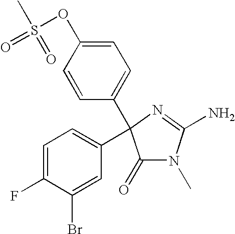

tert-Butylperoxide (23.4 mL, 151.5 mmol, 70 wt % in water) was added to a solution of 4-[4-(3-bromo-4-fluorophenyl)-1-methyl-5-oxo-2-thioxoimidazolidin-4-yl]phenyl methanesulfonate (4.77 g, 10.1 mmol) in a mixture of methanol/ammonium hydroxide (25:75 mL). The reaction was stirred at room temperature for 3 h and at 8° C. overnight. The reaction mixture was concentrated and the residue was dissolved in chloroform. The organic phase was washed with water, dried over magnesium sulfate and the solvent was evaporated. The residue was purified by column chromatography, using ethyl acetate/methanol (20:1+1% triethylamine) as the eluent, to give 2.6 g (60% yield) of the title compound. ¹H NMR (CDCl₃) δ 7.72 (dd, J=6.6, 2.3 Hz, 1H), 7.49-7.56 (m, 2H), 7.38-7.45 (m, 1H), 7.19-7.25 (m, 2H), 7.05 (t, J=8.5 Hz, 1H), 3.11-3.14 (m, 3H), 3.10 (s, 3H): MS (ES) m/z 453.96, 455.98 [M−H]⁻.

### Example 6

### 4-{2-Amino-4-[4-fluoro-3-(4-methoxypyrimidin-2-yl)phenyl]-1-methyl-5-oxo-4,5-dihydro-1H-imidazol-4-yl}phenyl methanesulfonate 0.25 acetate

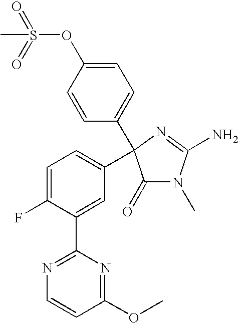

Anhydrous tetrahydrofuran (3 mL) was added to 4-[2-amino-4-(3-bromo-4-fluorophenyl)-1-methyl-5-oxo-4,5-dihydro-1H-imidazol-4-yl]phenyl methanesulfonate (80 mg, 0.18 mmol), 4-methoxy-2-(tributylstannyl)pyrimidine (described in: Darabantu M. Tetrahedron 2005, 61, 11, 2897-2905, 105 mg, 0.26 mmol) and bis(triphenylphosphine)palladium(II) dichloride (6 mg, 0.009 mmol) and the mixture was irradiated under an atmosphere of argon in a microwave at 130° C. for 12 h. When cooled to room temperature, the solvent was evaporated, dimethyl sulfoxide (1 mL) was added and the mixture was filtered. The product was purified by preparative HPLC to give 22 mg (11% yield) of the title compound: ¹H-NMR (DMSO-d₆) δ 8.63 (d, J=5.8 Hz, 1H), 8.22 (dd, J=7.4, 2.4 Hz, 1H), 7.65-7.59 (m, 1H), 7.59-7.54 (m, 2H), 7.32-7.25 (m, 3H), 6.92 (d, J=5.8 Hz, 1H), 6.77 (br s, 2H), 3.94 (s, 3H), 3.35 (s, 3H), 3.00 (s, 3H), 1.87 (s, 0.51H); MS (ES) m/z 484 [M−H]⁻.

### Example 7

### 3-{2-Amino-4-[4-fluoro-3-(4-methoxypyrimidin-2-yl)phenyl]-1-methyl-5-oxo-4,5-dihydro-1H-imidazol-4-yl}phenyl methanesulfonate 0.5 acetate

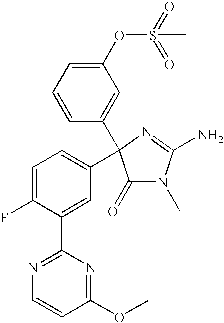

Anhydrous tetrahydrofuran (3 mL) was added to 3-[2-amino-4-(3-bromo-4-fluorophenyl)-1-methyl-5-oxo-4,5-dihydro-1H-imidazol-4-yl]phenyl methanesulfonate (96 mg, 0.21 mmol), 4-methoxy-2-(tributylstannyl)pyrimidine (described in: Darabantu M. Tetrahedron 2005, 61, 11, 2897-2905, 121 mg, 0.30 mmol) and bis(triphenylphosphine)palladium(II) dichloride (15 mg, 0.021 mmol), and the mixture was irradiated under an atmosphere of argon in a microwave at 130° C. for 13 h. When cooled to room temperature the solvent was evaporated, dimethyl sulfoxide (1 mL) was added and the mixture was filtered. The product was purified by preparative HPLC to give 9 mg (9% yield) of the title compound: ¹H-NMR (CDCl₃) δ 8.51 (d, J=5.8 Hz, 1H), 8.20 (dd, J=7.1, 2.5 Hz, 1H), 7.63-7.53 (m, 2H), 7.43-7.35 (m, 2H), 7.22-7.11 (m, 2H), 6.67 (d, J=5.8 Hz, 1H), 4.01 (s, 3H), 3.14 (s, 3H), 3.10 (s, 3H), 2.03 (s, 1.21H); MS (ES) m/z 484 [M−H]⁻.

### Example 8

### 2-Bromo-4-ethynyl-1-fluorobenzene

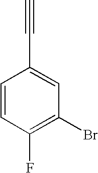

To a mixture of 2-bromo-1-fluoro-4-iodobenzene (11.30 g, 37.6 mmol), copper(I) iodide (715 mg, 3.76 mmol), bis(triphenylphosphine)palladium(II) dichloride (1.32 g, 1.90 mmol) and piperidine (5.21 mL, 5.26 mmol) in anhydrous tetrahydrofuran (150 mL) ethynyl(trimethyl)silane (7.43 mL, 5.26 mmol) was added under an atmosphere of argon. The reaction mixture was stirred at room temperature for 0.5 h. The mixture was filtered and the solvent was evaporated in vacuo. Purification by column chromatography, using 0-20% ethyl acetate in heptane as the eluent, gave the trimethylsilyl protected product. To remove the trimethylsilyl group, potassium carbonate (15.6 g, 113 mmol) and methanol (100 mL) were added to the product and the mixture was stirred at room temperature for 45 min. The slurry was filtered and the solvent was evaporated in vacuo. Water was added and the mixture was extracted with ethyl acetate. The combined organic extracts were washed with aqueous saturated sodium chloride, dried over magnesium sulfate, filtered and the solvent was evaporated in vacuo to give 6.70 g (90% yield) of the title compound: ¹H-NMR (DMSO-d₆) δ 7.85 (dd, J=6.6, 2.1 Hz, 1H), 7.58-7.52 (m, 1H), 7.40 (t, J=8.8 Hz, 1H), 4.30 (s, 1H); MS (EI) m/z 198, 200 [M+•].

### Example 9

### 5-[(3-Bromo-4-fluorophenyl)ethynyl]-2-methoxyphenyl acetate

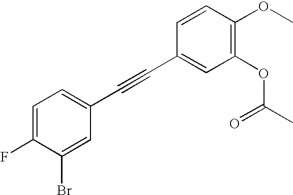

A solution of 2-bromo-4-ethynyl-1-fluorobenzene (3.90 g, 19.6 mmol) in anhydrous tetrahydrofuran (10 mL) was added dropwise to a solution of 5-iodo-2-methoxyphenyl acetate (described in: Noda, Y. Tetrahedron Letters 1997, 38, 35, 6225-6228, 4.58 g, 15.7 mmol), copper(I) iodide (187 mg, 0.98 mmol) and bis(triphenylphosphine)palladium(II) dichloride (688 mg, 0.98 mmol) in a 2:1 mixture of tetrahydrofuran and triethylamine (100 mL). The reaction was stirred at room temperature under an atmosphere of argon for 3 h. The reaction mixture was filtered and the solvent was evaporated in vacuo. The product was purified by column chromatography, using 30% ethyl acetate in heptane as the eluent, to give 4.95 g (69% yield) of the title compound: ¹H-NMR (DMSO-d₆): δ 7.91 (dd, J=6.8, 2.01 Hz, 1H), 7.61-7.56 (m, 1H), 7.48-7.41 (m, 2H), 7.32 (d, J=2.0 Hz, 1H), 7.19 (d, J=8.5 Hz, 1H), 3.82 (s, 3H), 2.27 (s, 3H); MS (ES) m/z 363, 365 [M+H]⁺.

### Example 10

### 5-[(3-Bromo-4-fluorophenyl)(oxo)acetyl]-2-methoxyphenyl acetate

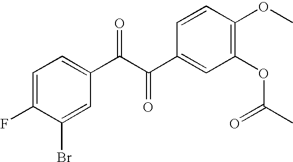

A solution of 5-[(3-bromo-4-fluorophenyl)ethynyl]-2-methoxyphenyl acetate (4.95 g, 13.6 mmol) and palladium(II) chloride (266 mg, 1.5 mmol) in anhydrous dimethyl sulfoxide (30 mL) was heated in an oil bath at 130° C. for 8 h. More palladium(II) chloride (10 mg, 0.056 mmol) was added and the reaction mixture was stirred for 5 h. When cooled to room temperature, water was added (100 mL) and the aqueous phase was extracted with diethyl ether. Undissolved material was removed by filtration and rinsed with diethyl ether to give 3.47 g of pure product. The combined organic extracts were washed with aqueous saturated sodium chloride, dried over magnesium sulfate, filtered and the solvent was evaporated in vacuo. Diethyl ether and a few drops of methanol was added to the product resulting in a slurry which was filtered. The solid was rinsed with diethyl ether to give a second crop. The two crops were pooled to give the title compound in 89% yield: ¹H-NMR (DMSO-d₆): δ 8.25 (dd, J=6.6, 2.1 Hz, 1H), 8.01-7.95 (m, 1H), 7.87 (dd, J=8.5, 2.26 Hz, 1H), 7.73 (d, J=2.3 Hz, 1H), 7.61 (t, J=8.5 Hz, 1H), 7.34 (d, J=8.8 Hz, 1H), 3.91 (s, 3H), 2.28 (s, 3H); MS (ES) m/z 395, 397 [M+H]⁺.

### Example 11

### 5-(3-Bromo-4-fluorophenyl)-5-(3-hydroxy-4-methoxyphenyl)-3-methyl-2-thioxoimidazolidin-4-one

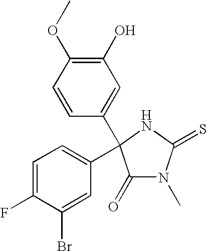

5-[(3-Bromo-4-fluorophenyl)(oxo)acetyl]-2-methoxyphenyl acetate (4.73 g, 12.0 mmol) and N-methylthiourea (2.16 g, 23.9 mmol) were divided equally into 4 microwave vials. Dimethyl sulfoxide (10 mL) and an aqueous solution of potassium hydroxide (5.1 mL, 1.2 M) was added to each vial. The vials were capped and irradiated in a microwave at 100° C. for 9 min. When cooled to room temperature the reaction mixtures were pooled, water (50 mL) was added and pH was adjusted to pH 5 with a 2 M aqueous solution of hydrochloric acid. The aqueous phase was extracted with chloroform and the combined organic extracts were washed with aqueous saturated sodium chloride, dried over magnesium sulfate, filtered and the solvent was reduced in vacuo. Water was added and the extractive work up was repeated with diethyl ether instead of chloroform. Purification by column chromatography, using 5-70% ethyl acetate in heptane as the eluent gave 3.49 g (69% yield) of the title compound: ¹H-NMR (DMSO-d₆): δ 11.53 (br s, 1H), 9.24 (s, 1H), 7.63 (dd, J=6.5, 2.3 Hz, 1H), 7.50-7.38 (m, 2H), 6.94 (d, J=8.3 Hz, 1H), 6.69 (d, J=2.5 Hz, 1H), 6.63 (dd, J=8.4, 2.4 Hz, 1H), 3.75 (s, 3H), 3.16 (s, 3H); MS (ES) m/z 423, 425 [M−H]⁻.

### Example 12

### 5-[4-(3-Bromo-4-fluorophenyl)-1-methyl-5-oxo-2-thioxoimidazolidin-4-yl]-2-methoxyphenyl trifluoromethanesulfonate

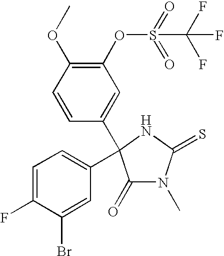

A solution of 5-(3-bromo-4-fluorophenyl)-5-(3-hydroxy-4-methoxyphenyl)-3-methyl-2-thioxoimidazolidin-4-one (515 mg, 1.2 mmol) and 1,1,1-trifluoro-N-phenyl-N-(trifluoromethylsulfonyl)methanesulfonamide (476 mg, 1.3 mmol) in anhydrous dichloromethane (5 mL) purged with argon was cooled to 0° C. Triethylamine (253 μL, 1.3 mmol) was added dropwise over 10 min followed by removal of the ice bath. The reaction mixture was stirred at room temperature for 2 h. Another 0.1 eqv of 1,1,1-trifluoro-N-phenyl-N-(trifluoromethylsulfonyl)methanesulfonamide was added and the mixture was stirred at room temperature over night. The solution was washed with water, aqueous saturated sodium chloride, dried over magnesium sulfate, filtered and the solvent was evaporated in vacuo. Purification by column chromatography, using 0-50% ethyl acetate in heptane as the eluent, gave the title compound in 93% yield: ¹H-NMR (DMSO-d₆) δ 11.69 (s, 1H), 7.59 (dd, J=6.5, 2.3 Hz, 1H), 7.50-7.40 (m, 3H), 7.39-7.31 (m, 2H), 3.91 (s, 3H), 3.17 (s, 3H); MS (ES) m/z 555, 557 [M−H]⁻.

### Example 13

### 5-[2-Amino-4-(3-bromo-4-fluorophenyl)-1-methyl-5-oxo-4,5-dihydro-1H-imidazol-4-yl]-2-methoxyphenyl trifluoromethanesulfonate

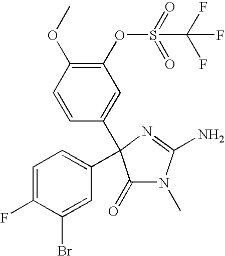

5-[4-(3-Bromo-4-fluorophenyl)-1-methyl-5-oxo-2-thioxoimidazolidin-4-yl]-2-methoxyphenyl trifluoromethanesulfonate (0.623 g, 1.10 mmol) was added to a mixture of methanol (6 mL) and aqueous ammonia (33%, 2 mL). Aqueous t-butyl hydroperoxide (70%, 1.68 mL, 16.8 mmol) was added, and the resulting mixture was stirred at room temperature for 15 h. The solvent was reduced in vacuo. Ethyl acetate was added and the organic phase was washed with water and aqueous saturated sodium chloride, dried over magnesium sulfate, filtered and the solvent was evaporated in vacuo. Purification by column chromatography, using a mixture of ethyl acetate:triethylamine:methanol, 98%:1%:1% to 94%:1%:5% respectively as the eluent, gave 367 mg (61% yield) of the title compound: ¹H-NMR (DMSO-d₆): δ 7.67 (dd, J=6.8, 2.3 Hz, 1H), 7.54 (dd, J=8.8, 2.0 Hz, 1H), 7.49-7.44 (m, 1H), 7.41 (d, J=2.0 Hz, 1H), 7.36-7.27 (m, 2H), 6.86 (br s, 2H), 3.86 (s, 3H), 2.98 (s, 3H); MS (ES) m/z 538, 540 [M−H]⁻.

### Example 14

### 5-[2-Amino-4-(4-fluoro-3-pyrimidin-5-ylphenyl)-1-methyl-5-oxo-4,5-dihydro-[H-imidazol-4-yl]-2-methoxyphenyl trifluoromethanesulfonate 0.5 acetate

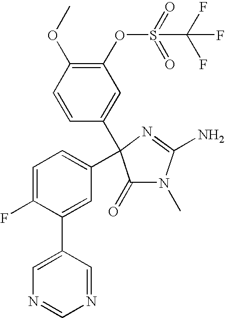

Anhydrous 1,4-dioxane (2.5 mL) was added to 5-[2-amino-4-(3-bromo-4-fluorophenyl)-1-methyl-5-oxo-4,5-dihydro-1H-imidazol-4-yl]-2-methoxyphenyl trifluoromethanesulfonate (109 mg, 0.20 mmol), pyrimidin-5-ylboronic acid (23 mg, 0.18 mmol), bis(triphenylphosphine)palladium(II) dichloride (16 mg, 0.02 mmol) and potassium carbonate (167 mg, 1.20 mmol). The vial was capped, purged with argon and heated at 100° C. in an oil bath for 30 h. When cooled to room temperature the mixture was filtered and the solvent was evaporated in vacuo. The product was purified by preparative HPLC to give 30 mg (28% yield) of the title compound: ¹H-NMR (DMSO-d₆) δ 9.23 (s, 1H), 8.92 (d, J=1.2 Hz, 1H), 7.65-7.59 (m, 2H), 7.58-7.52 (m, 2H), 7.49 (d, J=2.0 Hz, 1H), 7.41-7.34 (m, 1H), 7.29 (d, J=8.8 Hz, 1H), 6.82 (br s, 2H), 3.86 (s, 3H), 2.99 (s, 3H), 1.88 (s, 1.61H); MS (ES) m/z 538 [M−H]⁻.

### Example 15

### 3-Bromo-5-[(4-methoxyphenyl)ethynyl]pyridine

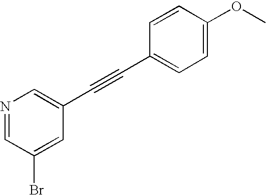

Bis(triphenylphosphine)palladium(II) chloride (6.3 mg, 0.009 mmol) was added to a solution of 3-bromo-5-iodopyridine (0.5 g, 1.76 mmol), 4-ethyne-anisole (0.26 g, 1.93 mmol), copper(I) iodide (2 mg, 0.009 mmol) in triethylamine (2.5 mL) and anhydrous tetrahydrofuran (10 mL) at 0° C. The mixture was stirred over night at room temperature and the solvent was evaporated. Ethyl acetate and water was added and the organic phase was collected, washed with brine, dried over sodium sulfate and concentrated. Purification by column chromatography, using 0-10% ethyl acetate in n-heptane as eluent, gave 0.49 g (96% yield) of the title compound: ¹H NMR (DMSO-d₆) δ 8.70 (m, 2H), 8.25-8.27 (m, 1H), 7.52-7.56 (m, 2H), 7.00-7.04 (m, 2H), 3.81 (s, 3H).

### Example 16

### 1-(5-Bromopyridin-3-yl)-2-(4-methoxyphenyl)ethane-1,2-dione

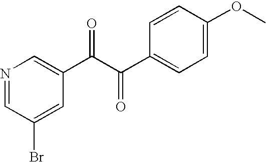

A mixture of 3-bromo-5-[(4-methoxyphenyl)ethynyl]pyridine (0.49 g, 1.7 mmol) and palladium dichloride (30 mg, 0.17 mmol) in dimethyl sulfoxide (17 mL) was heated at 120° C. for 5 h. Upon cooling to room temperature, water was added and the resulting mixture extracted with ethyl acetate. The combined organic phases were washed with water and brine, dried over sodium sulfate and concentrated to give 0.53 g (98% yield) of the title compound: ¹H NMR (DMSO-d₆) δ 9.06 (d, J=2.3 Hz, 1H), 9.02 (d, J=1.5 Hz, 1H), 8.50-8.52 (m, 1H), 7.97-8.01 (m, 2H), 7.13-7.17 (m, 2H), 3.90 (s, 3H).

### Example 17

### 5-(5-Bromopyridin-3-yl)-5-(4-methoxyphenyl)-3-methyl-2-thioxoimidazolidin-4-one

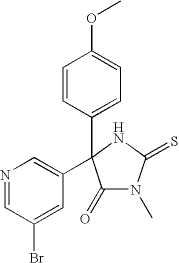

1-(5-Bromopyridin-3-yl)-2-(4-methoxyphenyl)ethane-1,2-dione (0.53 g, 1.66 mmol) and N-methylthiourea (0.3 g, 3.3 mmol) in dimethyl sulfoxide (15 mL) was heated to 100° C. Potassium hydroxide (1.2 M in water, 2.8 mL, 3.3 mmol) was added and the mixture was kept at 100° C. for 30 min. Water was added, the pH adjusted to neutral using hydrochloric acid (2 M) and the mixture was extracted with ethyl acetate. The combined organic phases were washed with water and brine, dried over sodium sulfate and concentrated to give 0.52 g (80% yield) of the title compound: ¹H NMR (DMSO-d₆) δ 8.75 (d, J=2.3 Hz, 1H), 8.53 (d, J=2.0 Hz, 1H), 7.95 (t, J=2.1 Hz, 1H), 7.19-7.23 (m, 2H), 6.98-7.02 (m, 2H), 3.76 (s, 3H), 3.17 (s, 3H).

### Example 18

### 5-(5-Bromopyridin-3-yl)-5-(4-hydroxyphenyl)-3-methyl-2-thioxoimidazolidin-4-one

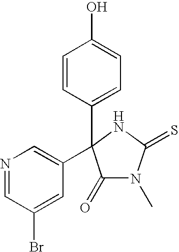

5-(5-Bromopyridin-3-yl)-5-(4-methoxyphenyl)-3-methyl-2-thioxoimidazolidin-4-one (0.52 g, 1.33 mmol) was dissolved in dichloromethane (15 mL) and cooled to 0° C. Boron tribromide (0.19 mL, 2 mmol) was added and the mixture was allowed to reach room temperature. Additional boron tribromide (0.19 mL, 2 mmol) was added and the mixture was left over night. Water and saturated ammonium hydroxide was added and the mixture was extracted with ethyl acetate. The combined organic extracts were washed with brine, dried over sodium sulfate and concentrated to give 0.49 g (97% yield) of the title compound: ¹H NMR (DMSO-d₆) δ 9.77 (s, 1H), 8.74 (d, J=2.0 Hz, 1H), 8.54 (d, J=2.0 Hz, 1H), 7.95 (t, J=2.1 Hz, 1H), 7.05-7.09 (m, 2H), 6.78-6.82 (m, 2H), 3.17 (s, 3H).

### Example 19

### 4-[4-(5-Bromopyridin-3-yl)-1-methyl-5-oxo-2-thioxoimidazolidin-4-yl]phenyl methanesulfonate

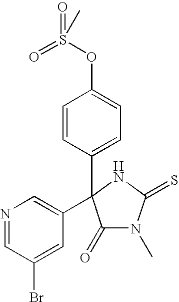

Methanesulfonylchloride (0.14 mL, 1.8 mmol) was added to 5-(5-bromopyridin-3-yl)-5-(4-hydroxyphenyl)-3-methyl-2-thioxoimidazolidin-4-one (0.49 g, 1.29 mmol) and triethylamine (0.35 mL, 2.5 mmol) in dichloromethane (8 mL). The mixture was stirred over night, saturated aqueous sodium bicarbonate was added and the mixture was extracted with ethyl acetate. The combined organic extracts were dried over sodium sulfate and concentrated. Purification by column chromatography, using 15-40% ethyl acetate in n-heptane as the eluent, gave 0.36 g (60% yield) of the title compound: ¹H NMR (DMSO-d₆) δ 11.72 (s, 1H), 8.77 (d, J=2.3 Hz, 1H), 8.55 (d, J=2.3 Hz, 1H), 8.00 (t, J=2.1 Hz, 1H), 7.44 (s, 4H), 3.41 (s, 3H), 3.18 (s, 3H).

### Example 20

### 4-[2-Amino-4-(5-bromopyridin-3-yl)-1-methyl-5-oxo-4,5-dihydro-1H-imidazol-4-yl]phenyl methanesulfonate

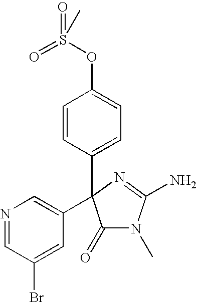

4-[4-(5-Bromopyridin-3-yl)-1-methyl-5-oxo-2-thioxoimidazolidin-4-yl]phenyl methanesulfonate (0.36 g, 0.73 mmol) was dissolved in a mixture of methanol (8 mL) and aqueous ammonium hydroxide (conc., 4 mL). tert-Butyl hydroperoxide (1.5 mL, 11 mmol, 70% in water) was added and the mixture was heated at 35° C. for 5 h. Most of the methanol was evaporated and water and saturated aqueous sodium bicarbonate was added. The mixture was extracted with ethyl acetate and the combined organic phases were washed with water and brine, dried over sodium sulfate and concentrated to give 0.34 g (quantitative yield) of the title compound: ¹H NMR (DMSO-d₆) δ 10.73 (s, 1H), 8.61-8.64 (m, 2H), 8.01 (t, J=2.0 Hz, 1H), 7.54-7.57 (m, 2H), 7.30-7.33 (m, 2H), 6.89 (br. s., 2H), 3.36 (s, 3H), 3.00 (s, 3H).

### Example 21

### 4-{2-Amino-4-[5-(2-fluoro-5-methoxyphenyl)pyridin-3-yl]-1-methyl-5-oxo-4,5-dihydro-1H-imidazol-4-yl}phenyl methanesulfonate 0.5 acetate

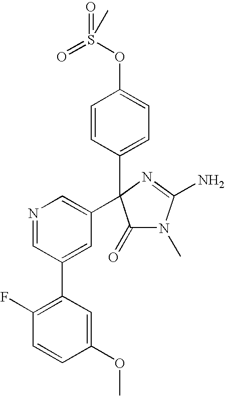

4-[2-Amino-4-(5-bromopyridin-3-yl)-1-methyl-5-oxo-4,5-dihydro-1H-imidazol-4-yl]phenyl methanesulfonate (100 mg, 0.22 mmol), 2-fluoro-5-methoxyphenylboronic acid (52 mg, 0.3 mmol), [1,1′-bis(diphenylphosphino)ferrocene]palladium(II) chloride dichloromethane adduct (18 mg, 0.022 mmol), potassium carbonate (182 mg, 1.23 mmol) and anhydrous tetrahydrofuran (3 mL) were mixed and irradiated under an atmosphere of argon in a microwave at 130° C. for 2 h. When cooled to room temperature the mixture was filtered and dimethyl sulfoxide (500 μL) was added. The solution was concentrated in vacuo to remove the tetrahydrofuran and purified by preparative HPLC to give 20 mg (18% yield) of the title compound: ¹H NMR (DMSO-d₆) δ 8.64-8.68 (m, 2H), 8.00-8.03 (m, 1H), 7.58-7.63 (m, 2H), 7.25-7.34 (m, 3H), 6.99-7.07 (m, 2H), 6.86 (br. s., 2H), 3.79 (s, 3H), 3.36 (s, 3H), 3.01 (s, 3H), 1.91 (s, 1.5H).

### Example 22

### 4-[2-amino-4-(2′-fluoro-3,3′-bipyridin-5-yl)-1-methyl-5-oxo-4,5-dihydro-1H-imidazol-4-yl]phenyl methanesulfonate 0.5 acetate

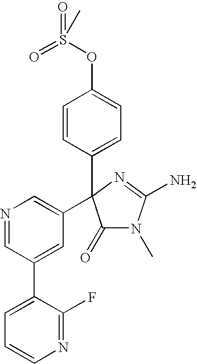

The title compound was synthesized as described for Example 21 in 24% yield starting from 2-fluoropyridine-3-boronic acid and 4-[2-amino-4-(5-bromopyridin-3-yl)-1-methyl-5-oxo-4,5-dihydro-1H-imidazol-4-yl]phenyl methanesulfonate: ¹H NMR (DMSO-d₆) δ 8.70-8.72 (m, 2H), 8.29-8.32 (m, 1H), 8.13-8.19 (m, 1H), 8.08 (q, J=1.9 Hz, 1H), 7.58-7.63 (m, 2H), 7.49-7.53 (m, 1H), 7.30-7.34 (m, 2H), 6.88 (br. s., 2H), 3.36 (s, 3H), 3.01 (s, 3H), 1.89 (s, 1.5H).

### Example 23

### 2-Chloro-4-[(4-methoxyphenyl)ethynyl]pyridine

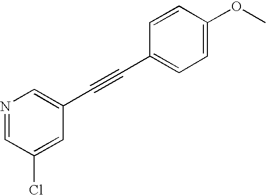

Tri-tert-butylphosphine (1.57 mL, 0.78 mmol, 10% solution in hexane) was added to 4-bromo-2-chloropyridine (2.5 g, 13 mmol), 4-ethyneanisole (1.72 g, 13 mmol), bis(benzonitrile)palladium(II) chloride (150 mg, 0.39 mmol) and copper(I) iodide (49 mg, 0.26 mmol) in triethylamine (4.5 mL) and anhydrous dioxane (20 mL) at 0° C. The mixture was stirred over night at room temperature, water was added and the mixture was extracted with dichloromethane. The combined organic phases were dried over sodium sulfate and concentrated. Purification by column chromatography, using 0-8% ethyl acetate in n-heptane as the eluent, gave 2.57 g (81% yield) of the title compound: ¹H NMR (DMSO-d₆) δ 8.42-8.44 (m, 1H), 7.65-7.67 (m, 1H), 7.55-7.59 (m, 2H), 7.51 (m, 1H), 7.01-7.05 (m, 2H), 3.81 (s, 3H); MS (ESI) m/z 244/246 [M+1]⁺.

### Example 24

### 1-(2-Chloropyridin-4-yl)-2-(4-methoxyphenyl)ethane-1,2-dione

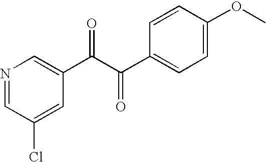

2-Chloro-4-[(4-methoxyphenyl)ethynyl]pyridine (1.96 g, 8 mmol) and palladium dichloride (141 mg, 0.8 mmol) in dimethyl sulfoxide (35 mL) was heated at 130° C. for 4 h. Upon cooling to room temperature water was added and the mixture extracted with ethyl acetate. The combined organic phases were washed with water and brine, dried over sodium sulfate and concentrated. Purification by column chromatography, using 0-15% ethyl acetate in n-heptane as the eluent, gave 0.86 g (39% yield) of the title compound: ¹H NMR (DMSO-d₆) δ 8.71 (d, J=5.0 Hz, 1H), 7.97-8.01 (m, 2H), 7.90-7.92 (m, 1H), 7.81-7.83 (m, 1H), 7.14-7.18 (m, 2H), 3.90 (s, 3H).

### Example 25

### 1-(2′-Fluoro-2,3′-bipyridin-4-yl)-2-(4-methoxyphenyl)ethane-1,2-dione

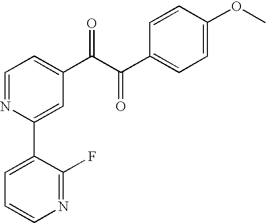

1-(2-Chloropyridin-4-yl)-2-(4-methoxyphenyl)ethane-1,2-dione (0.2 g, 0.72 mmol), 2-fluoropyridine-3-boronic acid (134 mg, 0.95 mmol) and tetrakis(triphenylphosphine)palladium (84 mg, 0.073 mmol) were dissolved in 1,2-dimethoxyethane (3.5 mL) and aqueous sodium carbonate (1.1 mL, 2.2 mmol, 2 M in water). The mixture was irradiated in a microwave at 130° C. for 45 min. When cooled to room temperature water was added and the mixture was extracted with ethyl acetate. The combined organic extracts were washed with brine, dried over sodium sulfate and concentrated. Purification by column chromatography, using 0-20% ethyl acetate in n-heptane as the eluent, gave 0.16 g (65% yield) of the title compound: ¹H NMR (DMSO-d₆) δ 9.03-9.05 (m, 1H), 8.53-8.58 (m, 1H), 8.35-8.38 (m, 1H), 8.26-8.28 (m, 1H), 7.98-8.02 (m, 2H), 7.85 (m, 1H), 7.56 (m, 1H), 7.15-7.19 (m, 2H), 3.90 (s, 3H); MS (ES) m/z 337 [M+1]⁺.

### Example 26

### 5-(2′-Fluoro-2,3′-bipyridin-4-yl)-5-(4-methoxyphenyl)-3-methyl-2-thioxoimidazolidin-4-one

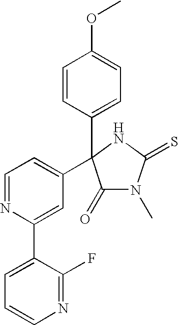

The title compound was synthesized as described for Example 17 in quantitative yield, starting from 1-(2′-fluoro-2,3′-bipyridin-4-yl)-2-(4-methoxyphenyl)ethane-1,2-dione: ¹H NMR (DMSO-d₆) δ 8.79-8.82 (m, 1H), 8.45-8.50 (m, 1H), 8.32-8.35 (m, 1H), 7.88-7.90 (m, 1H), 7.51-7.56 (m, 1H), 7.46 (m, 1H), 7.24-7.28 (m, 2H), 6.98-7.02 (m, 2H), 3.75 (s, 3H), 3.18 (s, 3H); MS (ES) m/z 409 [M+1]⁺.

### Example 27

### 5-(2′-Fluoro-2,3′-bipyridin-4-yl)-5-(4-hydroxyphenyl)-3-methyl-2-thioxoimidazolidin-4-one

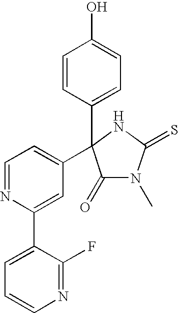

The title compound was synthesized as described for Example 18 in quantitative yield, starting from 5-(2′-fluoro-2,3′-bipyridin-4-yl)-5-(4-methoxyphenyl)-3-methyl-2-thioxoimidazolidin-4-one but using 4.5 equiv of boron tribromide: MS (ES) m/z 395 [M+1]⁺.

### Example 28

### 4-[4-(2′-Fluoro-2,3′-bipyridin-4-yl)-1-methyl-5-oxo-2-thioxoimidazolidin-4-yl]phenyl methanesulfonate

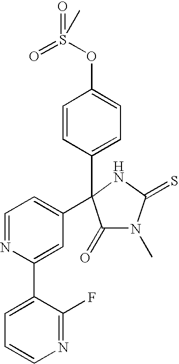

The title compound was synthesized as described for Example 19 in 78% yield, starting from 5-(2′-fluoro-2,3′-bipyridin-4-yl)-5-(4-methoxyphenyl)-3-methyl-2-thioxoimidazolidin-4-one: ¹H NMR (DMSO-d₆) δ 11.87 (s, 1H), 8.81-8.84 (m, 1H), 8.48 (m, 1H), 8.32-8.35 (m, 1H), 7.91-7.93 (m, 1H), 7.52-7.56 (m, 1H), 7.44-7.50 (m, 5H), 3.40 (s, 3H), 3.19 (s, 3H); MS (ES) m/z 473 [M+1]⁺.

### Example 29

### 4-[2-Amino-4-(2′-fluoro-2,3′-bipyridin-4-yl)-1-methyl-5-oxo-4,5-dihydro-1H-imidazol-4-yl]phenyl methanesulfonate 0.25 acetate

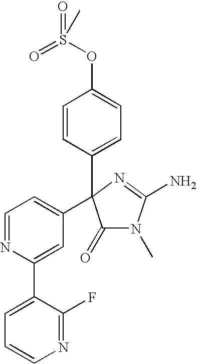

4-[4-(2′-Fluoro-2,3′-bipyridin-4-yl)-1-methyl-5-oxo-2-thioxoimidazolidin-4-yl]phenyl methanesulfonate (0.27 g, 0.57 mmol) was dissolved in methanol (8 mL) and aqueous ammonium hydroxide (saturated, 4 mL). tert-Butyl hydroperoxide (1.2 mL, 8.6 mmol, 70% in water) was added and the mixture was heated at 35° C. for 3 h. The mixture was filtered and purified by preparative HPLC to give 0.078 g (28% yield) of the title compound: ¹H NMR (DMSO-d₆) δ 8.67-8.70 (m, 1H), 8.47 (m, 1H), 8.29-8.32 (m, 1 H), 8.01-8.03 (m, 1H), 7.59-7.63 (m, 2H), 7.49-7.55 (m, 2H), 7.30-7.34 (m, 2H), 6.90 (br. s., 2H), 3.35 (s, 3H), 3.00 (s, 3H), 1.90 (s, 0.7H); MS (ES) m/z 456 [M+1]⁺.

### Example 30

### 3-{4-[(4-Methoxyphenyl)(oxo)acetyl]pyridin-2-yl}benzonitrile

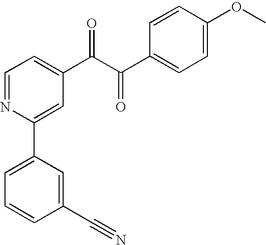

The title compound was synthesized as described for Example 25 in 67% yield, starting from 1-(2-chloropyridin-4-yl)-2-(4-methoxyphenyl)ethane-1,2-dione and 3-cyanobenzene boronic acid: ¹H NMR (DMSO-d₆) δ 8.99 (m, 1H), 8.59-8.62 (m, 1H), 8.47-8.51 (m, 1H), 8.45-8.47 (m, 1H), 7.98-8.02 (m, 2H), 7.95-7.97 (m, 1H), 7.81 (m, 1H), 7.74 (t, J=7.8 Hz, 1H), 7.15-7.19 (m, 2H), 3.90 (s, 3H); MS (ES) m/z 343 [M+1]⁺.

### Example 31

### 3-{4-[4-(4-Methoxyphenyl)-1-methyl-5-oxo-2-thioxoimidazolidin-4-yl]pyridin-2-yl}benzonitrile

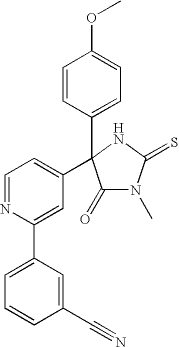

The title compound was synthesized as described for Example 17 in 28% yield, starting from 3-{4-[(4-methoxyphenyl)(oxo)acetyl]pyridin-2-yl}benzonitrile. Purification by column chromatography, using 0-40% ethyl acetate in n-heptane as the eluent: ¹H NMR (DMSO-d₆) δ 8.76-8.79 (m, 1H), 8.39-8.41 (m, 1H), 8.32-8.35 (m, 1H), 8.00-8.02 (m, 1H), 7.93-7.96 (m, 1H), 7.75 (t, J=7.9 Hz, 1H), 7.49 (m, 1H), 7.22-7.26 (m, 2H), 6.97-7.01 (m, 2H), 3.74 (s, 3H), 3.19 (s, 3H); MS (ES) m/z 415 [M+1]⁺.

### Example 32

### 3-{4-[4-(4-Hydroxyphenyl)-1-methyl-5-oxo-2-thioxoimidazolidin-4-yl]pyridin-2-yl}benzonitrile

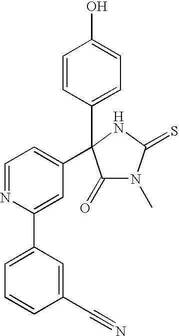

The title compound was synthesized as described for Example 18 in quantitative yield, starting from 3-{4-[4-(4-methoxyphenyl)-1-methyl-5-oxo-2-thioxoimidazolidin-4-yl]pyridin-2-yl}benzonitrile but using 4 equiv of boron tribromide: ¹H NMR (DMSO-d₆) δ 8.75-8.77 (m, 1H), 8.39-8.41 (m, 1H), 8.31-8.35 (m, 1H), 8.00-8.02 (m, 1H), 7.93-7.96 (m, 1H), 7.74 (t, J=7.9 Hz, 1H), 7.49 (m, 1H), 7.08-7.12 (m, 2H), 6.77-6.81 (m, 2H), 3.18 (s, 3H).

### Example 33

### 4-{4-[2-(3-Cyanophenyl)pyridin-4-yl]-1-methyl-5-oxo-2-thioxoimidazolidin-4-yl}phenyl methanesulfonate

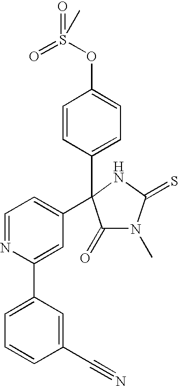

The title compound was synthesized as described for Example 19 in 95% yield, starting from 3-{4-[4-(4-hydroxyphenyl)-1-methyl-5-oxo-2-thioxoimidazolidin-4-yl]pyridin-2-yl}benzonitrile: MS (ES) m/z 479 [M+1]⁺.

### Example 34

### 4-{2-Amino-4-[2-(3-cyanophenyl)pyridin-4-yl]-1-methyl-5-oxo-4,5-dihydro-1H-imidazol-4-yl}phenyl methanesulfonate 0.25 acetate

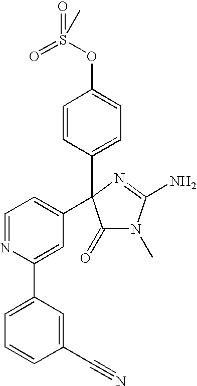

The title compound was synthesized as described for Example 29 in 48% yield, starting from 4-{4-[2-(3-cyanophenyl)pyridin-4-yl]-1-methyl-5-oxo-2-thioxoimidazolidin-4-yl}phenyl methanesulfonate: ¹H NMR (DMSO-d₆) δ 8.63-8.66 (m, 1H), 8.35-8.37 (m, 1H), 8.25-8.29 (m, 1H), 8.05-8.07 (m, 1H), 7.90-7.93 (m, 1H), 7.72 (t, J=7.9 Hz, 1H), 7.60-7.65 (m, 2H), 7.52 (m, 1H), 7.29-7.33 (m, 2H), 3.35 (s, 3H), 3.01 (s, 3H), 1.90 (s, 0.7H); MS (ES) m/z 462 [M+1]⁺.

### Example 35

### 1-(2-Chloropyridin-4-yl)-2-(4-hydroxyphenyl)ethane-1,2-dione

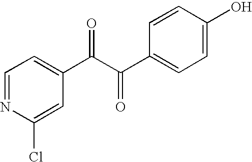

The title compound was synthesized as described for Example 18 in 89% yield, starting from 1-(2-chloropyridin-4-yl)-2-(4-methoxyphenyl)ethane-1,2-dione but using 5 eqv of boron tribromide: MS (ES) m/z 262 [M+1]⁺.

### Example 36

### 1-(4-Hydroxyphenyl)-2-[2-(3-methoxyphenyl)pyridin-4-yl]ethane-1,2-dione

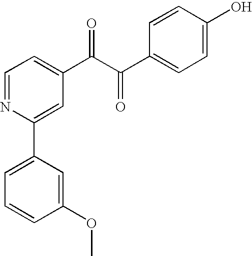

The title compound was synthesized as described for Example 25 in 43% yield, starting from 1-(2-chloropyridin-4-yl)-2-(4-hydroxyphenyl)ethane-1,2-dione and 3-methoxybenzene boronic acid: ¹H NMR (DMSO-d₆) δ 8.93-8.96 (m, 1H), 8.26-8.28 (m, 1H), 7.86-7.90 (m, 2H), 7.70 (m, 1H), 7.65-7.69 (m, 2H), 7.44 (t, J=8.2 Hz, 1H), 7.05-7.09 (m, 1H), 6.94-6.98 (m, 2H), 3.84 (s, 3H).

### Example 37

### 5-(4-Hydroxyphenyl)-5-[2-(3-methoxyphenyl)pyridin-4-yl]-3-methyl-2-thioxoimidazolidin-4-one

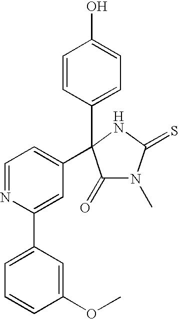

The title compound was synthesized as described for Example 17 in quantitative yield, starting from 1-(4-hydroxyphenyl)-2-[2-(3-methoxyphenyl)pyridin-4-yl]ethane-1,2-dione: ¹H NMR (DMSO-d₆) δ 11.66 (s, 1H), 9.74 (s, 1H), 8.70-8.72 (m, 1H), 7.88-7.90 (m, 1H), 7.56-7.58 (m, 1H), 7.52-7.55 (m, 1H), 7.40-7.45 (m, 2H), 7.08-7.12 (m, 2H), 7.03-7.06 (m, 1H), 6.77-6.81 (m, 2H), 3.82 (s, 3H), 3.18 (s, 3H); MS (ES) m/z 406 [M+1]⁺.

### Example 38

### 4-{4-[2-(3-Methoxyphenyl)pyridin-4-yl]-1-methyl-5-oxo-2-thioxoimidazolidin-4-yl}phenyl methanesulfonate

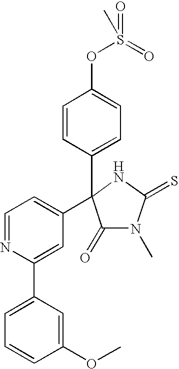

The title compound was synthesized as described for Example 19 in 46% yield, starting from 5-(4-hydroxyphenyl)-5-[2-(3-methoxyphenyl)pyridin-4-yl]-3-methyl-2-thioxoimidazolidin-4-one. Purification by column chromatography, using 0-50% ethyl acetate in n-heptane as the eluent: ¹H NMR (DMSO-d₆) δ 11.83 (s, 1H), 8.74 (d, J=5.3 Hz, 1H), 7.90-7.93 (m, 1H), 7.54-7.59 (m, 2H), 7.42-7.49 (m, 6H), 7.03-7.07 (m, 1H), 3.83 (s, 3H), 3.40 (s, 3H), 3.20 (s, 3H).

### Example 39

### 4-{2-Amino-4-[2-(3-methoxyphenyl)pyridin-4-yl]-1-methyl-5-oxo-4,5-dihydro-1H-imidazol-4-yl}phenyl methanesulfonate 0.25 acetate

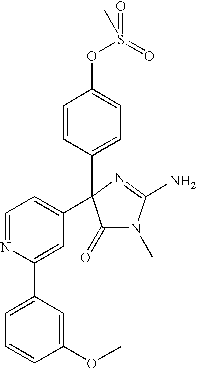

The title compound was synthesized as described for Example 29 in 16% yield, starting from 4-{4-[2-(3-methoxyphenyl)pyridin-4-yl]-1-methyl-5-oxo-2-thioxoimidazolidin-4-yl}phenyl methanesulfonate: ¹H NMR (DMSO-d₆) δ 8.60 (d, J=5.0 Hz, 1H), 7.94-7.96 (m, 1H), 7.59-7.63 (m, 2H), 7.50-7.52 (m, 1H), 7.39-7.48 (m, 3H), 7.29-7.33 (m, 2H), 7.00-7.04 (m, 1H), 6.87 (br. s., 2H), 3.81 (s, 3H), 3.35 (s, 3H), 3.01 (s, 3H), 1.89 (s, 0.9H); MS (ES) m/z 467 [M+1]⁺.

### Example 40

### [4-Methoxy-3-(trifluoromethyl)benzyl](triphenyl)phosphonium bromide

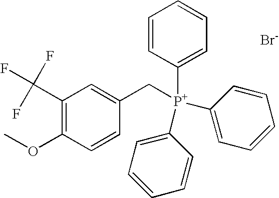

A mixture of 4-(bromomethyl)-1-methoxy-2-(trifluoromethyl)benzene (5 g, 18 mmol) and triphenylphosphine (4.9 g, 18 mmol) in anhydrous toluene (15 mL) was heated at 85° C. over night. After the mixture was cooled, the precipitate was collected by filtration, washed with toluene and diethyl ether, and dried in vacuo at room temperature over night to afford 9 g (94% yield) of the title compound: ¹H-NMR (DMSO-d₆) δ 7.95-7.89 (m, 3H), 7.79-7.73 (m, 6H), 7.73-7.65 (m, 6H), 7.36-7.31 (m, 1H), 7.19 (d, J=8.8 Hz, 1H), 7.02-6.99 (m, 1H), 5.18 (d, J=15.1 Hz, 2H), 3.84 (s, 3H); MS (ES) m/z 451 M⁺.

### Example 41

### (4-Methoxy-3-methylbenzyl)(triphenyl)phosphonium chloride

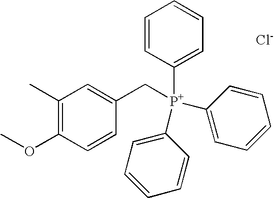

The title compound was synthesized as described for Example 40 in 60% yield, starting from 4-(chloromethyl)-1-methoxy-2-methylbenzene: ¹H-NMR (DMSO-d₆) δ 7.94-7.87 (m, 3H), 7.78-7.71 (m, 6H), 7.70-7.62 (m, 6H), 6.86-6.78 (m, 2H), 6.58-6.55 (m, 1H), 5.02 (d, J=15.1 Hz, 2H), 3.72 (s, 3H), 1.91 (s, 3H); MS (ES) m/z 397 M⁺.

### Example 42

### [4-(Difluoromethoxy)benzyl](triphenyl)phosphonium bromide

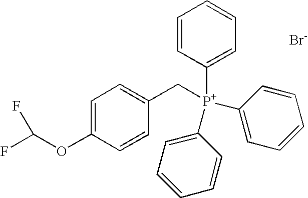

The title compound was synthesized as described for Example 40 in 96% yield, starting from 1-(bromomethyl)-4-(difluoromethoxy)benzene: ¹H-NMR (DMSO-d₆) δ 7.95-7.88 (m, 3H), 7.79-7.72 (m, 6H), 7.71-7.63 (m, 6H), 7.20 (t, J=73.9 Hz, 1H), 7.08-6.98 (m, 4H), 5.16 (d, J=15.6 Hz, 2H); MS (ESI) m/z 419 M⁺.

### Example 43

### Triphenyl[4-(trifluoromethoxy)benzyl]phosphonium bromide

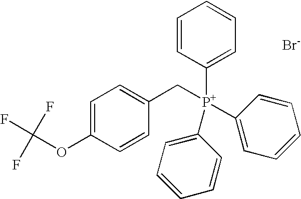

The title compound was synthesized as described for Example 40 in 93% yield, starting from 1-(bromomethyl)-4-(trifluoromethoxy)benzene: ¹H-NMR (DMSO-d₆) δ 7.95-7.88 (m, 3H), 7.78-7.72 (m, 6H), 7.72-7.64 (m, 6H), 7.26 (d, J=8.8 Hz, 2H) 7.09 (dd, J=8.8, 2.5 Hz, 2H), 5.22 (d, J=15.8 Hz, 2H); MS (ESI) m/z 437 M⁺.

### Example 44

### 1-(3-Bromophenyl)-2-[4-methoxy-3-(trifluoromethyl)phenyl]ethane-1,2-dione

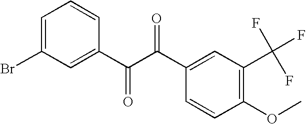

A mixture of [4-methoxy-3-(trifluoromethyl)benzyl](triphenyl)phosphonium bromide (9 g, 16.9 mmol) in anhydrous toluene (60 mL) at 0° C. is treated dropwise with n-butyllithium (6.9 mL, 2.5 M in hexanes, 17.2 mmol), the reaction mixture was allowed to warm up to room temperature and stirred for 2 h. The mixture was cooled to 0° C. and treated with a solution of 3-bromobenzoyl chloride (1.85 g, 8.4 mmol) in anhydrous toluene (5 mL), allowed to warm up to room temperature and stirred for 2 h. Water (25 mL) was added and the reaction mixture was concentrated in vacuo. The resulting residue was dispersed in acetone (150 mL) and water (25 mL), treated with magnesium sulfate (8.6 g, 72 mmol) and potassium permanganate (2.5 g, 15.9 mmol) and stirred vigorously at 45° C. over night. The mixture was filtered, the filtrate was diluted with ethyl acetate, washed with water and brine, dried over magnesium sulfate and the solvent was evaporated. This residue was purified by column chromatography, using heptane/ethyl acetate (50:50) as the eluent, to afford 1.9 g (58% yield) of title compound: ¹H-NMR (DMSO-d₆) δ 8.24 (dd, J=8.8, 2.3 Hz, 1H), 8.20-8.18 (m, 1H), 8.11 (t, J=1.8 Hz, 1H), 8.02-7.97 (m, 1H), 7.97-7.93 (m 1 H), 7.57 (t, J=7.9 Hz, 1H), 7.47 (d, J=8.8 Hz, 1H), 4.04 (s, 3H).

### Example 45

### 1-(3-Bromophenyl)-2-(4-methoxy-3-methylphenyl)ethane-1,2-dione

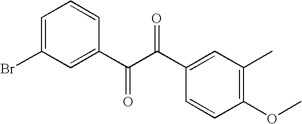

The title compound was synthesized as described for Example 44 in 55% yield, starting from (4-methoxy-3-methylbenzyl)(triphenyl)phosphonium chloride: ¹H-NMR (DMSO-d₆) δ 8.04-8.02 (m, 1H), 8.02-7.97 (m, 1H), 7.89-7.84 (m, 1H), 7.82-7.76 (m, 2H), 7.58 (t, J=7.9 Hz, 1H), 7.15 (d, J=8.5 Hz, 1H), 3.92 (s, 3H), 2.20 (s, 3H).

### Example 46

### 1-(3-Bromophenyl)-2-[4-(difluoromethoxy)phenyl]ethane-1,2-dione

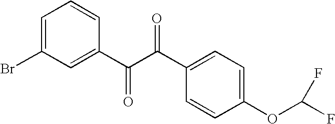

The title compound was synthesized as described for Example 44 in 60% yield, starting from [4-(difluoromethoxy)benzyl](triphenyl)phosphonium bromide: ¹H-NMR (DMSO-d₆) δ 8.10-8.07 (m, 1H), 8.07-8.02 (m, 2H), 8.02-7.98 (m, 1H), 7.94-7.89 (m, 1H), 7.58 (t, J=7.9 Hz, 1H), 7.47 (t, J=73.0 Hz, 1H), 7.42-7.36 (m, 2H); MS (ESI) m/z 353/355 [M-1]⁻.

### Example 47

### 1-(3-Bromophenyl)-2-[4-(trifluoromethoxy)phenyl]ethane-1,2-dione

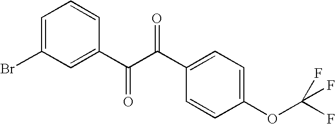

The title compound was synthesized as described for Example 44 in 73% yield, starting from triphenyl[4-(trifluoromethoxy)benzyl]phosphonium bromide (described in: Maya, A. et al. J. Med. Chem. 2005, 48, 556-568): ¹H-NMR (DMSO-d₆) δ 8.15-8.10 (m 3H), 8.03-7.99 (m, 1H), 7.97-7.93 (m, 1H), 7.63-7.57 (m, 3H); MS (ESI) m/z 371/373 [M−1]⁻.

### Example 48

### 2-Amino-5-(3-bromophenyl)-5-[4-methoxy-3-(trifluoromethyl)phenyl]-3-methyl-3,5-dihydro-4H-imidazol-4-one

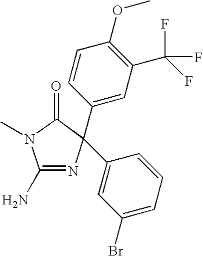

A mixture of 1-(3-bromophenyl)-2-[4-methoxy-3-(trifluoromethyl)phenyl]ethane-1,2-dione (1.9 g, 4.9 mmol) and 1-methylguanidine hydrochloride (2.4 g, 22 mmol) in dioxane (50 mL) and ethanol (50 mL) was stirred at room temperature for 15 min and a solution of sodium carbonate (2.3 g, 22 mmol) in water (8 mL) was added. The resulting mixture was heated at 85° C. for 45 min, cooled to room temperature, and concentrated in vacuo. The resulting residue was partitioned between dichloromethane and water. The organic phase was separated, washed with water and brine, dried over sodium sulfate and concentrated in vacuo. Purification by column chromatography, using acetonitrile/triethylamine (95:5) as the eluent, afforded 1.6 g (74% yield) of the title compound: ¹H-NMR (DMSO-d₆) δ 7.75-7.67 (m, 2H), 7.59-7.55 (m, 1H), 7.47-7.41 (m, 2H), 7.29 (t, J=7.9 Hz, 1H), 7.24 (d, J=8.5 Hz, 1H), 6.82 (br s, 2H), 3.85 (s, 3H), 2.98 (s, 3H); MS (ESI) m/z 442/444 [M+1]⁺.

### Example 49

### 2-Amino-5-(3-bromophenyl)-5-(4-methoxy-3-methylphenyl)-3-methyl-3,5-dihydro-4H-imidazol-4-one

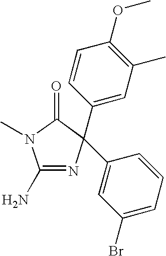

The title compound was synthesized as described for Example 48 in 79% yield, starting from 1-(3-bromophenyl)-2-(4-methoxy-3-methylphenyl)ethane-1,2-dione: ¹H-NMR (DMSO-d₆) δ 7.59-7.55 (m, 1H), 7.43 (t, J=8.8 Hz, 2H), 7.26 (t, J=7.9 Hz, 1H), 7.20 (dd, J=8.5, 2.0 Hz, 1H), 7.17-7.14 (m, 1H), 6.85 (d, J=8.8 Hz, 1H), 6.67 (br s, 2H), 3.74 (s, 3H), 2.97 (s, 3H), 2.07 (s, 3H); MS (ESI) m/z 388, 390 [M+1]⁺.

### Example 50

### 2-Amino-5-(3-bromophenyl)-5-[4-(difluoromethoxy)phenyl]-3-methyl-3,5-dihydro-4H-imidazol-4-one

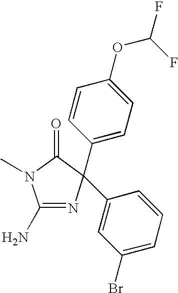

The title compound was synthesized as described for Example 48 in 66% yield, starting from 1-(3-bromophenyl)-2-[4-(difluoromethoxy)phenyl]ethane-1,2-dione: ¹H-NMR (DMSO-d₆) δ 7.62-7.58 (m, 1H), 7.50-7.41 (m, 4H), 7.28 (t, J=7.9 Hz, 1H), 7.17 (t, J=74.2 Hz, 1H), 7.15-7.08 (m, 2H), 6.75 (br s, 2H), 2.98 (s, 3H); MS (ESI) m/z 410, 412 [M+1]⁺.

### Example 51

### 2-Amino-5-(3-bromophenyl)-3-methyl-5-[4-(trifluoromethoxy)phenyl]-3,5-dihydro-4H-imidazol-4-one

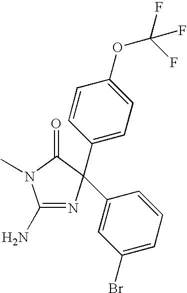

The title compound was synthesized as described for Example 48 in 46% yield, starting from 1-(3-bromophenyl)-2-[4-(trifluoromethoxy)phenyl]ethane-1,2-dione: ¹H-NMR (DMSO-d₆) δ 7.62 (t, J=1.8 Hz, 1H), 7.58-7.52 (m, 2H), 7.50-7.43 (m, 2H), 7.35-7.26 (m, 3H), 6.97 (br s, 2H), 2.99 (s, 3H); MS (ESI) m/z 428, 430 [M+1]⁺.

### Example 52

### 2-Amino-5-(3-bromophenyl)-5-(4-methoxyphenyl)-3-methyl-3,5-dihydro-4H-imidazol-4-one

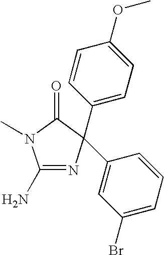

The title compound was synthesized as described for Example 48 in 94% yield, starting from 1-(3-bromophenyl)-2-(4-methoxyphenyl)ethane-1,2-dione (described in: Buck J. S, and Ide W. S. J. Am. Chem. Soc. 1930, 52, 4107-4109): ¹H-NMR (DMSO-d₆) δ 7.60-7.56 (m, 1H), 7.47-7.40 (m, 2H), 7.35-7.29 (m, 2H), 7.26 (t, J=7.9 Hz, 1H), 6.89-6.83 (m, 2H), 6.68 (br s, 2H), 3.71 (s, 3H), 2.97 (s, 3H); MS (ESI) m/z 374, 376 [M+1]⁺.

### Example 53

### 3-Methoxy-5-(4,4,5,5-tetramethyl-1,3,2-dioxaborolan-2-yl)phenol

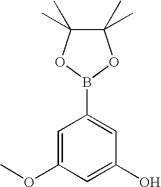

Anhydrous 1,2-dimethoxyethane (2×15 mL) was added to two vials each charged with 3-chloro-5-methoxyphenol (1.18 g, 7.5 mmol), tris(dibenzylideneaceton)dipalladium(0) (0.200 mg, 0.21 mmol), tricyclohexylphosphine (0.300 mg, 1.05 mmol), potassium acetate (1.1 mg, 11 mmol) and 4,4,4′,4′,5,5,5′,5′-octamethyl-2,2′-bi-1,3,2-dioxaborolane (2.08 g, 8.2 mmol). The vials were capped, purged with argon and irradiated in a microwave at 150° C. for 2 h. When cooled to room temperature the reaction mixtures were pooled, diluted with water and extracted with diethyl ether. The combined organics were concentrated in vacuo and purified by column chromatography, using 0-10% acetonitrile in dichloromethane as the eluent, to give 2.6 g (69% yield) of the title compound: ¹H-NMR (DMSO-d₆) δ 9.36 (s, 1H), 6.69 (d, J=2.3 Hz, 1H), 6.61 (d, J=2.0 Hz, 1H), 6.41 (t, J=2.4 Hz, 1H), 3.69 (s, 3H), 1.27 (s, 12H); MS (ESI) m/z 251 [M+1]⁺.

### Example 54

### 3-Methoxy-5-(4,4,5,5-tetramethyl-1,3,2-dioxaborolan-2-yl)phenyl methanesulfonate

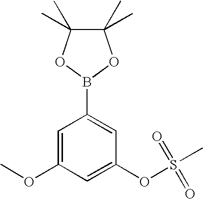

To a stirred solution of 3-methoxy-5-(4,4,5,5-tetramethyl-1,3,2-dioxaborolan-2-yl)phenol (2.6 g, 10.4 mmol) in dichloromethane (50 mL) was added triethylamine (3.1 g, 31.2 mmol) followed by methanesulfonyl chloride (1.2 mL, 15.6 mmol) at 0° C. under an atmosphere of argon. The reaction mixture was stirred at 0° C. for 25 min and then allowed to reach room temperature and stirred for 18 h. The solvent was evaporated and the residue was purified by column chromatography, using a gradient of dichloromethane/acetonitrile (100:0 to 95:5) as the eluent, to give 2.1 g (62% yield) of the title compound: ¹H-NMR (CDCl₃) δ 7.30 (d, J=2.3 Hz, 1H), 7.28 (d, J=2.0 Hz, 1H), 6.96 (t, J=2.4 Hz, 1H), 3.86 (s, 3H), 3.16 (s, 3H), 1.35 (s, 12H); MS (ESI) m/z 329 [M+1]⁺.

### Example 55

### 3-Chloro-5-(4,4,5,5-tetramethyl-1,3,2-dioxaborolan-2-yl)phenol

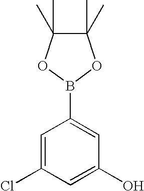

3-Bromo-5-chlorophenol (5 g, 19.9 mmol, described in: Maleczka R. E. et. al. J. Am. Chem. Soc. 2003, 125, 7792-7793), 4,4,4′,4′,5,5,5′,5′-octamethyl-2,2′-bi-1,3,2-dioxaborolane (6.06 g, 23.9 mmol), [1,1′-bis(diphenylphosphino)ferrocene]palladium(II) chloride dichloromethane adduct (487 mg, 0.6 mmol), potassium acetate (5.86 g, 59.7 mmol), 1,2-dimethoxyethane (60 mL) and water (4 mL) were divided into four microwave vials and irradiated in a microwave at 150° C. for 15 min each. When cooled to room temperature the mixtures were pooled, diluted with brine and extracted with diethyl ether. The combined organics were dried over sodium sulfate and concentrated in vacuo. Purified by column chromatography, using dichloromethane/acetonitrile (95:5) as the eluent, to give 1.43 g (28% yield) of the title compound: ¹H NMR (DMSO-d₆) δ 9.89 (s, 1H) 7.02 (s, 2H) 6.91 (s, 1H) 1.28 (s, 12H); MS (ESI) m/z 253 [M−H]⁻.

### Example 56

### 3-Chloro-5-(4,4,5,5-tetramethyl-1,3,2-dioxaborolan-2-yl)phenyl methanesulfonate

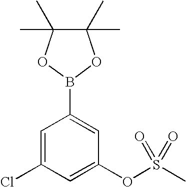

Methanesulfonyl chloride (122 μL, 0.79 mmol) was added dropwise to a cooled (0° C.) mixture of 3-chloro-5-(4,4,5,5-tetramethyl-1,3,2-dioxaborolan-2-yl)phenol (200 mg, 0.79 mmol) and triethylamine (0.4 mL, 3.14 mmol) in anhydrous dichloromethane (1.5 mL). The reaction mixture was allowed to reach room temperature and stirred for 1 h. Dichloromethane (10 mL) was added and the organic phase was washed with water, dried over sodium sulfate and concentrated in vacuo to give 200 mg (86% yield) of the crude title compound: ¹H NMR (CDCl₃) δ 7.75 (d, J=1.5 Hz, 2H), 7.57 (d, J=1.8 Hz, 2H), 7.41 (t, J=2.1 Hz, 1H), 3.18 (s, 3H), 1.35 (s, 12H); GC-MS (EI) m/z 332 [M]⁺.

### Example 57

### 3′-[2-Amino-4-(4-methoxyphenyl)-1-methyl-5-oxo-4,5-dihydro-1H-imidazol-4-yl]-5-methoxybiphenyl-3-yl methanesulfonate

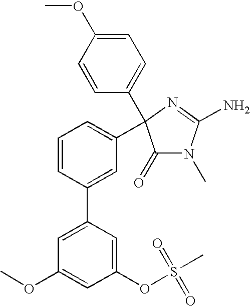

2-Amino-5-(3-bromophenyl)-5-(4-methoxyphenyl)-3-methyl-3,5-dihydro-4H-imidazol-4-one (100 mg, 0.27 mmol), 3-methoxy-5-(4,4,5,5-tetramethyl-1,3,2-dioxaborolan-2-yl)phenyl methanesulfonate (113 mg, 0.34 mmol), [1,1′-bis(diphenylphosphino)ferrocene]palladium(II) chloride dichloromethane adduct (22 mg, 0.027 mmol) and potassium carbonate (222 mg, 1.61 mmol) in anhydrous tetrahydrofuran (3 mL) were mixed and irradiated under an argon atmosphere in a microwave at 150° C. for 4 h. When cooled to room temperature the mixture was filtered and dimethyl sulfoxide (500 μL) was added. The solution was concentrated in vacuo to remove tetrahydrofuran and purified by preparative HPLC to give 33 mg (33% yield) of the title compound: ¹H-NMR (DMSO-d₆) δ 7.69 (br s, 1H), 7.57-7.52 (d, J=7.5 Hz, 1H), 7.52-7.47 (d, J=7.8 Hz, 1H), 7.44-7.33 (m, 3H), 7.09-7.01 (m, 2H), 6.95 (t, J=2.1 Hz, 1H), 6.89-6.82 (m, 2H), 6.64 (br s, 2H), 3.84 (s, 3H), 3.70 (s, 3H), 3.42 (s, 3H), 2.98 (s, 3H); MS (ES) m/z 496 [M+H]⁺.

TABLE 1

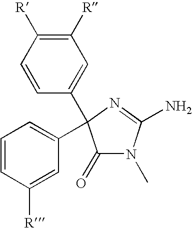

Representative examples synthesized as described for 3′-[2-amino-4-(4-
methoxyphenyl)-1-methyl-5-oxo-4,5-dihydro-1H-imidazol-4-yl]-5-methoxybiphenyl-3-yl
methanesulfonate.

| | | | | | | ¹H-NMR |
|---|---|---|---|---|---|---|
| | | | | | m/z | (DMSO-d₆) |
| Ex | Chemical name | R′ | R″ | R′′′ | [M + H]⁺ | δ ppm |
| 58 | 3′-{2-Amino-1-methyl-5-oxo-4-[4-(trifluoromethoxy)phenyl]-4,5-dihydro-1H-imidazol-4-yl}-5-methoxybiphenyl-3-ylmethanesulfonate | OCF₃ | H | 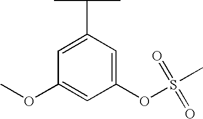 | 550 | 7.74-7.71 (m, 1H), 7.64-7.54(m, 3H), 7.54-7.50 (m, 1H),7.43 (t, J = 7.8 Hz, 1H), 7.31(d, J = 8.3 Hz, 2H), 7.10-7.03(m, 2H), 6.96 (t, J = 2.3 Hz,1H), 6.76 (br s, 2H), 3.84 (s,3H), 3.42 (s, 3H), 3.00 (s, 3H). |
| 59 | 3′-{2-Amino-1-methyl-5-oxo-4-[4-(trifluoromethoxy)phenyl]-4,5-dihydro-1H-imidazol-4-yl}-5-chlorobiphenyl-3-ylmethanesulfonate | OCF₃ | H | 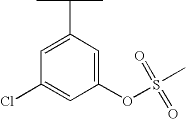 | 554 | 7.78-7.75 (m, 1H), 7.65-7.54(m, 5H), 7.52 (t, J = 2.0 Hz,1H); 7.50-7.42 (m, 2H), 7.31 (d,J = 8.5 Hz, 2H), 6.77 (br s,2H), 3.47 (s, 3H), 3.00 (s, 3H). |
| 60 | 3′-{2-Amino-4-[4-methoxy-3-(trifluoromethyl)phenyl]-1-methyl-5-oxo-4,5-dihydro-1H-imidazol-4-yl}-5-methoxybiphenyl-3-yl methanesulfonate | OCH₃ | CF₃ | 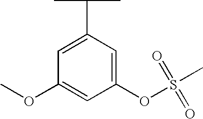 | 564 | 7.80-7.74 (m, 2H), 7.70-7.66(m, 1H), 7.59-7.53 (m, 1H),7.51-7.39 (m, 2H), 7.26-7.19(m, 1H), 7.09-7.03 (m, 2H),6.96 (t, J = 2.1 Hz, 1H), 6.79(br s, 2H), 3.85 (s, 6H), 3.42(s, 3H), 2.99 (s, 3H). |
| 61 | 3′-{2-Amino-4-[4-methoxy-3-(trifluoromethyl)phenyl]-1-methyl-5-oxo-4,5-dihydro-1H-imidazol-4-yl}-5-chlorobiphenyl-3-ylmethanesulfonate | OCH₃ | CF₃ | 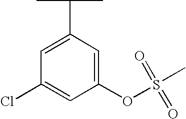 | 568 | 7.80-7.70 (m, 3H), 7.64-7.58(m, 2H), 7.55-7.50 (m, 2H),7.50-7.42 (m, 2H), 7.23 (d, J =8.8 Hz, 1H), 6.80 (br s,2H), 3.85 (s, 3H), 3.47 (s, 3H),2.99 (s, 3H). |
| 62 | 3′-[2-Amino-4-(4-methoxy-3-methylphenyl)-1-methyl-5-oxo-4,5-dihydro-1H-imidazol-4-yl]-5-methoxybiphenyl-3-ylmethanesulfonate 0.25acetate | OCH₃ | CH₃ | 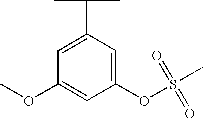 | 510 | 7.70-7.67 (m, 1H), 7.56-7.52(m, 1H), 7.51-7.47 (m, 1H),7.43-7.37 (m, 1H), 7.28-7.22(m, 1H), 7.22-7.18 (m, 1H),7.09-7.02 (m, 2H), 6.95 (t, J =2.3 Hz, 1H), 6.84 (d, J = 8.5Hz, 1H), 3.84 (s, 3H), 3.73 (s,3H), 3.42 (s, 3H), 2.98 (s,3H), 2.08 (s, 3H), 1.91 (s,0.7H). |
| 63 | 3′-{2-Amino-4-[4-(difluoromethoxy)phenyl]-1-methyl-5-oxo-4,5-dihydro-1H-imidazol-4-yl}-5-methoxybiphenyl-3-yl methanesulfonate 0.25acetate | OCHF₂ | H | 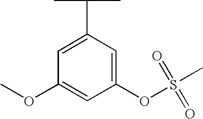 | 532 | 7.73 (in 1H), 7.58-7.48 (m,4H), 7.42 (t, J = 7.8 Hz, 1H),7.16 (t, J = 74.2 Hz, 1H), 7.14-7.09 (m, 2H), 7.09-7.03 (m,2H), 6.96 (t, J = 2.1 Hz, 1H),3.84 (s, 3H), 3.42 (s, 3H),2.99 (s, 3H), 1.90 (s, 1.1H). |
| 64 | 3′-{2-Amino-4-[4-(difluoromethoxy)phenyl]-1-methyl-5-oxo-4,5-dihydro-1H-imidazol-4-yl}-5-chlorobiphenyl-3-ylmethanesulfonate | OCHF₂ | H | 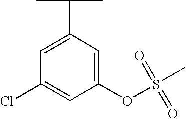 | 536 | 7.78-7.74 (m, 1H), 7.64-7.41(m, 8H), 7.16 (t, J = 74.2 Hz,1H), 7.11 (d, J = 8.5 Hz, 2H),6.74 (br s, 2H), 3.47 (s, 3H),2.99 (s, 3H). |

### Example 65

### 3-Methoxy-5-(4,4,5,5-tetramethyl-1,3,2-dioxaborolan-2-yl)benzonitrile

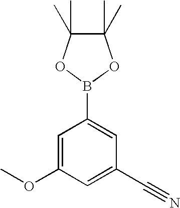

Anhydrous 1,2-dimethoxyethane (2.5 mL) was added to 3-chloro-5-methoxybenzonitrile (254 mg, 1.5 mmol, described in U.S. Pat. Appl. Publ., 2005234236, 20 Oct. 2005), tris(dibenzylideneaceton)dipalladium(0) (41 mg, 0.05 mmol), tricyclohexylphosphine (50 mg, 0.18 mmol), potassium acetate (221 mg, 2.25 mmol) and 4,4,4′,4′,5,5,5′,5′-octamethyl-2,2′-bi-1,3,2-dioxaborolane (419 mg, 1.65 mmol). The vial was capped, purged with argon and irradiated in a microwave at 150° C. for 1 h. When cooled to room temperature the mixture was diluted with water and extracted with diethyl ether. The combined organics were concentrated in vacuo and purified by column chromatography, using 0-2% acetonitrile in dichloromethane as the eluent, to give 185 mg (48% yield) of the title compound: MS (EI) m/z 259 [M−H]⁻.

### Example 66

### 3-Hydroxy-5-(4,4,5,5-tetramethyl-1,3,2-dioxaborolan-2-yl)benzonitrile

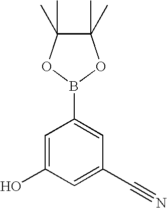

The title compound was synthesized as described for Example 65 in 85% yield, starting from 3-chloro-5-hydroxybenzonitrile (described in U.S. Pat. Appl. Publ., 2005234236, 20 Oct. 2005): MS (ES) m/z 244 [M−H]⁻.

### Example 67

### 3-Cyano-5-(4,4,5,5-tetramethyl-1,3,2-dioxaborolan-2-yl)phenyl methanesulfonate

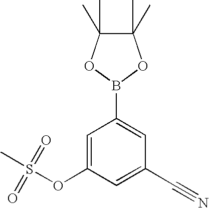

The title compound was synthesized as described for Example 56 in 95% yield, starting from 3-hydroxy-5-(4,4,5,5-tetramethyl-1,3,2-dioxaborolan-2-yl)benzonitrile: ¹H NMR (DMSO-d₆) δ 8.06 (dd, J=2.5, 1.5 Hz, 1H), 7.98-7.96 (m, 1H), 7.81 (dd, J=2.5, 0.8 Hz, 1H), 3.47 (s, 3H), 1.32 (s, 12H); MS (EI) m/z 323 [M+∩].

### Example 68

### 2-Amino-5-(3-bromo-4-fluorophenyl)-5-[4-(difluoromethoxy)phenyl]-3-methyl-3,5-dihydro-4H-imidazol-4-one

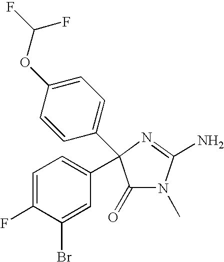

The title compound was synthesized as described for Example 48 in 59% yield, starting from 1-(3-bromo-4-fluorophenyl)-2-[4-(difluoromethoxy)phenyl]ethane-1,2-dione (prepared as described in Example 44 starting from [4-(difluoromethoxy)benzyl](triphenyl)phosphonium bromide and 3-bromo-4-fluorobenzoyl chloride): ¹H NMR (DMSO-d₆) δ 7.70 (dd, J=6.8, 2.2 Hz, 1H), 7.52-7.47 (m, 1H), 7.47-7.42 (m, 2H), 7.32 (t, J=8.8 Hz, 1H), 7.15-7.09 (m, 2H), 7.17 (t, J=74.0 Hz, 1H), 6.78 (br s, 2H), 2.98 (s, 3H); MS (ES) m/z 428, 430 [M+1]⁺.

### Example 69

### 5′-{2-Amino-4-[4-(difluoromethoxy)phenyl]-1-methyl-5-oxo-4,5-dihydro-1H-imidazol-4-yl}-2′-fluoro-5-methoxybiphenyl-3-yl methanesulfonate hydrochloride

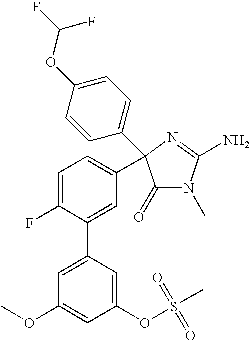

The title compound was synthesized as described for Example 57 in 31% yield, starting from 2-amino-5-(3-bromo-4-fluorophenyl)-5-[4-(difluoromethoxy)phenyl]-3-methyl-3,5-dihydro-4H-imidazol-4-one and 3-methoxy-5-(4,4,5,5-tetramethyl-1,3,2-dioxaborolan-2-yl)phenyl methanesulfonate. The base was dissolved in anhydrous dichloromethane and treated with 1 M hydrochloric acid in diethyl ether (0.7 mL) and the resulting mixture was concentrated to give the title compound: ¹H NMR (DMSO-d₆) δ 11.68 (br s, 1H), 9.65 (br s, 2H), 7.62-7.57 (m, 1H), 7.47-7.41 (m, 4H), 7.28-7.24 (m, 2H), 7.13-7.08 (m, 2H), 7.45-7.08 (m, 1H), 7.06-7.03 (m, 1H), 3.85 (s, 3H), 3.43 (s, 3H), 3.18 (s, 3H); MS (ES) m/z 550 [M+1]⁺.

### Example 70

### 5′-{2-Amino-4-[4-(difluoromethoxy)phenyl]-1-methyl-5-oxo-4,5-dihydro-1H-imidazol-4-yl}-5-cyano-2′-fluorobiphenyl-3-yl methanesulfonate hydrochloride

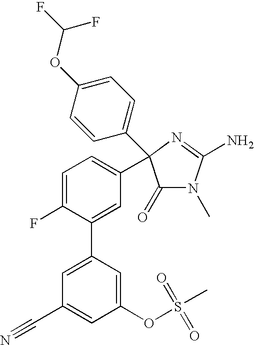

The title compound was synthesized as described for Example 57 in 10% yield starting from 2-amino-5-(3-bromo-4-fluorophenyl)-5-[4-(difluoromethoxy)phenyl]-3-methyl-3,5-dihydro-4H-imidazol-4-one and 3-cyano-5-(4,4,5,5-tetramethyl-1,3,2-dioxaborolan-2-yl)phenyl methanesulfonate. The base was dissolved in anhydrous dichloromethane and treated with 1 M hydrochloric acid in diethyl ether (0.5 mL) and the resulting mixture was concentrated to give the title compound: ¹H NMR (DMSO-d₆) δ 10.48 (s, 1H), 10.13 (s, 1H), 7.61-7.56 (m, 1H), 7.49-7.45 (m, 1H), 7.45-7.38 (m, 4H), 7.26-7.21 (m, 3H), 7.21-7.18 (m, 1H), 7.41-7.06 (m, 1H), 3.09 (s, 3H), 3.01 (s, 3H); MS (ES) m/z 545 [M+1]⁺.

### Example 71

### 4-[2-Amino-4-(3′-cyano-6-fluoro-5′-methoxybiphenyl-3-yl)-1-methyl-5-oxo-4,5-dihydro-1H-imidazol-4-yl]phenyl methanesulfonate hydrochloride

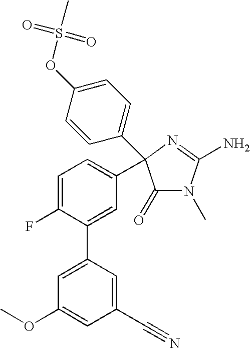

The title compound was synthesized as described for Example 57 in 36% yield starting from 4-[2-amino-4-(3-bromo-4-fluorophenyl)-1-methyl-5-oxo-4,5-dihydro-1H-imidazol-4-yl]phenyl methanesulfonate and 3-methoxy-5-(4,4,5,5-tetramethyl-1,3,2-dioxaborolan-2-yl)benzonitrile. The base was dissolved in anhydrous dichloromethane and treated with 1 M hydrochloric acid in diethyl ether (1 mL) and resulting mixture was concentrated to give the title compound: ¹H NMR (DMSO-d₆) δ 11.73 (br s, 1H), 9.69 (br s, 2H), 7.67-7.62 (m, 1H), 7.60-7.57 (m, 1H), 7.55-7.49 (m, 3H), 7.48-7.42 (m, 5H), 3.87 (s, 3H), 3.41 (s, 3H), 3.19 (s, 3H); MS (ES) m/z 509 [M+1]⁺.

### Example 72

### 5-Bromopyridine-3-yl methanesulfonate

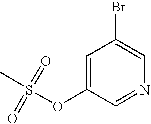

The title compound was synthesized as described for Example 56 in quantitative yield, starting from 5-bromopyridine-3-ol: ¹H NMR (DMSO-d₆) δ 8.76 (d, J=2.0 Hz, 1H), 8.64 (d, J=2.3 Hz, 1H), 8.23 (dd, J=2.4, 2.0 Hz, 1H), 3.52 (s, 3H); MS (EI) m/z 251/253 [M+.]

### Example 73

### 5-(5-{2-Amino-4-[4-(difluoromethoxy)phenyl]-1-methyl-5-oxo-4,5-dihydro-1H-imidazol-4-yl}-2-fluorophenyl)pyridin-3-yl methanesulfonate hydrochloride

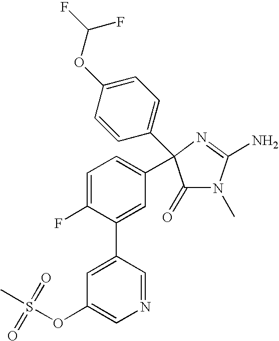

5-Bromopyridine-3-yl methanesulfonate (126 mg, 0.5 mmol), hexamethyldistannane (327 mg, 1 mmol) and tetrakis(triphenylphosphine)palladium(0) (15 mg, 0.013 mmol) were dissolved in 0.75 mL of anhydrous tetrahydrofuran and irradiated in a microwave for 1 h at 130° C. 2-Amino-5-(3-bromo-4-fluorophenyl)-5-[4-(difluoromethoxy)phenyl]-3-methyl-3,5-dihydro-4H-imidazol-4-one (107 mg, 0.25 mmol), silver(I) oxide (58 mg, 0.25 mmol) and tetrakis(triphenylphosphine)palladium(0) (29 mg, 0.025 mmol) were dissolved in 4 mL of anhydrous N,N-dimethylformamide and heated to 100° C. under argon. The mixture was stirred at 100° C. for 5 min and then the stannane solution was added. The resulting mixture was stirred at 100° C. for 1.5 h under an atmosphere of argon and then at room temperature over night. When cooled to room temperature the mixture was filtered through celite, rinsed with ethyl acetate and the filtrate was concentrated in vacuo. The residue was purified by column chromatography, using 10% 0.1 M ammonia in methanol and 90% dichloromethane as the eluent, followed by preparative HPLC. The acetonitrile was removed in vacuo and the residue diluted with saturated sodium hydrogencarbonate and extracted with dichloromethane. The combined organic phases were treated with 1 M hydrochloric acid in diethyl ether (0.5 mL) and resulting mixture was concentrated to give 28 mg (20% yield) of the title compound: ¹H NMR (DMSO-d₆) δ 11.76 (br s, 1H), 9.69 (br s, 2H), 8.83-8.77 (m, 1H), 8.72-8.68 (m, 1H), 8.08-8.03 (m, 1H), 7.73-7.69 (m, 1H), 7.54-7.44 (m, 4H), 7.29-7.25 (m, 2H), 7.54-7.08 (m, 1H), 3.53 (s, 3H), 3.19 (s, 3H); MS (ES) m/z 521 [M+1]⁺.

### Example 74

### 2-Amino-5-(3-bromo-4-fluorophenyl)-5-(4-hydroxyphenyl)-3-methyl-3,5-dihydro-4H-imidazol-4-one

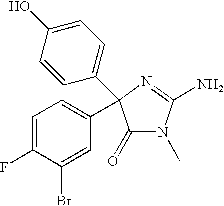

tert-Butylperoxide (2.05 mL, 15 mmol, 70 wt % in water) was added to a solution of 5-(3-bromo-4-fluorophenyl)-5-(4-hydroxyphenyl)-3-methyl-2-thioxoimidazolidin-4-one (395 mg, 1 mmol) in a mixture of methanol/ammonium hydroxide (15:4 mL). The reaction was stirred at room temperature overnight, then concentrated and the residue dissolved in dichloromethane. Purification by column chromatography, using 0-5% 0.1 M ammonia in methanol in dichloromethane as the eluent, gave 226 mg (60% yield) of the title compound: MS (ES) m/z 378, 380 [M+1]⁺.

### Example 75

### 2-Amino-5-(4-fluoro-3-pyrimidin-5-ylphenyl)-5-(4-hydroxyphenyl)-3-methyl-3,5-dihydro-4H-imidazol-4-one

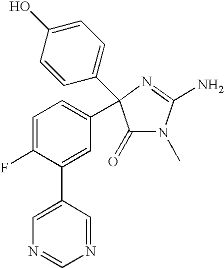

The title compound was synthesized as described for Example 57 in 77% yield, starting from 2-amino-5-(3-bromo-4-fluorophenyl)-5-(4-hydroxyphenyl)-3-methyl-3,5-dihydro-4H-imidazol-4-one and 5-bromopyrimidine: ¹H NMR (DMSO-d₆) δ 9.48 (br s, 1H), 9.23 (s, 1H), 8.96-8.93 (m, 2H), 7.66-7.62 (m, 1H), 7.56-7.49 (m, 1H), 7.43-7.35 (m, 1H), 7.23-7.17 (m, 2H), 6.75-6.68 (m, 2H), 3.03 (s, 3H); MS (ES) m/z 378 [M+1]⁺.

### Example 76

### 4-[2-Amino-4-(3-fluoro-4-pyrimidin-5-ylphenyl)-1-methyl-5-oxo-4,5-dihydro-1H-imidazol-4-yl]phenyl trifluoromethanesulfonate hydrochloride

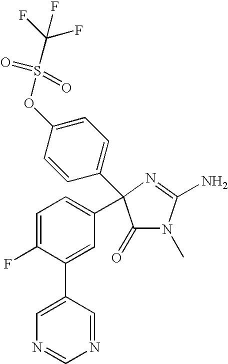

A mixture of 2-amino-5-(4-fluoro-3-pyrimidin-5-ylphenyl)-5-(4-hydroxyphenyl)-3-methyl-3,5-dihydro-4H-imidazol-4-one (75 mg, 0.2 mmol), N-phenyl-bis(trifluoromethanesulfonimide) (71 mg, 0.2 mmol), anhydrous potassium carbonate (166 mg, 1.2 mmol) in anhydrous tetrahydrofuran (3 mL) were irradiated in a microwave at 120° C. for 7 min. The mixture was filtrated, the solvent evaporated and the residue purified by preparative HPLC. The acetonitrile was removed in vacuo, the residue diluted with saturated aqueous sodium hydrogen carbonate and extracted with ethyl acetate. The combined organics were dried over sodium sulfate and filtered. Hydrochloric acid (1 M in diethyl ether, 0.5 mL) was added to the filtrate. The mixture was stirred at room temperature for 5 min and the solvent was evaporated to give 63 mg (58% yield) of the title compound: ¹H NMR (DMSO-d₆) δ 11.82 (br s, 1H), 9.82-9.65 (m, 2H), 9.26 (s, 1H), 9.04 (s, 2H), 7.76-7.73 (m, 1H), 7.65-7.61 (m, 4H), 7.56-7.50 (m, 1H), 7.48-7.43 (m, 1H), 3.19 (s, 3H); MS (ES) m/z 510 [M+1]⁺.

### Example 77

### 2-[(E)-2-Cyclopropylvinyl]-1,3,2-benzodioxaborole

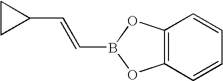

Ethynylcyclopropane (500 mg, 7.56 mmol) and 1,3,2-benzodioxaborole (1.361 g, 11.35 mmol) were dissolved in tetrahydrofuran (35 mL) and heated at reflux for 3 h. Upon cooling to room temperature the solvent was evaporated and the residue purified by column chromatography, using 0-60% ethyl acetate in heptane as the eluent. Residual 1,3,2-benzodioxaborole was precipitated from methanol by the addition of heptane and removed by filtration. The residue was concentrated to give 640 mg (46% yield) of the title compound: ¹H NMR (CDCl₃) δ 7.22-7.19 (m, 2H), 7.08-7.05 (m, 2H), 6.46 (dd, J=17.7, 9.5 Hz, 1H), 5.85 (d, J=17.7 Hz, 1H), 1.73-1.62 (m, 1H), 0.97-0.91 (m, 2H), 0.69-0.63 (m, 2H).

### Example 78

### 4-(2-Amino-4-{3-[(E)-2-cyclopropylvinyl]-4-fluorophenyl}-1-methyl-5-oxo-4,5-dihydro-1H-imidazol-4-yl)phenyl methanesulfonate

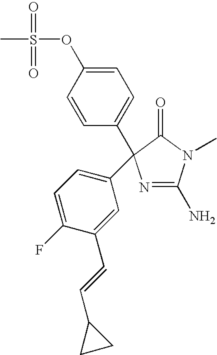

4-[2-Amino-4-(3-bromo-4-fluorophenyl)-1-methyl-5-oxo-4,5-dihydro-1H-imidazol-4-yl]phenyl methanesulfonate (100 mg, 0.22 mmol), 2-[(E)-2-cyclopropylvinyl]-1,3,2-benzodioxaborole (49 mg, 0.26 mmol), [1,1′-bis(diphenylphosphino)ferrocene]palladium(II) chloride dichloromethane adduct (18 mg, 0.022 mmol) and potassium carbonate (181 mg, 1.31 mmol) were mixed in a vial. The vial was evacuated and filled with argon (repeated twice) before tetrahydrofuran (1.4 mL) was added. The resulting mixture was irradiated in a microwave at 130° C. for 2 h. Upon cooling the mixture was diluted with water and extracted with ethyl acetate. The organic extract was washed with brine and the combined aqueous layers were extracted once more with ethyl acetate. The combined organic extracts were dried over magnesium sulfate, filtered and concentrated. Purification by column chromatography, using a gradient of 0-10% 0.1 M ammonia in methanol in dichloromethane as the eluent, gave 43 mg (44% yield) of the title compound: ¹H NMR (DMSO-d₆) δ 7.56 (dd, J=7.6, 2.3 Hz, 1H), 7.52-7.47 (m, 2H), 7.35-7.25 (m, 3H), 7.10 (dd, J=10.7, 8.7 Hz, 1H), 6.76 (br s, 2H), 6.52 (d, J=15.9 Hz, 1H), 5.73 (dd, J=15.9, 9.4 Hz, 1H), 3.35 (s, 3H), 2.98 (s, 3H), 1.68-1.57 (m, 1H), 0.84-0.77 (m, 2H), 0.57-0.50 (m, 2H); MS (ES) m/z 444 [M+1]⁺.

### Example 79

### 4-{2-Amino-4-[3-(2-cyclopropylethyl)-4-fluorophenyl]-1-methyl-5-oxo-4,5-dihydro-1H-imidazol-4-yl}phenyl methanesulfonate

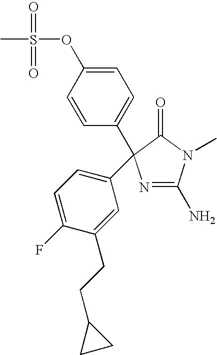

A mixture of 4-(2-amino-4-{3-[(E)-2-cyclopropylvinyl]-4-fluorophenyl}-1-methyl-5-oxo-4,5-dihydro-1H-imidazol-4-yl)phenyl methanesulfonate (100 mg, 0.23 mmol) and palladium on charcoal (30 mg, 10 wt %) in ethyl acetate (10 mL) was divided into two equal parts. The first part was hydrogenated at atmospheric pressure for 18 h, filtered through diatomaceous earth and concentrated. The second part was hydrogenated at atmospheric pressure for 3.25 h. The reaction vessel was purged with nitrogen and left to stand for 66 h before more palladium on charcoal (10 mg, 10 wt %) was added and the mixture was hydrogenated for an additional 6 h. The mixture was filtered through diatomaceous earth and the filtrate was concentrated. The two reactions were pooled and purified by column chromatography, using a gradient of 0-10% 0.1 M ammonia in methanol in dichloromethane as the eluent, followed by preparative HPLC to give 43 mg (42% yield) of the title compound: ¹H NMR (CDCl₃) δ 7.56-7.50 (m, 2H), 7.32-7.20 (m, 4H), 6.95 (m, 1H), 3.13 (s, 3H), 3.13 (s, 3H), 2.74-2.67 (m, 2H), 1.50-1.42 (m, 2H), 0.72-0.60 (m, 1H), 0.41-0.35 (m, 2H), 0.03-0.03 (m, 2H); MS (ES) m/z 446 [M+1]⁺.

### Example 80

### (3-Bromo-4-fluorophenyl)(2-phenyl-1,3-dithian-2-yl)methanol

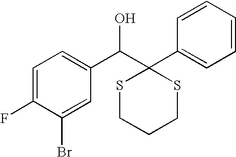

To a cooled (−78° C.) solution of 2-phenyl-1,3-dithiane (9.50 g, 48.39 mmol) in tetrahydrofuran (150 mL) was slowly added n-butyllithium (1.6 M in hexanes, 33 mL, 53.2 mmol) under an atmosphere of nitrogen. The resulting solution was stirred for 20 min before 3-bromo-4-fluorobenzaldehyde (10.31 g, 50.80 mmol) in tetrahydrofuran (10 mL) was added. The reaction mixture was allowed to slowly reach room temperature during 2 h before the reaction was quenched with saturated aqueous ammonium chloride (70 mL). The phases were separated and the aqueous layer was extracted with dichloromethane. The combined organic extracts were dried over magnesium sulfate, filtered and concentrated to give 19.95 g of the crude title compound, used without further purification: MS (ES) m/z 381/383 [M−18]⁺.

### Example 81

### 1-(3-Bromo-4-fluorophenyl)-2-phenylethane-1,2-dione

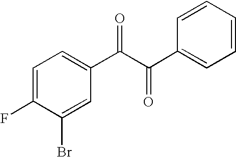

(3-Bromo-4-fluorophenyl)(2-phenyl-1,3-dithian-2-yl)methanol (19.52 g, 49.96 mmol) was dissolved in dichloromethane (450 mL) under an atmosphere of nitrogen and tert-butanol (15.76 mL, 168 mmol) was added. 1,1,1-Tris(acetyloxy)-1λ⁵,2-benziodoxol-3(1H)-one (50.89 g, 120 mmol) was added in portions and the resulting mixture was stirred at room temperature for 20 h. Sodium thiosulfate (50 g) in saturated aqueous sodium hydrogen carbonate (500 mL) was added and the phases were separated. The aqueous layer was extracted twice with dichloromethane and the combined organic extracts were dried over magnesium sulfate, filtered and concentrated. Purification by column chromatography, using a gradient of 0-20% ethyl acetate in heptane as the eluent, gave 11.32 g (74% yield) of the title compound: MS (ES) m/z 305/307 [M−1]⁻.

### Example 82

### 5-(3-Bromo-4-fluorophenyl)-3-methyl-5-phenyl-2-thioxoimidazolidin-4-one

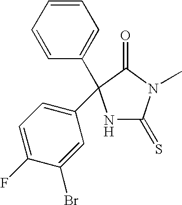

1-(3-Bromo-4-fluorophenyl)-2-phenylethane-1,2-dione (11.32 g, 36.86 mmol) and N-methylthiourea (6.65 g, 73.72 mmol) were dissolved in dimethyl sulfoxide (37 mL) and the resulting solution was heated at 100° C. Potassium hydroxide (1.2 M in water, 43 mL, 51.60 mmol) was added dropwise, keeping the temperature at 100° C. and the resulting mixture was stirred for 15 min. After cooling to room temperature water (250 mL) was added followed by addition of 2 M hydrochloric acid until pH 2 at which the product precipitated. The water/dimethyl sulfoxide mixture was decanted and the residue was dissolved in dichloromethane. Potassium hydroxide (0.25 M in water) was added and the pH of the aqueous layer was adjusted to 4 by careful addition of 2 M hydrochloric acid. The phases were separated and the aqueous layer was extracted with dichloromethane. The aqueous layer was combined with the previously decanted water/dimethyl sulfoxide mixture and extracted with dichloromethane. The combined organic extracts were dried over magnesium sulfate, filtered and concentrated to give 14.37 g of the crude title compound, used without further purification: MS (ES) m/z 377/379 [M−1]⁻.

### Example 83

### 2-Amino-5-(3-bromo-4-fluorophenyl)-3-methyl-5-phenyl-3,5-dihydro-4H-imidazol-4-one

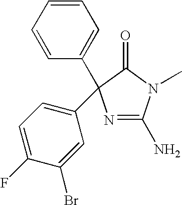

To a solution of 5-(3-bromo-4-fluorophenyl)-3-methyl-5-phenyl-2-thioxoimidazolidin-4-one (14.30 g, 37.7 mmol) in methanol (190 mL) and ammonium hydroxide (60 mL) was added aqueous tert-butyl hydroperoxide (70%, 5.48 mL) and the resulting mixture was stirred at 35° C. for 2 h. Additional aqueous tert-butyl hydroperoxide (70%, 76 mL) was added and the mixture was stirred for another 45 min until all starting material was consumed. The methanol was evaporated and the residue was diluted with dichloromethane and water. The phases were separated and the aqueous layer was extracted with chloroform. The combined organic extracts were dried over magnesium sulfate, filtered and concentrated. Purification by column chromatography, using a gradient of 0-10% 0.1 M ammonia in methanol in dichloromethane as the eluent, gave 5.31 g (39% yield) of the title compound: MS (ES) m/z 362/364 [M+1]⁺.

### Example 84

### 5′-(2-Amino-1-methyl-5-oxo-4-phenyl-4,5-dihydro-1H-imidazol-4-yl)-2′-fluoro-5-methoxybiphenyl-3-yl methanesulfonate

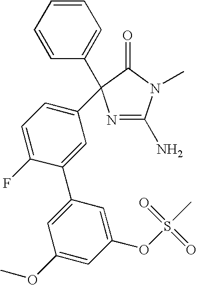

A mixture of 2-amino-5-(3-bromo-4-fluorophenyl)-3-methyl-5-phenyl-3,5-dihydro-4H-imidazol-4-one (4.18 g, 11.54 mmol), 3-methoxy-5-(4,4,5,5-tetramethyl-1,3,2-dioxaborolan-2-yl)phenyl methanesulfonate (4.55 g, 13.86 mmol), [1,1′-bis(diphenylphosphino)ferrocene]palladium(II) chloride dichloromethane adduct (942 mg, 1.15 mmol) and potassium carbonate (9.57 g, 69.24 mmol) in tetrahydrofuran (65 mL) were divided into four vials. Each vial was irradiated in a microwave at 130° C. for 3 h. Upon cooling the reaction mixtures were pooled, diluted with water and extracted with ethyl acetate. The organic extract was washed with brine and the combined aqueous layers were extracted with ethyl acetate. The combined organic extracts were dried over magnesium sulfate, filtered and concentrated. Purification by column chromatography, using a gradient of 0-5% 0.1 M ammonia in methanol in dichloromethane as the eluent, gave 4.81 g (86% yield) of the title compound: MS (ES) m/z 484 [M+1]⁺.

### Example 85

### Chromatographic preparation of the enantiomers of 5′-(2-amino-1-methyl-5-oxo-4-phenyl-4,5-dihydro-1H-imidazol-4-yl)-2′-fluoro-5-methoxybiphenyl-3-yl methanesulfonate

5′-(2-Amino-1-methyl-5-oxo-4-phenyl-4,5-dihydro-1H-imidazol-4-yl)-2′-fluoro-5-methoxybiphenyl-3-yl methanesulfonate (2.4 g, 4.96 mmol) was dissolved in ethanol (64 mL) and the resulting solution was divided into eight equal portions. Chiral separation was carried out on a Chiralpak AD column (50×300 mm), using ethanol with 0.1% diethyl amine/heptane (20/80) as eluent at a flow rate of 100 mL/min. The separation was monitored at 254 nm and the two isomers were collected and concentrated in vacuo.

Isomer 1, the first isomer to elute (820 mg, 1.70 mmol, 68% yield): ¹H NMR (DMSO-d₆) δ 7.60 (dd, J=7.6, 2.3 Hz, 1H), 7.55-7.49 (m, 1H), 7.46-7.41 (m, 2H), 7.35-7.20 (m, 4H), 7.03-6.97 (m, 3H), 6.75 (br s, 2H), 3.83 (s, 3H), 3.42 (s, 3H), 2.99 (s, 3H); MS (ES) m/z 484 [M+1].

Isomer 2, the last isomer to elute, (773 mg, 1.60 mmol, 64% yield): ¹H NMR (DMSO-d₆) δ 7.60 (dd, J=7.6, 2.3 Hz, 1H), 7.55-7.49 (m, 1H), 7.46-7.41 (m, 2H), 7.35-7.20 (m, 4H), 7.03-6.97 (m, 3H), 6.76 (br s, 2H), 3.83 (s, 3H), 3.42 (s, 3H), 3.00 (s, 3H); MS (ES) m/z 484 [M+1].

### Example 86

### 3-{2-Amino-4-[3-(5-chloro-2-fluoropyridin-3-yl)-4-fluorophenyl]-1-methyl-5-oxo-4,5-dihydro-1H-imidazol-4-yl}phenyl methanesulfonate

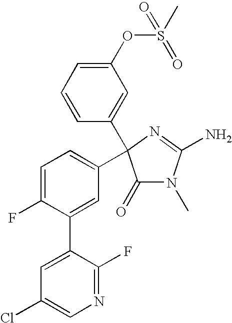

3-[2-Amino-4-(3-bromo-4-fluorophenyl)-1-methyl-5-oxo-4,5-dihydro-1H-imidazol-4-yl]phenyl methanesulfonate (94 mg, 0.206 mmol), (5-chloro-2-fluoropyridin-3-yl)boronic acid (47 mg, 0.268 mmol), [1,1′-bis(diphenylphosphino)ferrocene]palladium(II) chloride dichloromethane adduct (17 mg, 0.021 mmol), potassium carbonate (171 mg, 0.975 mmol), and anhydrous tetrahydrofuran (3 mL) was irradiated in a microwave at 130° C. for 2 h. When cooled to room temperature the mixture was filtered and dimethyl sulfoxide (500 μL) was added. The solution was concentrated in vacuo to remove the tetrahydrofuran and purified by preparative HPLC to give 6.5 mg (6.2% yield) of the title compound: ¹H NMR (DMSO-d₆) δ 8.36 (dd, J=2.6, 1.1 Hz, 1H), 8.17 (dd, J=8.2, 2.6 Hz, 1H), 7.62-7.54 (m, 2H), 7.51-7.45 (m, 1H), 7.44-7.37 (m, 2H), 7.35-7.27 (m, 2H), 7.25-7.18 (m, 1H), 3.31 (s, 3H), 2.95 (s, 3H); MS (ES) m/z 507 [M+1]⁺.

TABLE 2
Representative examples synthesized as described for 3-{2-Amino-4-[3-(5-chloro-
2-fluoropyridin-3-yl)-4-fluorophenyl]-1-methyl-5-oxo-4,5-dihydro-1H-imidazol-4-
yl}phenyl methanesulfonate. All reactions were analyzed using LC-MS and those which
showed a low level of conversion was irradiated for another hour at 130° C.

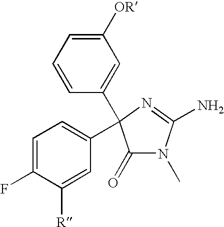

| | | | | | ¹H-NMR |
|---|---|---|---|---|---|
| | | | | m/z | (DMSO-d₆) |
| Ex | Chemical name | R′ | R″ | [M + H]⁺ | δ ppm |
| 87 | 3-{2-Amino-4-[4-fluoro-3-(5-fluoropyridin-3-yl)phenyl]-1-methyl-5-oxo-4,5-dihydro-1H-imidazol-4-yl}phenylmethanesulfonate | 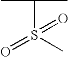 | 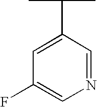 | 473 | No data |
| 88 | 3-{2-Amino-4-[4-fluoro-3-(2-fluoropyridin-3-yl)phenyl]-1-methyl-5-oxo-4,5-dihydro-1H-imidazol-4-yl}phenylmethanesulfonate | 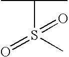 | 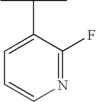 | 473 | 8.35-8.28 (m, 1H) 8.05-7.96(m, 1H) 7.63-7.56 (m, 3H)7.54-7.42 (m, 4H) 7.38-7.30(m, 1H) 7.26 (dd, J = 7.5, 2.0Hz, 1H) 3.37 (s, 3H) 3.00 (s,3H). |
| 89 | 3-[2-Amino-4-(3′,5′-dichloro-6-fluorobiphenyl-3-yl)-1-methyl-5-oxo-4,5-dihydro-1H-imidazol-4-yl]phenyl methanesulfonate | 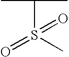 | 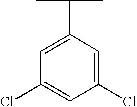 | 422 | 7.68 (t, J = 1.9 Hz, 1H) 7.64-7.48 (m, 5H) 7.47-7.39 (m,3H) 7.36-7.28 (m, 1H) 7.23 (s,1H) 3.35 (s, 3H) 2.99 (s, 3H). |
| 90 | 3-[2-Amino-4-(4-fluoro-3-pyridin-3-ylphenyl)-1-methyl-5-oxo-4,5-dihydro-1H-imidazol-4-yl]phenylmethanesulfonate | 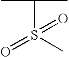 | 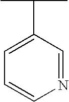 | 455 | 8.66 (s, 1H) 8.61 (dd, J = 4.81.8 Hz, 1H) 7.88 (dd, J = 7.92.1 Hz, 1H) 7.62 (dd, J = 7.62.4 Hz, 1H) 7.57-7.39 (m,5H) 7.32 (t, 1H) 7.25 (dd, J =7.6, 2.1 Hz, 1H) 3.36 (s, 3H)2.99 (s, 3H). |
| 91 | 3-[2-Amino-4-(3′-chloro-6-fluorobiphenyl-3-yl)-1-methyl-5-oxo-4,5-dihydro-1H-imidazol-4-yl]phenyl methanesulfonate | 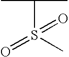 | 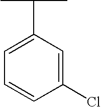 | 488 | 7.60 (dd, J = 7.6, 2.1 Hz, 1H)7.57-7.38 (m, 9H) 7.34-7.20(m, 2H) 3.36 (s, 3H) 2.99 (s,3H). |
| 92 | 3-[2-Amino-4-(3′-chloro-6-fluoro-5′-methoxybiphenyl-3-yl)-1-methyl-5-oxo-4,5-dihydro-1H-imidazol-4-yl]phenyl methanesulfonate | 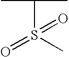 | 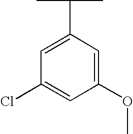 | 518 | 7.60-7.49 (m, 4H) 7.48-7.39(m, 2H) 7.32-7.20 (m, 2H)7.10 (t, J = 2.0 Hz, 1H) 7.05 (d,J = 1.2 Hz, 1H) 6.95 (s, 1H)3.81 (s, 3H) 3.37-3.34 (m,3H) 2.99 (s, 3H). |
| 93 | 3-[2-Amino-4-(6-fluoro-3′-methoxybiphenyl-3-yl)-1-methyl-5-oxo-4,5-dihydro-1H-imidazol-4-yl]phenyl methanesulfonate | 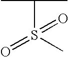 | 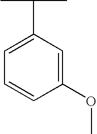 | 484 | 7.63-7.33 (m, 7H) 7.33-7.21(m, 2H) 7.06-6.92 (m, 3H)3.78 (s, 3H) 3.36 (s, 3H) 2.99(s, 3H). |
| 94 | 3-[2-Amino-4-(4-fluoro-3-pyrimidin-5-ylphenyl)-1-methyl-5-oxo-4,5-dihydro-1H-imidazol-4-yl]phenyl methanesulfonate | 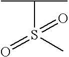 | 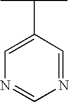 | 456 | 9.23 (s, 1H) 8.93 (s, 2H) 7.68(dd, J = 7.5, 2.3 Hz, 1H) 7.61-7.56 (m, 1H) 7.56-7.51 (m,1H) 7.48-7.33 (m, 4H) 7.28-7.23 (m, 1H) 3.35 (s, 3H) 2.99(s, 3H). |
| 95 | 3-{2-Amino-4-[3-(5-chloro-2-fluoropyridin-3-yl)-4-fluorophenyl]-1-methyl-5-oxo-4,5-dihydro-1H-imidazol-4-yl}phenyl propane-1-sulfonate | 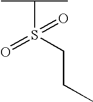 | 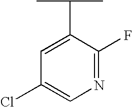 | 535 | 8.51-8.33 (m, 1H) 8.23 (dd, J =8.0, 2.5 Hz, 1H) 7.68-7.31(m, 7H) 7.28-7.20 (m, 1H)3.50-3.42 (m, 2H) 3.01 (s,3H) 1.86-1.76 (m, 2H) 1.04-0.96 (m, 3H). |
| 96 | 3-{2-Amino-4-[4-fluoro-3-(2-fluoropyridin-3-yl)phenyl]-1-methyl-5-oxo-4,5-dihydro-1H-imidazol-4-yl}phenyl propane-1-sulfonate | 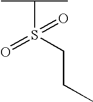 | 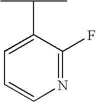 | 501 | 8.34-8.29 (m, 1H) 8.05-7.95(m, 1H) 7.61-7.55 (m, 2H)7.54-7.39 (m, 5H) 7.37-7.30(m, 1H) 7.25-7.19 (m, 1H)3.49-3.43 (m, 2H) 3.00 (s,3H) 1.86-1.74 (m, 2H) 0.99 (t, J =7.4 Hz, 3H). |
| 97 | 3-[2-Amino-4-(4-fluoro-3-pyridin-3-ylphenyl)-1-methyl-5-oxo-4,5-dihydro-1H-imidazol-4-yl]phenylpropane-1-sulfonate | 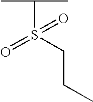 | 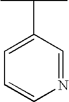 | 483 | 8.66 (s, 1H) 8.61 (dd, J = 4.91.6 Hz, 1H) 7.88 (dd, J = 7.91.9 Hz, 1H) 7.62 (dd, J = 7.62.4 Hz, 1H) 7.58-7.48 (m,4H) 7.46-7.40 (m, 2H) 7.32 (dd,J = 10.4, 8.7 Hz, 1H) 7.21 (dd,J = 8.0, 1.5Hz, 1H) 3.50-3.40(m, 2H) 2.99 (s, 3H) 1.84-1.74 (m, 2H) 0.99 (t, J = 7.4Hz, 3H). |
| 98 | 3-[2-Amino-4-(3′-chloro-6-fluorobiphenyl-3-yl)-1-methyl-5-oxo-4,5-dihydro-1H-imidazol-4-yl]phenyl propane-1-sulfonate | 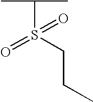 | 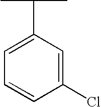 | 516 | 7.60 (dd, J = 7.5, 2.3 Hz, 1H)7.56-7.46 (m, 6H) 7.45-7.38(m, 3H) 7.29 (dd, J = 10.5, 8.8Hz, 1H) 7.20 (dd, J = 7.5, 2.0Hz, 1H) 3.51-3.40 (m, 2H)2.99 (s, 3H) 1.86-1.74 (m,2H) 0.98 (t, J= 7.4 Hz, 3H). |
| 99 | 3-[2-Amino-4-(3′-chloro-6-fluoro-5′-methoxybiphenyl-3-yl)-1-methyl-5-oxo-4,5-dihydro-1H-imidazol-4-yl]phenyl propane-1-sulfonate | 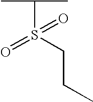 | 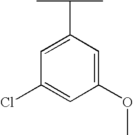 | 564 | 7.61-7.15 (m, 8H) 7.10 (t, J =2.1 Hz, 1H) 7.05 (s, 1H) 6.95(s, 1H) 3.81 (s, 3H) 3.51-3.39(m, 2H) 2.99 (s, 3H) 1.86-1.72 (m, 2H) 0.99 (t, 3H). |
| 100 | 3-[2-Amino-4-(3′,5′-dichloro-6-fluorobiphenyl-3-yl)-1-methyl-5-oxo-4,5-dihydro-1H-imidazol-4-yl]phenyl propane-1-sulfonate | 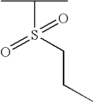 | 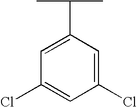 | 550 | 7.80-7.13 (m, 11H) 3.51-3.42(m, 2H) 3.00 (s, 3H) 1.88-1.72 (m, 2H) 1.00 (t, J = 7.4Hz, 3H). |
| 101 | 3-[2-Amino-4-(6-fluoro-3′-methoxybiphenyl-3-yl)-1-methyl-5-oxo-4,5-dihydro-1H-imidazol-4-yl]phenyl propane-1-sulfonate | 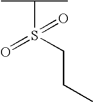 | 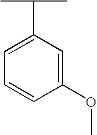 | 512 | 7.73-7.33 (m, 8H) 7.27 (t, J =1.9 Hz, 2H) 7.00 (dd, J = 11.3,2.26 Hz, 2H) 3.79 (s, 3H) 3.55-3.42 (m, 2H) 3.00 (s, 3H)1.87-1.74 (m, 2H) 1.01 (q, J =7.4 Hz, 3H). |
| 102 | 3-[2-Amino-4-(4-fluoro-3-pyrimidin-5-ylphenyl)-1-methyl-5-oxo-4,5-dihydro-1H-imidazol-4-yl]phenyl propane-1-sulfonate | 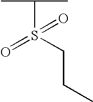 | 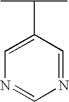 | 484 | 9.23 (s, 1H) 8.93 (d, J = 1.3 Hz,2H) 7.67 (dd, J = 7.5, 2.5 Hz,1H) 7.64-7.51 (m, 2H) 7.48-7.31 (m, 4H) 7.22 (dd, J = 7.5,2.0 Hz, 1H) 3.50-3.43 (m,2H) 3.00 (s, 3H) 1.85-1.74 (m,2H) 0.99 (t,J = 7.4 Hz, 3H). |
| 103 | 3-{2-Amino-4-[3-(5-chloro-2-fluoropyridin-3-yl)-4-fluorophenyl]-1-methyl-5-oxo-4,5-dihydro-1H-imidazol-4-yl}phenyl propane-2-sulfonate | 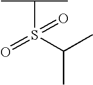 | 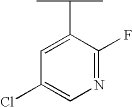 | 535 | 8.49-8.37 (m, 1H) 8.20 (dd, J =8.2, 2.6 Hz, 1H) 7.65-7.57(m, 2H) 7.55-7.49 (m, 1H)7.47-7.31 (m, 4H) 7.20 (d, J =8.3 Hz, 1H) 3.74-3.65 (m,1H) 2.99 (s, 3H) 1.40 (m, 6H). |
| 104 | 3-{2-Amino-4-[4-fluoro-3-(2-fluoropyridin-3-yl)phenyl]-1-methyl-5-oxo-4,5-dihydro-1H-imidazol-4-yl}phenyl propane-2-sulfonate | 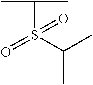 | 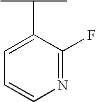 | 501 | 8.31 (d, J = 4.8 Hz, 1H) 8.05-7.95 (m, 1H) 7.62-7.40 (m,8H) 7.33 (t, J = 9.4 Hz, 1H) 7.24-7.17 (m, 1H) 3.74-3.63 (m,2H) 2.99 (s, 3H) 1.43-1.37 (m,2H). |
| 105 | 3-[2-Amino-4-(4-fluoro-3-pyridin-3-ylphenyl)-1-methyl-5-oxo-4,5-dihydro-1H-imidazol-4-yl]phenylpropane-2-sulfonate | 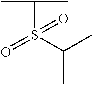 | 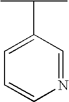 | 483 | 8.65 (s, 1H) 8.61 (dd, J = 4.8,1.8 Hz, 1H) 7.91-7.84 (m,1H) 7.61 (dd, J = 7.8, 2.3 Hz,1H) 7.57-7.36 (m, 5H) 7.33 (dd,J = 10.3, 8.53 Hz, 1H) 7.22-7.17 (m, 1H) 7.13 (t, J = 9.0Hz, 1H) 3.74-3.65 (m, 1H)2.99 (s, 3H) 1.40 (d, J = 6.8 Hz,6H). |
| 106 | 3-[2-Amino-4-(3′-chloro-6-fluorobiphenyl-3-yl)-1-methyl-5-oxo-4,5-dihydro-1H-imidazol-4-yl]phenyl propane-2-sulfonate | 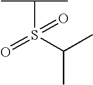 | 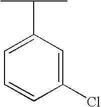 | 516 | 7.60 (dd, J = 7.5, 2.0 Hz, 1H)7.57-7.39 (m, 9H) 7.30 (dd, J =10.5, 8.8 Hz, 1H) 7.22-7.18(m, 1H) 3.75-3.65 (m, 1H)3.00 (s, 3H) 1.40 (d, J = 6.8 Hz,6H). |
| 107 | 3-[2-Amino-4-(3′-chloro-6-fluoro-5′-methoxybiphenyl-3-yl)-1-methyl-5-oxo-4,5-dihydro-1H-imidazol-4-yl]phenyl propane-2-sulfonate | 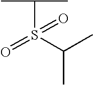 | 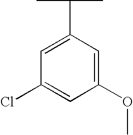 | 546 | 7.61-7.24 (m, 7H) 7.23-7.17(m, 1H) 7.11-7.08 (m, 1H)7.04 (s, 1H) 6.95 (s, 1H) 3.76-3.65 (m, 1H) 3.00 (s, 3H) 1.40(d, J = 6.8 Hz, 6H). |
| 108 | 3-[2-Amino-4-(6-fluoro-3′-methoxybiphenyl-3-yl)-1-methyl-5-oxo-4,5-dihydro-1H-imidazol-4-yl]phenyl propane-2-sulfonate | 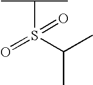 | 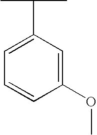 | 512 | No data |
| 109 | 3-[2-Amino-4-(3′,5′-dichloro-6-fluorobiphenyl-3-yl)-1-methyl-5-oxo-4,5-dihydro-1H-imidazol-4-yl]phenyl propane-2-sulfonate | 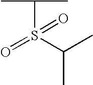 | 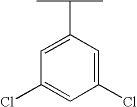 | 550 | No data |
| 110 | 3-{2-Amino-4-[4-fluoro-3-(2-fluoropyridin-3-yl)phenyl]-1-methyl-5-oxo-4,5-dihydro-1H-imidazol-4-yl}phenyldimethylsulfamate | 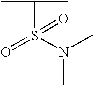 | 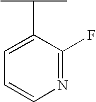 | 502 | 8.32 (d, J = 4.8 Hz, 1H) 8.03-7.96 (m, 1H) 7.63-7.40 (m,6H) 7.40-7.31 (m, 2H) 7.25-7.18 (m, 1H) 3.00 (s, 3H) 2.83(s, 6H). |
| 111 | 3-[2-Amino-4-(4-fluoro-3-pyridin-3-ylphenyl)-1-methyl-5-oxo-4,5-dihydro-1H-imidazol-4-yl]phenyldimethylsulfamate | 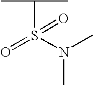 | 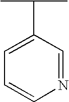 | 484 | No data |
| 112 | 3-[2-Amino-4-(6-fluoro-3′-methoxybiphenyl-3-yl)-1-methyl-5-oxo-4,5-dihydro-1H-imidazol-4-yl]phenyl dimethylsulfamate | 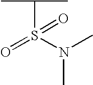 | 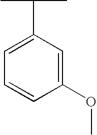 | 513 | No data |
| 113 | 3-[2-Amino-4-(3′,5′-dichloro-6-fluorobiphenyl-3-yl)-1-methyl-5-oxo-4,5-dihydro-1H-imidazol-4-yl]phenyl dimethylsulfamate | 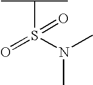 | 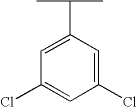 | 551 | No data |

### Example 114

### 2-(3-Methoxyphenyl)-1,3-dithiane

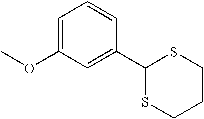

Propane-1,3-dithiol (7.56 g, 70 mmol) was added to a solution of 3-methoxybenzaldehyde (10 g, 73.5 mmol) in anhydrous dichloromethane (50 mL). The mixture was stirred for 15 min and then cooled to −78° C. Trifluoro(1,1′-oxydiethane)boron (28 mL, 220.5 mmol) was added and the reaction was stirred at −78° C. for 15 min before the cooling bath was removed and the reaction was allowed to reach room temperature. The solution was washed with water two times before the solvent was evaporated in vacuo. The product was purified by column chromatography, using a gradient of 0 to 40% ethyl acetate in n-heptane as the eluent, to give 6.52 g (39% yield) the title compound: ¹H NMR (CDCl₃) δ 7.27 (t, J=7.8 Hz, 1H), 7.14-7.01 (m, 2H), 6.91-6.81 (m, 1H), 5.17 (s, 1H), 3.84 (s, 3H), 3.19-3.01 (m, 2H), 3.00-2.87 (m, 2H), 2.27-2.11 (m, 1H), 2.04-1.89 (m, 1H).

### Example 115

### (3-Bromo-4-fluorophenyl)[2-(3-methoxyphenyl)-1,3-dithian-2-yl]methanol

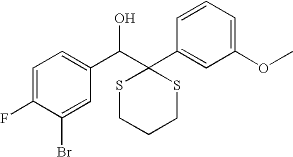

2-(3-Methoxyphenyl)-1,3-dithiane (6.52 g, 28.8 mmol) was dissolved in anhydrous tetrahydrofuran (30 mL) under an atmosphere of argon and cooled to −78° C. n-Butyl lithium (12.7 mL, 2.5 M) was added dropwise and the resulting solution was stirred at −78° C. for 20 min and was then treated with 3-bromo-4-fluorobenzaldehyde (6.15 g, 30.3 mmol). The reaction was stirred for another 20 min at −78° C. and was then allowed to reach room temperature and stirred for 3 days. Aqueous ammonium chloride was added and the mixture was extracted with dichloromethane. The combined organic phases were evaporated in vacuo and the product was purified by column chromatography, using a gradient of 0 to 50% ethyl acetate in n-heptane as the eluent, to give 9.41 g (76% yield) the title compound: ¹H NMR (CDCl₃) δ 7.39-7.19 (m, 3H), 7.03 (dd, J=6.6, 2.0 Hz, 2H), 6.94-6.77 (m, 3H), 4.90 (s, 1H), 3.75 (s, 3H), 2.84-2.62 (m, 4H), 2.02-1.88 (m, 2H); MS (ES) m/z 411, 413 [M-water]⁺.

### Example 116

### 1-(3-Bromo-4-fluorophenyl)-2-(3-methoxyphenyl)ethane-1,2-dione

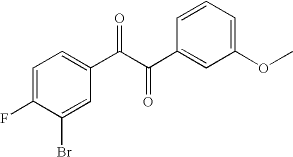

(3-Bromo-4-fluorophenyl)[2-(3-methoxyphenyl)-1,3-dithian-2-yl]methanol (13.57 g, 31.6 mmol) was dissolved in dichloromethane (50 mL) and tert-butanol (8.18 g, 110.6 mmol) under an atmosphere of nitrogen. Dess-Martin Periodinane (30.2 g, 71.1 mmol) was added and the reaction was stirred over night. Sodium thiosulfate (40 mL, 1 M) was added and the layers were separated. The organic phase was washed with aqueous sodium hydrogen carbonate and the solvent was evaporated. Purification by column chromatography, using a gradient of 0 to 100% ethyl acetate in n-heptane as the eluent, gave 7.55 g (71% yield) the title compound: MS (ES) m/z 335, 337 [M−1]⁻.

### Example 117

### 5-(3-Bromo-4-fluorophenyl)-5-(3-methoxyphenyl)-3-methyl-2-thioxoimidazolidin-4-one

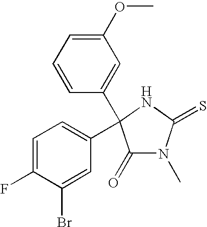

1-(3-Bromo-4-fluorophenyl)-2-(3-methoxyphenyl)ethane-1,2-dione (7.55 g, 22.4 mmol) and N-methylthiourea (4.0 g, 44.8 mmol) were dissolved in dimethyl sulfoxide (50 mL). The solution was heated to 100° C. and potassium hydroxide (38 mL, 1.2 M) was added dropwise. After the addition the reaction was stirred at 100° C. for 5 min and was then allowed to cool to room temperature. The solution was diluted with water and acidified using concentrated hydrochloric acid followed by extraction with dichloromethane. The combined organic phases were washed with water and concentrated in vacuo to give 8.46 g (92% yield) of the title compound: ¹H NMR (CDCl₃) δ 7.50 (dd, J=6.3, 2.3 Hz, 1H), 7.27-7.17 (m, 3H), 7.05 (m, 1H), 6.87-6.73 (m, 3H), 3.71 (s, 3H), 3.25 (s, 3H); MS (ES) m/z 409, 411 [M+1]⁺.

### Example 118

### 5-(3-Bromo-4-fluorophenyl)-5-(3-hydroxyphenyl)-3-methyl-2-thioxoimidazolidin-4-one

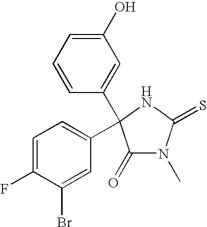

5-(3-Bromo-4-fluorophenyl)-5-(3-methoxyphenyl)-3-methyl-2-thioxoimidazolidin-4-one (8.46 g, 20.68 mmol) was dissolved in anhydrous dichloromethane and was cooled to 0° C. Boron tribromide (10.36 g, 41.37 mmol) was added and the reaction was stirred at 0° C. for 1.5 h. The mixture was washed with water and the solvent was evaporated. Purification by column chromatography, using a gradient of 0 to 50% ethyl acetate in n-heptane as the eluent, gave 5.27 g of the title compound: ¹H NMR (CDCl₃) δ 7.85 (s, 1H), 7.58 (dd, J=6.3, 2.3 Hz, 1H), 7.34-7.28 (m, 2H), 7.15 (t, J=8.3 Hz, 1H), 6.90-6.82 (m, 2H), 6.80-6.75 (m, 1H), 3.35 (s, 3H); MS (ES) m/z 393, 395 [M−1]⁻.

### Example 119

### 3-[4-(3-Bromo-4-fluorophenyl)-1-methyl-5-oxo-2-thioxoimidazolidin-4-yl]phenyl methanesulfonate

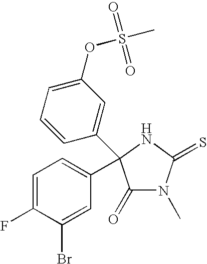

5-(3-Bromo-4-fluorophenyl)-5-(3-hydroxyphenyl)-3-methyl-2-thioxoimidazolidin-4-one (1.65 g, 4.17 mmol) and triethylamine (1.27 g, 12.52 mmol) were dissolved in dichloromethane (30 mL) and was cooled to 0° C. Methanesulfonyl chloride (717 mg, 6.26 mmol) was added in three portions and the reaction was allowed to reach room temperature and stirred for 2 h. The solvents were evaporated and the product was purified by column chromatography, using a gradient of 0 to 100% ethyl acetate in n-heptane as the eluent, to give 1.48 g of the title compound: MS (ES) m/z 471, 473 [M−1]⁻.

### Example 120

### 3-[4-(3-Bromo-4-fluorophenyl)-1-methyl-5-oxo-2-thioxoimidazolidin-4-yl]phenyl propane-1-sulfonate

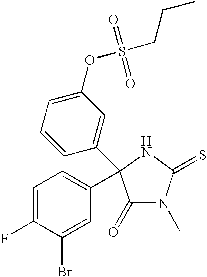

The title compound was synthesized as described for Example 119 in 65% yield, starting from 1-propanesulfonyl chloride: MS (ES) m/z 499, 501 [M−1]⁻.

### Example 121

### 3-[4-(3-Bromo-4-fluorophenyl)-1-methyl-5-oxo-2-thioxoimidazolidin-4-yl]phenyl propane-2-sulfonate

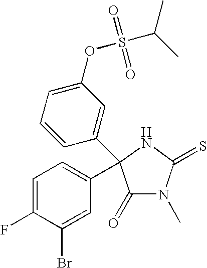

The title compound was synthesized as described for Example 119 in 50% yield, starting from 2-propanesulphonyl chloride: MS (ES) m/z 499, 501 [M−1]⁻.

### Example 122

### 3-[4-(3-Bromo-4-fluorophenyl)-1-methyl-5-oxo-2-thioxoimidazolidin-4-yl]phenyl dimethylsulfamate

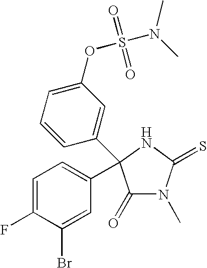

The title compound was synthesized as described for Example 119 in 74% yield, starting from N,N-dimethylsulfamoyl chloride: MS (ES) m/z 500, 502 [M−1]⁻.

### Example 123

### 3-[2-Amino-4-(3-bromo-4-fluorophenyl)-1-methyl-5-oxo-4,5-dihydro-1H-imidazol-4-yl]phenyl methanesulfonate

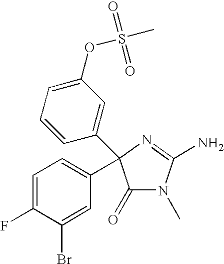

3-[4-(3-Bromo-4-fluorophenyl)-1-methyl-5-oxo-2-thioxoimidazolidin-4-yl]phenyl methanesulfonate (1.48 g, 3.13 mmol) was dissolved in methanol (30 mL) and ammonium hydroxide (33%, 10 mL) and tert-butyl hydroperoxide (4.23 g, 46.9 mmol) was added. The mixture was stirred at room temperature over night and was then concentrated until approximately 50% of the volume remained. Water was added and the mixture was extracted with dichloromethane. The combined organic phases were washed with water and concentrated in vacuo. The product was purified by column chromatography, using a gradient of 0 to 10% 1:9 ammonium hydroxide/methanol in dichloromethane as the eluent, to give 940 mg (66% yield) of the title compound: ¹H NMR (CDCl₃) δ 7.76 (dd, J=6.6, 2.3 Hz, 1H), 7.52 (d, J=7.8 Hz, 1H), 7.49-7.43 (m, 1H), 7.39 (t, J=8.1 Hz, 1H), 7.37-7.35 (m, 1H), 7.23-7.17 (m, 1H), 7.07 (t, J=8.5 Hz, 1H), 3.16 (s, 3H), 3.11 (s, 3H); MS (ES) m/z 556, 558 [M+1]⁺.

### Example 124

### 3-[2-Amino-4-(3-bromo-4-fluorophenyl)-1-methyl-5-oxo-4,5-dihydro-1H-imidazol-4-yl]phenyl propane-1-sulfonate

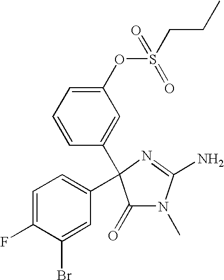

The title compound was synthesized as described for Example 123 in 90% yield, starting from 3-[4-(3-bromo-4-fluorophenyl)-1-methyl-5-oxo-2-thioxoimidazolidin-4-yl]phenyl propane-1-sulfonate: ¹H NMR (CDCl₃) δ 7.77 (dd, J=6.7, 2.4 Hz, 1H), 7.47 (dd, J=8.6, 2.3 Hz, 1H), 7.48 (td, J=8.9, 7.4 Hz, 1H), 7.38 (t, J=8.0 Hz, 1H), 7.29 (t, J=2.0 Hz, 1H), 7.17 (dd, J=7.7, 1.9 Hz, 1H), 7.07 (t, J=8.5 Hz, 1H), 3.28-3.18 (m, 2H), 3.09 (s, 3H), 2.06-1.96 (m, 2H), 1.12 (t, J=7.5 Hz, 3H): MS (ES) m/z 484, 486 [M+1]⁺.

### Example 125

### 3-[2-Amino-4-(3-bromo-4-fluorophenyl)-1-methyl-5-oxo-4,5-dihydro-1H-imidazol-4-yl]phenyl propane-2-sulfonate

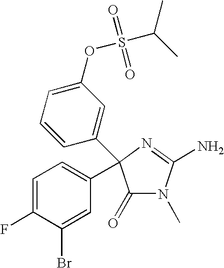

The title compound was synthesized as described for Example 123 in 73% yield starting from 3-[4-(3-bromo-4-fluorophenyl)-1-methyl-5-oxo-2-thioxoimidazolidin-4-yl]phenyl propane-2-sulfonate: ¹H NMR (CDCl₃) δ 7.76 (dd, 1H), 7.51-7.43 (m, 2H), 7.37 (t, 1H), 7.31-7.29 (m, 1H), 7.20-7.15 (m, 1H), 7.07 (t, J=8.3 Hz, 1H), 3.53-3.42 (m, 1H), 3.10 (s, 3H), 1.56 (s, 6H): MS (ES) m/z 484, 486 [M+1]⁺.

### Example 126

### 3-[2-Amino-4-(3-bromo-4-fluorophenyl)-1-methyl-5-oxo-4,5-dihydro-1H-imidazol-4-yl]phenyl dimethylsulfamate

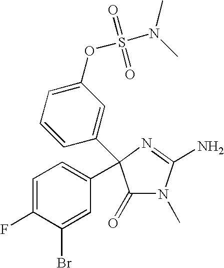

The title compound was synthesized as described for Example 123 in 59% yield, starting from 3-[4-(3-bromo-4-fluorophenyl)-1-methyl-5-oxo-2-thioxoimidazolidin-4-yl]phenyl dimethylsulfamate: ¹H NMR (CDCl₃) δ 7.74 (dd, J=6.4, 2.4 Hz, 1H), 7.50-7.33 (m, 4H), 7.27-7.23 (m, 1H), 7.10 (t, J=8.5 Hz, 1H), 3.21 (s, 3H), 2.97 (s, 6H): MS (ES) m/z 485, 487 [M+1]⁺.

### Example 127

### 4-[4-(3-Bromo-4-fluorophenyl)-1-methyl-5-oxo-2-thioxoimidazolidin-4-yl]phenyl propane-1-sulfonate

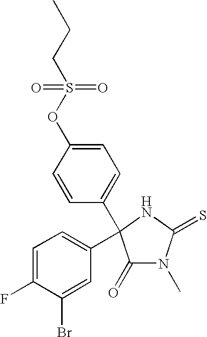

The title compound was synthesized as described for Example 119 in 77% yield, starting from 5-(3-bromo-4-fluorophenyl)-5-(4-hydroxyphenyl)-3-methyl-2-thioxoimidazolidin-4-one and 1-propanesulphonyl chloride: MS (ES) m/z 499, 501 [M−1]⁻.

### Example 128

### 4-[2-Amino-4-(3-bromo-4-fluorophenyl)-1-methyl-5-oxo-4,5-dihydro-1H-imidazol-4-yl]phenyl propane-1-sulfonate

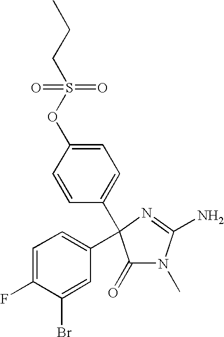

The title compound was synthesized as described for Example 123 in 73% yield, starting from 4-[4-(3-bromo-4-fluorophenyl)-1-methyl-5-oxo-2-thioxoimidazolidin-4-yl]phenyl propane-1-sulfonate: MS (ES) m/z 482, 484 [M−1]⁻.

### Example 129

### 4-{2-Amino-4-[4-fluoro-3-(5-methoxypyridin-3-yl)phenyl]-1-methyl-5-oxo-4,5-dihydro-1H-imidazol-4-yl}phenyl propane-1-sulfonate hydrochloride

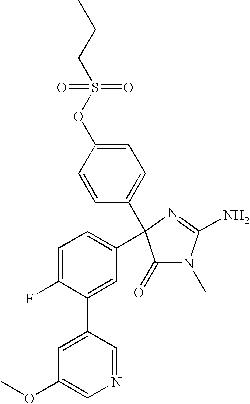

Palladium-tetrakis(triphenylphosphine) (50 mg, 0.042 mmol), silver oxide (400 mg, 1.70 mmol) and 4-[2-amino-4-(3-bromo-4-fluorophenyl)-1-methyl-5-oxo-4,5-dihydro-1H-imidazol-4-yl]phenyl propane-1-sulfonate (820 mg, 1.70 mmol) was dissolved in anhydrous N,N-dimethylformamide (15 mL) and stirred at 130° C. for 1 h. 3-Bromo-5-methoxypyridine (634 mg, 3.38 mmol), palladium-tetrakis(triphenylphosphine) (50 mg, 0.042 mmol) and hexamethylditin (1104 mg, 3.38 mmol) was dissolved in anhydrous tetrahydrofuran (15 mL) and irradiated in a microwave at 130° C. for 1 h. Half of the mixture was added to the N,N-dimethylformamide solution and the resulting mixture was stirred for 2 h at 130° C. Palladium-tetrakis(triphenylphosphine) (50 mg, 0.042 mmol) was added to the reaction together with the rest of the tetrahydrofuran solution. The reaction was stirred at 130° C. over night and was then allowed to cool to room temperature, filtered and purified using preparative HPLC followed by column chromatography, using a gradient of 0 to 10% 1:9 ammonium hydroxide/methanol in dichloromethane as the eluent, to give 76.3 mg of the base which was dissolved in 1 M hydrochloric acid in diethyl ether. The solvent was evaporated to give 71.4 mg (7.6% yield) of the title compound: ¹H NMR (DMSO-d₆) δ 8.48-8.24 (m, 2H), 7.71-7.27 (m, 8H), 3.89 (s, 3H), 3.58-3.47 (m, 2H), 1.88-1.81 (m, 2H), 1.06-0.98 (m, 3H); MS (ES) m/z 513 [M+1]⁺.

### Example 130

### 3-{2-Amino-4-[4-fluoro-3-(5-methoxypyridin-3-yl)phenyl]-1-methyl-5-oxo-4,5-dihydro-1H-imidazol-4-yl}phenyl propane-1-sulfonate hydrochloride

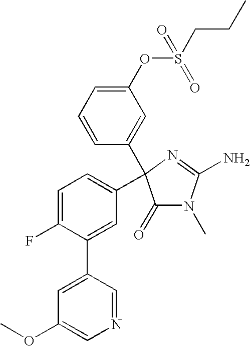

The title compound was synthesized as described for Example 129 in 3.7% yield, starting from 3-[2-amino-4-(3-bromo-4-fluorophenyl)-1-methyl-5-oxo-4,5-dihydro-1H-imidazol-4-yl]phenyl propane-1-sulfonate: ¹H NMR (DMSO-d₆) δ 8.43-8.23 (m, 2H), 7.65-7.31 (m, 8H), 3.88 (s, 3H), 3.58-3.49 (m, 2H), 3.18 (s, 3H), 1.87-1.73 (m, 2H), 1.03-0.95 (m, 3H); MS (ES) m/z 511, 513[M−1]⁻.

### Example 131

### 3-[4-(3-Bromo-4-fluorophenyl)-1-methyl-5-oxo-2-thioxoimidazolidin-4-yl]phenyl benzenesulfonate

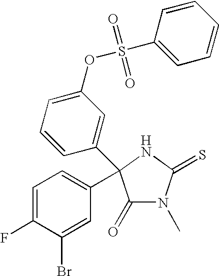

5-(3-Bromo-4-fluorophenyl)-5-(3-hydroxyphenyl)-3-methyl-2-thioxoimidazolidin-4-one (150 mg, 0.38 mmol) and triethylamine (0.16 mL, 1.14 mmol) were dissolved in anhydrous dichloromethane (5 mL) and was cooled to 0° C. Benzenesulfonyl chloride (53 μL, 0.42 mmol) was added and the reaction was allowed to reach room temperature and stirred for 1 h under an atmosphere of argon. The solvents were evaporated and the residue slurried in ethyl acetate. The precipitate was filtered off and the filtrate concentrated in vacuo to give 203 mg (quantitative yield) of the title compound: MS (ES) m/z 533, 535 [M−1]⁻.

### Example 132

### 3-[2-Amino-4-(3-bromo-4-fluorophenyl)-1-methyl-5-oxo-4,5-dihydro-1H-imidazol-4-yl]phenyl benzenesulfonate

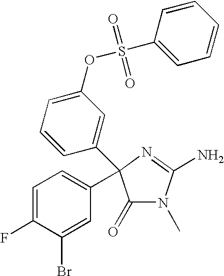

The title compound was synthesized as described for Example 74 in 43% yield, starting from 3-[4-(3-bromo-4-fluorophenyl)-1-methyl-5-oxo-2-thioxoimidazolidin-4-yl]phenyl benzenesulfonate: MS (ES) m/z 518, 520 [M+1]⁺.

### Example 133

### 3-[2-Amino-4-(4-fluoro-3-pyrimidin-5-ylphenyl)-1-methyl-5-oxo-4,5-dihydro-1H-imidazol-4-yl]phenyl benzenesulfonate hydrochloride

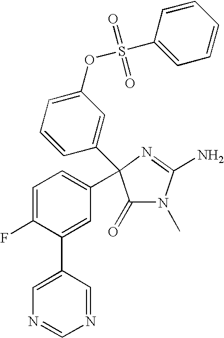

A mixture of 3-[2-amino-4-(3-bromo-4-fluorophenyl)-1-methyl-5-oxo-4,5-dihydro-1H-imidazol-4-yl]phenyl benzenesulfonate (85 mg, 0.16 mmol), pyrimidin-5-ylboronic acid (26 mg, 0.21 mmol), [1,1′-bis(diphenylphosphino)ferrocene]palladium(II) chloride dichloromethane adduct (13 mg, 0.02 mmol), potassium carbonate (133 mg, 0.96 mmol) and anhydrous tetrahydrofuran (3 mL) was irradiated in a microwave at 130° C. for 3 h. When cooled to room temperature the mixture was diluted with water and extracted with diethyl ether. The combined organics were concentrated and purified by HPLC. The acetonitrile was removed in vacuo, the residue diluted with saturated aqueous sodium hydrogen carbonate and extracted with ethyl acetate. The combined organics were dried over sodium sulfate, filtered and hydrochloric acid (1 M in diethyl ether, 0.3 mL) was added to the filtrate. The mixture was stirred at room temperature for 5 min and the solvent was evaporated to give 48 mg (54% yield) of the title compound: ¹H NMR (DMSO-d₆) δ 11.77 (br s, 1H), 9.73 (br s, 2H), 9.27 (s, 1H), 9.04 (s, 2H), 7.82-7.76 (m, 3H), 7.66-7.59 (m, 3H), 7.54-7.47 (m, 2H), 7.46-7.40 (m, 1H), 7.29-7.23 (m, 1H), 7.22-7.16 (m, 1H), 7.01-6.95 (m, 1H), 3.17 (s, 3H); MS (ES) m/z 518 [M+1]⁺.

### Example 134

### 4-{2-Amino-4-[4-fluoro-3-(2-fluoropyridin-3-yl)phenyl]-5-oxo-4,5-dihydro-1H-imidazol-4-yl}phenyl methanesulfonate

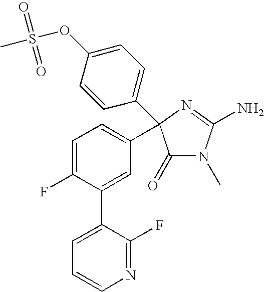

A mixture of 4-[2-Amino-4-(3-bromo-4-fluorophenyl)-1-methyl-5-oxo-4,5-dihydro-1H-imidazol-4-yl]phenyl methanesulfonate (2.74 g, 6 mmol), 2-fluoropyridine-3-boronic acid (1.1 g, 7.8 mmol), [1,1′-bis(diphenylphosphino)ferrocene]palladium(II) chloride dichloromethane adduct (490 mg, 0.6 mmol), potassium carbonate (4.98 g, 36 mmol) and anhydrous tetrahydrofuran (51 mL) was irradiated in a microwave at 130° C. for 3 h. When cooled to room temperature the mixture was diluted with water and extracted with dichloromethane. The aqueous phase was acidified with 1 M hydrochloric acid and extracted with ethyl acetate. The combined organics were concentrated and purified by column chromatography, using 0-10% 0.1 M ammonia in methanol in dichloromethane as the eluent. The product was dissolved in dichloromethane and stirred with active charcoal at room temperature for 10 min, filtered through celite and concentrated to give 1.75 g (62% yield) of the title compound: MS (ES) m/z 473 [M+1]⁺.

### Example 135

### (R) and (S) 4-{2-Amino-4-[4-fluoro-3-(2-fluoropyridin-3-yl)phenyl]-5-oxo-4,5-dihydro-1H-imidazol-4-yl}phenyl methanesulfonate hydrochloride Chromatographic separation of the enantiomers of 4-{2-amino-4-[4-fluoro-3-(2-fluoropyridin-3-yl)phenyl]-5-oxo-4,5-dihydro-1H-imidazol-4-yl}phenyl methanesulfonate hydrochloride

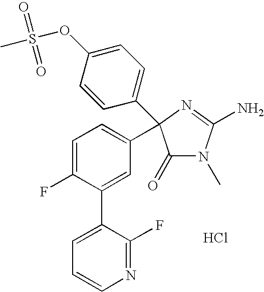

4-{2-Amino-4-[4-fluoro-3-(2-fluoropyridin-3-yl)phenyl]-5-oxo-4,5-dihydro-1H-imidazol-4-yl}phenyl methanesulfonate (3.7 g) was dissolved in methanol and the resulting solution was injected (20 mg in 0.8 mL methanol per injection) on a Chiralpak OD column (21.2×250 mm), using 20% (methanol with 0.1% diethyl amine) and 80% carbon dioxide as eluent at a flow rate of 50 mL/min. Detection was monitored at 220 nm and the two isomers were collected. To each of the collected solutions were added hydrochloric acid (1 M in diethyl ether, 2.5 mL). The resulting mixtures were stirred for 5 min at room temperature and concentrated to dryness to give the title compounds.

Isomer 1, the first isomer to elute (748 mg, 1.58 mmol, 40% yield): ¹H NMR (DMSO-d₆) δ 11.58 (br s, 1H), 9.66 (br s, 2H), 8.37-8.32 (m, 1H), 8.08-8.03 (m, 1H), 7.60-7.57 (m, 1H), 7.55-7.44 (m, 7H), 3.41 (s, 3H), 3.18 (s, 3H).

Isomer 2, the second isomer to elute (740 mg, 1.57 mmol, 40% yield): ¹H NMR (DMSO-d₆) δ 11.70 (br s, 1H), 9.70 (br s, 2H), 8.37-8.33 (m, 1H), 8.10-8.04 (m, 1H), 7.61-7.57 (m, 1H), 7.55-7.44 (m, 7H), 3.41 (s, 3H), 3.18 (s, 3H).

### Example 136

### 5-{5-[2-Amino-4-(4-hydroxyphenyl)-1-methyl-5-oxo-4,5-dihydro-1H-imidazol-4-yl]-2-fluorophenyl}nicotinonitrile

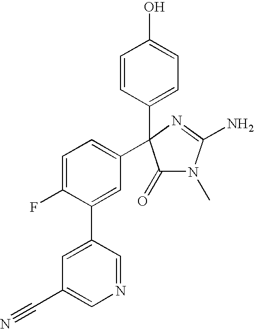

5-Bromonicotinonitrile (201 mg, 1.1 mmol), hexamethyldistannane (230 μL, 1.1 mmol) and tetrakis(triphenylphosphine)palladium(0) (32 mg, 0.028 mmol) was dissolved in anhydrous tetrahydrofuran (2.5 mL) and irradiated in a microwave at 130° C. for 30 min. After cooling to room temperature 2-amino-5-(3-bromo-4-fluorophenyl)-5-(4-hydroxyphenyl)-3-methyl-3,5-dihydro-4H-imidazol-4-one (190 mg, 0.5 mmol) and additional tetrakis(triphenylphosphine)palladium(0) (32 mg, 0.028 mmol) were added and the mixture was irradiated in a microwave at 130° C. for 3 h. Silica was added and the solvent was evaporated. Purification by column chromatography, using a gradient of 0-10% 0.1 M ammonia in methanol in dichloromethane as the eluent, gave 106 mg (53% yield) of the title compound: ¹H NMR (DMSO-d₆) δ 9.05-9.08 (m, 1H), 8.95-8.98 (m, 1H), 8.46-8.49 (m, 1H), 7.62-7.66 (m, 1H), 7.49-7.55 (m, 1H), 7.37-7.44 (m, 1H), 7.17-7.22 (m, 2H), 6.70-6.75 (m, 2H), 3.05 (s, 3H); MS (ES) m/z 402 [M+1]⁺.

### Example 137

### 2-Amino-5-(4-fluoro-3-pyrazin-2-ylphenyl)-5-(4-hydroxyphenyl)-3-methyl-3,5-dihydro-4H-imidazol-4-one

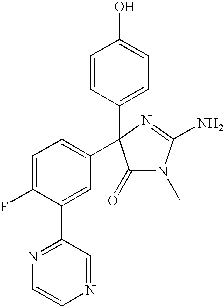

2-Amino-5-(3-bromo-4-fluorophenyl)-5-(4-hydroxyphenyl)-3-methyl-3,5-dihydro-4H-imidazol-4-one (190 mg, 0.5 mmol), tetrakis(triphenylphosphine)palladium(0) (29 mg, 0.025 mmol) and 2-tributylstannylpyrazine (221 mg, 0.6 mmol) in tetrahydrofuran (3 mL) were irradiated in a microwave at 130° C. for 2 h. Silica was added and the solvent was evaporated. Purification by column chromatography, using a gradient of 0-10% 0.1 M ammonia in methanol in dichloromethane as the eluent, gave 167 mg (88% yield) of the title compound: ¹H NMR (DMSO-d₆) δ 8.99-9.02 (m, 1H), 8.78-8.81 (m, 1H), 8.65 (d, J=2.5 Hz, 1H), 8.08-8.12 (m, 1H), 7.58-7.63 (m, 1H), 7.29-7.36 (m, 1H), 7.17-7.23 (m, 2H), 6.65-6.68 (m, 2H), 2.97 (s, 3H); MS (ES) m/z 378 [M+1]⁺.

### Example 138

### 2-Amino-5-[4-fluoro-3-(5-fluoropyridin-3-yl)phenyl]-5-(4-hydroxyphenyl)-3-methyl-3,5-dihydro-4H-imidazol-4-one

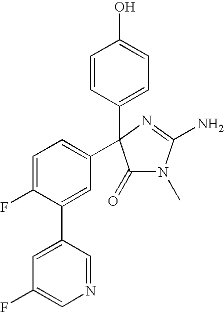

The title compound was synthesized as described for example 136 in 62% yield, starting from 2-amino-5-(3-bromo-4-fluorophenyl)-5-(4-hydroxyphenyl)-3-methyl-3,5-dihydro-4H-imidazol-4-one and 5-bromo-3-fluoropyridine: ¹H NMR (DMSO-d₆) δ 9.34 (br s, 1H), 8.63 (d, J=2.8 Hz, 1H), 8.51-8.55 (m, 1H), 7.83-7.89 (m, 1H), 7.60-7.64 (m, 1H), 7.52-7.57 (m, 1H), 7.27-7.35 (m, 1H), 7.20-7.26 (m, 2H), 6.65-6.69 (m, 2H), 2.97 (s, 3H); MS (ES) m/z 395 [M+1]⁺.

### Example 139

### 4-[2-Amino-4-(3-bromo-4-fluorophenyl)-1-methyl-5-oxo-4,5-dihydro-1H-imidazol-4-yl]phenyl trifluoromethanesulfonate

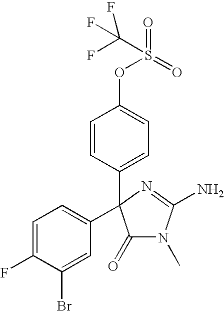

2-Amino-5-(3-bromo-4-fluorophenyl)-5-(4-hydroxyphenyl)-3-methyl-3,5-dihydro-4H-imidazol-4-one (1.02 g, 2.7 mmol) and triethylamine (0.4 mL, 3 mmol) in dichloromethane (20 mL) was cooled to 0° C. N-Phenyl-bis(trifluoromethanesulfonimide) (1.07 g, 3 mmol) was added and the mixture was stirred at room temperature for 72 h. Water and saturated aqueous sodium bicarbonate was added and the mixture was extracted with ethyl acetate. The organic phases were pooled, washed with brine, dried over magnesium sulfate and concentrated. Purification by column chromatography, using a gradient of 0-3% 0.1 M ammonia in methanol in dichloromethane as the eluent, gave 0.85 g (62% yield) of the title compound: ¹H NMR (DMSO-d₆) δ 7.71-7.74 (m, 1H), 7.58-7.62 (m, 2H), 7.48-7.53 (m, 1H), 7.43-7.47 (m, 2H), 7.32-7.37 (m, 1H), 6.84 (br s, 2H), 2.99 (s, 3H); MS (ES) m/z 508, 510 [M−1]⁻.

### Example 140

### 4-{2-Amino-4-[3-(5-chloropyridin-3-yl)-4-fluorophenyl]-1-methyl-5-oxo-4,5-dihydro-1H-imidazol-4-yl}phenyl trifluoromethanesulfonate 0.75 acetate

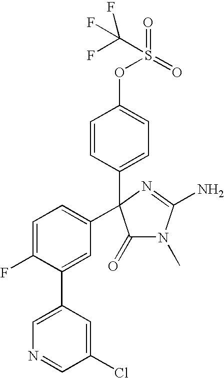

4-[2-Amino-4-(3-bromo-4-fluorophenyl)-1-methyl-5-oxo-4,5-dihydro-1H-imidazol-4-yl]phenyl trifluoromethanesulfonate (100 mg, 0.196 mmol), potassium carbonate (162 mg, 1.17 mmol), 5-chloropyridine-3-boronic acid (30 mg, 0.186 mmol) and [1,1′-bis(diphenylphosphino)ferrocene]palladium(II) chloride dichloromethane adduct (16 mg, 0.0192 mmol) was heated to 100° C. in anhydrous dioxane (4 mL) overnight and then irradiated in a microwave at 120° C. for 1 h. Silica was added and the solvent was evaporated. Purification by column chromatography, using a gradient of 0-4% 0.1 M ammonia in methanol in dichloromethane as the eluent, followed by preparative HPLC and freeze drying afforded 26 mg (23% yield) of the title compound: ¹H NMR (DMSO-d₆) δ 8.68 (d, J=2.3 Hz, 1H), 8.63 (t, J=1.6 Hz, 1H), 8.05-8.08 (m, 1H), 7.65-7.70 (m, 3H), 7.57-7.61 (m, 1H), 7.42-7.47 (m, 2H), 7.33-7.39 (m, 1H), 2.99 (s, 3H), 1.89 (s, 2.3H); MS (ES) m/z 541 [M−1]⁻.

### Example 141

### 4-[2-Amino-4-(4-fluoro-3-pyridin-3-ylphenyl)-1-methyl-5-oxo-4,5-dihydro-1H-imidazol-4-yl]phenyl trifluoromethanesulfonate 0.5 acetate

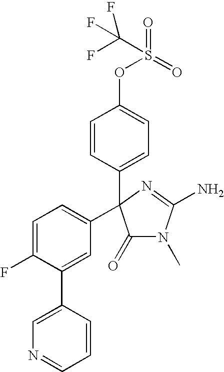

The title compound was synthesized as described for example 140 in 57% yield, starting from 3-pyridine boronic acid: ¹H NMR (DMSO-d₆) δ 8.65-8.69 (m, 1H), 8.60-8.63 (m, 1H), 7.87-7.92 (m, 1H), 7.63-7.70 (m, 3H), 7.50-7.58 (m, 2H), 7.42-7.47 (m, 2H), 7.30-7.36 (m, 1H), 2.99 (s, 3H), 1.90 (s, 1.7H); MS (ES) m/z 507 [M−1]⁻.

### Example 142

### 4-{2-Amino-4-[4-fluoro-3-(2-fluoropyridin-3-yl)phenyl]-1-methyl-5-oxo-4,5-dihydro-1H-imidazol-4-yl}phenyl trifluoromethanesulfonate 0.75 acetate

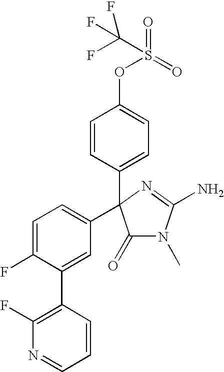

The title compound was synthesized as described for example 140 in 20% yield, starting from 2-fluoropyridine-3-boronic acid in tetrahydrofuran/water (4:1, 5 mL) and the mixture was irradiated in a microwave at 150° C. for 15 min: ¹H NMR (DMSO-d₆) δ 8.30-8.34 (m, 1H), 7.99-8.04 (m, 1H), 7.63-7.67 (m, 2H), 7.57-7.62 (m, 2H), 7.42-7.51 (m, 3H), 7.31-7.37 (m, 1H), 2.99 (s, 3H), 1.89 (s, 1.9H); MS (ES) m/z 525 [M−1]⁻.

### Example 143

### 4-[2-Amino-4-(4-fluoro-3-pyrazin-2-ylphenyl)-1-methyl-5-oxo-4,5-dihydro-1H-imidazol-4-yl]phenyl trifluoromethanesulfonate 0.5 acetate

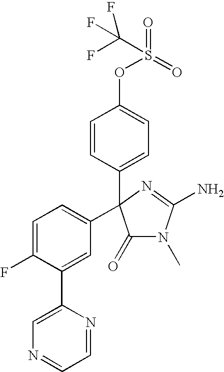

2-Amino-5-(4-fluoro-3-pyrazin-2-ylphenyl)-5-(4-hydroxyphenyl)-3-methyl-3,5-dihydro-4H-imidazol-4-one (167 mg, 0.44 mmol), and triethylamine (64 μl, 0.46 mmol) was dissolved in dichloromethane (10 mL) and N,N-dimethylformamide (10 drops). N-phenyl-bis(trifluoromethanesulfonimide) (157 mg, 0.44 mmol) was added and the mixture was stirred overnight. The mixture was concentrated and methanol (3 mL) was added. Purification by preparative HPLC and freeze drying afforded 67 mg (28% yield) of the title compound: ¹H NMR (DMSO-d₆) δ 9.00-9.03 (m, 1H), 8.79-8.82 (m, 1H), 8.66 (d, J=2.5 Hz, 1H), 8.14-8.18 (m, 1H), 7.62-7.67 (m, 3H), 7.43-7.48 (m, 2H), 7.34-7.40 (m, 1H), 3.00 (s, 3H), 1.90 (s, 1.2H); MS (ES) m/z 508 [M−1]⁻.

### Example 144

### 4-{2-Amino-4-[3-(5-cyanopyridin-3-yl)-4-fluorophenyl]-1-methyl-5-oxo-4,5-dihydro-1H-imidazol-4-yl}phenyl trifluoromethanesulfonate 0.5 acetate

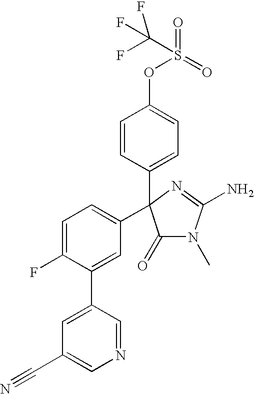

The title compound was synthesized as described for example 143 in 23% yield, starting from 5-{5-[2-amino-4-(4-hydroxyphenyl)-1-methyl-5-oxo-4,5-dihydro-1H-imidazol-4-yl]-2-fluorophenyl}nicotinonitrile: ¹H NMR (DMSO-d₆) δ 9.05-9.08 (m, 1H), 8.95-8.98 (m, 1H), 8.46-8.49 (m, 1H), 7.65-7.72 (m, 3H), 7.58-7.63 (m, 1H), 7.42-7.48 (m, 2H), 7.34-7.40 (m, 1H), 2.99 (s, 3H), 1.90 (s, 1.7H); MS (ES) m/z 532 [M−1]⁻.

### Example 145

### 4-{2-amino-4-[4-fluoro-3-(5-fluoropyridin-3-yl)phenyl]-1-methyl-5-oxo-4,5-dihydro-1H-imidazol-4-yl}phenyl trifluoromethanesulfonate 0.5 acetate

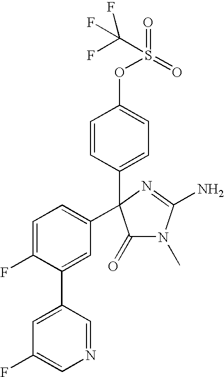

The title compound was synthesized as described for example 143 in 42% yield, starting from 2-amino-5-[4-fluoro-3-(5-fluoropyridin-3-yl)phenyl]-5-(4-hydroxyphenyl)-3-methyl-3,5-dihydro-4H-imidazol-4-one: ¹H NMR (DMSO-d₆) δ 8.64 (d, J=2.5 Hz, 1H), 8.54-8.57 (m, 1H), 7.86-7.91 (m, 1H), 7.65-7.70 (m, 3H), 7.56-7.61 (m, 1H), 7.42-7.47 (m, 2H), 7.33-7.38 (m, 1H), 2.99 (s, 3H), 1.90 (s, 1.6H); MS (ES) m/z 525 [M−1]⁻.

### Example 146

### 2-Amino-5-[4-fluoro-3-(5-methoxypyridin-3-yl)phenyl]-5-(4-hydroxyphenyl)-3-methyl-3,5-dihydro-4H-imidazol-4-one

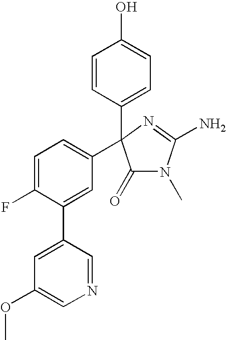

The title compound was synthesized as described for example 136 in 60% yield, starting from 2-amino-5-(3-bromo-4-fluorophenyl)-5-(4-hydroxyphenyl)-3-methyl-3,5-dihydro-4H-imidazol-4-one and 5-bromo-3-methoxypyridine: ¹H NMR (DMSO-d₆) δ 8.33 (d, J=2.8 Hz, 1H), 8.21-8.23 (m, 1H), 7.56-7.60 (m, 1H), 7.49-7.54 (m, 1H), 7.40-7.44 (m, 1H), 7.25-7.31 (m, 1H), 7.20-7.25 (m, 2H), 6.65-6.70 (m, 2H), 3.87 (s, 3H), 2.97 (s, 3H); MS (ES) m/z 405 [M−1]⁻.

### Example 147

### 2-Amino-5-[3-(4-chloropyridin-2-yl)-4-fluorophenyl]-5-(4-hydroxyphenyl)-3-methyl-3,5-dihydro-4H-imidazol-4-one

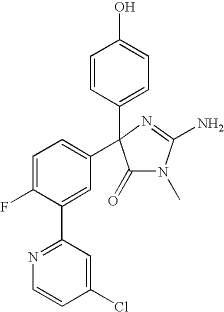

The title compound was synthesized as described for example 136 in 50% yield, starting from 2-amino-5-(3-bromo-4-fluorophenyl)-5-(4-hydroxyphenyl)-3-methyl-3,5-dihydro-4H-imidazol-4-one and 2-bromo-4-chloropyridine (described in Eur. J. Org. Chem. 2001, 3, 603-606): ¹H NMR (DMSO-d₆) δ 8.67-8.71 (m, 1H), 7.79-7.84 (m, 1H), 7.54-7.60 (m, 1H), 7.38-7.45 (m, 1H), 7.24-7.32 (m, 1H), 7.15-7.23 (m, 2H), 7.07-7.13 (m, 1H), 6.64-6.70 (m, 2H), 2.97 (s, 3H); MS (ES) m/z 411, 413 [M+1]⁺.

### Example 148

### 4-{2-Amino-4-[4-fluoro-3-(5-methoxypyridin-3-yl)phenyl]-1-methyl-5-oxo-4,5-dihydro-1H-imidazol-4-yl}phenyl trifluoromethanesulfonate 0.5 acetate

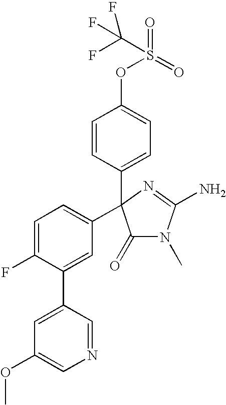

The title compound was synthesized as described for example 143 in 30% yield, starting from 2-amino-5-[4-fluoro-3-(5-methoxypyridin-3-yl)phenyl]-5-(4-hydroxyphenyl)-3-methyl-3,5-dihydro-4H-imidazol-4-one: ¹H NMR (DMSO-d₆) δ 8.34 (d, J=2.8 Hz, 1H), 8.23-8.25 (m, 1H), 7.62-7.69 (m, 3H), 7.53-7.57 (m, 1H), 7.43-7.47 (m, 3H), 7.30-7.36 (m, 1H), 3.87 (s, 3H), 2.99 (s, 3H), 1.91 (s, 1.7H); MS (ES) m/z 537 [M−1]⁻.

### Example 149

### 4-{2-Amino-4-[3-(4-chloropyridin-2-yl)-4-fluorophenyl]-1-methyl-5-oxo-4,5-dihydro-1H-imidazol-4-yl}phenyl trifluoromethanesulfonate 0.75 acetate

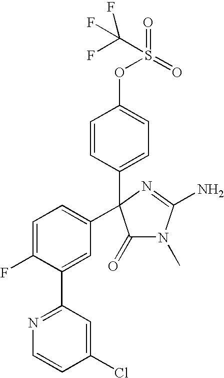

The title compound was synthesized as described for example 143 in 21% yield, starting from 2-amino-5-[3-(4-chloropyridin-2-yl)-4-fluorophenyl]-5-(4-hydroxyphenyl)-3-methyl-3,5-dihydro-4H-imidazol-4-one: ¹H NMR (DMSO-d₆) δ 8.68-8.71 (m, 1H), 8.14 (dd, J=7.6, 2.4 Hz, 1H), 7.82-7.85 (m, 1H), 7.59-7.65 (m, 3H), 7.56-7.59 (m, 1H), 7.43-7.47 (m, 2H), 7.30-7.36 (m, 1H), 3.00 (s, 3H), 1.90 (s, 1.9H); MS (ES) m/z 541, 543 [M−1]⁻.

### Example 150

### 2-(4-Methoxyphenyl)-1,3-dithiane

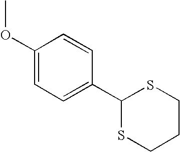

The title compound was synthesized as described for Example 114 in 93% yield, starting from propane-1,3-dithiol and 4-methoxybenzaldehyde: ¹H NMR (CDCl₃) δ 7.48-7.38 (m, 2H), 6.90-6.85 (m, 2H), 3.80 (s, 3H), 3.11-3.02 (m, 2H), 2.91 (dt, J=14.7, 3.7 Hz, 2H), 2.21-2.13 (m, 1H), 1.98-1.86 (m, 1H).

### Example 151

### (3-Bromo-4-fluorophenyl)[2-(4-methoxyphenyl)-1,3-dithian-2-yl]methanol

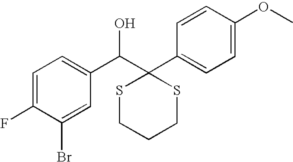

The title compound was synthesized as described for Example 115 in 75% yield, starting from 2-(4-methoxyphenyl)-1,3-dithiane and 3-bromo-4-fluorobenzaldehyde: ¹H NMR (CDCl₃) δ 7.57-7.54 (m, 2H), 7.01 (dd, J=6.7, 2.2 Hz, 1H), 6.91-6.83 (m, 3H), 6.81-6.76 (m, 1H), 4.90 (br s, 1H), 3.84 (s, 3H), 2.80-2.63 (m, 4H), 1.97-1.89 (m, 2H).

### Example 152

### 1-(3-Bromo-4-fluorophenyl)-2-(4-methoxyphenyl)ethane-1,2-dione

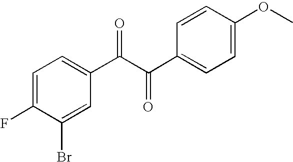

A solution of (3-bromo-4-fluorophenyl)[2-(4-methoxyphenyl)-1,3-dithian-2-yl]methanol (20 g, 47 mmol) in ethyl acetate (340 mL) was added dropwise to a solution of N-bromosuccinimide (125 g, 705 mmol) in aqueous sodium hydrogen carbonate (690 mL, 10%-solution), at 0° C. over 60 min. The reaction mixture was stirred for 30 min at 0° C. Aqueous sodium sulfite (180 mL, 2 M) was added and the layers were separated. The aqueous phase was extracted with ethyl acetate. The organic phases were combined and washed with water and brine, dried over magnesium sulfate and concentrated in vacuo to give 27 g. Recrystallization from 2-propanol gave 13.7 g (86% yield) of the title compound: ¹H NMR (CDCl₃) δ 8.24 (dd, J=6.6, 2.0 Hz, 1H), 7.98-7.94 (m, 3H), 7.25 (t, J=8.3 Hz, 1H), 7.03-6.98 (m, 2H), 3.91 (s, 3H).

### Example 153

### 5-(3-Bromo-4-fluorophenyl)-5-(4-methoxyphenyl)-3-ethyl-2-thioxoimidazolidin-4-one

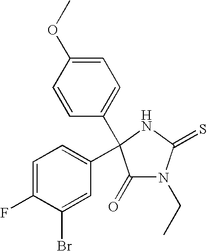

The title compound was synthesized as described for Example 117 in 99% yield, starting from 1-(3-bromo-4-fluorophenyl)-2-(4-methoxyphenyl)ethane-1,2-dione and N-ethylthiourea: ¹H NMR (DMSO-d₆) δ 11.59 (s, 1H), 7.68 (dd, J=6.5, 2.3 Hz, 1H), 7.47 (t, J=8.7 Hz, 1H), 7.42-7.37 (m, 1H), 67.18-7.13 (m, 2H), 7.02-6.97 (m, 2H), 3.82-3.74 (m, 2H), 3.75 (s, 3H), 1.13 (t, J=7.0 Hz, 3H); MS (ES) m/z 423.05 [M−1]⁻.

### Example 154

### 5-(3-Bromo-4-fluorophenyl)-5-(4-hydroxyphenyl)-3-ethyl-2-thioxoimidazolidin-4-one

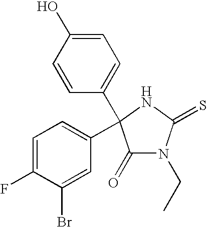

The title compound was synthesized as described for Example 118 in 81% yield, starting from 5-(3-bromo-4-fluorophenyl)-5-(4-methoxyphenyl)-3-ethyl-2-thioxoimidazolidin-4-one: ¹H NMR (DMSO-d₆) δ 11.53 (s, 1H), 9.74 (s, 1H), 7.58 (J=6.5, 2.3 Hz, 1H), 7.46 (t, J=8.5 Hz, 1H), 7.42-7.37 (m, 1H), 7.04-6.99 (m, 2H), 6.81-6.75 (m, 2H), 3.82-3.73 (m, 2H), 1.12 (t, J=7.2 Hz, 3H); MS (ES) m/z 407.07 [M−1]⁻.

### Example 155

### 4-[4-(3-Bromo-4-fluorophenyl)-1-ethyl-5-oxo-2-thioxoimidazolidin-4-yl]phenyl methanesulfonate

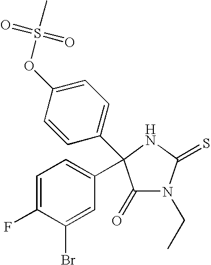

The title compound was synthesized as described for Example 119 in 80% yield, starting from 5-(3-bromo-4-fluorophenyl)-5-(4-hydroxyphenyl)-3-ethyl-2-thioxoimidazolidin-4-one: ¹H NMR (CDCl₃) δ 7.53 (dd, J=6.5, 2.3 Hz, 1H), 7.38-7.32 (m, 4H), 7.29-7.24 (m, 1H), 7.18 (t, J=8.2 Hz, 1H), 3.96 (q, J=7.2, 2H), 3.22 (s, 3H), 1.3 (t, J=7.2 Hz, 3H); MS (ES) m/z 485.08 [M−1]⁻.

### Example 156

### 4-[2-Amino-4-(3-bromo-4-fluorophenyl)-1-ethyl-5-oxo-4,5-dihydro-1H-imidazol-4-yl]phenyl methanesulfonate

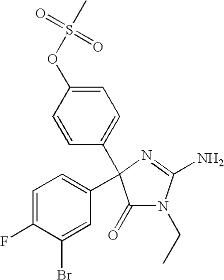

The title compound was synthesized as described for Example 5 in 53% yield, starting from 4-[4-(3-bromo-4-fluorophenyl)-1-ethyl-5-oxo-2-thioxoimidazolidin-4-yl]phenyl methanesulfonate: ¹H NMR (DMSO-d₆) δ 7.73 (dd, J=6.9, 2.1 Hz, 1H), 7.55-7.48 (m, 3 H), 7.36 (t, J=8.7 Hz, 1H), 7.33-7.29 (m, 2H), 6.85 (s, 2H), 3.53 (q, J=7.2 Hz, 2H), 3.38 (s, 3H), 1.26 (t, J=7.0 Hz, 3H): MS (ES) m/z 469.87, 471.82 [M−H]⁺.

### Example 157

### 5-(3-Bromo-4-fluorophenyl)-5-(4-methoxyphenyl)-3-propyl-2-thioxoimidazolidin-4-one

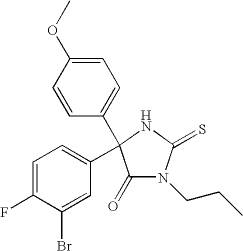

The title compound was synthesized as described for Example 117 in 87% yield, starting from 1-(3-bromo-4-fluorophenyl)-2-(4-methoxyphenyl)ethane-1,2-dione and N-propylthiourea (0.28 g, 2.37 mmol): ¹H NMR (DMSO-d₆) δ 11.60 (s, 1H), 7.58 (dd, J=6.5, 2.3 Hz, 1H), 7.47 (t, J=8.7 Hz, 1H), 7.42-7.37 (m, 1H), 7.19-7.14 (m, 2H), 7.02-6.97 (m, 2H), 3.78-3.68 (m, 2H), 3.75 (s, 3H) 1.58 (m, 2H), 0.79 (t, J=7.4 Hz, 3H); MS (ES) m/z 435.12 [M−1]⁻.

### Example 158

### 5-(3-Bromo-4-fluorophenyl)-5-(4-hydroxyphenyl)-3-propyl-2-thioxoimidazolidin-4-one

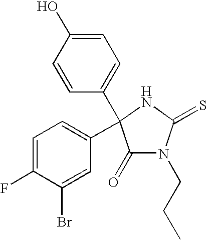

The title compound was synthesized as described for Example 118 in 82% yield, starting from 5-(3-bromo-4-fluorophenyl)-5-(4-methoxyphenyl)-3-propyl-2-thioxoimidazolidin-4-one: ¹H NMR (DMSO-d₆) δ 11.54 (s, 1H), 9.74 (s, 1H), 7.59 (J=6.5, 2.3 Hz, 1H), 7.47 (t, J=8.6 Hz, 1H), 7.42-7.37 (m, 1H), 7.05-7.01 (m, 2H), 6.81-6.77 (m, 2H), 3.76-3.67 (m, 2H), 1.65-1.54 (m, 2H), 0.79 (t, J=7.5 Hz, 3H); MS (ES) m/z 421.10, 423.05 [M−1]⁻.

### Example 159

### 4-[4-(3-Bromo-4-fluorophenyl)-1-propyl-5-oxo-2-thioxoimidazolidin-4-yl]phenyl methanesulfonate

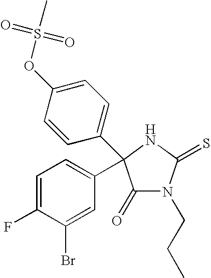

The title compound was synthesized as described for Example 119 in 29% yield, starting from 5-(3-bromo-4-fluorophenyl)-5-(4-hydroxyphenyl)-3-propyl-2-thioxoimidazolidin-4-one: ¹H NMR (DMSO-d₆) δ 11.72 (s, 1H), 7.63 (dd, J=6.5, 2.3 Hz, 1H), 7.50 (t, J=8.7 Hz, 1H), 7.46-7.35 (m, 4H), 3.73 (t, J=7.15 Hz, 2H), 3.41 (s, 3H), 1.67-1.55 (m, 2H), 0.79 (t, J=7.5 Hz, 3H); MS (ES) m/z 499.03, 501.99 [M−1]⁻.

### Example 160

### 4-[2-Amino-4-(3-bromo-4-fluorophenyl)-1-propyl-5-oxo-4,5-dihydro-1H-imidazol-4-yl]phenyl methanesulfonate

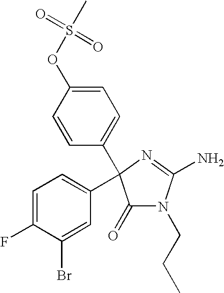

The title compound was synthesized as described for Example 5 in 52% yield, starting from 4-[4-(3-bromo-4-fluorophenyl)-1-propyl-5-oxo-2-thioxoimidazolidin-4-yl]phenyl methanesulfonate: ¹H NMR (CDCl₃) δ 7.69 (dd, J=6.6, 2.3 Hz, 1H), 7.53-7.48 (m, 2H), 7.44-7.38 (m, 1H), 7.25-7.20 (m, 2H), 7.05 (t, J=8.5 Hz, 1H), 3.47 (t, J=7.3 Hz, 2H), 3.13 (s, 3H), 1.70-1.59 (m, 2H), 0.92 (t, J=7.5 Hz, 3H).

### Example 161

### 4-[2-Amino-4-(3′,5′-dichloro-6-fluorobiphenyl-3-yl)-1-ethyl-5-oxo-4,5-dihydro-1H-imidazol-4-yl]phenyl methanesulfonate

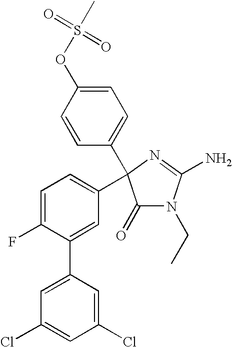

4-[2-Amino-4-(3-bromo-4-fluorophenyl)-1-ethyl-5-oxo-4,5-dihydro-1H-imidazol-4-yl]phenyl methanesulfonate (0.1 g, 0.21 mmol), (3,5-dichlorophenyl)boronic acid (52 mg, 0.27 mmol), [1,1′-bis(diphenylphosphino)ferrocene]dichloropalladium(II) (15 mg, 0.021 mmol), potassium carbonate (174 mg, 1.26 mmol), and anhydrous tetrahydrofuran (3 mL) was irradiated in a microwave at 130° C. for 2 h. When cooled to room temperature the mixture was filtered and dimethyl sulfoxide (500 μL) was added. The solution was concentrated in vacuo to remove the tetrahydrofuran and purified by preparative HPLC to give 25 mg (22% yield) of the title compound: ¹H NMR (CDCl₃) δ 7.55-7.48 (m, 4H) 7.39 (t, J=1.5 Hz, 2H), 7.34 (t, J=1.9 Hz, 1H), 7.25-7.20 (m, 2H), 7.11 (dd, J=10.0, 8.5 Hz, 1H), 5.44 (br s, 2H), 3.64-3.54 (m, 2H), 3.12 (s, 3H), 1.25 (t, J=7.2 Hz, 3H); MS (ES) m/z 535.87, 537.89 [M+1]⁺.

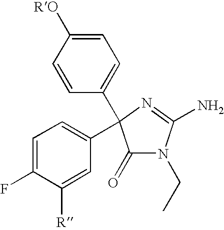

TABLE 3
Representative examples synthesized as described for 4-{2-Amino-4-[3′,5′-
dichloro-6-fluorobiphenyl-3-yl)-4-fluorophenyl]-1-ethyl-5-oxo-4,5-dihydro-1H-imidazol-
4-yl}phenyl methanesulfonate (all reactions were analyzed using LC-MS and those which
showed a low level of conversion was irradiated in a microwave at 130° C. for another
hour).

| | | | | | ¹H-NMR |
|---|---|---|---|---|---|
| | | | | m/z | (CDCl₃) |
| Ex | Chemical name | R′ | R″ | [M + H]⁺ | δ ppm |
| 162 | 4-[2-Amino-4-(4-fluoro-3-pyrimidin-5-ylphenyl)-1-ethyl-5-oxo-4,5-dihydro-1H-imidazol-4-yl]phenyl methanesulfonate | 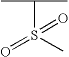 | 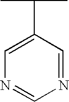 | 469.94 | 9.21 (s, 1H), 8.91 (d, J = 1.5Hz, 2H), 7.62-7.54 (m, 4H),7.28-7.17 (m, 3H), 5.03 (br s,2H), 3.60 (q, J = 7.3 Hz, 2H),3.14 (s, 3H), 1.27 (t, J = 7.3 Hz,3H). |
| 163 | 4-[2-Amino-4-(6-fluoro-3′-methoxybiphenyl-3-yl)-1-ethyl-5-oxo-4,5-dihydro-1H-imidazol-4-yl]phenyl methanesulfonate 0.25acetate | 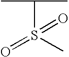 | 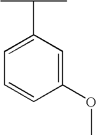 | 498.0 | 7.58-7.54 (m, 3H), 7.45-7.40(m, 1H), 7.33 (t, J = 8.0 Hz,1H), 7.25-7.21 (m, 2H) 7.12-7.04 (m, 3H), 6.91 (dd, J = 7.3,1.8 Hz, 1H), 3.83 (s, 3H), 3.63-3.52 (m, 3H), 3.13 (s, 3H),1.25 (t, J = 7.2 Hz, 3H). |
| 164 | 4-[2-Amino-4-(3′-chloro-6-fluorobiphenyl-3-yl)-1-ethyl-5-oxo-4,5-dihydro-1H-imidazol-4-yl]phenyl methanesulfonate | 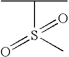 | 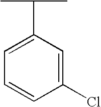 | 501.96 | 7.57-7.52 (m, 3H), 7.50-7.45(m, 2H), 7.41-7.36 (m, 1H)7.35-7.32 (m, 2H), 7.25-7.21(m, 2H), 7.10 (dd, J = 10.1, 8.8Hz, 1H), 3.65-3.50 (m, 2H),3.13 (s, 3H), 1.25 (t, J = 7.3 Hz,3H). |

### Example 165

### 4-[2-Amino-4-(4-fluoro-3-pyrimidin-5-ylphenyl)-1-propyl-5-oxo-4,5-dihydro-1H-imidazol-4-yl]phenyl methanesulfonate 0.5 acetate

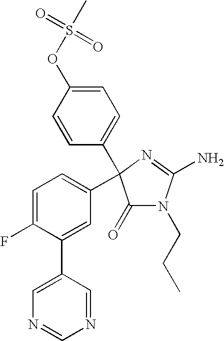

The title compound was synthesized as described for Example 161 in 6% yield, starting from 4-[2-amino-4-(3-bromo-4-fluorophenyl)-1-propyl-5-oxo-4,5-dihydro-1H-imidazol-4-yl]phenyl methanesulfonate and pyrimidine-5-boronic acid: ¹H NMR (CDCl₃) δ 9.21 (s, 1H), 8.92 (d, J=1.5 Hz, 2H), 7.61-7.54 (m, 4H) 7.28-7.24 (m, 2H), 7.20 (t, J=9.6 Hz, 1H), 4.88 (br s, 2H), 3.52 (t, J=7.2 Hz, 2H), 3.14 (s, 3H), 1.73-1.62 (m, 2H), 0.94 (t, J=7.5 Hz, 3H); MS (ES) m/z 483.96 [M+1]⁺.

### Example 166

### 4-Methoxy-2-(4,4,5,5-tetramethyl-1,3,2-dioxaborolan-2-yl)benzonitrile

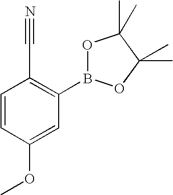

A mixture of tris(dibenzylideneacetone)dipalladium (33 mg, 0.0357 mmol) and tricyclohexylphosphine (47 mg, 0167 mmol) in anhydrous dioxane (2.5 mL) was stirred for 30 min at room temperature under an atmosphere of argon. 4,4,4′,4′,5,5,5′,5′-Octamethyl-2,2′-bi-1,3,2-dioxaborolane (333 mg, 1.31 mmol) potassium acetate (175 mg, 1.785 mmol) and a solution of 2-chloro-4-methoxybenzonitrile (200 mg, 1.19 mmol) in anhydrous dioxane (2.5 mL) were sequentially added and the resulting mixture was irradiated in a microwave at 120° C. for 15 h. Concentration of the reaction mixture and purification by column chromatography, using a gradient of 0-30% ethyl acetate in heptane as the eluent, gave 238 mg (77% yield) of the title compound: ¹H NMR (CDCl₃) □7.63 (d, J=8.6 Hz, 1 H), 7.35 (d, J=2.8 Hz, 1H), 7.01 (dd, J=8.6, 2.8 Hz, 1H), 3.88 (s, 3H), 1.39 (s, 12H); MS (EI) m/z

### Example 167

### 4-[2-Amino-4-(2′-cyano-6-fluoro-5′-methoxybiphenyl-3-yl)-1-methyl-5-oxo-4,5-dihydro-1H-imidazol-4-yl]phenyl methanesulfonate

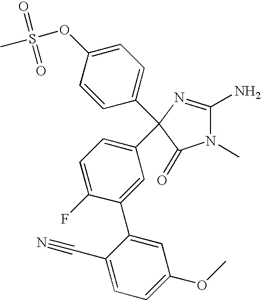

The title compound was synthesized as described for Example 21, in 34% yield, starting from 4-[2-amino-4-(3-bromo-4-fluorophenyl)-1-methyl-5-oxo-4,5-dihydro-1H-imidazol-4-yl]phenyl methanesulfonate and 4-methoxy-2-(4,4,5,5-tetramethyl-1,3,2-dioxaborolan-2-yl)benzonitrile: ¹H NMR (CDCl₃) □7.65-7.68 (m, 1H), 7.60-7.64 (m, 2H), 7.47-7.54 (m, 2H), 7.21-7.26 (m, 2H), 7.15 (t, J=9.1 Hz, 1H), 6.94-6.99 (m, 2H), 3.87 (s, 3H), 3.12 (s, 3H), 3.10 (s, 3H); MS (ESI) m/z 509.0 [M+H]⁺, 507.2 [M−H]⁻.

### Example 168

### 4-{2-Amino-4-[6-fluoro-5′-methoxy-2′-(trifluoromethoxy)biphenyl-3-yl]-1-methyl-5-oxo-4,5-dihydro-1H-imidazol-4-yl}phenyl methanesulfonate

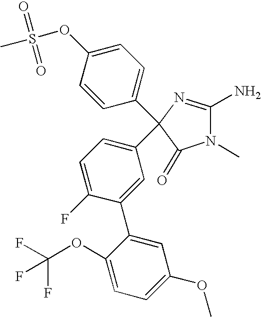

The title compound was synthesized as described for Example 21 in 28% yield, starting from 4-[2-amino-4-(3-bromo-4-fluorophenyl)-1-methyl-5-oxo-4,5-dihydro-1H-imidazol-4-yl]phenyl methanesulfonate and 2-[5-methoxy-2-(trifluoromethoxy)phenyl]-4,4,5,5-tetramethyl-1,3,2-dioxaborolane: ¹H NMR (CDCl₃) □7.53-7.57 (m, 2H), 7.48-7.52 (m, 2H), 7.23-7.27 (m, 2H), 7.21-7.23 (m, 1H), 7.08-7.13 (m, 1H), 6.90-6.93 (m, 1H), 6.87-6.89 (m, 1H), 3.82 (s, 3H), 3.12 (s, 6H); MS (ESI) m/z 568.4 [M+H]⁺, 566.4 [M−H]⁻.

### Example 169

### 4-{2-amino-4-[3-(4-chloropyridin-3-yl)-4-fluorophenyl]-1-methyl-5-oxo-4,5-dihydro-1H-imidazol-4-yl}phenyl methanesulfonate hydrochloride

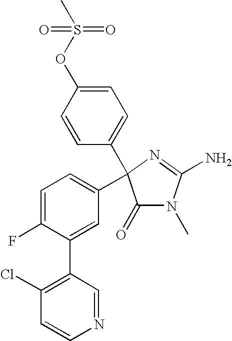

4-[2-Amino-4-(3-bromo-4-fluorophenyl)-1-methyl-5-oxo-4,5-dihydro-1H-imidazol-4-yl]phenyl methanesulfonate (100 mg, 0.22 mmol), 4-chloro-3-(4,4,5,5-tetramethyl-1,3,2-dioxaborolan-2-yl)pyridine (68 mg, 0.285 mmol), [1,1′-bis(diphenylphosphino)ferrocene]palladium(II) chloride dichloromethane adduct (18 mg, 0.022 mmol), potassium carbonate (181 mg, 1.31 mmol), and anhydrous tetrahydrofuran (4 mL) was irradiated in a microwave at 130° C. for 3 h. When cooled to room temperature the mixture was filtered through celite and concentrated in vacuo. Purification by column chromatography, using a gradient with increasing polarity (0 to 10%) ammonium hydroxide in methanol (1:9) in dichloromethane as the eluent, followed by preparative HPLC. The resulting solid was dissolved in 2 M hydrochloric acid in diethyl ether (26 μL) and 2 mL dichloromethane. The solvents were evaporated to give 19 mg (16% yield) of the title compound: ¹H NMR (DMSO-d₆) □8.70-8.56 (m, 2H), 7.75 (d, J=5.8 Hz, 2H), 7.59-7.42 (m, 6H), 3.43 (s, 3H), 3.18 (s, 3H); MS (ESI) m/z 490 [M+1]⁺.

### Example 170

### 3-{2-Amino-4-[3-(4-chloropyridin-3-yl)phenyl]-1-methyl-5-oxo-4,5-dihydro-1H-imidazol-4-yl}phenyl methanesulfonate hydrochloride

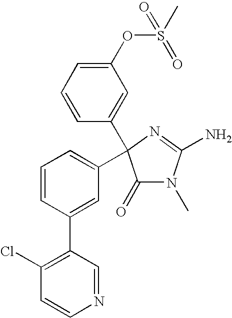

The title compound was synthesized as described for Example 169 in 16% yield, starting from using 3-[2-amino-4-(3-bromophenyl)-1-methyl-5-oxo-4,5-dihydro-1H-imidazol-4-yl]phenyl methanesulfonate: ¹H NMR (DMSO-d₆) □8.56-8.70 (m, 2H), 7.75 (d, J=5.8 Hz, 2H), 7.59-7.42 (m, 6H), 3.43 (s, 3H), 3.18 (s, 3H); MS (ESI) m/z 471[M+1]⁺.

### Example 171

### 3-[(3-Bromophenyl)ethynyl]phenol

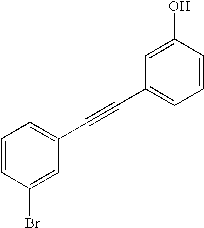

A solution of 1-bromo-3-ethynylbenzene (16.5 g, 90.9 mmol, described in Wettergren, J., Minidis, A. B. E. Tetrahedron Letters. 2003, 44, 7611-7612) in anhydrous tetrahydrofuran (25 mL) was added to a solution of 3-iodophenol (19 g, 86.4 mmol), bis(triphenylphosphine)palladium(II) chloride (383 mg, 0.55 mmol), copper(I) iodide (104 mg, 0.55 mmol) and triethylamine (75 mL, 538 mmol) in anhydrous tetrahydrofuran (125 mL) at 0° C. under an atmosphere of argon. The mixture was stirred for 10 min at 0° C., allowed to reach room temperature and stirred overnight. The solvents were evaporated in vacuo and the residue partitioned between dichloromethane and water. The organic phase was washed with water and brine, dried over magnesium sulfate and concentrated in vacuo. Purification by column chromatography, using 50% dichloromethane in heptane as the eluent, followed by recrystallization in dichloromethane gave 17.16 g (69% yield) of the title compound: ¹H NMR (DMSO-d₆) δ 9.72 (s, 1H), 7.77-7.74 (m, 1H), 7.64-7.60 (m, 1H), 7.57-7.54 (m, 1H), 7.41-7.36 (m, 1H), 7.26-7.20 (m, 1H), 7.01-6.96 (m, 1H), 6.94-6.91 (m, 1H), 6.84 (ddd, J=8.3, 2.5, 1.0 Hz, 1H); MS (ES) m/z 271,273 [M−H]⁻.

### Example 172

### 1-(3-Bromophenyl)-2-(3-hydroxyphenyl)ethane-1,2-dione

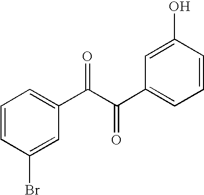

A solution of 3-[(3-bromophenyl)ethynyl]phenol (17.16 g, 62.83 mmol) and palladium(II) dichloride (1.11 g, 6.28 mmol) in dimethyl sulfoxide (300 mL) was stirred at 140° C. for 5 h. When cooled to room temperature the mixture was diluted with water and extracted with diethyl ether. The combined organics were washed with water and brine, dried over magnesium sulfate and concentrated to give 19.4 g (quantitative yield) of the title compound: MS (ES) m/z 303,305 [M−H]⁻.

### Example 173

### 5-(3-Bromophenyl)-5-(3-hydroxyphenyl)-3-methyl-2-thioxoimidazolidin-4-one

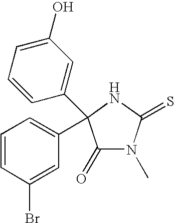

N-Methylthiourea (11.2 g, 124 mmol) was added to a solution of 1-(3-bromophenyl)-2-(3-hydroxyphenyl)ethane-1,2-dione (19.4 g, 62 mmol) in dimethyl sulfoxide (62 mL) and heated to 100° C. An aqueous solution of potassium hydroxide (1.5 M, 58 mL, 86.6 mmol) was added slowly and the resulting solution was stirred at 100° C. for 10 min. When cooled to room temperature the mixture was diluted with water (300 mL), 6 M hydrochloric acid (50 mL) was added and the aqueous phase was extracted with chloroform. The combined organics were washed with water and brine, dried over sodium sulfate and concentrated in vacuo. Purification by column chromatography, using 25% ethyl acetate in heptane as the eluent, gave 16.33 g (70% yield) of the title compound: MS (ES) m/z 375,377 [M−H]⁻.

### Example 174

### 3-[4-(3-Bromophenyl)-1-methyl-5-oxo-2-thioxoimidazolidin-4-yl]phenyl methanesulfonate

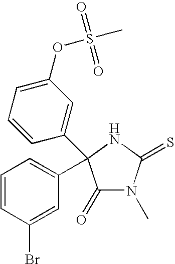

Methanesulfonyl chloride (0.39 mL, 4.97 mmol) was added to a cooled (0° C.) solution of 5-(3-bromophenyl)-5-(3-hydroxyphenyl)-3-methyl-2-thioxoimidazolidin-4-one (1.25 g, 3.31 mmol) and triethylamine (1.4 mL, 9.94 mmol) in anhydrous dichloromethane (30 mL) under an atmosphere of argon. The resulting mixture was allowed to reach room temperature and stirred for 1.5 h. The solvents were evaporated in vacuo and the residue was dissolved in ethyl acetate which resulted in the precipitation of triethylamine salt. The salt was filtered off, the filtrate was concentrated and purified by column chromatography, using 5-40% ethyl acetate in heptane as the eluent, to give 940 mg (62% yield) of the title compound: ¹H NMR (DMSO-d₆) δ 11.72 (s, 1H), 7.65-7.61 (m, 1H), 7.58 (t, J=8.0 Hz, 1H), 7.50 (m, 1H), 7.45-7.39 (m, 2H), 7.39-7.33 (m, 2H), 7.30-7.26 (m, 1H), 3.41 (s, 3H), 3.18 (s, 3H); MS (ES) m/z 453,455 [M−H]⁻.

### Example 175

### 3-[2-Amino-4-(3-bromophenyl)-1-methyl-5-oxo-4,5-dihydro-1H-imidazol-4-yl]phenyl methanesulfonate

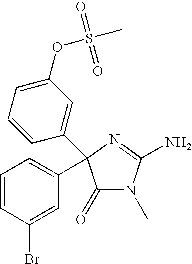

An aqueous solution of t-butyl hydroperoxide (70%, 3 mL, 31 mmol) was added to a solution of 3-[4-(3-bromophenyl)-1-methyl-5-oxo-2-thioxoimidazolidin-4-yl]phenyl methanesulfonate (940 mg, 2.06 mmol) in methanol (30 mL) and aqueous ammonia (33%, 6 mL) and the resulting mixture was stirred at room temperature for 6 h. The methanol was removed in vacuo, the residue was diluted with water (75 mL) and extracted with chloroform. The combined organic phases were washed with brine, dried over sodium sulfate and concentrated in vacuo to give 890 mg (99% yield) of the title compound: MS (ES) m/z 438, 440 [M+H]⁺.

### Example 176

### 3-[2-Amino-4-(2′-cyano-5′-methoxybiphenyl-3-yl)-1-methyl-5-oxo-4,5-dihydro-1H-imidazol-4-yl]phenyl methanesulfonate hydrochloride

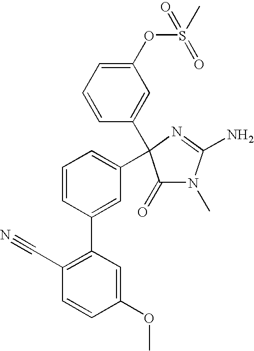

3-[2-Amino-4-(3-bromophenyl)-1-methyl-5-oxo-4,5-dihydro-1H-imidazol-4-yl]phenyl methanesulfonate (100 mg, 0.228 mmol), 4-methoxy-2-(4,4,5,5-tetramethyl-1,3,2-dioxaborolan-2-yl)benzonitrile (77 mg, 0.297 mmol), [1,1′-bis(diphenylphosphino)ferrocene]palladium(II) chloride dichloromethane adduct (19 mg, 0.023 mmol), potassium carbonate (189 mg, 1.37 mmol), tetrahydrofuran (3 mL) and water (1 mL) was irradiated in a microwave at 150° C. for 15 min. When cooled to room temperature the mixture was filtered through celite and concentrated in vacuo. Purification by column chromatography, using a gradient with increasing polarity (0 to 10%) ammonium hydroxide in methanol (1:9) in dichloromethane as the eluent, followed by preparative HPLC. The resulting residue was dissolved in 2 M hydrochloric acid in diethyl ether (82 μL) and dichloromethane (2 mL). The solvents were evaporated to give 77 mg (64% yield) of the title compound: ¹H NMR (DMSO-d₆) δ 9.65 (m, 2H), 7.90 (d, J=8.6 Hz, 1H), 7.74-7.54 (m, 3H), 7.51-7.39 (m, 3H), 7.16 (dd, J=8.6, 2.53 Hz, 1H), 7.10 (d, J=2.3 Hz, 1H), 3.41 (s, 3H), 3.41 (s, 3H), 3.18 (s, 3H); MS (ESI) m/z 492 [M+1]⁺.

### Example 177

### 3-[2-Amino-4-(2′-cyano-6-fluoro-5′-methoxybiphenyl-3-yl)-1-methyl-5-oxo-4,5-dihydro-1H-imidazol-4-yl]phenyl methanesulfonate hydrochloride

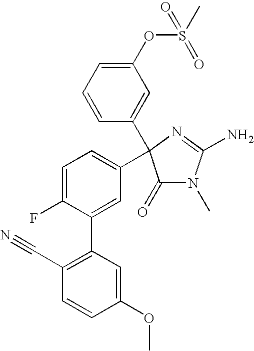

The title compound was synthesized as described for Example 176 in 24% yield, starting from 3-[2-amino-4-(3-bromo-4-fluorophenyl)-1-methyl-5-oxo-4,5-dihydro-1H-imidazol-4-yl]phenyl methanesulfonate and 4-methoxy-2-(4,4,5,5-tetramethyl-1,3,2-dioxaborolan-2-yl)benzonitrile: ¹H NMR (CD₃OD) □7.76 (d, J=8.6 Hz, 1H), 7.57-7.30 (m, 7H), 7.12 (dd, J=8.7, 2.6 Hz, 1H), 7.04 (d, J=2.3 Hz, 1H), 3.88 (s, 3H), 3.26 (s, 3H), 3.22 (s, 3H); MS (ESI) m/z 510 [M+1]⁺.

### Example 178

### 6-[(3-Bromophenyl)ethynyl]-2,3-dihydro-1,4-benzodioxine

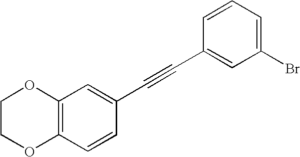

To a mixture of bis(triphenylphosphine)palladium(II) dichloride (134 mg, 0.191 mmol), copper(I) iodide (36 mg, 0.191 mmol) and triethylamine (15 mL) in anhydrous tetrahydrofuran (65 mL) under an atmosphere of argon were sequentially added a solution of 6-iodobenzodioxane (5.00 g, 19.08 mmol) in anhydrous tetrahydrofuran (15 mL) and a solution of (3-bromophenyl)acetylene (3.63 g, 20.03 mmol) in anhydrous tetrahydrofuran (10 mL). The resulting solution was stirred at room temperature for 3 days. The crude mixture was diluted with ethyl acetate, washed sequentially with 1 M hydrochloric acid, water and saturated aqueous sodium hydrogen carbonate, dried over magnesium sulfate and concentrated in vacuo. Purification by column chromatography, using ethyl acetate in heptane (0-15%) as the eluent, gave the title compound in 94% yield: ¹H NMR (CDCl₃) □7.67 (t, J=1.6 Hz, 1H), 7.42-7.46 (m, 2H), 7.21 (t, J=8.0 Hz, 1H), 7.01-7.06 (m, 2H), 6.84 (d, J=7.8 Hz, 1H), 4.26-4.31 (m, 4H); MS (EI) m/z 314, 316 [M+•].

### Example 179

### 1-(3-Bromophenyl)-2-(2,3-dihydro-1,4-benzodioxin-6-yl)ethane-1,2-dione

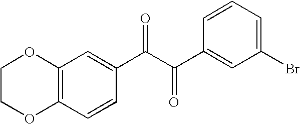

A mixture of 6-[(3-bromophenyl)ethynyl]-2,3-dihydro-1,4-benzodioxine (2.80 g, 8.88 mmol) and palladium(II) dichloride (158 mg, 0.89 mmol) in dimethyl sulfoxide (75 mL) was heated at 150° C. under an atmosphere of argon for 5 h. Water was added after cooling to room temperature. The mixture was extracted with diethyl ether, the combined extracts were washed with brine, dried over sodium sulfate and evaporated. The residue was dissolved in diethyl ether and treated with active charcoal, filtered and evaporated to give 3.20 g of crude product (100% yield), used without further purification in the following reaction: ¹H NMR (CDCl₃) □8.11 (t, J=1.8 Hz, 1H), 7.88 (m, 1H), 7.77 (m, 1H), 7.52 (d, J=2.0 Hz, 1H), 7.48 (dd, J=8.6, 2.0 Hz, 1H), 7.38 (t, J=8.0 Hz, 1H), 6.96 (d, J=8.6 Hz, 1H), 4.35 (m, 2H), 4.30 (m, 2H).

### Example 180

### 5-(3-Bromophenyl)-5-(2,3-dihydro-1,4-benzodioxin-6-yl)-3-methyl-2-thioxoimidazolidin-4-one

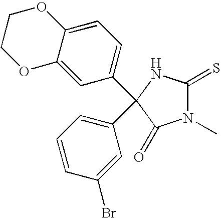

To a solution of crude 1-(3-bromophenyl)-2-(2,3-dihydro-1,4-benzodioxin-6-yl)ethane-1,2-dione (3.20 g, 8.88 mmol) in dimethyl sulfoxide (70 mL) was added N-methylthiourea (2.36 g, 26.21 mmol). The solution was heated to 100° C. and 1.2 M aqueous potassium hydroxide (15.2 mL, 18.2 mmol) was added dropwise over 5 min. Heating was continued for another 10 min and then the solution was cooled to room temperature. Water (100 mL) was added and pH was adjusted to 5-6 by addition of 1 M hydrochloric acid. The mixture was extracted with dichloromethane. The combined extracts were washed with water, dried over sodium sulfate and passed through a plug of Silica in a glass filter funnel and evaporated to give 3.48 g (93% yield) of the title compound: ¹H NMR (CDCl₃) □7.51 (m, 2H), 7.31 (m, 1H), 7.25 (m, 1H), 6.86 (d, J=8.6 Hz, 1H), 6.78 (d, J=2.3 Hz, 1H), 6.73 (m, 1H), 4.26 (m, 4H), 3.32 (s, 3H); MS (ESI) m/z 417.3, 419.3 [M−H]⁻.

### Example 181

### 2-Amino-5-(3-bromophenyl)-5-(2,3-dihydro-1,4-benzodioxin-6-yl)-3-methyl-3,5-dihydro-4H-imidazol-4-one

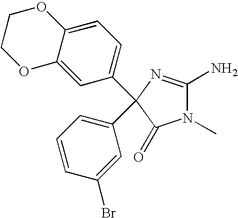

A methanolic (100 mL) solution of 5-(3-bromophenyl)-5-(2,3-dihydro-1,4-benzodioxin-6-yl)-3-methyl-2-thioxoimidazolidin-4-one (3.47 g, 8.28 mmol), 70% aqueous tert-butyl hydroperoxide (11.9 mL, 124 mmol) and 33% aqueous ammonia (20 mL) was stirred at room temperature over night. The methanol was removed in vacuo and the aqueous residue was extracted with dichloromethane. The combined extracts were washed with water, dried over sodium sulfate and evaporated. Purification by column chromatography, using a gradient with increasing concentration (0-100%) of a mixture of methanol in dichloromethane (1:4) in heptane as the eluent, gave 2.6 g (78% yield) of the title compound: ¹H NMR (CDCl₃) □7.66 (t, J=1.9 Hz, 1H), 7.41-7.44 (m, 1H), 7.38-7.41 (m, 1H), 7.17 (t, J=8.0 Hz, 1H), 6.93 (d, J=2.3 Hz, 1H), 6.85-6.89 (m, 1H), 6.79 (d, 1H), 4.22 (m, 4H), 3.10 (s, 3H); MS (ESI) m/z 402.2, 404.4 [M+H]⁺, 400.3, 402.3 [M−H]⁻.

### Example 182

### 3′-[2-Amino-4-(2,3-dihydro-1,4-benzodioxin-6-yl)-1-methyl-5-oxo-4,5-dihydro-1H-imidazol-4-yl]-5-methoxybiphenyl-3-yl methanesulfonate

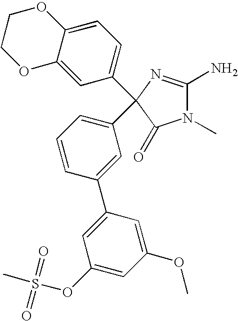

A mixture of 3-methoxy-5-(4,4,5,5-tetramethyl-1,3,2-dioxaborolan-2-yl)phenyl methanesulfonate (81 mg, 0.247 mmol), [1,1′-bis(diphenylphosphino)ferrocene]palladium(II) chloride dichloromethane adduct (20 mg, 0.025 mmol), potassium carbonate (171 mg, 1.235 mmol) and anhydrous tetrahydrofuran (3 mL) was irradiated with microwaves at 130° C. for 3 h, under an argon atmosphere. When cooled to room temperature the mixture was diluted with dichloromethane and filtered through a pad of silica. The filtrate was concentrated in vacuo and purified by preparative HPLC to give 20 mg (15% yield) of the title compound: ¹H NMR (CDCl₃) □7.70 (m, 1H), 7.53-7.57 (m, 1H), 7.43-7.47 (m, 1H), 7.36-7.41 (m, 1H), 7.03-7.06 (m, 2H), 6.97 (d, J=2.3 Hz, 1H), 6.90-6.93 (m, 1H), 6.78-6.82 (m, 2H), 4.22 (s, 4H), 3.85 (s, 3H), 3.15 (s, 3H), 3.11 (s, 3H); MS (ESI) m/z 524.4 [M+H]⁺, 522.4 [M−H]⁻.

### Example 183

### 4-[4-(3-Bromo-4-fluorophenyl)-1-methyl-5-oxo-2-thioxoimidazolidin-4-yl]phenyl benzenesulfonate

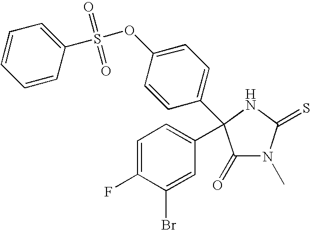

The title compound was synthesized as described for Example 131 in quantitative yield, starting from 5-(3-bromo-4-fluorophenyl)-5-(4-hydroxyphenyl)-3-methyl-2-thioxoimidazolidin-4-one: MS (ES) m/z 533, 535 [M−1]⁻.

### Example 184

### 4-[2-Amino-4-(3-bromo-4-fluorophenyl)-1-methyl-5-oxo-4,5-dihydro-1H-imidazol-4-yl]phenyl benzenesulfonate

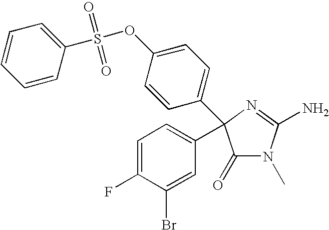

The title compound was synthesized as described for Example 20 in 30% yield, starting from 4-[4-(3-bromo-4-fluorophenyl)-1-methyl-5-oxo-2-thioxoimidazolidin-4-yl]phenyl benzenesulfonate: MS (ES) m/z 518, 520 [M+1]⁺.

### Example 185

### 4-[2-Amino-4-(4-fluoro-3-pyrimidin-5-ylphenyl)-1-methyl-5-oxo-4,5-dihydro-1H-imidazol-4-yl]phenyl benzenesulfonate hydrochloride

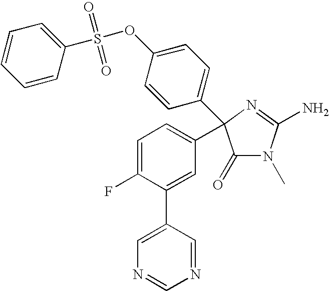

The title compound was synthesized as described for Example 133 in 59% yield, starting from 4-[2-amino-4-(3-bromo-4-fluorophenyl)-1-methyl-5-oxo-4,5-dihydro-1H-imidazol-4-yl]phenyl benzenesulfonate: ¹H NMR (DMSO-d₆) □11.74 (br s, 1H), 9.70 (br s, 2H), 9.26 (s, 1H), 9.02 (s, 2H), 7.93-7.87 (m, 2H), 7.86-7.80 (m, 1H), 7.72-7.64 (m, 3H), 7.55-7.48 (m, 1H), 7.48-7.42 (m, 2H), 7.43-7.37 (m, 1H), 7.20-7.13 (m, 2H), 3.17 (s, 3H); MS (ES) m/z 518, 520 [M+1]⁺.

### Example 186

### 2-Amino-5-[4-fluoro-3-(1,3-thiazol-5-yl)phenyl]-5-(4-hydroxyphenyl)-3-methyl-3,5-dihydro-4H-imidazol-4-one

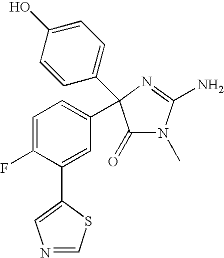

A solution of 2-amino-5-(3-bromo-4-fluorophenyl)-5-(4-hydroxyphenyl)-3-methyl-3,5-dihydro-4H-imidazol-4-one (151 mg, 0.4 mmol), 5-(tributylstannyl)-1,3-thiazole (195 mg, 0.52 mmol) and tetrakis(triphenylphosphine)palladium(0) (23 mg, 0.02 mmol) in anhydrous N,N-dimethylformamide (3 mL) was stirred at 100° C. for 22 h. Additional 5-(tributylstannyl)-1,3-thiazole (85 mg, 0.23 mmol) dissolved in anhydrous N,N-dimethylformamide (0.3 mL) was added and the stirring was continued for 16 h. The volatiles were removed in vacuo and the residue partitioned between brine (20 mL) and dichloromethane (20 mL). The organic phase was separated and the aqueous phase extracted with dichloromethane. The combined organics were concentrated and purified by column chromatography, using 0-5% ammonia in methanol (0.1 M) in dichloromethane as the eluent, to give 69 mg (45% yield) of the title compound: MS (ES) m/z 383 [M+1]⁺.

### Example 187

### 4-{2-Amino-4-[4-fluoro-3-(1,3-thiazol-5-yl)phenyl]-1-methyl-5-oxo-4,5-dihydro-1H-imidazol-4-yl}phenyl trifluoromethanesulfonate hydrochloride

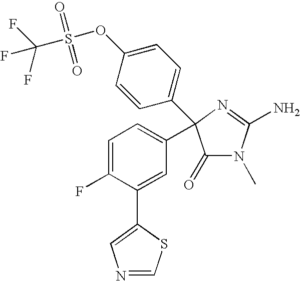

The title compound was synthesized as described for Example 76 in 53% yield, starting from 4-[2-amino-4-(3-bromo-4-fluorophenyl)-1-methyl-5-oxo-4,5-dihydro-1H-imidazol-4-yl]phenyl methanesulfonate: ¹H NMR (DMSO-d₆) □11.86 (br s, 1H), 9.75 (br s, 2H), 9.24 (s, 1H), 8.36 (s, 1H), 7.89-7.83 (m, 1H), 7.67-7.58 (m, 4H), 7.55-7.48 (m, 1H), 7.46-7.40 (m, 1H), 3.19 (s, 3H); MS (ES) m/z 515 [M+1]⁺.

### Example 188

### 4-{2-Amino-4-[4-fluoro-3-(1,3-thiazol-5-yl)phenyl]-1-methyl-5-oxo-4,5-dihydro-1H-imidazol-4-yl}phenyl methanesulfonate hydrochloride

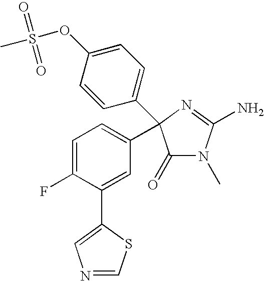

The title compound was synthesized as described for Example 186 in 53% yield, starting from 4-[2-amino-4-(3-bromo-4-fluorophenyl)-1-methyl-5-oxo-4,5-dihydro-1H-imidazol-4-yl]phenyl methanesulfonate: ¹H NMR (DMSO-d₆) □11.85 (br s, 1H), 9.71 (br s, 2H), 9.24 (s, 1H), 8.36 (s, 1H), 7.91-7.85 (m, 1H), 7.54-7.48 (m, 2H), 7.47-7.41 (m, 4H), 3.42 (s, 3H), 3.20 (s, 3H); MS (ES) m/z 461 [M+1]⁺.

### Example 189

### 2-(3-Bromo-4-fluorophenyl)-1,3-dithiane

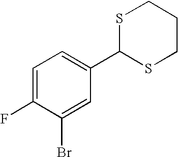

Boron trifluoride-diethyl etherate (10.82 mL, 88 mmol) was added dropwise to a solution of 3-bromo-4-fluorobenzaldehyde (8.93 g, 44 mmol) and 1,3-propanedithiol (4.45 mL, 44 mmol) in dichloromethane (50 mL) at 0° C. under an atmosphere of argon, and the resulting mixture was stirred for 1.5 h. Saturated aqueous sodium bicarbonate was added and the aqueous phase extracted with dichloromethane. The combined organic phases were washed with aqueous potassium hydroxide (1.5 M) and water, dried over magnesium sulfate and the solvent was evaporated to give 12.7 g (98% yield) of the title compound: MS (ES) m/z 293 [M−H]⁻.

### Example 190

### 1-(3-Bromo-4-fluorophenyl)-2-(tetrahydro-2H-pyran-4-yl)ethane-1,2-dione

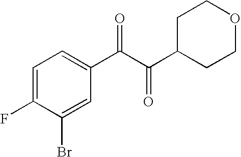

Lithium bis(trimethylsilyl)amide (1 M in hexanes, 45 mL) was added dropwise to a solution of 2-(3-bromo-4-fluorophenyl)-1,3-dithiane (12.7 g, 43 mmol) in anhydrous tetrahydrofuran at 0° C. under an atmosphere of argon. The mixture was stirred for 15 min at 0° C., a solution of tetrahydro-2H-pyran-4-carbaldehyde (4.8 g, 43 mmol) in anhydrous tetrahydrofuran (10 mL) was added and the resulting mixture was allowed to reach room temperature overnight. Saturated aqueous ammonium chloride was added, the tetrahydrofuran removed in vacuo and the residue extracted with dichloromethane. The combined organics were dried over magnesium sulfate, concentrated and purified by column chromatography, using a gradient of ethyl acetate (0-10%) in heptane as the eluent, to give 4.55 g. This intermediate was dissolved in ethyl acetate (100 mL) and added dropwise over 40 min to a slurry of N-bromosuccinimide (28 g, 158 mmol) in aqueous sodium bicarbonate (10%, 200 mL) at 0° C. The resulting mixture was stirred for 1.5 h at 0° C. and aqueous sodium sulfite (2 M, 160 mL) was added over 30 min. The organic phase was separated and the aqueous phase was extracted with ethyl acetate. The combined organic phases were dried over magnesium sulfate and concentrated. Succinimide was removed by recrystallization from 2-propanol and the crude product was purified by column chromatography, using a gradient of ethyl acetate (0-30%) in heptane as the eluent, to give 630 mg (5% yield) of the title compound: MS (ES) m/z 312, 314 [M−H]⁻.

### Example 191

### 2-Amino-5-(3-bromo-4-fluorophenyl)-3-methyl-5-(tetrahydro-2H-pyran-4-yl)-3,5-dihydro-4H-imidazol-4-one

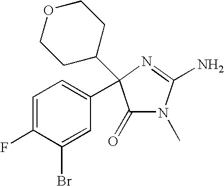

A mixture of 1-(3-bromo-4-fluorophenyl)-2-(tetrahydro-2H-pyran-4-yl)ethane-1,2-dione (630 mg, 2 mmol) and N-methylguanidine hydrochloride (330 mg, 3 mmol) in dioxane (2.3 mL) and ethanol (2.3 mL) was stirred at 30° C. for 15 min and a solution of sodium carbonate (320 mg, 3 mmol) in water (1.2 mL) was added. The resulting mixture was heated at 85° C. for 1 h, cooled to room temperature, filtered and concentrated in vacuo. The resulting residue was purified by column chromatography, using a gradient of 0-5% ammonia in methanol (0.1 M) in dichloromethane as the eluent, to give 316 mg (43% yield) of the title compound: ¹H NMR (DMSO-d₆) □7.84 (dd, J=7.0, 2.0 Hz, 1H), 7.67-7.62 (m, 1H), 7.34 (t, J=8.7 Hz, 1H), 6.75 (br s, 2H), 3.82-3.70 (m, 2H), 3.26-3.19 (m, 1H), 3.18-3.09 (m, 1H), 2.90 (s, 3H), 2.18-2.08 (m, 1H), 1.41-1.29 (m, 1H), 1.29-1.18 (m, 1H), 1.14-1.08 (m, 1H), 0.97-0.90 (m, 1H); MS (ES) m/z 368,370[M−H]⁻.

### Example 192

### 5′-[2-Amino-1-methyl-5-oxo-4-(tetrahydro-2H-pyran-4-yl)-4,5-dihydro-1H-imidazol-4-yl]-2′-fluoro-5-methoxybiphenyl-3-yl methanesulfonate hydrochloride

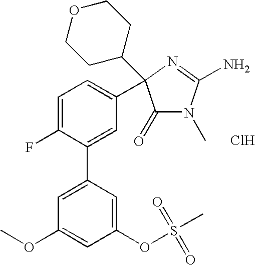

2-Amino-5-(3-bromo-4-fluorophenyl)-3-methyl-5-(tetrahydro-2H-pyran-4-yl)-3,5-dihydro-4H-imidazol-4-one (93 mg, 0.25 mmol), 3-methoxy-5-(4,4,5,5-tetramethyl-1,3,2-dioxaborolan-2-yl)phenyl methanesulfonate (90 mg, 0.28 mmol), [1,1′-bis(diphenylphosphino)ferrocene]palladium(II) chloride dichloromethane adduct (10 mg, 0.013 mmol) and potassium carbonate (104 mg, 0.75 mmol) in tetrahydrofuran (1.6 mL) and water (0.4 mL) were mixed and irradiated under an argon atmosphere in a microwave at 130° C. for 15 min. When cooled to room temperature the mixture was diluted with brine and extracted with ethyl acetate. The combined organics were concentrated and purified by preparative HPLC. The acetonitrile was removed in vacuo and the residue diluted with saturated sodium hydrogencarbonate and extracted with dichloromethane. The combined organic phases were treated with hydrochloric acid (1 M in diethyl ether, 0.4 mL) and the resulting mixture was concentrated to give 75 mg (57% yield) of the title compound: ¹H NMR (DMSO-d₆) □11.36 (br s, 1H), 9.46 (br s, 2H), 7.73-7.64 (m, 2H), 7.50-7.42 (m, 1H), 7.18-7.11 (m, 2H), 7.07-7.03 (m, 1H), 3.87 (s, 3H), 3.85-3.79 (m, 2H), 3.44 (s, 3H), 3.28-3.21 (m, 2H), 3.11 (s, 3H), 2.71-2.54 (m, 1H), 1.41-1.26 (m, 3H), 1.15-1.06 (m, 1H); MS (ES) m/z 492 [M+H]⁺.

### Example 193

### 5′-[2-Amino-1-methyl-5-oxo-4-(tetrahydro-2H-pyran-4-yl)-4,5-dihydro-1H-imidazol-4-yl]-5-chloro-2′-fluorobiphenyl-3-yl methanesulfonate hydrochloride

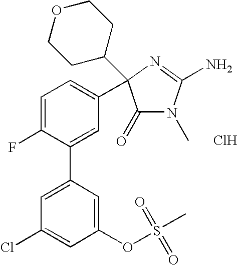

The title compound was synthesized as described for Example 192 in 17% yield, starting from 2-Amino-5-(3-bromo-4-fluorophenyl)-3-methyl-5-(tetrahydro-2H-pyran-4-yl)-3,5-dihydro-4H-imidazol-4-one and 3-chloro-5-(4,4,5,5-tetramethyl-1,3,2-dioxaborolan-2-yl)phenyl methanesulfonate: ¹H NMR (DMSO-d₆) □11.25 (br s, 1H), 9.47 (br s, 2H), 7.75-7.66 (m, 3H), 7.64-7.61 (m, 1H), 7.57-7.53 (m, 1H), 7.52-7.45 (m, 1H), 3.89-3.79 (m, 2H), 3.50 (s, 3H), 3.29-3.22 (m, 2H), 3.12 (s, 3H), 2.70-2.58 (m, 1H), 1.40-1.27 (m, 3H), 1.14-1.05 (m, 1H); MS (ES) m/z 496 [M+H]⁺.

### Example 194

### 3-[2-Amino-4-(3-cyclopent-1-en-1-yl-4-fluorophenyl)-1-methyl-5-oxo-4,5-dihydro-1H-imidazol-4-yl]phenyl methanesulfonate hydrochloride

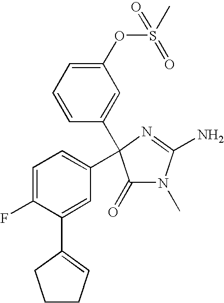

3-[2-Amino-4-(3-bromo-4-fluorophenyl)-1-methyl-5-oxo-4,5-dihydro-1H-imidazol-4-yl]phenyl methanesulfonate (100 mg, 0.22 mmol), cyclopenten-1-ylboronic acid (31, 0.28 mmol), potassium carbonate (181 mg, 1.31 mmol) and [1,1′-bis(diphenylphosphino)ferrocene]palladium(II) chloride dichloromethane adduct (18 mg, 0.02 mmol) were dissolved in tetrahydrofuran (4 mL) and water 1 (mL) and irradiated in a microwave at 150° C. for 15 min. When cooled to room temperature the mixture was filtered through celite, concentrated in vacuo and purified by preparative HPLC. Dichloromethane (1 mL) and hydrochloric acid (1 M in diethyl ether, 100 μL) was added. The solvents were evaporated to give 41 mg (45% yield) of the title compound: ¹H NMR (CD₃OD) δ 7.57 (t, J=8.1 Hz, 1H), 7.46-7.38 (m, 2H), 7.36-7.23 (m, 3H), 7.22-7.15 (m, 1H), 6.43-6.28 (m, 1H), 3.29 (s, 3H), 3.25 (s, 3H), 2.71-2.61 (m, 2H), 2.59-2.50 (m, 2H), 1.18 (t, 2H); MS (ESI) m/z 442 [M−1]⁻.

### Example 195

### 3-[2-Amino-4-(3-cyclopent-1-en-1-yl-phenyl)-1-methyl-5-oxo-4,5-dihydro-1H-imidazol-4-yl]phenyl methanesulfonate hydrochloride

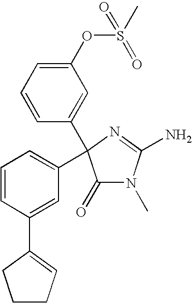

The title compound was synthesized as described for Example 194 in 16% yield, starting from 3-[2-amino-4-(3-bromophenyl)-1-methyl-5-oxo-4,5-dihydro-1H-imidazol-4-yl]phenyl methanesulfonate: ¹H NMR (CD₃OD) δ 7.57 (t, J=8.1 Hz, 1H), 7.46-7.39 (m, 2H), 7.36-7.23 (m, 3H), 7.22-7.15 (m, 1H), 6.43-6.28 (m, 1H), 3.29 (s, 3H), 3.25 (s, 3H), 2.71-2.61 (m, 2H), 2.59-2.50 (m, 2H), 1.18 (t, 2H); MS (ESI) m/z 426 [M+1]⁺.

### Example 196

### 2-Amino-5-(3-cyclopent-1-en-1-ylphenyl)-5-(3-hydroxyphenyl)-3-methyl-3,5-dihydro-4H-imidazol-4-one

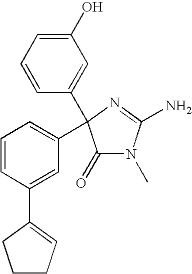

The title compound was synthesized as described for Example 194 in 74% yield, starting from 2-amino-5-(3-bromophenyl)-5-(3-hydroxyphenyl)-3-methyl-3,5-dihydro-4H-imidazol-4-one, except that the product was purified by column chromatography, using 0 to 10% {ammonium hydroxide/methanol (1:9) in dichloromethane as the eluent and was not converted to the hydrochloric acid: MS (ESI) m/z 348 [M+1]⁺.

### Example 197

### 3-[2-Amino-4-(3-cyclopent-1-en-1-ylphenyl)-1-methyl-5-oxo-4,5-dihydro-1H-imidazol-4-yl]phenyl trifluoromethanesulfonate hydrochloride

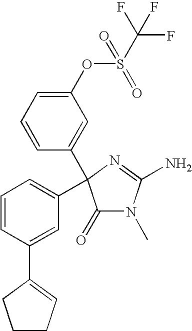

2-Amino-5-(3-cyclopent-1-en-1-ylphenyl)-5-(3-hydroxyphenyl)-3-methyl-3,5-dihydro-4H-imidazol-4-one (150 mg, 0.43 mmol), N-phenyltrifluoromethanesulfonimide (168 mg, 0.47 mmol) and potassium carbonate (358 mg, 2.59 mmol) were dissolved in anhydrous tetrahydrofuran (5 mL) and irradiated in a microwave at 120° C. for 7 min. When cooled to room temperature the mixture was filtered through celite, concentrated in vacuo and purified by preparative HPLC. Dichloromethane (1 mL) and hydrochloric acid (1 M in diethyl ether, 73 μL) was added. The solvents were evaporated to give 33 mg (16% yield) of the title compound: ¹H NMR (CD₃OD) δ 7.57-7.46 (m, 2H), 7.43-7.34 (m, 3H), 7.33-7.24 (m, 2H), 7.21-7.151 (m, 1H), 6.15 (t, J=2.3 Hz, 1H), 3.13 (s, 3H), 2.68-2.60 (m, 2H), 2.54-2.46 (m, 2H), 2.05-1.94 (m, 2H); MS (ESI) m/z 480 [M+1]⁺.

### Example 198

### 4-Bromo-2,6-diethylpyridine 1-oxide

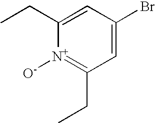

2,6-Diethyl-pyridine-N-oxide (12 g, 79.4 mmol) was dissolved in sulfuric acid (conc., 48 mL) and cooled to 0° C. Nitric acid (conc., 38 mol) was added dropwise and the resulting mixture was heated at 90° C. for 2 h. The reaction mixture was poured onto ice, neutralized using sodium hydroxide (15%) and extracted with dichloromethane. The combined organic phases were washed with brine and dried over sodium sulfate. The resulting solution was split into ten portions that were evaporated separately and then instantly dissolved in acetic acid (10 mL each). The ten solutions were combined, acetyl bromide (70 mL) was added dropwise and the reaction was heated at 90° C. overnight. The reaction mixture was poured onto ice and neutralized using sodium hydroxide (15%) and extracted with ethyl acetate. The combined organic phases were washed with brine, dried over sodium sulfate and concentrated in vacuo. Purification by column chromatography, using a gradient of ethyl acetate (0 to 30%) in heptane as the eluent, gave 4.3 g (24% yield) of the title compound: MS (ESI) m/z 230 [M+1]⁺.

### Example 199

### 4-Bromo-2,6-diethylpyridine

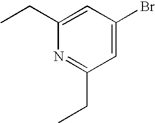

Phosphorous tribromide (2 mL) was added to a solution of 4-bromo-2,6-diethylpyridine 1-oxide (4.3 g, 18.7 mmol) in dichloromethane (30 mL). The reaction mixture was stirred at room temperature for 3 h and poured onto ice. The solution was made basic using sodium hydroxide (15%) and extracted with ethyl acetate. The combined organic phases were washed with brine, dried over sodium sulfate and concentrated in vacuo to give 2.9 g (72% yield) of the title compound: MS (ESI) m/z 215 [M+1]⁺.

### Example 200

### 2,6-Diethyl-4-[(trimethylsilyl)ethynyl]pyridine

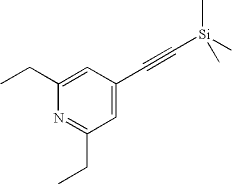

4-Bromo-2,6-diethylpyridine (2.9 g, 13.55 mmol), dichlorobis(triphenylphosphine)palladium (477 mg, 0.68 mmol) and copper(I) iodide (130 mg, 0.68 mmol) were placed in a vial and flushed with argon. Ethynyl(trimethyl)silane (2.34 mL, 16.9 mmol) and anhydrous N,N-dimethylformamide (20 mL) were added followed by diisopropylamine (5.75 mL, 40.7 mmol). The reaction was divided into two 25 mL vials and irradiated in a microwave at 100° C. for 15 min. Water was added and the mixture was extracted with dichloromethane. The combined organic phases were concentrated in vacuo and purified by column chromatography, using a gradient of ethyl acetate (0 to 30%) in n-heptane as the eluent, to give 1.64 g (63% yield) of the title compound: MS (ESI) m/z 232 [M+1]⁺.

### Example 201

### 2-Methyl-4-[(trimethylsilyl)ethynyl]pyridine

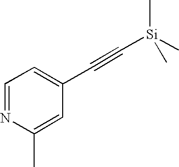

The title compound was synthesized as described for Example 200 in 63% yield, starting from 4-bromo-2-methylpyridine: MS (ESI) m/z 426 [M+1]⁺.

### Example 202

### 4-[(3-Bromo-4-fluorophenyl)ethynyl]-2,6-diethylpyridine

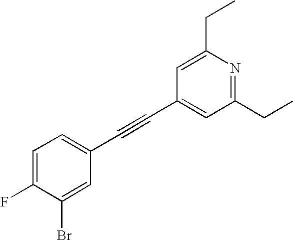

A suspension of 2,6-diethyl-4-[(trimethylsilyl)ethynyl]pyridine (2.61 g, 11.3 mmol) and potassium carbonate (7.78 g, 56.4 mmol) in methanol (20 mL) was stirred at room temperature for 2 h. The methanol was evaporated, water was added and the mixture was extracted with dichloromethane. The combined organic phases were dried over sodium sulfate and concentrated in vacuo. The residue was dissolved in anhydrous tetrahydrofuran (10 mL) and added to a solution of 2-bromo-1-fluoro-4-iodobenzene (3.39 g, 11.3 mmol), dichlorobis(triphenylphosphine)palladium (39 mg, 0.056 mmol) and copper(I) iodide (11 mg, 0.056 mmol in anhydrous tetrahydrofuran (30 mL) and triethylamine (15 mL). The reaction mixture was stirred at room temperature for 3 h, neutralized using hydrochloric acid (2 M) and extracted with dichloromethane. The combined organic phases were concentrated in vacuo and the residue was purified by column chromatography, using a gradient of ethyl acetate (0 to 40%) in n-heptane as the eluent, to give 2.4 g (64% yield) of the title compound: MS (ESI) m/z 334 [M+1]⁺.

### Example 203

### 4-Ethynyl-2-methylpyridine

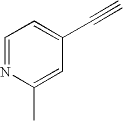

A suspension of 2-methyl-4-[(trimethylsilyl)ethynyl]pyridine (1.63 g, 8.62 mmol) and potassium carbonate (5.95 g, 43.1 mmol) in methanol (30 mL) was stirred for 2 h at 60° C. The methanol was evaporated in vacuo, water was added and the mixture was extracted with dichloromethane. The combined organic phases were dried over sodium sulfate and concentrated in vacuo to give 890 mg (88% yield) of the title compound: MS (ESI) m/z 426 [M+1]⁺.

### Example 204

### 4-[(3-Bromo-4-fluorophenyl)ethynyl]-2-methylpyridine

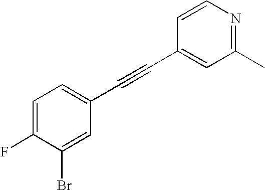

2-Bromo-1-fluoro-4-iodobenzene (890 mg, 7.6 mmol), dichlorobis(triphenylphosphine)palladium (27 mg, 0.038 mmol) and copper(I) iodide (7 mg, 0.038 mmol) were dissolved in anhydrous tetrahydrofuran (30 mL) and triethylamine (15 mL). 4-Ethynyl-2-methylpyridine (890 mg, 7.6 mmol) was added, the reaction stirred at room temperature for 3 h and then neutralized using hydrochloric acid (2 M). The solution was extracted with dichloromethane and the combined organic phases were concentrated in vacuo. Purification by column chromatography, using a gradient of ethyl acetate (0 to 40%) in n-heptane as the eluent, gave 1.4 g (63% yield) of the title compound: MS (ESI) m/z 426 [M+1]⁺.

### Example 205

### 1-(3-Bromo-4-fluorophenyl)-2-(2,6-diethylpyridin-4-yl)ethane-1,2-dione

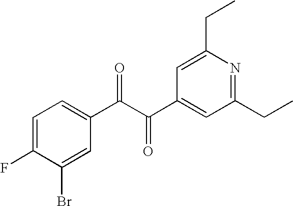

4-[(3-Bromo-4-fluorophenyl)ethynyl]-2,6-diethylpyridine (2.4 g, 7.29 mmol) and palladium(II) chloride (128 mg, 0.72 mmol) were dissolved in anhydrous dimethyl sulfoxide (35 mL) and stirred at 150° C. for 3 h. The reaction was allowed to cool to room temperature and diluted with water. The mixture was extracted with dichloromethane, the combined organic phases were washed with water before being concentrated onto silica. Purification by column chromatography, using a gradient of ethyl acetate (0 to 20%) in n-heptane as the eluent, gave 1.2 g (45% yield) of the title compound: MS (ESI) m/z 365 [M+1]⁺.

### Example 206

### 1-(3-Bromo-4-fluorophenyl)-2-(2-methylpyridin-4-yl)ethane-1,2-dione

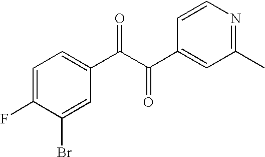

The title compound was synthesized as described for Example 205 in 90% yield, starting form 4-[(3-bromo-4-fluorophenyl)ethynyl]-2-methylpyridine. The product was purified by column chromatography, using a gradient of ethyl acetate (0 to 100%) in n-heptane as the eluent: MS (ESI) m/z 426 [M+1]⁺.

### Example 207

### 2-Amino-5-(3-bromo-4-fluorophenyl)-5-(2,6-diethylpyridin-4-yl)-3-methyl-3,5-dihydro-4H-imidazol-4-one

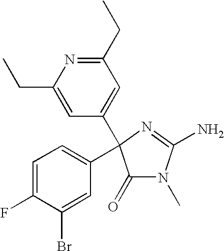

A solution of sodium carbonate (737 mg, 6.95 mmol) in water (10 mL) was added to a solution of 1-(3-bromo-4-fluorophenyl)-2-(2-methylpyridin-4-yl)ethane-1,2-dione (560 mg, 1.74 mmol) and methylguanidine hydrochloride (761 mg, 6.95 mmol) in a mixture of ethanol and dioxane (1: 1, 25 mL). The reaction was heated at 85° C. for 45 min and then allowed to cool to room temperature. The solution was extracted with dichloromethane and the combined organic phases were concentrated in vacuo. Purification by column chromatography, using a gradient of 0 to 10% ammonium hydroxide/methanol (1:9) in dichloromethane as the eluent, gave 283 mg (43% yield) of the title compound: MS (ESI) m/z 419, 421 [M+1]⁺.

### Example 208

### 2-Amino-5-(3-bromo-4-fluorophenyl)-3-methyl-5-(2-methylpyridin-4-yl)-3,5-dihydro-4H-imidazol-4-one

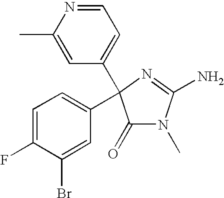

The title compound was synthesized as described for Example 207 in 43% yield, starting from 1-(3-bromo-4-fluorophenyl)-2-(2-methylpyridin-4-yl)ethane-1,2-dione: MS (ESI) m/z 426 [M+1]⁺.

### Example 209

### 5′-[2-Amino-4-(2,6-diethylpyridin-4-yl)-1-methyl-5-oxo-4,5-dihydro-1H-imidazol-4-yl]-2′-fluoro-5-methoxybiphenyl-3-yl methanesulfonate hydrochloride

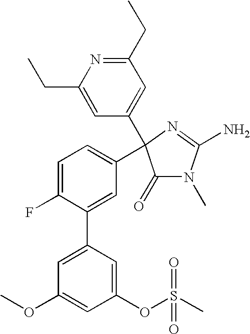

The title compound was synthesized as described for Example 194 in 29% yield, starting from 2-amino-5-(3-bromo-4-fluorophenyl)-5-(2,6-diethylpyridin-4-yl)-3-methyl-3,5-dihydro-4H-imidazol-4-one and 3-methoxy-5-(4,4,5,5-tetramethyl-1,3,2-dioxaborolan-2-yl)phenyl methanesulfonate: ¹H NMR (CD₃OD) δ 7.48-7.40 (m, 3H), 7.36-7.29 (m, 2H), 7.07-7.02 (m, 2H), 6.97 (t, J=2.3 Hz, 1H), 3.86 (s, 3H), 3.28 (s, 3H), 3.26 (s, 3H), 2.83 (q, J=7.7 Hz, 4H), 1.27 (t, J=7.6 Hz, 6H); MS (ESI) m/z 541 [M+1]⁺.

### Example 210

### 5′-[2-Amino-4-(2,6-diethylpyridin-4-yl)-1-methyl-5-oxo-4,5-dihydro-1H-imidazol-4-yl]-5-chloro-2′-fluorobiphenyl-3-yl methanesulfonate hydrochloride

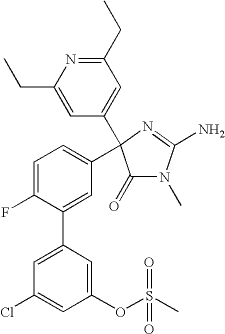

The title compound was synthesized as described for Example 194 in 8% yield, starting from 2-amino-5-(3-bromo-4-fluorophenyl)-5-(2,6-diethylpyridin-4-yl)-3-methyl-3,5-dihydro-4H-imidazol-4-one and 3-chloro-5-(4,4,5,5-tetramethyl-1,3,2-dioxaborolan-2-yl)phenyl methanesulfonate: ¹H NMR (CD₃OD) δ 7.82 (s, 2H), 7.61 (d, J=1.5 Hz, 1H), 7.57-7.51 (m, 2H), 7.47 (q, J=1.5 Hz, 2H), 7.39 (t, J=9.7 Hz, 1H), 3.31 (s, 3H), 3.30 (s, 3H), 3.05 (q, J=7.5 Hz, 4H), 1.38 (t, J=7.6 Hz, 6H); MS (ESI) m/z 545, 457 [M+1]⁺.

### Example 211

### 5′-[2-Amino-1-methyl-4-(2-methylpyridin-4-yl)-5-oxo-4,5-dihydro-1H-imidazol-4-yl]-2′-fluoro-5-methoxybiphenyl-3-yl methanesulfonate hydrocloride

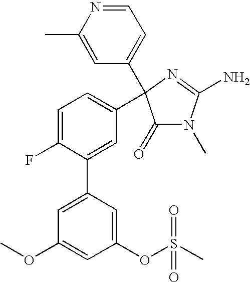

The title compound was synthesized as described for Example 194 in 57% yield, starting from 2-amino-5-(3-bromo-4-fluorophenyl)-3-methyl-5-(2-methylpyridin-4-yl)-3,5-dihydro-4H-imidazol-4-one and 3-methoxy-5-(4,4,5,5-tetramethyl-1,3,2-dioxaborolan-2-yl)phenyl methanesulfonate: ¹H NMR (DMSO-d₆) δ 8.60 (d, J=5.6 Hz, 1H), 7.61 (d, J=7.1 Hz, 1H), 7.53-7.35 (m, 4H), 7.12 (d, J=15.4 Hz, 2H), 7.05 (t, J=2.3 Hz, 1H), 3.85 (s, 3H), 3.44 (s, 3H), 3.17 (s, 3H), 2.55 (s, 3H); MS (ESI) m/z 426 [M+1]⁺.

### Example 212

### 3-[4-(3-Bromo-4-fluorophenyl)-1-methyl-5-oxo-2-thioxoimidazolidin-4-yl]phenyl 4-(1,1-dimethylpropyl)benzenesulfonate

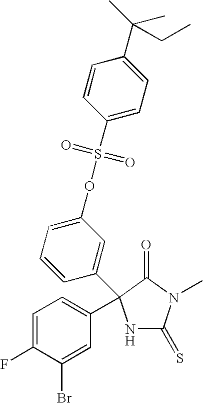

To a stirred solution of 5-(3-bromo-4-fluorophenyl)-5-(3-hydroxyphenyl)-3-methyl-2-thioxoimidazolidin-4-one (329 mg, 0.83 mmol) and triethylamine (150 μL, 1.08 mmol) in dichloromethane (3 mL) was added a solution of 4-(1,1-dimethylpropyl)benzenesulfonyl chloride (227 mg, 0.92 mmol) in dichloromethane (1 mL) at 0° C. under an atmosphere of nitrogen. Upon reaching room temperature the resulting mixture was stirred overnight. The reaction mixture was concentrated in vacuo to give the title compound which was used without further purification: MS (ESI) m/z 605, 607 [M+H]⁺.

### Examples 213-222

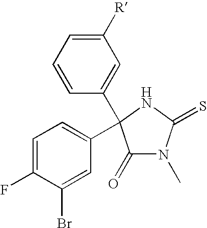

TABLE 4
Representative examples synthesized as described for 3-[4-(3-bromo-4-
fluorophenyl)-1-methyl-5-oxo-2-thioxoimidazolidin-4-yl]phenyl 4-(1,1-
dimethylpropyl)benzenesulfonate

| | | | MS (ES) |
|---|---|---|---|
| | | | m/z |
| Ex | Chemical name | R′ | [M + H]⁺ |
| 213 | 3-[4-(3-Bromo-4-fluorophenyl)-1-methyl-5-oxo-2-thioxoimidazolidin-4-yl]phenyl 2,5-dimethoxybenzenesulfonate | 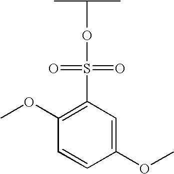 | 595, 597 |
| 214 | 3-[4-(3-Bromo-4-fluorophenyl)-1-methyl-5-oxo-2-thioxoimidazolidin-4-yl]phenyl 2,6-difluorobenzenesulfonate | 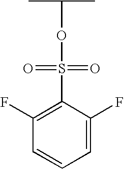 | 571, 573 |
| 215 | 3-[4-(3-Bromo-4-fluorophenyl)-1-methyl-5-oxo-2-thioxoimidazolidin-4-yl]phenyl pyridine-2-sulfonate | 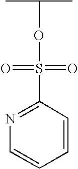 | 536, 538 |
| 216 | 3-[4-(3-Bromo-4-fluorophenyl)-1-methyl-5-oxo-2-thioxoimidazolidin-4-yl]phenyl 4-(1H-pyrazol-1-yl)benzenesulfonate | 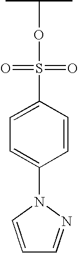 | 601, 603 |
| 217 | 3-[4-(3-Bromo-4-fluorophenyl)-1-methyl-5-oxo-2-thioxoimidazolidin-4-yl]phenyl indane-5-sulfonate | 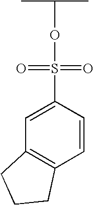 | 575, 577 |
| 218 | 3-[4-(3-Bromo-4-fluorophenyl)-1-methyl-5-oxo-2-thioxoimidazolidin-4-yl]phenyl 3-methyl-2-oxo-2,3-dihydro-1,3-benzoxazole-6-sulfonate | 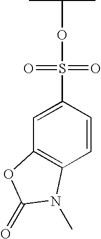 | 606, 608 |
| 219 | 3-[4-(3-Bromo-4-fluorophenyl)-1-methyl-5-oxo-2-thioxoimidazolidin-4-yl]phenyl 5-chloro-1,3-dimethyl-1H-pyrazole-4-sulfonate | 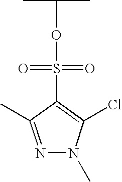 | 587, 589 |
| 220 | 3-[4-(3-Bromo-4-fluorophenyl)-1-methyl-5-oxo-2-thioxoimidazolidin-4-yl]phenyl 5-chlorothiophene-2-sulfonate | 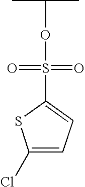 | 575, 577 |
| 221 | 3-[4-(3-Bromo-4-fluorophenyl)-1-methyl-5-oxo-2-thioxoimidazolidin-4-yl]phenyl 1-methyl-1H-imidazole-4-sulfonate | 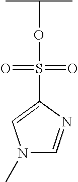 | 539, 541 |
| 222 | 3-[4-(3-Bromo-4-fluorophenyl)-1-methyl-5-oxo-2-thioxoimidazolidin-4-yl]phenyl 6-chloroimidazo[2,1-b][1,3]thiazole-5-sulfonate | 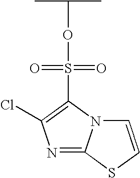 | 515, 517 |

### Example 223

### 3-[2-Amino-4-(3-bromo-4-fluorophenyl)-1-methyl-5-oxo-4,5-dihydro-1H-imidazol-4-yl]phenyl 4-(1,1-dimethylpropyl)benzenesulfonate

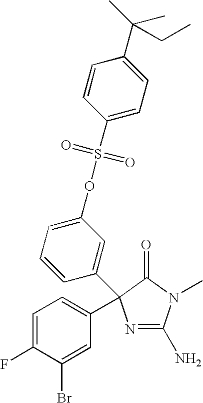

To a stirred solution of 3-[4-(3-bromo-4-fluorophenyl)-1-methyl-5-oxo-2-thioxoimidazolidin-4-yl]phenyl 4-(1,1-dimethylpropyl)benzenesulfonate in methanol (3.75 mL) was added ammonium hydroxide (1.25 mL) and tert-butyl hydroperoxide (70% in water, 1.20 mL, 12.4 mmol) and the resulting mixture was stirred at 35° C. for 2 h. The mixture was allowed to cool down to room temperature and concentrated in vacuo. The residue was partitioned between chloroform and a mixture of brine and water (1:1). The organic phase was dried over magnesium sulfate, filtered and concentrated. Approximately ¼ of the crude material was purified by preparative HPLC. Remaining ¾ was used without further purification: ¹H NMR (DMSO-d₆) δ 7.72 (m, 2H), 7.61-7.55 (m, 3H), 7.41 (m, 1H), 7.36-7.29 (m, 3H), 7.15 (m, 1H), 6.93 (m, 1H), 6.81 (br s, 2H), 2.96 (s, 3H), 1.62 (q, J=7.5 Hz, 2H), 1.25 (s, 6H), 0.57 (t, J=7.5 Hz, 3H); MS (ESI) m/z 588, 590 [M+H]⁺.

### Examples 224-233

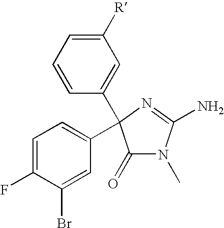

TABLE 5
Representative examples synthesized as described for 3-[2-amino-4-(3-bromo-4-
fluorophenyl)-1-methyl-5-oxo-4,5-dihydro-1H-imidazol-4-yl]phenyl 4-(1,1-
dimethylpropyl)benzenesulfonate

| | | | | MS (ES) | ¹H-NMR |
|---|---|---|---|---|---|
| | SM | | | m/z | (DMSO-d₆) |
| Ex | (Ex) | Chemical name | R′ | [M + H]⁺ | δ ppm |
| 224 | 213 | 3-[2-Amino-4-(3-bromo-4-fluorophenyl)-1-methyl-5-oxo-4,5-dihydro-1H-imidazol-4-yl]phenyl 2,5-dimethoxybenzenesulfonate | 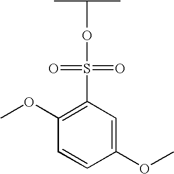 | 578, 580 | 7.59 (m, 1H), 7.38-7.23(m, 6H), 7.09-7.04 (m,2H), 7.01 (m, 1H), 6.80 (brs, 2H), 3.91 (s, 3H), 3.67(s, 3H), 2.95 (s, 3H). |
| 225 | 214 | 3-[2-Amino-4-(3-bromo-4-fluorophenyl)-1-methyl-5-oxo-4,5-dihydro-1H-imidazol-4-yl]phenyl 2,6-difluorobenzenesulfonate | 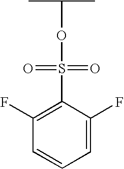 | 554, 556 | 7.94-7.84 (m, 1H), 7.56(m, 1H), 7.46 7.28 (m,6H), 7.15 (m, 1H), 7.08 (m,1H), 6.82 (br s, 2H), 2.95(s, 3H). |
| 226 | 215 | 3-[2-Amino-4-(3-bromo-4-fluorophenyl)-1-methyl-5-oxo-4,5-dihydro-1H-imidazol-4-yl]phenyl pyridine-2-sulfonate | 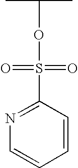 | 519, 521 | 8.82 (d, J = 4.3 Hz, 1H),8.08 (m, 1H), 7.93 (m,1H), 7.81 (m, 1H), 7.58 (m,1H), 7.39-7.29 (m, 4H),7.12-7.06 (m, 1H), 6.94(m, 1H), 6.80 (br s, 2H),2.95 (s, 3H). |
| 227 | 216 | 3-[2-Amino-4-(3-bromo-4-fluorophenyl)-1-methyl-5-oxo-4,5-dihydro-1H-imidazol-4-yl]phenyl 4-(1H-pyrazol-1-yl)benzenesulfonate | 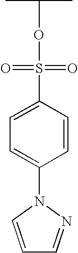 | 584, 586 | 8.70 (d, J = 2.5 Hz, 1H),8.10 (m, 2H), 7.88 (m,3H), 7.57 (m, 1H), 7.43-7.33 (m, 2H), 7.28 (m,1H), 7.16 (m, 1H), 7.07-7.01 (m, 2H), 6.78 (br s,2H), 6.66 (m, 1H), 2.89 (s,3H). |
| 228 | 217 | 3-[2-Amino-4-(3-bromo-4-fluorophenyl)-1-methyl-5-oxo-4,5-dihydro-1H-imidazol-4-yl]phenyl indane-5-sulfonate | 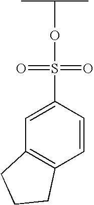 | 558, 560 | 7.64 (m, 1H), 7.59 (m,1H), 7.48 (m, 1H), 7.43-7.27 (m, 5H), 7.03-6.97(m, 2H), 6.80 (br s, 2H),3.00-2.87 (m, 4H), 2.95(s, 3H), 2.06 (m, 2H). |
| 229 | 218 | 3-[2-Amino-4-(3-bromo-4-fluorophenyl)-1-methyl-5-oxo-4,5-dihydro-1H-imidazol-4-yl]phenyl 3-methyl-2-oxo-2,3-dihydro-1,3-benzoxazole-6-sulfonate | 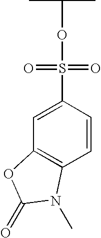 | 589, 591 | 7.78 (m, 1H), 7.64 (m,1H), 7.57 (m, 1H), 7.43 (m,1H), 7.40-7.24 (m, 4H),7.03 (m, 1H), 6.92 (m,1H), 6.78 (br s, 2H), 3.40(s, 3H), 2.94 (s, 3H). |
| 230 | 219 | 3-[2-Amino-4-(3-bromo-4-fluorophenyl)-1-methyl-5-oxo-4,5-dihydro-1H-imidazol-4-yl]phenyl 5-chloro-1,3-dimethyl-1H-pyrazole-4-sulfonate | 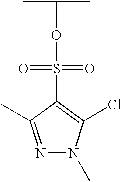 | 570, 572 | 7.63 (m, 1H), 7.44-7.30(m, 4H), 7.07-7.03 (m,2H), 6.83 (br s, 2H), 3.78(s, 3H), 2.98 (s, 3H), 1.94(s, 3H). |
| 231 | 220 | 3-[2-Amino-4-(3-bromo-4-fluorophenyl)-1-methyl-5-oxo-4,5-dihydro-1H-imidazol-4-yl]phenyl 5-chlorothiophene-2-sulfonate | 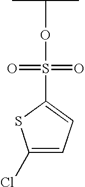 | 558, 560 | 7.66 (m, 1H), 7.63 (m,1H), 7.49-7.29 (m, 5H),7.11 (m, 1H), 7.08 (m,1H), 6.83 (br s, 2H), 2.97(s, 3H). |
| 232 | 221 | 3-[2-Amino-4-(3-bromo-4-fluorophenyl)-1-methyl-5-oxo-4,5-dihydro-1H-imidazol-4-yl]phenyl 1-methyl-1H-imidazole-4-sulfonate | 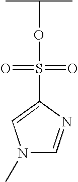 | 522, 524 | 7.92 (m, 2H), 7.65 (m,1H), 7.44-7.30 (m, 4H),7.04 (m, 1H), 6.98 (m,1H), 6.81 (br s, 2H), 3.68(s, 3H), 2.97 (s, 3H). |
| 233 | 222 | 3-[2-Amino-4-(3-bromo-4-fluorophenyl)-1-methyl-5-oxo-4,5-dihydro-1H-imidazol-4-yl]phenyl 6-chloroimidazo[2,1-b][1,3]thiazole-5-sulfonate | 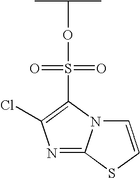 | 598, 600 | 7.73 (m, 1H), 7.64-7.57(m, 2H), 7.43-7.26 (m,4H), 7.06 (m, 1H), 7.00 (m,1H), 6.80 (br s, 2H), 2.96(s, 3H). |

### Example 234

### 3-[2-Amino-4-(4-fluoro-3-pyrimidin-5-ylphenyl)-1-methyl-5-oxo-4,5-dihydro-1H-imidazol-4-yl]phenyl 4-(1,1-dimethylpropyl)benzenesulfonate

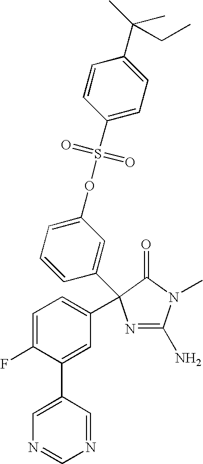

3-[2-Amino-4-(3-bromo-4-fluorophenyl)-1-methyl-5-oxo-4,5-dihydro-1H-imidazol-4-yl]phenyl 4-(1,1-dimethylpropyl)benzenesulfonate, pyrimidin-5-ylboronic acid (89 mg, 0.72 mmol), [1,1′-bis(diphenylphosphino)ferrocene]palladium(II) chloride dichloromethane adduct (49 mg, 0.06 mmol) and potassium carbonate (498 mg, 3.60 mmol) were suspended in tetrahydrofuran (3.5 mL) under an atmosphere of argon. The resulting slurry was irradiated in a microwave at 130° C. for 3 h. Upon cooling to room temperature the mixture was filtered and purified by preparative HPLC to give 18.3 mg (3.8% yield from 5-(3-bromo-4-fluorophenyl)-5-(3-hydroxyphenyl)-3-methyl-2-thioxoimidazolidin-4-one) of the title compound: ¹H NMR (DMSO-d₆) δ 9.24 (s, 1H), 8.93 (m, 2H), 7.75 (m, 2H), 7.62-7.55 (m, 3H), 7.52-7.43 (m, 2H), 7.41-7.26 (m, 3H), 6.90 (m, 1H), 6.79 (br s, 2H), 2.97 (s, 3H), 1.61 (q, J=7.4 Hz, 2H), 1.24 (s, 6H), 0.56 (t, J=7.4 Hz, 3H); MS (ESI) m/z 588 [M+H]⁺.

### Examples 235-243

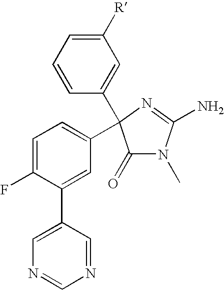

TABLE 6
Representative examples synthesized as described for 3-[2-amino-4-(4-fluoro-3-
pyrimidin-5-ylphenyl)-1-methyl-5-oxo-4,5-dihydro-1H-imidazol-4-yl]phenyl 4-(1,1-
dimethylpropyl)benzenesulfonate

| | | | | MS (ES) | ¹H-NMR |
|---|---|---|---|---|---|
| | SM | | | m/z | (DMSO-d₆) |
| Ex | (Ex) | Chemical name | R′ | [M + H]⁺ | δ ppm |
| 235 | 224 | 3-[2-Amino-4-(4-fluoro-3-pyrimidin-5-ylphenyl)-1-methyl-5-oxo-4,5-dihydro-1H-imidazol-4-yl]phenyl 2,5-dimethoxybenzenesulfonate | 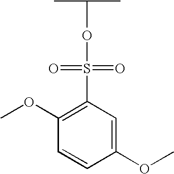 | 578 | 9.23 (s, 1H), 8.91 (m, 2H),7.57 (m, 1H), 7.42-7.35(m, 2H), 7.34-7.23 (m,4H), 7.18 (m, 1H), 7.07-7.01 (m, 2H), 6.77 (br s,2H), 3.90 (s, 3H), 3.65 (s,3H), 2.96 (s, 3H). |
| 236 | 225 | 3-[2-Amino-4-(4-fluoro-3-pyrimidin-5-ylphenyl)-1-methyl-5-oxo-4,5-dihydro-1H-imidazol-4-yl]phenyl 2,6-difluorobenzenesulfonate | 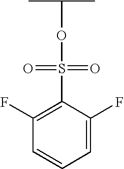 | 554 | 9.23 (s, 1H), 8.91 (m, 2H),7.86 (m, 1H), 7.58-7.48(m, 2H), 7.45-7.30 (m,5H), 7.20 (m, 1H), 7.11 (m,1H), 6.78 (br s, 2H), 2.96 (s,3H). |
| 237 | 226 | 3-[2-Amino-4-(4-fluoro-3-pyrimidin-5-ylphenyl)-1-methyl-5-oxo-4,5-dihydro-1H-imidazol-4-yl]phenyl pyridine-2-sulfonate | 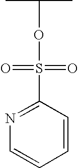 | 519 | No data |
| 238 | 227 | 3-[2-Amino-4-(4-fluoro-3-pyrimidin-5-ylphenyl)-1-methyl-5-oxo-4,5-dihydro-1H-imidazol-4-yl]phenyl 4-(1H-pyrazol-1-yl)benzenesulfonate | 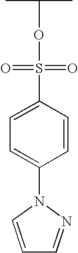 | 584 | 9.22 (s, 1H), 8.89 (m, 2H),8.68 (m, 1H), 8.07 (m, 2H),7.90-7.85 (m, 3H), 7.57(m, 1H), 7.48 (m, 1H), 7.39-7.32 (m, 2H), 7.19 (m,1H), 7.12 (m, 1H), 7.03 (m,1H), 6.74 (br s, 2H), 6.64(m,1H), 2.90 (s, 3H). |
| 239 | 228 | 3-[2-Amino-4-(4-fluoro-3-pyrimidin-5-ylphenyl)-1-methyl-5-oxo-4,5-dihydro-1H-imidazol-4-yl]phenyl indane-5-sulfonate | 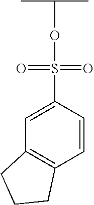 | 558 | 9.24 (s, 1H), 8.92 (m, 2H),7.65 (m, 1H), 7.58 (m, 1H),7.50 (m, 1H), 7.47-7.42(m, 2H), 7.42-7.31 (m,3H), 7.13 (m, 1H), 6.97 (m,1H), 6.78 (br s, 2H), 2.96 (s,3H), 2.90 (m, 4H), 2.04 (m,2H). |
| 240 | 229 | 3-[2-Amino-4-(4-fluoro-3-pyrimidin-5-ylphenyl)-1-methyl-5-oxo-4,5-dihydro-1H-imidazol-4-yl]phenyl 3-methyl-2-oxo-2,3-dihydro-1,3-benzoxazole-6-sulfonate | 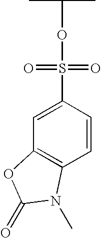 | 589 | 9.23 (s, 1H), 8.91 (m, 2H),7.78 (m, 1H), 7.65 (m, 1H),7.57 (m, 1H), 7.48-7.38(m, 3H), 7.38-7.28 (m,2H), 7.05 6.99 (m, 2H),6.75 (br s, 2H), 3.38 (s,3H), 2.94 (s, 3H). |
| 241 | 230 | 3-[2-Amino-4-(4-fluoro-3-pyrimidin-5-ylphenyl)-1-methyl-5-oxo-4,5-dihydro-1H-imidazol-4-yl]phenyl 5-chloro-1,3-dimethyl-1H-pyrazole-4-sulfonate | 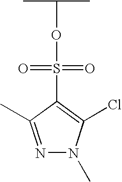 | 570 | 9.23 (s, 1H), 8.93 (m, 2H),7.58 (m, 1H), 7.52-7.45(m, 2H), 7.41-7.33 (m,2H), 7.12 (m, 1H), 7.04 (m,1H), 6.80 (br s, 2H), 3.76 (s,3H), 2.99 (s, 3H), 1.90 (s,3H). |
| 242 | 231 | 3-[2-Amino-4-(4-fluoro-3-pyrimidin-5-ylphenyl)-1-methyl-5-oxo-4,5-dihydro-1H-imidazol-4-yl]phenyl-5-chlorothiophene-2-sulfonate | 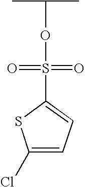 | 558 | No data |
| 243 | 221 | 3-[2-Amino-4-(4-fluoro-3-pyrimidin-5-ylphenyl)-1-methyl-5-oxo-4,5-dihydro-1H-imidazol-4-yl]phenyl 1-methyl-1H-imidazole-4-sulfonate | 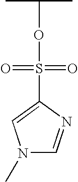 | 522 | 9.23 (s, 1H), 8.94 (m, 2H),7.92 (m, 2H), 7.63 (m, 1H),7.52-7.31 (m, 4H), 7.09(m, 1H), 7.02 (m, 1H), 6.78(br s, 2H), 3.68 (s, 3H),2.98 (s, 3H). |

### Example 244

### 4-(Dimethoxymethyl)-1-ethyl-2-nitrobenzene

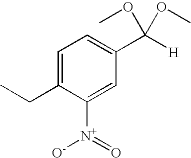

4-Ethyl-3-nitrobenzaldehyde (3.0 g, 16.74 mmol) was dissolved in methanol (80 mL), Dowex 50H+ (1.7 g) was added and the resulting mixture was stirred at room temperature under an atmosphere of nitrogen overnight. The resin was filtered off and the residue concentrated to give 3.90 g (quantitative yield) of the title compound: ¹H NMR (CDCl₃) δ 7.98 (d, J=1.8 Hz, 1H), 7.60 (dd, J=8.0 Hz, 1.8 Hz, 1H), 7.38 (d, J=8.0 Hz, 1H), 5.43 (s, 1H), 3.34 (s, 6H), 2.92 (q, J=7.5 Hz, 2H), 1.29 (t, J=7.5 Hz, 3H).

### Example 245

### 5-(Dimethoxymethyl)-2-ethylaniline

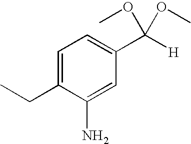

Platinum(IV) oxide (20 mg, 0.09 mmol) was added to a solution of 4-(dimethoxymethyl)-1-ethyl-2-nitrobenzene (970 mg, 4.31 mmol) in methanol (20 mL). The resulting mixture was hydrogenated at atmospheric pressure and room temperature for 1 h. Additional platinum(IV) oxide (20 mg) was added and the mixture was hydrogenated overnight. The mixture was filtered through diatomaceous earth and concentrated to give 778 mg (92% yield) of the title compound: ¹H NMR (DMSO-d₆) δ 6.89 (d, J=7.7 Hz, 1H), 6.65 (d, J=1.7 Hz, 1H), 6.51 (dd, J=7.7 Hz, 1.7 Hz, 1H), 5.19 (s, 1H), 4.86 (br s, 2H), 3.19 (s, 6H), 2.41 (q, J=7.5 Hz, 2H), 1.11 (t, J=7.5 Hz, 3H).

### Example 246

### 4-Ethyl-3-hydroxybenzaldehyde

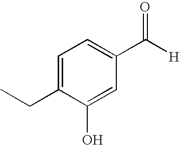

5-(Dimethoxymethyl)-2-ethylaniline (2.34 g, 11.98 mmol) was dissolved in sulfuric acid (35%, 12 mL) and ice (12 g) was added. The mixture was cooled to 0° C. and a solution of sodium nitrite (1.04 g, 15.10 mmol) in water (12 mL) was slowly added under the surface of the mixture. Upon complete addition the mixture was stirred for 5 min before excess sodium nitrite was decomposed by addition of a few crystals of urea. The cooling bath was removed and a solution of copper(II) nitrate trihydrate in water (420 mL) was added to the still cold mixture. Copper(I) oxide (1.59 g, 11.14 mmol) was added under vigorous stirring and the resulting mixture was stirred at room temperature for 1 h. The product was extracted from the reaction mixture with diethyl ether and then from the organic extract with aqueous sodium hydroxide (1 M). The combined aqueous layers were acidified with hydrochloric acid (2 M) and the product was re-extracted with diethyl ether. The combined organic extracts were dried over magnesium sulfate, filtered and concentrated. Purification by column chromatography, using a gradient of ethyl acetate (0-50%) in heptane as the eluent, gave 625 mg (35% yield) of the title compound: ¹H NMR (DMSO-d₆) δ 9.89 (s, 1H), 9.85 (s, 1H), 7.31 (m, 2H), 7.25 (m, 1H), 2.60 (q, J=7.5 Hz, 2H), 1.14 (t, J=7.5 Hz, 3H); MS (ESI) m/z 149 [M−1]⁻.

### Example 247

### 3-{[tert-Butyl(diphenyl)silyl]oxy}-4-ethylbenzaldehyde

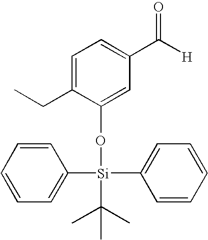

tert-Butyl(chloro)diphenylsilane (1.74 mL, 6.68 mmol) was added dropwise to a solution of 4-ethyl-3-hydroxybenzaldehyde (912 mg, 6.07 mmol), N,N-dimethylpyridin-4-amine (37 mg, 0.30 mmol) and triethylamine (0.93 mL, 6.68 mmol) in dichloromethane (30 mL) under an atmosphere of nitrogen and the resulting mixture was stirred at room temperature for 20 h. The temperature was raised to 40° C. for 6 h and the reaction mixture was then allowed to slowly reach room temperature overnight. More tert-butyl(chloro)diphenylsilane (315 μL, 1.21 mmol) was added and the mixture was stirred at reflux overnight before another 315 μL tert-butyl(chloro)diphenylsilane (1.21 mmol) followed by a few crystals N,N-dimethylpyridin-4-amine were added. The mixture was stirred at 35° C. for 3 days before saturated aqueous ammonium chloride was added. The phases were separated and the aqueous phase was extracted with dichloromethane. The combined organic extracts were dried over magnesium sulfate, filtered and concentrated. Purification by column chromatography, using a gradient of ethyl acetate (0-30%) in heptane as the eluent, gave 2.30 g (98% yield) of the title compound: ¹H NMR (CDCl₃) δ 9.51 (s, 1H), 7.75-7.70 (m, 5H), 7.48-7.35 (m, 8H), 2.90 (q, J=7.5 Hz, 2H), 1.34 (t, J=7.5 Hz, 3H), 1.14 (s, 9H); MS (ESI) m/z 389 [M+1]⁺.

### Example 248

### [2-(3-Bromo-4-fluorophenyl)-1,3-dithian-2-yl] (3-{[tert-butyl(diphenyl)silyl]oxy}-4-ethylphenyl)methanol

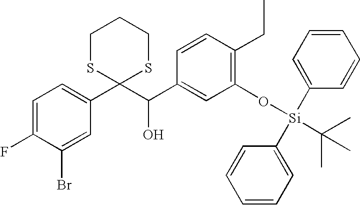

2-(3-Bromo-4-fluorophenyl)-1,3-dithiane (0.96 g, 3.27 mmol) was dissolved in tetrahydrofuran (20 mL) under an atmosphere of nitrogen and the resulting solution was cooled to 0° C. Lithium diisopropylamine (1.4 M in heptane/tetrahydrofuran/ethylbenzene, 2.57 mL, 3.60 mmol) was added over 2 min followed by addition of 3-{[tert-butyl(diphenyl)silyl]oxy}-4-ethylbenzaldehyde (1.27 g, 3.27 mmol) in tetrahydrofuran (5 mL). The mixture was stirred for 15 min and saturated aqueous ammonium chloride was added. The phases were separated and the organic phase was dried over magnesium sulfate, filtered and concentrated. Purification by column chromatography, using a gradient of ethyl acetate (0-100%) in heptane as the eluent, gave 1.10 g (49% yield) of the title compound: MS (ESI) m/z 665 [M−18]⁻.

### Example 249

### 1-(3-Bromo-4-fluorophenyl)-2-(3-{[tert-butyl(diphenyl)silyl]oxy}-4-ethylphenyl)ethane-1,2-dione

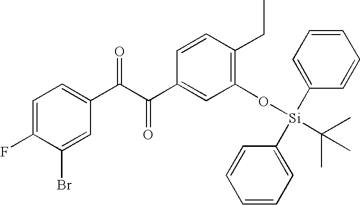

tert-Butanol (418 mg, 5.65 mmol) was added to a solution of [2-(3-bromo-4-fluorophenyl)-1,3-dithian-2-yl](3-{[tert-butyl(diphenyl)silyl]oxy}-4-ethylphenyl)methanol (1.10 g, 1.61 mmol) in dichloromethane (15 mL) under an atmosphere of nitrogen. 1,1,1-Tris(acetyloxy)-1λ⁵,2-benziodoxol-3(1H)-one (1.71 g, 4.03 mmol) was added in portions and the resulting mixture was stirred overnight. Sodium thiosulphate (1.6 g) in saturated aqueous sodium hydrogen carbonate (15 mL) was added and the phases were separated. The aqueous phase was extracted with dichloromethane, the combined organic extracts were dried over magnesium sulfate, filtered and concentrated. Purification by column chromatography, using a gradient of ethyl acetate (0-20%) in heptane as the eluent, gave 639 mg (67% yield) of the title compound: MS (ESI) m/z 589, 591 [M+1]⁺.

### Example 250

### 5-(3-Bromo-4-fluorophenyl)-5-(4-ethyl-3-hydroxyphenyl)-3-methyl-2-thioxoimidazolidin-4-one

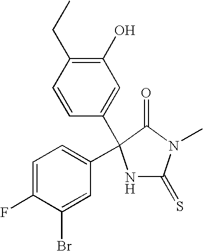

1-(3-Bromo-4-fluorophenyl)-2-(3-{[tert-butyl(diphenyl)silyl]oxy}-4-ethylphenyl)ethane-1,2-dione (620 mg, 1.05 mmol) and N-methylthiourea (190 mg, 2.10 mmol) were dissolved in dimethyl sulfoxide (2 mL) and the resulting solution was heated at 100° C. Aqueous potassium hydroxide (1.2 M, 1.23 mL, 1.47 mmol) was added dropwise, and the resulting mixture was stirred for 20 min. After cooling to room temperature, water (5 mL) was added followed by addition of hydrochloric acid (2 M) to pH 5. The mixture was extracted with dichloromethane followed by ethyl acetate. The combined organic extracts were dried over magnesium sulfate, filtered and concentrated. Purification by column chromatography, using a gradient of ethyl acetate (0-40%) in heptane as the eluent, gave 249 mg (58% yield) of the title compound: MS (ESI) m/z 421, 423 [M+1]⁺.

### Example 251

### 5-[4-(3-Bromo-4-fluorophenyl)-1-methyl-5-oxo-2-thioxoimidazolidin-4-yl]-2-ethylphenyl methanesulfonate

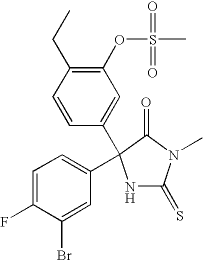

To a solution of 5-(3-bromo-4-fluorophenyl)-5-(4-ethyl-3-hydroxyphenyl)-3-methyl-2-thioxoimidazolidin-4-one (125 mg, 0.30 mmol) in dichloromethane (2 mL) was added triethyl amine (82 μL, 0.59 mmol) followed by methanesulfonyl chloride (34 μL, 0.44 mmol) at 0° C. under an atmosphere of nitrogen. After 1 h, additional triethylamine (41 μL, 0.30 mmol) and methanesulfonyl chloride (23 μL, 30 mmol) were added and the resulting mixture was stirred for 1 h. The mixture was concentrated and then ethyl acetate was added. The formed precipitate was filtered off and the residue purified by column chromatography, using a gradient of ethyl acetate (0-40%) in heptane as the eluent, to give 58 mg (39% yield) of the title compound: MS (ESI) m/z 498, 500 [M−1]⁻.

### Example 252

### 5-[2-Amino-4-(3-bromo-4-fluorophenyl)-1-methyl-5-oxo-4,5-dihydro-1H-imidazol-4-yl]-2-ethylphenyl methanesulfonate

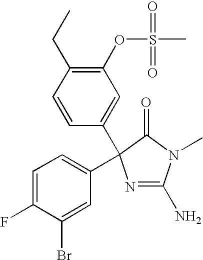

To two stirred solutions of 5-[4-(3-bromo-4-fluorophenyl)-1-methyl-5-oxo-2-thioxoimidazolidin-4-yl]-2-ethylphenyl methanesulfonate (77 mg, 0.15 mmol and 58 mg, 0.12 mmol respectively) in methanol (1.125 mL and 750 μL respectively) were ammonium hydroxide (375 μL and 250 μL respectively) and tert-butyl hydroperoxide (70% in water, 223 μL, 2.30 mmol and 170 μL, 1.74 mmol respectively) added and the resulting mixtures were stirred at 35° C. for 3 and 2.5 h respectively. The mixtures were allowed to cool to room temperature and were concentrated in vacuo. The residues were combined and partitioned between chloroform and a mixture of brine and water (1:1) and the aqueous layer was extracted with chloroform. The combined organic extracts were dried over magnesium sulfate, filtered and concentrated to give 146 mg (quantitative yield) of the title compound which was used without further purification: MS (ESI) m/z 483, 485 [M+1]⁺.

### Example 253

### 5-[2-Amino-4-(4-fluoro-3-pyrimidin-5-ylphenyl)-1-methyl-5-oxo-4,5-dihydro-1H-imidazol-4-yl]-2-ethylphenyl methanesulfonate 0.5 acetate

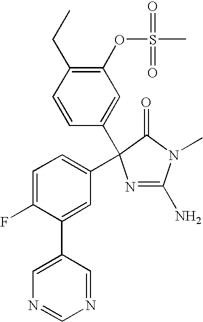

5-[2-Amino-4-(3-bromo-4-fluorophenyl)-1-methyl-5-oxo-4,5-dihydro-1H-imidazol-4-yl]-2-ethylphenyl methanesulfonate (72 mg, 0.149 mmol), pyrimidin-5-ylboronic acid (22 mg, 0.178 mmol), [1,1′-bis(diphenylphosphino)ferrocene]palladium(II) chloride dichloromethane adduct (6 mg, 0.007 mmol) and potassium carbonate (62 mg, 0.45 mmol) were suspended in a mixture of tetrahydrofuran/water (4: 1, 1 mL) and the resulting slurry was irradiated in a microwave at 130° C. 15 min. Additional pyrimidin-5-ylboronic acid (9.2 mg, 0.075 mmol) and [1,1′-bis(diphenylphosphino)ferrocene]palladium(II) chloride dichloromethane adduct (6 mg, 0.007 mmol) were added and the mixture was irradiated in a microwave at 130° C. for another 30 min. Upon cooling to room temperature, water and ethyl acetate were added and the phases were separated. The aqueous phase was extracted with ethyl acetate, and the combined organic extracts were dried over magnesium sulfate, filtered and concentrated. Purification by preparative HPLC gave 11.2 mg (15% yield) of the title compound: ¹H NMR (CDCl₃) δ 9.20 (s, 1H), 8.92 (d, J=1.4 Hz, 2H), 7.64-7.59 (m, 2H), 7.46 (dd, J=8.1, 1.9 Hz, 1H), 7.36 (m, 1H), 7.29 (d, J=8.1 Hz, 1H), 7.19 (m, 1H), 3.22 (s, 3H), 3.12 (s, 3H), 2.71 (q, J=7.6 Hz, 2H), 1.23 (t, J=7.6 Hz, 3H); MS (ESI) m/z 484 [M+1]⁺.

### Example 254

### 2-Chloro-4-methoxy-1-(trifluoromethoxy)benzene

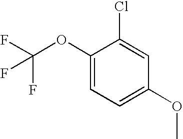

A mixture of 3-chloro-4-(trifluoromethoxy)phenol (1.00 g, 4.71 mmol), dimethylsulfate (490 μL, 5.18 mmol), tetrabutylammonium sulfate, 2 M aqueous sodium hydroxide (2.59 mL, 5.18 mmol) and dichloromethane (25 mL) was vigorously stirred at room temperature for 18 h. The mixture was diluted with dichloromethane, washed with water, dried over sodium sulfate and concentrated in vacuo to give 780 mg (73% yield) of the title compound: ¹H NMR (CDCl₃) □7.22-7.26 (m, 1H), 6.99 (d, J=2.8 Hz, 1H), 6.81 (dd, J=9.1, 3.0 Hz, 1H), 3.82 (s, 3H).

### Example 255

### 2-[5-Methoxy-2-(trifluoromethoxy)phenyl]-4,4,5,5-tetramethyl-1,3,2-dioxaborolane

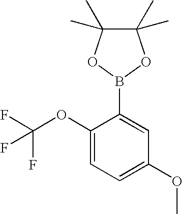

A mixture of tris(dibenzylideneacetone)dipalladium (40 mg, 0.044 mmol), tricyclohexylphosphine (57 mg, 0.203 mmol), 4,4,4′,4′,5,5,5′,5′-Octamethyl-2,2′-bi-1,3,2-dioxaborolane (407 mg, 1.60 mmol), potassium acetate (214 mg, 2.18 mmol) and 2-chloro-4-methoxy-1-(trifluoromethoxy)benzene (330 mg, 1.46 mmol) in anhydrous dioxane (5 mL) was added to a microwave vial. The vial was sealed and purged with argon and the resulting mixture was irradiated in a microwave at 120° C. for 15 h. The cooled mixture was filtered through a short plug of silica gel and washed with dichloromethane. The filtrate was concentrated and the residue was purified by column chromatography, using a gradient of 0-100% ethyl acetate in heptane followed by 10% MeOH in dichloromethane as the eluent to give the title compound: ¹H NMR (CDCl₃) □7.25 (d, J=3.3 Hz, 1H), 7.16 (m, 1H), 6.97 (dd, J=9.0, 3.2 Hz, 1H), 3.83 (s, 3H), 1.36 (s, 12H); MS (EI) m/z 318 [M+□].

### Example 256

### 4-[4-(3-Bromo-4-fluorophenyl)-1-methyl-5-oxo-2-thioxoimidazolidin-4-yl]phenyl 4-(1,1-dimethylpropyl)benzenesulfonate

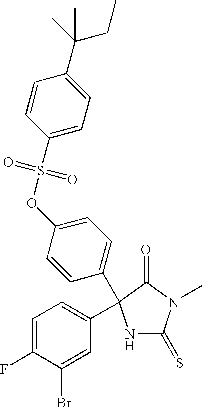

The title compound was synthesized as described for Example 212 starting from 5-(3-bromo-4-fluorophenyl)-5-(4-hydroxyphenyl)-3-methyl-2-thioxoimidazolidin-4-one, and was used without further purification: MS (ESI) m/z 602.99, 605.01 [M−H]⁻.

### Examples 257-265

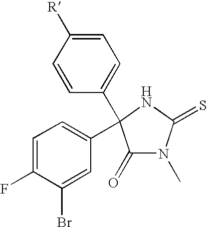

TABLE 7
Representative examples synthesized as described for 3-[4-(3-bromo-4-
fluorophenyl)-1-methyl-5-oxo-2-thioxoimidazolidin-4-yl]phenyl 4-(1,1-
dimethylpropyl)benzenesulfonate

| | | | MS (ES) |
|---|---|---|---|
| | | | m/z |
| Ex | Chemical name | R′ | [M − H]⁻ |
| 257 | 4-[4-(3-Bromo-4-fluorophenyl)-1-methyl-5-oxo-2-thioxoimidazolidin-4-yl]phenyl 2,5-dimethoxybenzenesulfonate | 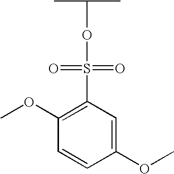 | 593, 595 |
| 258 | 4-[4-(3-Bromo-4-fluorophenyl)-1-methyl-5-oxo-2-thioxoimidazolidin-4-yl]phenyl 2,6-difluorobenzenesulfonate | 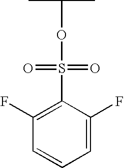 | 569, 571 |
| 259 | 4-[4-(3-Bromo-4-fluorophenyl)-1-methyl-5-oxo-2-thioxoimidazolidin-4-yl]phenyl indane-5-sulfonate | 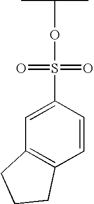 | 573, 575 |
| 260 | 4-[4-(3-Bromo-4-fluorophenyl)-1-methyl-5-oxo-2-thioxoimidazolidin-4-yl]phenyl 3-methyl-2-oxo-2,3-dihydro-1,3-benzoxazole-6-sulfonate | 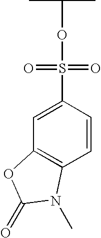 | 604, 606 |
| 261 | 4-[4-(3-Bromo-4-fluorophenyl)-1-methyl-5-oxo-2-thioxoimidazolidin-4-yl]phenyl 5-chloro-1,3-dimethyl-1H-pyrazole-4-sulfonate | 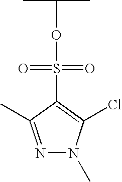 | 585, 587 |
| 262 | 4-[4-(3-Bromo-4-fluorophenyl)-1-methyl-5-oxo-2-thioxoimidazolidin-4-yl]phenyl 1-methyl-1H-imidazole-4-sulfonate | 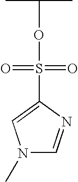 | 537, 539 |
| 263 | 4-[4-(3-Bromo-4-fluorophenyl)-1-methyl-5-oxo-2-thioxoimidazolidin-4-yl]phenyl 6-chloroimidazo[2,1-b][1,3]thiazole-5-sulfonate | 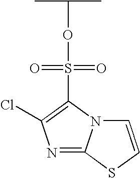 | 613, 615 |
| 264 | 4-[4-(3-Bromo-4-fluorophenyl)-1-methyl-5-oxo-2-thioxoimidazolidin-4-yl]phenyl 4-methoxybenzenesulfonate | 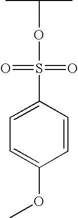 | 563, 565 |
| 265 | 4-[4-(3-Bromo-4-fluorophenyl)-1-methyl-5-oxo-2-thioxoimidazolidin-4-yl]phenyl3,5-dimethylisoxazole-4-sulfonate | 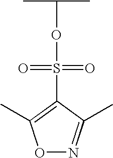 | 552, 554 |

### Example 266

### 4-[2-Amino-4-(3-bromo-4-fluorophenyl)-1-methyl-5-oxo-4,5-dihydro-1H-imidazol-4-yl]phenyl 4-(1,1-dimethylpropyl)benzenesulfonate

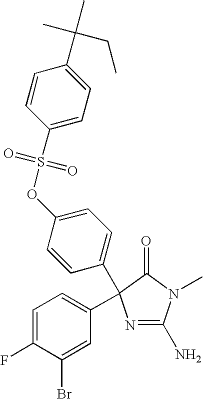

The title compound was synthesized as described for Example 223 starting from 4-[4-(3-bromo-4-fluorophenyl)-1-methyl-5-oxo-2-thioxoimidazolidin-4-yl]phenyl 4-(1,1-dimethylpropyl)benzenesulfonate, and was used without further purification: MS (ESI) m/z 588, 590 [M+H]⁺.

### Examples 267-275

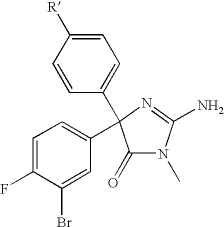

TABLE 8
Representative examples synthesized as described for 3-[2-amino-4-(3-bromo-4-
fluorophenyl)-1-methyl-5-oxo-4,5-dihydro-1H-imidazol-4-yl]phenyl 4-(1,1-
dimethylpropyl)benzenesulfonate

| | | | | MS (ES) | ¹H-NMR |
|---|---|---|---|---|---|
| | SM | | | m/z | (DMSO-d₆) |
| Ex | (Ex) | Chemical name | R′ | [M + H]⁺ | δ ppm |
| 267 | 257 | 4-[2-Amino-4-(3-bromo-4-fluorophenyl)-1-methyl-5-oxo-4,5-dihydro-1H-imidazol-4-yl]phenyl 2,5-dimethoxybenzenesulfonate | 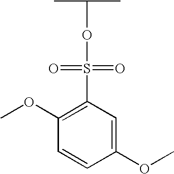 | 578, 580 | No data |
| 268 | 258 | 4-[2-Amino-4-(3-bromo-4-fluorophenyl)-1-methyl-5-oxo-4,5-dihydro-1H-imidazol-4-yl]phenyl 2,6-difluorobenzenesulfonate | 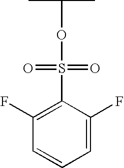 | 554, 556 | 7.87-8.00 (m, 1H), 7.65(dd, J = 6.8, 2.0 Hz, 1H),7.37-7.53 (m, 5H), 7.33(t, J = 8.7 Hz, 1H), 7.07-7.19 (m, 2H), 6.81 (br s,2H), 2.98 (s, 3H) |
| 269 | 259 | 4-[2-Amino-4-(3-bromo-4-fluorophenyl)-1-methyl-5-oxo-4,5-dihydro-1H-imidazol-4-yl]phenyl indane-5-sulfonate | 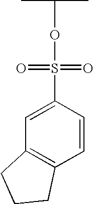 | 558, 560 | 7.65-7.72 (m, 2H), 7.60(d, J = 7.8 Hz, 1H), 7.44-7.52 (m, 2H), 7.42 (d,J = 8.8 Hz, 2H), 7.34 (t,J = 8.8 Hz, 1H), 7.02 (d,J = 8.8 Hz, 2H), 6.80 (br s,2H), 2.98 (s, 3H), 2.89-3.02 (m,4H), 2.07 (q, 2H) |
| 270 | 260 | 4-[2-Amino-4-(3-bromo-4-fluorophenyl)-1-methyl-5-oxo-4,5-dihydro-1H-imidazol-4-yl]phenyl 3-methyl-2-oxo-2,3-dihydro-1,3-benzoxazole-6-sulfonate | 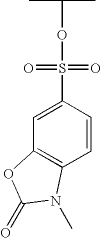 | 589, 591 | 7.89 (s, 1H), 7.73 (dd,J = 8.4, 1.6 Hz, 1H), 7.68(dd, J = 6.8, 2.0 Hz, 1H),7.37-7.51 (m, 4H), 7.33(t, J = 8.8 Hz, 1H), 6.97-7.06 (m, 2H), 6.80 (br s,2H), 2.98 (s, 3H). |
| 271 | 261 | 4-[2-Amino-4-(3-bromo-4-fluorophenyl)-1-methyl-5-oxo-4,5-dihydro-1H-imidazol-4-yl]phenyl 5-chloro-1,3-dimethyl-1H-pyrazole-4-sulfonate | 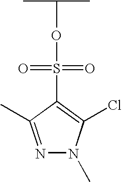 | 570, 572 | 7.62 (dd, J = 6.8, 2.3 Hz,1H), 7.41-7.48 (m, 3H),7.35 (t, J = 8.7 Hz, 1H),7.07 (d, J = 8.8 Hz, 2H),6.82 (br s, 2H), 3.80 (s,3H), 3.00 (s, 3H), 2.04 (s,3H) |
| 272 | 262 | 4-[2-Amino-4-(3-bromo-4-fluorophenyl)-1-methyl-5-oxo-4,5-dihydro-1H-imidazol-4-yl]phenyl 1-methyl-1H-imidazole-4-sulfonate | 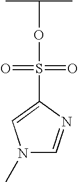 | 522, 524 | 8.01 (s, 1H), 7.95 (s, 1H),7.71 (dd, J = 6.8, 2.3 Hz,1H), 7.47-7.53 (m, 1H),7.42 (d, J = 8.8 Hz, 2H),7.34 (t, J = 8.8 Hz, 1H)7.05 (d, J = 8.8 Hz, 2H)6.80 (br s, 2H), 3.70 (s,3H), 2.99 (s, 3H) |
| 273 | 263 | 4-[2-Amino-4-(3-bromo-4-fluorophenyl)-1-methyl-5-oxo-4,5-dihydro-1H-imidazol-4-yl]phenyl 6-chloroimidazo[2,1-b][1,3]thiazole-5-sulfonate | 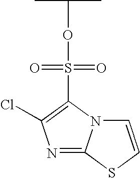 | 598, 600 | 7.80 (d, J = 3.8 Hz, 1H),7.64 (d, J = 3.8 Hz, 1H),7.59 (d, J = 6.0 Hz, 1H),7.42 (d, J = 8.3 Hz, 2H),7.34 (d, J = 7.0 Hz, 2H),7.06 (d, J = 8.3 Hz,2H),6.80 (br s, 2H), 2.99 (s,3H) |
| 274 | 264 | 4-[2-Amino-4-(3-bromo-4-fluorophenyl)-1-methyl-5-oxo-4,5-dihydro-1H-imidazol-4-yl]phenyl4-methoxybenzenesulfonate | 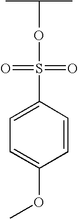 | [M − H]⁻ 548.3 | 7.72-7.77 (m, 2H), 7.67(dd, J = 6.6, 2.3 Hz, 1H),7.36-7.43 (m, 3H), 7.05(t, J = 8.46 Hz, 1H), 6.92-6.99 (m, 4H), 3.89 (s,3H), 3.12 (s, 3H) |
| 275 | 265 | 4-[2-Amino-4-(3-bromo-4-fluorophenyl)-1-methyl-5-oxo-4,5-dihydro-1H-imidazol-4-yl]phenyl 3,5-dimethylisoxazole-4-sulfonate | 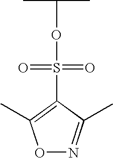 | [M − H]⁻ 537.3 | 7.63 (dd, J = 6.6, 2.3 Hz,1H), 7.45-7.51 (m, 2H),7.34-7.40 (m, 1H), 6.98-7.08 (m, 3H), 5.43 (br s,2H), 3.12 (s, 3H), 2.40 (s,3H), 2.29-2.32 (m, 3H) |

### Example 276

### 4-[2-Amino-4-(4-fluoro-3-pyrimidin-5-ylphenyl)-1-methyl-5-oxo-4,5-dihydro-1H-imidazol-4-yl]phenyl 4-(1,1-dimethylpropyl)benzenesulfonate

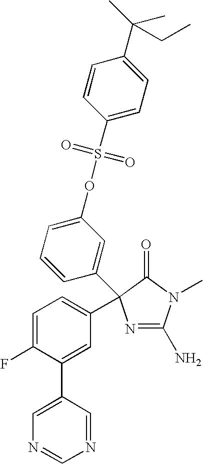

The title compound was synthesized as described for Example 234 in 4% yield (calculated from 5-(3-bromo-4-fluorophenyl)-5-(4-hydroxyphenyl)-3-methyl-2-thioxoimidazolidin-4-one), starting from 4-[2-amino-4-(3-bromo-4-fluorophenyl)-1-methyl-5-oxo-4,5-dihydro-1H-imidazol-4-yl]phenyl 4-(1,1-dimethylpropyl)benzenesulfonate: ¹H NMR (DMSO-d₆) □9.19 (s, 1H), 8.88 (s, 1H), 7.73 (d, J=8.5 Hz, 2H), 7.53-7.60 (m, 3H), 7.46-7.52 (m, 1H), 7.43 (d, J=8.5 Hz, 2H), 7.31 (t, J=9.4 Hz, 1H), 6.93 (d, J=8.8 Hz, 2H), 6.71 (br s, 1H), 2.94 (s, 2H), 2.50 (s, 3H), 1.58 (q, J=7.4 Hz, 2H), 1.22 (s, 6H), 0.50 (t, 3H); MS (ESI) m/z 588 [M+H]⁺.

### Examples 277-280

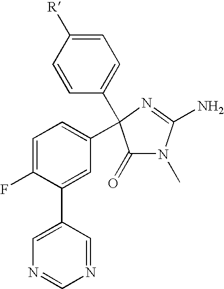

TABLE 9
Representative examples synthesized as described for 3-[2-amino-4-(4-fluoro-3-
pyrimidin-5-ylphenyl)-1-methyl-5-oxo-4,5-dihydro-1H-imidazol-4-yl]phenyl 4-(1,1-
dimethylpropyl)benzenesulfonate

| | | | | MS (ES) | ¹H-NMR |
|---|---|---|---|---|---|
| | SM | | | m/z | (DMSO-d₆) |
| Ex | (Ex) | Chemical name | R′ | [M + H]⁺ | δ ppm |
| 277 | 267 | 4-[2-Amino-4-(4-fluoro-3-pyrimidin-5-ylphenyl)-1-methyl-5-oxo-4,5-dihydro-1H-imidazol-4-yl]phenyl 2,5-dimethoxybenzenesulfonate | 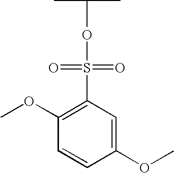 | 578 | 9.24 (s, 1H), 8.94 (d, J = 1.0Hz, 2H), 7.66 (dd, J = 7.4,2.1 Hz, 1H), 7.53-7.59 (m,1H), 7.49 (d, J = 8.8 Hz,2H), 7.28-7.41 (m, 3H),7.14 (d, J = 3.0 Hz, 1H), 7.05(d, J = 8.8 Hz, 2H), 6.78 (brs, 2H), 3.90 (s, 3H), 3.72(s, 3H), 2.98 (s, 3H), 1.92(s, 1H) |
| 278 | 268 | 4-[2-Amino-4-(4-fluoro-3-pyrimidin-5-ylphenyl)-1-methyl-5-oxo-4,5-dihydro-1H-imidazol-4-yl]phenyl 2,6-difluorobenzenesulfonate | 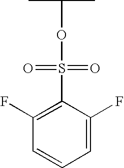 | 554 | 9.25 (s, 1H), 8.93 (s, 2H),7.87-8.00 (m, 1H), 7.62(dd, J = 7.5, 2.0 Hz, 1H),7.50-7.59 (m, 3H), 7.32-7.48 (m, 3H), 7.13 (d, J = 8.8Hz, 2H), 6.78 (br s, 2H),2.99 (s, 3H). |
| 279 | 274 | 4-[2-amino-4-(4-fluoro-3-pyrimidin-5-ylphenyl)-1-methyl-5-oxo-4,5-dihydro-1H-imidazol-4-yl]phenyl 4-methoxybenzenesulfonate | 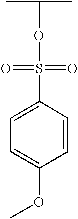 | [M − H]⁻ 546.4 | 9.14 (s, 1H), 8.81 (d, J = 1.3Hz, 2H), 7.66-7.71 (m,2H), 7.46-7.52 (m, 2H),7.35-7.40 (m, 2H), 7.10 (t,J = 9.6 Hz, 1H), 6.86-6.92(m, 4H), 3.81 (s, 3H),3.05(s, 3H), 2.02 (s, 1H) |
| 280 | 275 | 4-[2-amino-4-(4-fluoro-3-pyrimidin-5-ylphenyl)-1-methyl-5-oxo-4,5-dihydro-1H-imidazol-4-yl]phenyl 3,5-dimethylisoxazole-4-sulfonate | 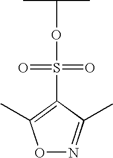 | [M − H]⁻ 535.4 | 9.22 (s, 1H), 8.90 (d,J = 1.3Hz, 2H), 7.52-7.63 (m,4H), 7.18 (dd, J = 10.0, 8.7 Hz,1H), 7.02-7.07 (m, 2H),3.15 (s, 3H), 2.41 (s, 3H),2.32 (s, 3H),2.10 (s, 1H),2.02 (s, 1H) |

### Example 281

### 4-[2-Amino-4-(4-fluoro-3-pyridin-3-ylphenyl)-1-methyl-5-oxo-4,5-dihydro-1H-imidazol-4-yl]phenyl 4-(1,1-dimethylpropyl)benzenesulfonate

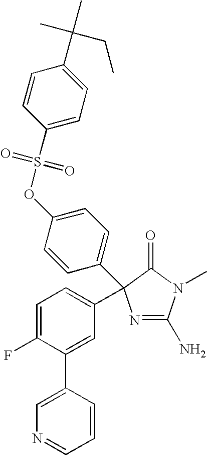

The title compound was synthesized as described for Example 234 in 4% yield (calculated from 5-(3-bromo-4-fluorophenyl)-5-(4-hydroxyphenyl)-3-methyl-2-thioxoimidazolidin-4-one), starting from 4-[2-amino-4-(3-bromo-4-fluorophenyl)-1-methyl-5-oxo-4,5-dihydro-1H-imidazol-4-yl]phenyl 4-(1,1-dimethylpropyl)benzenesulfonate and 3-pyridineboronic acid: ¹H NMR (DMSO-d₆) δ 8.58-8.66 (m, 2H), 7.87 (d, J=8.0 Hz, 1H), 7.76 (d, J=8.5 Hz, 2H), 7.42-7.62 (m, 7H), 7.30 (dd, J=10.3, 8.8 Hz, 1H), 6.97 (d, J=8.8 Hz, 2H), 6.74 (br s, 2H), 1.60 (q, J=7.4 Hz, 2H), 2.98 (s, 3H), 1.24 (s, 6H), 0.53 (t, J=7.4 Hz, 3H); MS (ESI) m/z 586.17 [M−H]⁻.

### Examples 282-287

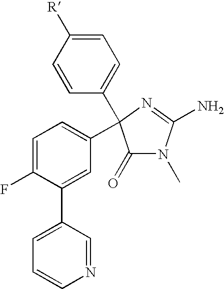

TABLE 10
Representative examples synthesized as described for 4-[2-amino-4-(4-fluoro-3-
pyridin-3-ylphenyl)-1-methyl-5-oxo-4,5-dihydro-1H-imidazol-4-yl]phenyl 4-(1,1-
dimethylpropyl)benzenesulfonate.

| | | | | MS (ES) | ¹H-NMR |
|---|---|---|---|---|---|
| | SM | | | m/z | (DMSO-d₆) |
| Ex | (Ex) | Chemical name | R′ | [M + H]⁺ | δ ppm |
| 282 | 267 | 4-[2-Amino-4-(4-fluoro-3-pyridin-3-ylphenyl)-1-methyl-5-oxo-4,5-dihydro-1H-imidazol-4-yl]phenyl 2,5-dimethoxybenzenesulfonate0.25 acetate | 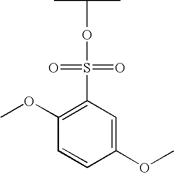 | 577.08 | 8.73 (br s, 1H), 8.59 (br s,1H), 7.82 (d, J = 7.8 Hz, 1H),7.55 (dd, J = 7.3, 2.3 Hz,1H), 7.42-7.50 (m, 3H),7.36 (dd, J = 7.1, 5.1 Hz,1H), 7.30 (d,J = 3.3 Hz, 1H),7.10-7.17 (m, 2H), 7.03-7.10 (m, 2H), 7.00 (d, J = 9.1Hz, 1H), 4.27 (br s, 2H),3.91 (s, 3H), 3.72-3.75 (m,3H), 3.10 (s, 3H), 2.01 (s,0.44H). |
| 283 | 269 | 3-[2-Amino-4-(4-fluoro-3-pyridin-3-ylphenyl)-1-methyl-5-oxo-4,5-dihydro-1H-imidazol-4-yl]phenyl indane-5-sulfonate 0.25 acetate | 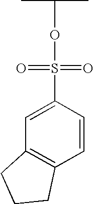 | [M + H]⁻ 556.10 | 8.64 (s, 1H), 8.61 (dd,J = 4.9, 1.6 Hz, 1H), 7.85-7.90 (m, 1H), 7.67 (s, 1H),7.57 (dd, J = 7.8, 1.8 Hz,2H), 7.42-7.54 (m, 5H),7.30 (dd, J = 10.3, 8.8 Hz,1H), 7.00 (d, J = 8.8 Hz, 2H),6.74 (br s, 2H), 2.97 (s,3H), 2.85-3.01 (m, 4H),2.04 (m, 2H), 1.90 (s,0.66H). |
| 284 | 270 | 4-[2-Amino-4-(4-fluoro-3-pyridin-3-ylphenyl)-1-methyl-5-oxo-4,5-dihydro-1H-imidazol-4-yl]phenyl 3-methyl-2-oxo-2,3-dihydro-1,3-benzoxazole-6-sulfonate 0.5acetate | 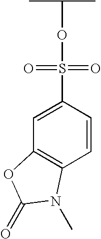 | 588.07 | 8.71 (s, 1H), 8.61 (dd,J = 4.9, 1.6 Hz, 1H), 7.85(dd, J = 7.8, 2.0 Hz, 1H),7.72 (dd, J = 8.2, 1.6 Hz,1H), 7.66 (d, J = 1.5 Hz, 1H),7.43-7.54 (m, 4H), 7.35-7.39 (m, 1H), 7.06-7.17(m, 2H), 6.94-6.99 (m,2H), 3.48 (s, 3H), 3.13 (s,3H), 2.81 (br s, 2H), 2.07 (s,1.5H). |
| 285 | 271 | 4-[2-Amino-4-(4-fluoro-3-pyridin-3-ylphenyl)-1-methyl-5-oxo-4,5-dihydro-1H-imidazol-4-yl]phenyl 5-chloro-1,3-dimethyl-1H-pyrazole-4-sulfonate 0.25 acetate | 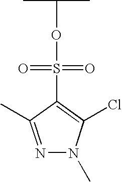 | 569.03 | 8.75 (s, 1H), 8.60 (dd,J = 4.9, 1.4 Hz, 1H), 7.84(dd, J = 8.0, 1.9 Hz, 1H),7.48-7.55 (m, 3H), 7.43-7.48 (m, 1H), 7.37 (dd,J = 8.3, 4.8 Hz, 1H), 7.14(dd, J = 10.0, 8.7 Hz, 1H),7.05-7.10 (m, 2H), 3.78 (s,3H), 3.15 (s, 3H), 2.56 (brs, 2H), 2.23 (s, 3H), 2.09(s, 1.1H) |
| 286 | 273 | 4-[2-Amino-4-(4-fluoro-3-pyridin-3-ylphenyl)-1-methyl-5-oxo-4,5-dihydro-1H-imidazol-4-yl]phenyl 6-chloroimidazo]2,1-b][1,3]thiazole-5-sulfonate 0.75acetate | 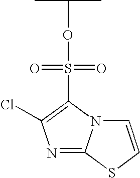 | 597.03 | 8.65 (s, 1H), 8.51-8.54 (m,1H), 7.76 (dd, J = 8.0, 1.9Hz, 1H), 7.57 (d, J = 4.6 Hz,1H), 7.32-7.42 (m, 4H),7.27-7.32 (m, 1H), 7.05(dd, J = 10.0, 8.7 Hz, 1H),6.92-6.97 (m, 2H), 6.89 (d,J = 4.3 Hz, 1H), 3.06 (s,3H), 2.01 (s, 1.96H). |
| 287 | 272 | 4-[2-Amino-4-(4-fluoro-3-pyridin-3-ylphenyl)-1-methyl-5-oxo-4,5-dihydro-1H-imidazol-4-yl]phenyl 1-methyl-1H-imidazole-4-sulfonate | 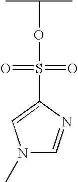 | 520.8 | 8.01 (s, 1H); 7.94 (s, 1H);7.39-7.58 (m, 5H); 7.28-7.36 (m, 1H); 7.13 (t, J = 8.9Hz, 3H); 7.01-7.06 (m,2H); 6.71 (br. s., 2H); 3.70(s, 3H); 2.98 (s, 3H). |

### Examples 288

### 4-[2-Amino-4-(4-fluorophenyl)-1-methyl-5-oxo-4,5-dihydro-1H-imidazol-4-yl]phenyl 3-methyl-2-oxo-2,3-dihydro-1,3-benzoxazole-6-sulfonate

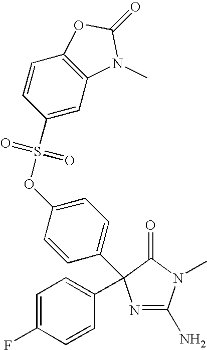

1 tie title compound was isolated as an additional product from the synthesis described in Example 284 and isolated after purification by preparative HPLC: ¹H NMR (CDCl₃) δ 7.75 (dd, J=8.3, 1.8 Hz, 1H), 7.64 (d, J=1.5 Hz, 1H), 7.36-7.45 (m, 4H), 6.92-7.10 (m, 5H), 3.49 (s, 3H), 3.15 (s, 3H), 2.96 (br s, 2H), 2.07 (s, 1H); MS (ESI) m/z 509.4 [M−H]⁻.

### Example 289

### 4-[4-(2′-Acetyl-6-fluoro-5′-methoxybiphenyl-3-yl)-2-amino-1-methyl-5-oxo-4,5-dihydro-1H-imidazol-4-yl]phenyl methanesulfonate

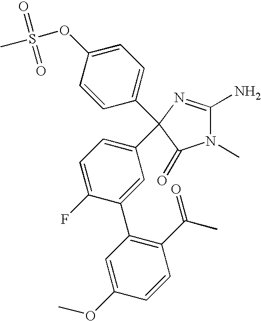

A mixture of tris(dibenzylideneacetone)dipalladium (50 mg, 0.054 mmol), tricyclohexylphosphine (70 mg, 0.252 mmol), octamethyl-2,2′-bi-1,3,2-dioxaborolane (503 mg, 1.98 mmol), potassium acetate (265 mg, 2.70 mmol) and 2-chloro-4-methoxyacetophenone (330 mg, 1.79 mmol) in anhydrous dioxane (5 mL) was irradiated in a microwave at 130° C. for 5 h under an atmosphere of argon. The cooled mixture was filtered through a plug of silica and concentrated. A mixture of one third of the above material, 4-[2-amino-4-(3-bromo-4-fluorophenyl)-1-methyl-5-oxo-4,5-dihydro-1H-imidazol-4-yl]phenyl methanesulfonate (83 mg, 0.182 mmol), [1,1′-bis(diphenylphosphino)ferrocene]palladium(II) chloride dichloromethane adduct (20 mg, 0.025 mmol) and potassium carbonate (126 mg, 0.909 mmol) in anhydrous tetrahydrofuran (5 mL) was irradiated in a microwave at 130° C. for 2 h under an atmosphere of argon. The cooled mixture was filtered, concentrated and purified by preparative HPLC to give 17 mg (17% yield) of the title compound: ¹H NMR (CDCl₃) □7.69 (d, J=8.6 Hz, 1H), 7.63 (m, 2H), 7.40 (m, 1H), 7.24 (m, 3H), 7.03 (m, 1H), 6.93 (dd, J=8.7, 2.6 Hz, 1H), 6.76 (m, 1H), 3.84 (s, 3H), 3.13 (s, 3H), 3.06 (s, 3H); MS (ES) m/z 526.0 [M+H]⁺, 524.1 [M−H]⁻.

### Example 290

### 4-Chloro-2-[(4-methoxyphenyl)ethynyl]pyridine

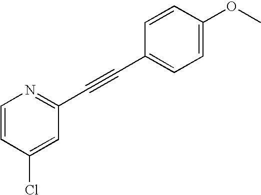

Tri-tert-butylphosphine (0.65 mL, 0.32 mmol, 10% solution in hexane) was added to 2-bromo-4-chloro-pyridine (1.05 g, 5.5 mmol; described in: Choppin, S.; Gros, P.; Fort, Y. Eur. J. Org. Chem. 2001, 3, 603-606), 4-ethyneanisole (0.79 g, 6 mmol), bis(benzonitrile)palladium(II) chloride (61 mg, 0.16 mmol) and copper(I) iodide (21 mg, 0.21 mmol) in triethylamine (1.9 mL) and anhydrous dioxane (20 mL) at 0° C. The mixture was stirred over night at room temperature. Water was added and the mixture was extracted with dichloromethane. The organic phases were pooled, dried over sodium sulfate and concentrated. Column chromatography using 0-25% ethyl acetate in n-heptane as eluent afforded the title compound 0.58 g (43% yield): ¹H NMR (400 MHz, DMSO-d₆) □ppm 8.54-8.56 (m, 1H), 7.78-7.79 (m, 1H), 7.53-7.59 (m, 3H), 7.01-7.05 (m, 2H), 3.81 (s, 3H).

### Example 291

### 1-(4-Chloropyridin-2-yl)-2-(4-methoxyphenyl)ethane-1,2-dione

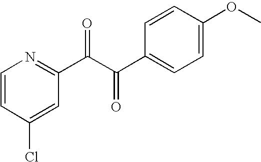

4-Chloro-2-[(4-methoxyphenyl)ethynyl]pyridine (0.68 g, 2.8 mmol) and palladium dichloride (50 mg, 0.28 mmol) was heated to 130° C. for 4.5 h in dimethyl sulfoxide (20 mL). On cooling to room temperature, water was added and the mixture was extracted with ethyl acetate. The organic phase was washed with water and brine, dried over sodium sulfate and concentrated. Column chromatography using 0-25% ethyl acetate in n-heptane as eluent afforded the title compound 0.25 g (32% yield): ¹H NMR (400 MHz, DMSO-d₆) □ppm 8.65-8.68 (m, 1H), 8.25-8.27 (m, 1H), 7.90-7.93 (m, 1H), 7.83-7.87 (m, 2H), 7.09-7.14 (m, 2H), 3.87 (s, 3H).

### Example 292

### 1-(4-Chloropyridin-2-yl)-2-(4-hydroxyphenyl)ethane-1,2-dione

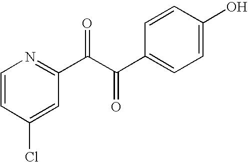

1-(4-Chloropyridin-2-yl)-2-(4-methoxyphenyl)ethane-1,2-dione (0.25 g, 0.9 mmol) was dissolved in dichloromethane (15 mL) and cooled to 0° C. Boron tribromide (0.25 mL, 2.7 mmol) was added and the mixture was allowed to reach room temperature and stirred for 5 h. Water and saturated sodium hydrogen carbonate (aq.) was added and the mixture was extracted with ethyl acetate. The organic extract was washed with brine, dried over sodium sulfate and concentrated to give the title compound which was used in next step without purification: ¹H NMR (400 MHz, DMSO-d₆) □ppm 8.65-8.68 (m, 1H), 8.23-8.25 (m, 1H), 7.89-7.93 (m, 1H), 7.72-7.76 (m, 2H), 6.89-6.94 (m, 2H).

### Example 293

### 1-(4-Hydroxyphenyl)-2-[4-(3-methoxyphenyl)pyridin-2-yl]ethane-1,2-dione

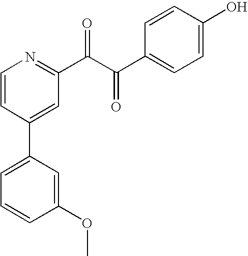

1-(4-Chloropyridin-2-yl)-2-(4-hydroxyphenyl)ethane-1,2-dione (0.9 mmol), 3-methoxybenzene boronic acid (0.18 g, 1.2 mmol) and tetrakis(triphenylphosphine)palladium (104 mg, 0.09 mmol) were dissolved in dimethoxy ethane (10 mL) and sodium carbonate (1.4 mL, 2.7 mmol, 2 M aq.). The mixture was heated at 130° C. for 1 h in a microwave. When cooled, water was added and the mixture was extracted with ethyl acetate. The organic extract was washed with brine, dried over sodium sulfate and concentrated. Column chromatography using 0-40% ethyl acetate in n-neptane as eluent afforded the title compound 0.173 g (58% yield): ¹H NMR (400 MHz, DMSO-d₆) □ppm 8.72-8.75 (m, 1H), 8.42-8.45 (m, 1H), 8.06-8.09 (m, 1H), 7.73-7.78 (m, 2H), 7.44-7.52 (m, 3H), 7.09-7.14 (m, 1H), 6.91-6.95 (m, 2H), 3.88 (s, 3H).

### Example 294

### 5-(4-hydroxyphenyl)-5-[4-(3-methoxyphenyl)pyridin-2-yl]-3-methyl-2-thioxoimidazolidin-4-one

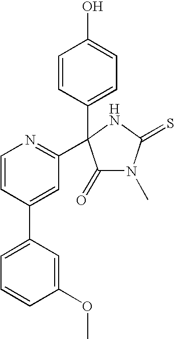

1-(4-Hydroxyphenyl)-2-[4-(3-methoxyphenyl)pyridin-2-yl]ethane-1,2-dione (0.173 g, 0.52 mmol) and N-methylthiourea (0.93 g, 1.04 mmol) was heated to 100° C. in dimethyl sulfoxide (5 mL). Potassium hydroxide (1.2 M aq., 0.9 mL, 1.1 mmol) was added and the mixture was kept at 100° C. for 30 min. Water was added, pH adjusted to 4 using hydrochloric acid (2M aq.), and the mixture was extracted with ethyl acetate. The organic phase was washed with water and brine, dried over sodium sulfate and concentrated to afford the title compound which was used in next step without any purification: ¹H NMR (400 MHz, DMSO-d₆) □ppm 9.67 (s, 1H), 8.59-8.62 (m, 1H), 7.67-7.70 (m, 1H), 7.36-7.42 (m, 1H), 7.23-7.30 (m, 3H), 7.09-7.13 (m, 2H), 6.99-7.03 (m, 1H), 6.77-6.82 (m, 2H), 3.77 (s, 3H), 3.13 (s, 3H).

### Example 295

### 4-{4-[4-(3-Methoxyphenyl)pyridin-2-yl]-1-methyl-5-oxo-2-thioxoimidazolidin-4-yl}phenyl methanesulfonate

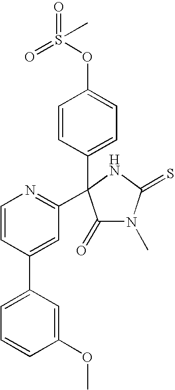

Methanesulfonylchloride (0.021 mL, 0.27 mmol) was added to 5-(4-hydroxyphenyl)-5-[4-(3-methoxyphenyl)pyridin-2-yl]-3-methyl-2-thioxoimidazolidin-4-one (0.1 g, 0.26 mmol) and triehylamine (0.04 mL, 0.28 mmol) in dichloromethane (5 mL). The mixture was stirred over night., then saturated sodium bicarbonate (aq.) was added and the mixture was extracted with ethyl acetate. The organic extract was dried over sodium sulfate and concentrated. Column chromatography using 0-40% ethyl acetate in n-heptane as eluent afforded the title compound 0.085 g (68% yield): ¹H NMR (400 MHz, DMSO-d₆) □ppm 11.58 (s, 1H), 8.65-8.69 (m, 1H), 7.74-7.77 (m, 1H), 7.63-7.67 (m, 2H), 7.41-7.48 (m, 3H), 7.33-7.36 (m, 1H), 7.16-7.21 (m, 2H), 7.04-7.08 (m, 1H), 3.81 (s, 3H), 3.41 (s, 3H), 3.19 (s, 3H).

### Example 296

### 4-{2-Amino-4-[4-(3-methoxyphenyl)pyridin-2-yl]-1-methyl-5-oxo-4,5-dihydro-1H-imidazol-4-yl}phenyl methanesulfonate 0.25 acetate

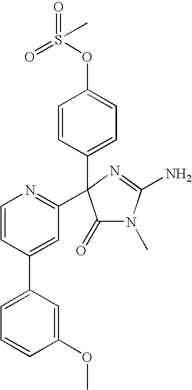

4-{4-[4-(3-Methoxyphenyl)pyridin-2-yl]-1-methyl-5-oxo-2-thioxoimidazolidin-4-yl}phenyl methanesulfonate (0.085 g, 0.2 mmol) was dissolved in methanol (4 mL) and conc. ammonium hydroxide (aq. 2 mL). Tert-butyl hydroperoxide (0.4 mL, 3 mmol, 70% aq.) was added and the mixture was heated to 35° C. for 5 h. On cooling to room temperature the mixture was filtered and purification by preparative HPLC gave 23 mg (24% yield) of the title compound: ¹H NMR (400 MHz, DMSO-d₆) □ppm 8.53-8.57 (m, 1H), 7.75-7.81 (m, 2H), 7.56-7.60 (m, 1H), 7.40 (t, J=7.91 Hz, 1H), 7.31-7.35 (m, 2H), 7.29 (br. s., 1H), 7.11-7.15 (m, 1H), 7.09-7.11 (m, 1H), 7.01-7.05 (m, 1H), 3.80 (s, 3H), 3.37 (s, 3H), 2.99 (s, 3H), 1.89 (s, 0.5H, acetate).

### Example 297

### 2-Amino-5-(4-hydroxyphenyl)-5-[4-(3-methoxyphenyl)pyridin-2-yl]-3-methyl-3,5-dihydro-4H-imidazol-4-one

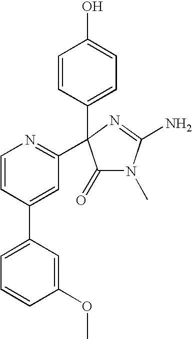

The compound was prepared as described in Example 296 starting from 5-(4-hydroxyphenyl)-5-[4-(3-methoxyphenyl)pyridin-2-yl]-3-methyl-2-thioxoimidazolidin-4-one. Column chromatography using a gradient of 0-9% 7 N ammonia (in methanol) in dichloromethane as eluent afforded the title compound 0.047 g (46% yield): ¹H NMR (400 MHz, DMSO-d₆) □ppm 8.49-8.56 (m, 1H), 7.50-7.56 (m, 1H), 7.37-7.48 (m, 3H), 7.12-7.20 (m, 1H), 7.00-7.10 (m, 3H), 6.70-6.76 (m, 2H), 3.79 (s, 3H), 2.97 (s, 3H).

### Example 298

### 4-{2-Amino-4-[4-(3-methoxyphenyl)pyridin-2-yl]-1-methyl-5-oxo-4,5-dihydro-1H-imidazol-4-yl}phenyl trifluoromethanesulfonate 0.5 acetate

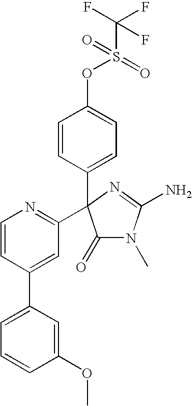

Using the method described for Example 143 starting with 2-amino-5-(4-hydroxyphenyl)-5-[4-(3-methoxyphenyl)pyridin-2-yl]-3-methyl-3,5-dihydro-4H-imidazol-4-one (0.047 g, 0.12 mmol) afforded the title compound 0.021 g (32% yield): ¹H NMR (400 MHz, DMSO-d₆) □ppm 8.54 (d, J=5.27 Hz, 1H), 7.84-7.89 (m, 2H), 7.58 (dd, J=5.14, 1.63 Hz, 1H), 7.46-7.51 (m, 2H), 7.40 (t, J=8.03 Hz, 1H), 7.26-7.31 (m, 1H), 7.09-7.15 (m, 2H), 7.01-7.05 (m, 1H), 3.80 (s, 3H), 3.00 (s, 3H), 1.91 (s, 1.5H, acetate).

### Example 299

### 4-[2-Amino-4-(3-cyclohex-1-en-1-yl-4-fluorophenyl)-1-methyl-5-oxo-4,5-dihydro-1H-imidazol-4-yl]phenyl methanesulfonate 0.5 acetate

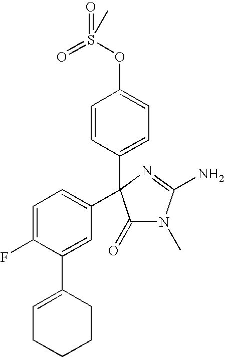

4-[2-Amino-4-(3-bromo-4-fluorophenyl)-1-methyl-5-oxo-4,5-dihydro-1H-imidazol-4-yl]phenyl methanesulfonate (320 mg, 0.7 mmol), 1-cyclohexenylboronic acid (95 mg, 0.75 mmol), [1,1′-bis(diphenylphosphino)ferrocene]palladium(II) chloride dichloromethane adduct (57 mg, 0.07 mmol) and potassium carbonate (0.58 g, 4.2 mmol) in anhydrous tetrahydrofuran (5 mL) were mixed and irradiated under an argon atmosphere in a microwave reactor at 140° C. for 4 h. Water and ethyl acetate was added, the organic phase was separated, dried over magnesium sulfate and concentrated. Column chromatography using 0-4% 7 N ammonia (in methanol) and dichloromethane as eluent followed by preparative HPLC gave 45 mg (13% yield) of the title compound: ¹H NMR (400 MHz, DMSO-d₆) □ppm 7.49-7.54 (m, 2H), 7.39-7.44 (m, 1H), 7.34-7.39 (m, 1H), 7.26-7.30 (m, 2H), 7.05-7.12 (m, 1H), 5.81-5.84 (m, 1H), 3.35 (s, 3H), 2.98 (s, 3H), 2.22-2.28 (m, 2H), 2.12-2.18 (m, 2H), 1.89 (s, 1.6H, acetate), 1.57-1.71 (m, 4H).

### Example 300

### 4-[2-Amino-4-(3-cyclohexyl-4-fluorophenyl)-1-methyl-5-oxo-4,5-dihydro-1H-imidazol-4-yl]phenyl methanesulfonate 0.5 acetate

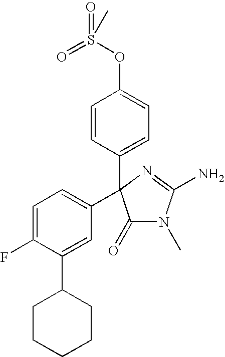

4-[2-Amino-4-(3-cyclohex-1-en-1-yl-4-fluorophenyl)-1-methyl-5-oxo-4,5-dihydro-1H-imidazol-4-yl]phenyl methanesulfonate (28 mg, 0.06 mmol) and palladium on carbon (10%, 5 mg) was shaken in ethyl acetate (8 mL) under a hydrogen atmosphere at atmospheric pressure over night. The mixture was filtered through a plug of celite and the product was purified using preparative HPLC to afford 19 mg (63% yield) of the title compound: ¹H NMR (400 MHz, DMSO-d₆) □ppm 7.48-7.53 (m, 2H), 7.43-7.47 (m, 1H), 7.30-7.35 (m, 1H), 7.25-7.29 (m, 2H), 7.03-7.08 (m, 1H), 3.35 (s, 3H), 2.98 (s, 3H), 2.70-2.78 (m, 1H), 1.90 (s, 1.8H, acetate), 1.66-1.81 (m, 5H), 1.29-1.41 (m, 4H), 1.15-1.29 (m, 1H).

### Example 301

### 4-{2-Amino-4-[4-fluoro-3-(4-methoxypyridin-2-yl)phenyl]-1-methyl-5-oxo-4,5-dihydro-1H-imidazol-4-yl}phenyl methanesulfonate 0.75 acetate

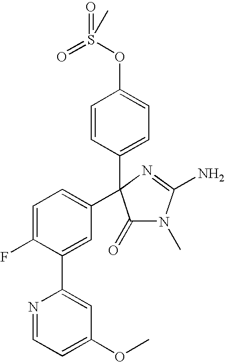

2-Chloro-4-methoxypyridine (92 uL, 0.8 mmol), hexamethyl distannane (165 uL, 0.8 mmol) and tetrakis(triphenylphosphine)palladium (28 mg, 0.024 mmol) were mixed and irradiated under an argon atmosphere in a microwave at 130° C. for 2 h. After cooling to room temperature 4-[2-amino-4-(3-bromo-4-fluorophenyl)-1-methyl-5-oxo-4,5-dihydro-1H-imidazol-4-yl]phenyl methanesulfonate (130 mg, 0.28 mmol) and tetrakis(triphenylphosphine)palladium (28 mg, 0.024 mmol) was added. The mixture was irradiated under an argon atmosphere in a microwave at 130° C. for 6 h. On cooling the mixture was filtered and the compound was purified using preparative HPLC to afford 9 mg (6% yield) of the title compound: ¹H NMR (400 MHz, DMSO-d₆) □ppm 8.52 (d, J=5.77 Hz, 1H), 8.10-8.14 (m, 1H), 7.53-7.60 (m, 3H), 7.25-7.31 (m, 4H), 6.99-7.02 (m, 1H), 3.86 (s, 3H), 3.35 (s, 3H), 2.99 (s, 3H), 1.88 (s, 2.1H, acetate).

### Example 302

### 4-((3-Bromophenyl)ethynyl)-2,6-dimethylpyridine

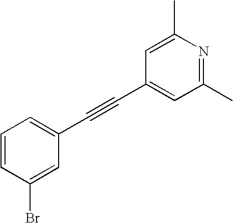

1-Bromo-3-iodobenzene (2.265 g, 8.00 mmol), palladium(II) dichlorobis(triphenylphosphine) (28 mg, 0.04 mmol) and copper(I) iodide (7.62 mg, 0.04 mmol) were dissolved in anhydrous tetrahydrofuran (15 mL) and triethylamine (10 mL). 4-Ethynyl-2,6-dimethylpyridine (1.05 g, 8.00 mmol) dissolved in anhydrous tetrahydrofuran (5 mL) was added. The reaction was stirred at room temperature over night under an atmosphere of nitrogen. The reaction was quenched with hydrochloric acid (2M aq.) and extracted with dichloromethane twice. The aqueous phase was then made basic using NaOH (15% aq.) and extracted with dichloromethane twice. The combined organic phases were concentrated in vacuo and the product was purified by column chromatography using a gradient of 0 to 30% ethyl acetate in heptane to give 1.5 μg (66% yield) of the title compound. MS (ESI) m/z 288 [M+1]⁺

### Example 303

### 1-(3-Bromophenyl)-2-(2,6-dimethylpyridin-4-yl)ethane-1,2-dione

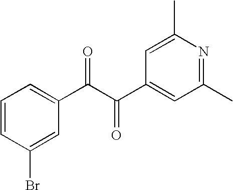

4-((3-Bromophenyl)ethynyl)-2,6-dimethylpyridine (1.15 g, 5.28 mmol) and palladium(II) chloride (94 mg, 0.53 mmol) was dissolved in anhydrous dimethyl sulfoxide (15 mL) and the reaction mixture was refluxed at 150° C. for 4 h. It was then allowed to reach room temperature and was diluted with water. The solution was extracted with dichloromethane three times and the combined organic phases were concentrated. The product was purified by column chromatography using a gradient eluent (0 to 100% ethyl acetate in heptane) to give 693 mg (41% yield) of the title compound. MS (ESI) m/z 320 [M+1]⁺

### Example 304

### 2-Amino-4-(3-bromophenyl)-4-(2,6-dimethylpyridin-4-yl)-1-methyl-1H-imidazol-5(4H)-one

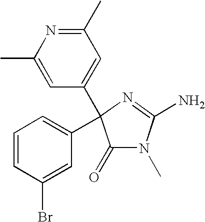

1-(3-Bromophenyl)-2-(2,6-dimethylpyridin-4-yl)ethane-1,2-dione (693 mg, 2.18 mmol), 1-methylguanidine, and hydrochloric acid (955 mg, 8.71 mmol) were dissolved in ethanol (15 mL) and acetonitrile (15 mL). Sodium carbonate (923 mg, 8.71 mmol) was dissolved in water (10 mL) and added. The reaction was heated to 85° C. for 45 min. The mixture was cooled to room temperature, diluted with water and extracted with dichloromethane three times. The combined organic phases were dried and concentrated and the product purified by column chromatography using a gradient eluent (0 to 10% {1:9 ammonium hydroxide/methanol} in dichloromethane) to give 293 mg (36% yield) of the title compound: MS (ESI) m/z 375 [M+1]⁺

### Example 305

### 3′-(2-Amino-4-(2,6-dimethylpyridin-4-yl)-1-methyl-5-oxo-4,5-dihydro-1H-imidazo-4-yl)-5-methoxybiphenyl-3-yl methanesulfonate hydrochloride

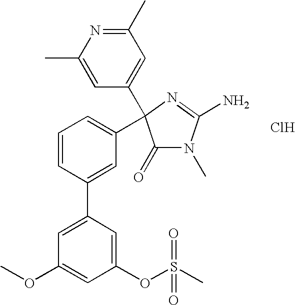

2-Amino-4-(3-bromophenyl)-4-(2,6-dimethylpyridin-4-yl)-1-methyl-1H-imidazol-5(4H)-one (100 mg, 0.27 mmol), 3-methoxy-5-(4,4,5,5-tetramethyl-1,3,2-dioxaborolan-2-yl)phenyl methanesulfonate (88 mg, 0.27 mmol), potassium carbonate (222 mg, 1.61 mmol), and [1,1′-bis(diphenylphosphino)ferrocene]palladium(II) chloride dichloromethane adduct (22 mg, 0.03 mmol) were mixed in tetrahydrofuran (4 mL) and water (1 mL) in a 10 mL microwave vial. The reaction was irradiated at 150° C. for 15 min in a microwave. When cooled to room temperature the mixture was filtered through celite and concentrated in vacuo. The product was purified by preparative HPLC and after evaporation of the solvents it was dissolved in hydrochloric acid in diethyl ether (1 M, 131 μL) and dichloromethane (1 mL). The mixture was then concentrated in vacuo to give 70 mg (49% yield) of the title compound: ¹H NMR (CD₃OD) □ppm 7.62-7.56 (m, 2H) 7.49-7.39 (m, 2H) 7.13 (s, 2H) 7.12-7.08 (m, 2H) 6.90 (t, J=2.15 Hz, 1H) 3.87 (s, 3H) 3.24 (s, 3H) 3.14 (s, 3H) 2.46 (s, 6H); MS (ESI) m/z 495 [M+1]⁺

### Example 306

### 3′-(2-Amino-4-(2,6-dimethylpyridin-4-yl)-1-methyl-5-oxo-4,5-dihydro-1H-imidazo-4-yl)-5-chlorobiphenyl-3-yl methanesulfonate hydrochloride

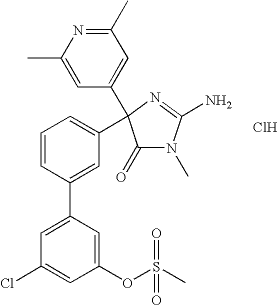

2-Amino-4-(3-bromophenyl)-4-(2,6-dimethylpyridin-4-yl)-1-methyl-1H-imidazol-5(4H)-one (100 mg, 0.27 mmol), 3-chloro-5-(4,4,5,5-tetramethyl-1,3,2-dioxaborolan-2-yl)phenyl methanesulfonate (116 mg, 0.35 mmol), potassium carbonate (222 mg, 1.61 mmol), and [1,1′-bis(diphenylphosphino)ferrocene]palladium(II) chloride dichloromethane adduct (22 mg, 0.03 mmol) were diluted in tetrahydrofuran (4 mL) and water (1 mL) in a 10 mL microwave vial. The reaction was irradiated at 150° C. for 15 min in a microwave. When cooled to room temperature the mixture was filtered through celite and concentrated in vacuo. The product was purified by preparative HPLC and after evaporation of the solvents it was dissolved in hydrochloric acid in diethyl ether (1M, 92 μL) and dichloromethane (1 mL). Concentration in vacuo gave 42 mg (29% yield) of the title compound: ¹H NMR (CD₃OD) □ppm 7.66-7.57 (m, 3H) 7.51-7.47 (m, 3H) 7.41 (t, J=2.02 Hz, 1H) 7.15 (s, 2H) 3.31 (s, 3H) 3.16 (s, 3H) 2.48 (s, 6H); MS (ESI) m/z 499 [M+1]⁺

### Example 307

### 5′-(2-Amino-1-methyl-4-(2-methylpyridin-4-yl)-5-oxo-4,5-dihydro-1H-imidazol-4-yl)-5-chloro-2′-fluorobiphenyl-3-yl methanesulfonate hydrochloride

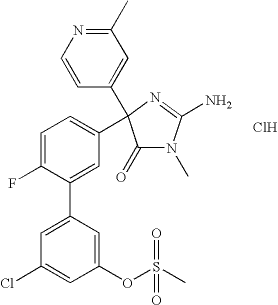

2-Amino-4-(3-bromo-4-fluorophenyl)-1-methyl-4-(2-methylpyridin-4-yl)-1H-imidazol-5(4H)-one (80 mg, 0.21 mmol), 3-chloro-5-(4,4,5,5-tetramethyl-1,3,2-dioxaborolan-2-yl)phenyl methanesulfonate (92 mg, 0.28 mmol), potassium carbonate (176 mg, 1.27 mmol) and [1,1′-bis(diphenylphosphino)ferrocene]palladium(II) chloride dichloromethane adduct (17 mg, 0.02 mmol)) were diluted in tetrahydrofuran (4 mL) and water (1 mL) in a 10 mL microwave vial. The reaction was irradiated at 150° C. for 15 min in a microwave. When cooled to room temperature the mixture was filtered through celite and concentrated in vacuo. The product was purified by preparative HPLC and after evaporation of the solvents it was diluted with hydrochloric acid in diethylether (1M, 61 μL) and dichloromethane (1 mL). Concentration in vacuo gave 29 mg (25% yield) of the title compound: ¹H NMR (CD₃OD) □ppm 8.36 (d, J=5.31 Hz, 1H) 7.56-7.47 (m, 3H) 7.45-7.41 (m, 2H) 7.33 (s, 1H) 7.30-7.20 (m, 2H) 3.29 (s, 3H) 3.13 (s, 3H) 2.50 (s, 3H); MS (ESI) m/z 503 [M+1]⁺

### Example 308

### 4-((3-Bromophenyl)ethynyl)-2-chloropyridine

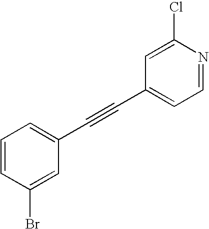

1-Bromo-3-ethynylbenzene (3.76 g, 20.77 mmol), palladium(II) dichlorobis(triphenylphosphine) (73 mg, 0.10 mmol) and copper(I) iodide (0.020 g, 0.10 mmol) were dissolved in anhydrous tetrahydrofuran (15 mL) and triethylamine (10 mL). 2-Chloro-4-iodopyridine (4.97 g, 20.77 mmol) dissolved in anhydrous tetrahydrofuran (5 mL) was added. The reaction was stirred at room temperature over night under an atmosphere of nitrogen. The reaction was quenched with hydrochloric acid (2M aq.) and extracted with dichloromethane twice. The aqueous phase was then made basic using NaOH (15% aq.) and extracted with dichloromethane twice. The combined organic phases were concentrated in vacuo and the product was purified by column chromatography using a gradient eluent (0 to 30% ethyl acetate in heptane) to give 5.38 g (89% yield) of the title compound: MS (ESI) m/z 294 [M+1]⁺

### Example 309

### 1-(3-Bromophenyl)-2-(2-chloropyridin-4-yl)ethane-1,2-dione

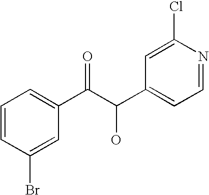

4-((3-Bromophenyl)ethynyl)-2-chloropyridine (5.38 g, 18.39 mmol) and palladium(II) chloride (326 mg, 1.84 mmol) was dissolved in anhydrous dimethyl sulfoxide (25 mL) and the mixture was stirred at 150° C. for 4 h. It was then allowed to reach room temperature and was then diluted with water. The solution was extracted with dichloromethane three times and the combined organic phases were concentrated. The product was purified by column chromatography using a gradient eluent (0 to 100% ethyl acetate in heptane) to give 3.69 g of the title compound: MS (ESI) m/z 326 [M+1]⁺

### Example 310

### 2-Amino-4-(3-bromophenyl)-4-(2-chloropyridin-4-yl)-1-methyl-1H-imidazol-5(4H)-one

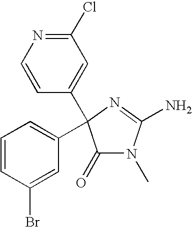

1-(3-Bromophenyl)-2-(2-chloropyridin-4-yl)ethane-1,2-dione (3.69 g, 11.37 mmol) and 1-methylguanidine hydrochloride (4.98 g, 45.48 mmol) were dissolved in ethanol (15 mL) and acetonitrile (15 mL). Sodium carbonate (4.82 g, 45.48 mmol) was dissolved in water (10 mL) and added and the reaction was then heated to 85° C. for 45 min. The mixture was cooled to room temperature, diluted with water and extracted with dichloromethane three times. The combined organic phases were dried and concentrated and the product purified by column chromatography using a gradient eluent (0 to 10% {1:9 ammonium hydroxide/methanol} in dichloromethane) to give 1.52 g (35% yield) of the title compound: MS (ESI) m/z 381 [M+1]⁺

### Example 311

### 3′-(2-Amino-4-(2-chloropyridin-4-yl)-1-methyl-5-oxo-4,5-dihydro-1H-imidazol-4-yl)-5-chlorobiphenyl-3-yl methanesulfonate hydrochloride

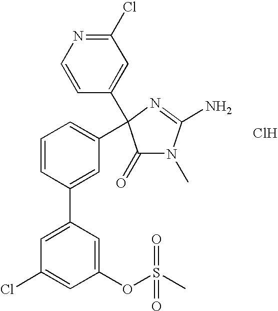

2-Amino-4-(3-bromophenyl)-4-(2-chloropyridin-4-yl)-1-methyl-1H-imidazol-5(4H)-one (100 mg, 0.26 mmol), 3-chloro-5-(4,4,5,5-tetramethyl-1,3,2-dioxaborolan-2-yl)phenyl methanesulfonate (114 mg, 0.34 mmol), potassium carbonate (218 mg, 1.58 mmol), and [1,1′-bis(diphenylphosphino)ferrocene]palladium(II) chloride dichloromethane adduct (22 mg, 0.03 mmol) were mixed in tetrahydrofuran (4 mL) and water (1 mL) in a 10 mL microwave vial. The reaction was irradiated at 150° C. for 15 min in a microwave. When cooled to room temperature the mixture was filtered through celite and concentrated in vacuo. The product was purified by preparative HPLC and after evaporation of the solvents it was diluted in hydrochloric acid in diethyl ether (1M, 68 μL) and dichloromethane (1 mL) and subsequently concentrated in vacuo to give 35 mg (25% yield) of the title compound: ¹H NMR (CD₃OD) □ppm 8.28 (d, J=5.31 Hz, 1H) 7.61 (s, 1H) 7.59-7.53 (m, 2H) 7.49-7.39 (m, 5H) 7.35 (t, J=1.89 Hz, 1H) 3.25 (s, 3H) 3.10 (s, 3H); MS (ESI) m/z 505 [M+1]⁺

### Example 312

### 2-Amino-5-(2-chloropyridin-4-yl)-5-[3-(3-methoxy-5-methylsulfonyloxy-phenyl)phenyl]-3-methyl-imidazol-4-one hydrochloride

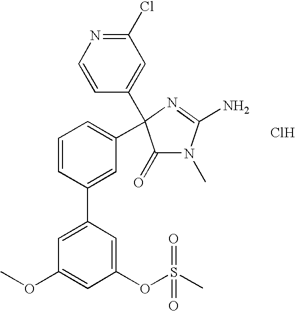

2-Amino-4-(3-bromophenyl)-4-(2-chloropyridin-4-yl)-1-methyl-1H-imidazol-5(4H)-one (100 mg, 0.26 mmol), 3-methoxy-5-(4,4,5,5-tetramethyl-1,3,2-dioxaborolan-2-yl)phenyl methanesulfonate (112 mg, 0.34 mmol), potassium carbonate (218 mg, 1.58 mmol), and [1,1′-bis(diphenylphosphino)ferrocene]palladium(II) chloride dichloromethane adduct (22 mg, 0.03 mmol) were mixed in tetrahydrofuran (4 mL) and water (1 mL) in a 10 mL microwave vial. The reaction was irradiated at 150° C. for 15 min in a microwave. When cooled to room temperature the mixture was filtered through celite and concentrated in vacuo. The product was purified by preparative HPLC and after evaporation of the solvents it was diluted in hydrochloric acid in diethyl ether (1M, 100 μL) and dichloromethane 1 mL) and concentrated in vacuo to give 42 mg (30% yield) of the title compound: ¹H NMR (CD₃OD) □ppm 8.33 (d, J=5.31 Hz, 1H) 7.65-7.57 (m, 2H) 7.52 (d, J=1.52 Hz, 1H) 7.49-7.43 (m, 3H) 7.16-7.04 (m, 2H) 6.90 (t, J=2.27 Hz, 1H) 3.87 (s, 3H) 3.25 (s, 3H) 3.15 (s, 3H); MS (ESI) m/z 501 [M+1]⁺

### Assays

Compounds were tested in at least one of the following assays:

β-Secretase Enzyme

The enzyme used in the IGEN Cleavage-, Fluorescent-, TR-FRET- and BiaCore assays is described as follows:

The soluble part of the human β-Secretase (AA1-AA460) was cloned into the ASP2-Fc10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

### IGEN Cleavage Assay

The enzyme was diluted to 43 μg/ml in 40 mM MES pH 5.0. The IGEN substrate was diluted to 12 μM in 40 mM MES pH 5.0. Compounds were diluted to the desired concentration in dimethyl sulfoxide (final dimethyl sulfoxide concentration in assay is 5%). The assay was performed in a 96 well PCR plate from Greiner (#650201). Compound in dimethyl sulfoxide (3 μL) and enzyme (27 μL) were added to the plate, and pre-incubated for 10 min. The reaction was started with substrate (30 μL). The final dilution of enzyme was 20 μg/ml and the final concentration of substrate was 6 μM. After 20 minutes reaction at room temperature (RT), the reaction was stopped by removing 10 μL of the reaction mix and diluting it 1:25 in 0.2 M Trizma-HCl, pH 8.0. The product was quantified by adding 50 μL of a 1:5000 dilution of the neoepitope antibody to 50 μL of the 1:25 dilution of the reaction mix (all antibodies and the streptavidin coated beads were diluted in PBS containing 0.5% BSA and 0.5% Tween20). Then, 100 μL of 0.2 mg/mL streptavidin coated beads (Dynabeads M-280) and a 1:5000 dilution of ruthenylated goat anti-rabbit (Ru-GαR) antibody was added. The mixture was measured for electro-chemiluminescence in a BioVeris M8 Analyzer after 2 hours of incubation with shaking at RT. The dimethyl sulfoxide control defined 100% activity level and 0% activity was defined by exclusion of the enzyme (using 40 mM MES pH 5.0 buffer instead).

### Fluorescent Assay

The enzyme was diluted to 52 μg/ml in 40 mM MES pH 5.0. The substrate (Dabcyl-Edans) was diluted to 30 μM in 40 mM MES pH 5.0. Compounds were diluted to the desired concentration in dimethyl sulfoxide (final dimethyl sulfoxide concentration in assay is 5%). The assay is done in a Corning 384 well round bottom, low volume, non-binding surface plate (Corning #3676). Enzyme (9 μL) together with 1 μL of compound in dimethyl sulfoxide were added to the plate and pre-incubated for 10 min. Substrate (10 μL) was added and the reaction proceeded in the dark at RT for 25 min. The final dilution of enzyme was 23 μg/ml, and the final concentration of substrate was 15 μM (Km of 25 μM). The fluorescence of the product was measured on a Victor II plate reader with an excitation wavelength of 360 nm and an emission wavelength of 485 nm using a protocol for labelled Edans peptide. The dimethyl sulfoxide control defined 100% activity level and 0% activity was defined by exclusion of the enzyme (using 40 mM MES pH 5.0 buffer instead).

### TR-FRET Assay

Enzyme was diluted to 6 μg/mL and the substrate (Europium)CEVNLDAEFK(Qsy7) to 200 nM in reaction buffer (NaAcetate, chaps, triton x-100, EDTA pH 4.5). Compounds were diluted to the desired concentration in dimethyl sulfoxide (final dimethyl sulfoxide concentration in assay is 5%). The assay was done in a Costar 384 well round bottom, low volume, non-binding surface plate (Corning #3676). Enzyme (9 μL) and 1 μL of compound in dimethyl sulfoxide was added to the plate, mixed and pre-incubated for 10 min. Substrate (10 μL) was added and the reaction proceeded in the dark for 15 min at RT. The reaction was stopped with the addition of 7 μL NaAcetate, pH 9. The fluorescence of the product was measured on a Victor II plate reader with an excitation wavelength of 340 nm and an emission wavelength of 615 nm. The final concentration of the enzyme was 2.7 μg/ml and the final concentration of the substrate was 100 nM (Km of 290 nM). The dimethyl sulfoxide control defined the 100% activity level and 0% activity was defined by exclusion of the enzyme (using reaction buffer instead).

### BACE Biacore Sensor Chip Preparation

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was coupled to channel 2 of the same chip. The two peptides were dissolved at 0.2 mg/mL in 20 mM sodium acetate pH 4.5, and then the solutions were centrifuged at 14 K rpm to remove any particulates. Carboxyl groups on the dextran layer were activated by injecting a one to one mixture of 0.5 M N-ethyl-N′ (3-dimethylaminopropyl)-carbodiimide and 0.5 M N-hydroxysuccinimide at 5 μL/min for 7 min. Then the stock solution of the control peptide was injected in channel 1 for 7 min at 5 μL/min., and then the remaining activated carboxyl groups were blocked by injecting 1 M ethanolamine for 7 min at 5 μL/min.

### BACE Biacore Assay Protocol

The BACE Biacore assay was done by diluting BACE to 0.5 μM in sodium acetate buffer at pH 4.5 (running buffer minus dimethyl sulfoxide). The diluted BACE was mixed with dimethyl sulfoxide or compound diluted in dimethyl sulfoxide at a final concentration of 5% dimethyl sulfoxide. The BACE/inhibitor mixture was incubated for 30 minutes at RT before being injected over channel 1 and 2 of the CM5 Biacore chip at a rate of 20 μL/min. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethyl sulfoxide control was defined as 100% and the effect of the compound was reported as percent inhibition of the dimethyl sulfoxide control.

### Beta-Secretase Whole Cell Assays

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

Cell Culture for HEK293-APP695 HEK293 cells stably expressing human wild-type APP (HEK293-APP695) were grown at 37° C., 5% CO₂ in DMEM containing 4500 g/L glucose, GlutaMAX and sodium pyruvate supplemented with 10% FBS, 1% non-essential amino acids and 0.1 mg/mL of the selection antibiotic zeocin.

### Aβ840 Release Assay

HEK293-APP695 cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) containing test compounds in a final dimethyl sulfoxide concentration of 1%. Cells were exposed to the test compounds for 24 h at 37° C., 5% CO₂. To quantify the amount of released Aβ, 100 μL cell medium was transferred to a round bottom polypropylene 96-well plate (assay plate). The cell plate was saved for the ATP assay, as described below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated beads (Dynabeads M-280) was added per well. The plate was vigorously shaken at RT for 1-2 hours. The plate was then measured for electro-chemiluminescence in a BioVeris M8 Analyzer.

### Cell Culture for SH-SY5Y

SH-SY5Y cells were grown 37° C. with 5% CO₂ in DMEM/F-121:1 containing GlutaMAX supplemented with 1 mM HEPES, 10% FBS and 1% non-essential amino acids.

sAPPβ Release Assay

SH-SY5Y cells were harvested at 80-90% confluence and seeded at a concentration of 1.5×10⁶ cells/mL, 100 mL cell suspension/well, onto a black clear flat bottom 96-well tissue culture plate. After 7 hours of incubation at 37° C., 5% CO₂, the cell medium was replaced with 90 μl cell culture medium with penicillin and streptomycin (100 U/mL, 100 μg/mL, respectively) containing test compounds in a final dimethyl sulfoxide concentration of 1%. Cells were exposed to the test compounds for 18 h at 37° C., 5% CO₂. To measure sAPPβ released into the cell medium, sAPPβ microplates from Meso Scale Discovery (MSD) were used and the assay was performed according to the manufacture's protocol. Briefly, 25 μL cell medium was transferred to a previously blocked MSD sAPPβ microplate. The cell plate was saved for the ATP assay, as described below. The sAPPβ was captured during shaking at RT for 1 hour, by antibodies spotted in the wells of the microplate. After multiple washes, SULFO-TAG labeled detection antibody was added (25 μL/well, final concentration 1 nM) to the assay plate and the plate was incubated with shaking at RT for 1 hour. Following multiple washes, 150 μl/well of Read Buffer T was added to the plate. After 10 minutes at RT the plate was read in the SECTOR™ Imager for electro-chemiluminescence.

### ATP Assay

As indicated above, after transferring medium for analysis of Aβ40 or sAPPβ from the cell plate, the plate was used to analyze cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at RT for 10 min. Two min after addition of 100 μL reconstituted ViaLight™ Plus ATP reagent, the luminescence was measured in a Wallac Victor² 1420 multilabel counter.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made. A □-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 □g/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 □l of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

### Results

Typical IC50 values for the compounds of the present invention are in the range of about 1 to about 2,500 nM. Biological data on final compounds are given below in Table 11.

TABLE 11

| Example No. | IC50 (nM) in TR-FRET assay |
|---|---|
| 6 | 834 |
| 7 | 1200 |
| 14 | 59 |
| 21 | 1910 |
| 22 | 1650 |
| 29 | 387 |
| 34 | 924 |
| 39 | 365 |
| 57 | 34 |
| 58 | 81 |
| 59 | 128 |
| 60 | 25 |
| 61 | 37 |
| 62 | 25 |
| 63 | 18 |
| 64 | 28 |
| 69 | 23 |
| 70 | 2420 |
| 71 | 41 |
| 73 | 54 |
| 76 | 118 |
| 78 | 725 |
| 79 | 552 |
| 84 | 159 and inactive; two enantiomers |
| 86 | 292 |
| 87 | 494 |
| 88 | 234 |
| 89 | 207 |
| 90 | 385 |
| 91 | 228 |
| 92 | 179 |
| 93 | 146 |
| 94 | 472 |
| 95 | 352 |
| 96 | 242 |
| 97 | 236 |
| 98 | 239 |
| 99 | 330 |
| 100 | 395 |
| 101 | 180 |
| 102 | 321 |
| 103 | 227 |
| 104 | 186 |
| 105 | 343 |
| 106 | 363 |
| 107 | 311 |
| 108 | 186 |
| 109 | 263 |
| 110 | 114 |
| 111 | 93 |
| 112 | 150 |
| 113 | 121 |
| 129 | 35 |
| 130 | 59 |
| 133 | 33 |
| 134 | 43 & 1300; two enantiomers |
| 140 | 116 |
| 141 | 107 |
| 142 | 140 |
| 143 | 214 |
| 144 | 321 |
| 145 | 150 |
| 148 | 40 |
| 149 | 163 |
| 161 | 1680 |
| 162 | 682 |
| 163 | 395 |
| 164 | 912 |
| 165 | 589 |
| 167 | 46 |
| 168 | 349 |
| 169 | 171 |
| 170 | 651 |
| 176 | 148 |
| 177 | 100 |
| 184 | 156 |
| 185 | 19 |
| 187 | 459 |
| 188 | 379 |
| 192 | 1260 |
| 193 | 1180 |
| 194 | 284 |
| 195 | 646 |
| 197 | 721 |
| 209 | 99 |
| 210 | 137 |
| 211 | 104 |
| 225 | 633 |
| 226 | 859 |
| 227 | 1460 |
| 228 | 788 |
| 229 | 299 |
| 230 | 129 |
| 231 | 859 |
| 232 | 594 |
| 233 | 321 |
| 234 | 82 |
| 235 | 589 |
| 236 | 65 |
| 237 | 124 |
| 238 | 46 |
| 239 | 19 |
| 240 | 20 |
| 241 | 20 |
| 242 | 31 |
| 243 | 26 |
| 253 | 96 |
| 276 | 183 |
| 277 | 183 |
| 278 | 65 |
| 279 | 23 |
| 280 | 234 |
| 281 | 79 |
| 282 | 59 |
| 283 | 30 |
| 284 | 60 |
| 285 | 122 |
| 286 | 68 |
| 287 | 1020 |
| 288 | No data available |
| 289 | 115 |
| 296 | 1350 |
| 298 | 874 |
| 299 | 330 |
| 300 | 410 |
| 301 | 53 |
| 305 | 101 |
| 306 | 89 |
| 307 | 104 |
| 311 | 24 |
| 312 | 26 |

1. A compound according to formula I: 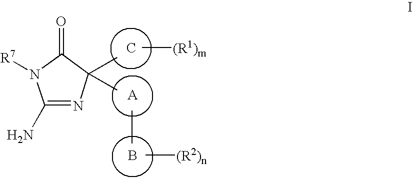
wherein
A is selected from phenyl and heteroaryl, wherein said phenyl or heteroaryl is optionally substituted with one or more R³;
B is selected from hydrogen, halo, cyano, phenyl, heteroaryl, heterocyclyl, C₃₋₆cycloalkyl, C₃₋₆cycloalkenyl, C₁₋₆alkyl, C₂₋₆alkenyl, C₀₋₆alkylC₃₋₆cycloalkyl, and C₂₋₆alkenylC₃₋₆cycloalkyl, wherein said phenyl, heteroaryl, heterocyclyl, C₃₋₆cycloalkyl, C₃₋₆cycloalkenyl, C₁₋₁₆alkyl, C₂₋₆alkenyl, C₀₋₆alkylC₃₋₆cycloalkyl or C₂₋₆alkenylC₃₋₆cycloalkyl, is optionally substituted with one or two R⁴;
C is selected from phenyl, heteroaryl and heterocyclyl, wherein said phenyl, heteroaryl or heterocyclyl is optionally substituted with one or two R⁵;
R¹ and R² is OSO₂R⁶;
R³ is selected from halo and cyano;
R⁴ is selected from halo, C₁₋₆alkyl, methoxy, cyano, acetyl and trifluoromethoxy;
R⁵ is selected from halo, C₁₋₆alkyl, methoxy, trifluoromethyl, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂;
said aryl being optionally fused with a 5 or 6 membered cyclyl or heterocyclyl group to form a bicyclic ring system; and said C₁₋₆alkyl, C₃₋₆cycloalkyl, aryl, heteroaryl, or bicyclic ring system is optionally substituted with one or more halogen, CN, NH₂, OH, COOH, OC₁₋₆alkyl, C₁₋₆alkylOH, SO₂H, C₁₋₆alkyl, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl or NC(O)(C₅₋₆heterocyclyl)₂;
R⁷ is C₁₋₆alkyl optionally substituted with halogen, CN, NH₂, OH, COOH, OC₁₋₆alkyl, SO₂H, C(O)C₁₋₆alkyl, C(O)OC₁₋₆alkyl, C(O)NH₂, C(O)NHC₁₋₆alkyl, C(O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(O)C₁₋₆alkyl, NC(O)(C₁₋₆alkyl)₂, aryl, Oaryl, C(O)aryl, C(O)Oaryl, C(O)NHaryl, C(O)N(aryl)₂, SO₂aryl, SO₂NHaryl, SO₂N(aryl)₂, NH(aryl), N(aryl)₂, NC(O)aryl, NC(O)(aryl)₂, heteroaryl, Oheteroaryl, C(O)heteroaryl, C(O)Oheteroaryl, C(O)NHheteroaryl, C(O)N(heteroaryl)₂, SO₂heteroaryl, SO₂NHheteroaryl, SO₂N(heteroaryl)₂, NH(heteroaryl), N(heteroaryl)₂, NC(O)heteroaryl, NC(O)(heteroaryl)₂, C₅₋₆heterocyclyl, OC₅₋₆heterocyclyl, C(O)C₅₋₆heterocyclyl, C(O)OC₅₋₆heterocyclyl, C(O)NHC₅₋₆heterocyclyl, C(O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(O)C₅₋₆heterocyclyl, NC(O)(C₅₋₆heterocyclyl)₂;
m=0 or 1;
n=0 or 1;
wherein one of m or n is at least 1;
provided that the following compounds are excluded:
4-[2-Amino-4-(3′-methoxybiphenyl-3-yl)-1-methyl-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl trifluoromethanesulfonate;
3′-(2-Amino-1-methyl-5-oxo-4-phenyl-4,5-dihydro-1H-imidazol-4-yl)biphenyl-3-yl trifluoromethanesulfonate;
3′-(2-Amino-1-methyl-5-oxo-4-phenyl-4,5-dihydro-1H-imidazol-4-yl)-5-methoxybiphenyl-2-yl trifluoromethanesulfonate;
3-[2-Amino-4-(3′-methoxybiphenyl-3-yl)-1-methyl-5-oxo-4,5-dihydro-1H-imidazol-4-yl]phenyl trifluoromethanesulfonate;
3-[2-Amino-4-(3′-methoxybiphenyl-3-yl)-1-methyl-5-oxo-4,5-dihydro-1H-imidazol-4-yl]phenyl methanesulfonate;
4-[2-Amino-1-methyl-5-oxo-4-(3-pyridin-3-ylphenyl)-4,5-dihydro-1H-imidazol-4-yl]phenyl methanesulfonate;
4-[2-Amino-4-(5′-chloro-2′-methoxybiphenyl-3-yl)-1-methyl-5-oxo-4,5-dihydro-1H-imidazol-4-yl]phenyl methanesulfonate;
4-[2-Amino-4-(3′-cyanobiphenyl-3-yl)-1-methyl-5-oxo-4,5-dihydro-1H-imidazol-4-yl]phenyl methanesulfonate;
4-{2-Amino-1-methyl-5-oxo-4-[3′-(trifluoromethoxy)biphenyl-3-yl]-4,5-dihydro-1H-imidazol-4-yl}phenyl methanesulfonate;
4-{2-Amino-4-[3-(2-chloropyridin-4-yl)phenyl]-1-methyl-5-oxo-4,5-dihydro-1H-imidazol-4-yl}phenyl methanesulfonate;
4-[2-Amino-4-(3′-cyanobiphenyl-3-yl)-1-methyl-5-oxo-4,5-dihydro-1H-imidazol-4-yl]phenyl propane-1-sulfonate;
4-[2-Amino-4-(2′,5′-dimethoxybiphenyl-3-yl)-1-methyl-5-oxo-4,5-dihydro-1H-imidazol-4-yl]phenyl propane-1-sulfonate;
4-[2-Amino-4-(2′-fluoro-3′-methoxybiphenyl-3-yl)-1-methyl-5-oxo-4,5-dihydro-1H-imidazol-4-yl]phenyl propane-1-sulfonate;
4-[2-Amino-4-(2′-fluoro-5′-methoxybiphenyl-3-yl)-1-methyl-5-oxo-4,5-dihydro-1H-imidazol-4-yl]phenyl propane-1-sulfonate;
4-[2-Amino-4-(3′-cyano-4′-fluorobiphenyl-3-yl)-1-methyl-5-oxo-4,5-dihydro-1H-imidazol-4-yl]phenyl propane-1-sulfonate;
4-[2-Amino-4-(5′-cyano-2′-fluorobiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyridin-3-ylphenyl)-4,5-dihydro-1H-imidazol-4-yl]phenyl propane-1-sulfonate;
4-{2-Amino-4-[3-(2-fluoropyridin-3-yl)phenyl]-1-methyl-5-oxo-4,5-dihydro-1H-imidazol-4-yl}phenyl propane-1-sulfonate;
4-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
4-{2-Amino-4-[3-(6-fluoropyridin-3-yl)phenyl]-1-methyl-5-oxo-4,5-dihydro-1H-imidazol-4-yl}phenyl propane-1-sulfonate;
4-[2-Amino-1-methyl-5-oxo-4-(3-pyrimidin-5-ylphenyl)-4,5-dihydro-1H-imidazol-4-yl]phenyl propane-1-sulfonate;
4-{2-Amino-4-[3-(2-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-3-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-5-fluoropyridin-3-yl)phenyl]-1-methyl-5-oxo-4,5-dihydro-1H-imidazol-4-yl}phenyl propane-1-sulfonate;
4-{2-Amino-4-[3-(2,6-difluoropyridin-3-yl)phenyl]-1-methyl-5-oxo-4,5-dihydro-1H-imidazol-4-yl}phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl propane-1-sulfonate;
4-[2-Amino-4-(4′-fluoro-3′-methoxybiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyrazin-2-ylphenyl)-4,5-dihydro-1H-imidazol-4-yl]phenyl propane-1-sulfonate;
3′-(2-Amino-1-methyl-5-oxo-4-phenyl-4,5-dihydro-1H-imidazol-4-yl)-5-methoxybiphenyl-3-yl methanesulfonate;
4-{2-Amino-4-[3-(5-methoxypyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl trifluoromethanesulfonate;
3-[2-Amino-1-methyl-5-oxo-4-(3-pyrimidin-5-ylphenyl)-4,5-dihydro-1H-imidazol-4-yl]phenyl trifluoromethanesulfonate;
3-{2-Amino-4-[3-(5-methoxypyridin-3-yl)phenyl]-1-methyl-5-oxo-4,5-dihydro-1H-imidazol-4-yl}phenyl trifluoromethanesulfonate;
4-[2-Amino-4-(3′,5′-dichlorodiphenyl-3-yl)-1-methyl-5-oxo-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl methanesulfonate;
3-{2-Amino-4-[3-(6-fluoropyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl methanesulfonate;
3-{2-Amino-4-[3-(5-chloro-2-fluoropyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-5-ylphenyl)-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl propane-1-sulfonate;
3-{2-Amino-4-[3-(6-fluoropyridin-3-yl)phenyl]-1-methyl-5-oxo-4,5-dihydro-1H-imidazol-4-yl}phenyl propane-1-sulfonate;
3-{2-Amino-4-[3-(2-fluoropyridin-3-yl)phenyl]-1-methyl-5-oxo-4,5-dihydro-1H-imidazol-4-yl}phenyl propane-1-sulfonate;
3-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-5-ylphenyl)-4,5-dihydro-1H-imidazol-4-yl]phenyl propane-1-sulfonate;
3-[2-Amino-1-methyl-5-oxo-4-(3-pyridin-3-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl propane-2-sulfonate;
3-{2-Amino-4-[3-(2-fluoropyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-5-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl dimethylsulfamate;
3-{2-Amino-4-[3-(5-chloro-2-fluoropyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-5-ylphenyl)-4,5-dihydro-1H-imidazol-4-yl]phenyl dimethylsulfamate;
3-{2-Amino-4-[3-(5-methoxypyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-2-ylphenyl)-4,5-dihydro-1H-imidazol-4-yl]phenyl methanesulfonate;
3-{2-Amino-4-[3-(5-methoxypyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-2-ylphenyl)-4,5-dihydro-1H-imidazol-4-yl]phenyl propane-1-sulfonate;
3-{2-Amino-4-[3-(5-methoxypyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-2-ylphenyl)-4,5-dihydro-1H-imidazol-4-yl]phenyl propane-2-sulfonate;
3-{2-Amino-4-[3-(5-methoxypyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-2-ylphenyl)-4,5-dihydro-1H-imidazol-4-yl]phenyl dimethylsulfamate;
3-[2-Amino-4-(3′-methoxybiphenyl-3-yl)-1-methyl-5-oxo-4,5-dihydro-1H-imidazol-4-yl]phenyl propane-2-sulfonate;
3′-(2-Amino-1-methyl-5-oxo-4-phenyl-4,5-dihydro-1H-imidazol-4-yl)-5-methoxybiphenyl-3-yl trifluoromethanesulfonate;
4-{2-Amino-4-[3-(5-methoxypyridin-3-yl)phenyl]-1-methyl-5-oxo-4,5-dihydro-1H-imidazol-4-yl}phenyl propane-1-sulfonate;
4-[2-Amino-4-(3′-cyano-6-fluorobiphenyl-3-yl)-1-methyl-5-oxo-4,5-dihydro-1H-imidazol-4-yl]phenyl methanesulfonate;
4-[2-Amino-4-(3′-cyano-6-fluorobiphenyl-3-yl)-5-oxo-4,5-dihydro-1H-imidazol-4-yl]phenyl methanesulfonate;
4-[2-Amino-4-(2′,6-difluoro-3′-methoxybiphenyl-3-yl)-5-oxo-4,5-dihydro-1H-imidazol-4-yl]phenyl methanesulfonate;
4-[2-Amino-4-(2′,6-difluoro-5′-methoxybiphenyl-3-yl)-5-oxo-4,5-dihydro-1H-imidazol-4-yl]phenyl methanesulfonate;
4-[2-Amino-4-(3′-cyano-4′,6-difluorobiphenyl-3-yl)-5-oxo-4,5-dihydro-1H-imidazol-4-yl]phenyl methanesulfonate;
4-[2-Amino-4-(5′-cyano-2′,6-difluorobiphenyl-3-yl)-5-oxo-4,5-dihydro-1H-imidazol-4-yl]phenyl methanesulfonate;
4-[2-Amino-4-(4-fluoro-3-pyridin-3-ylphenyl)-5-oxo-4,5-dihydro-1H-imidazol-4-yl]phenyl methanesulfonate;
4-{2-Amino-4-[4-fluoro-3-(2-fluoropyridin-3-yl)phenyl]-5-oxo-4,5-dihydro-1H-imidazol-4-yl}phenyl methanesulfonate (racemate);
4-{2-Amino-4-[3-(5-chloro-2-fluoropyridin-3-yl)-4-fluorophenyl]-5-oxo-4,5-dihydro-1H-imidazol-4-yl}phenyl methanesulfonate;
4-{2-Amino-4-[4-fluoro-3-(6-fluoropyridin-3-yl)phenyl]-5-oxo-4,5-dihydro-1H-imidazol-4-yl}phenyl methanesulfonate;
4-{2-Amino-4-[4-fluoro-3-(2-fluoropyridin-4-yl)phenyl]-5-oxo-4,5-dihydro-1H-imidazol-4-yl}phenyl methanesulfonate;
4-{2-Amino-4-[3-(2-chloro-3-fluoropyridin-4-yl)-4-fluorophenyl]-5-oxo-4,5-dihydro-1H-imidazol-4-yl}phenyl methanesulfonate;
4-{2-Amino-4-[3-(2-chloro-5-fluoropyridin-3-yl)-4-fluorophenyl]-5-oxo-4,5-dihydro-1H-imidazol-4-yl}phenyl methanesulfonate;
4-{2-Amino-4-[3-(2,6-difluoropyridin-3-yl)-4-fluorophenyl]-5-oxo-4,5-dihydro-1H-imidazol-4-yl}phenyl methanesulfonate;
4-{2-Amino-4-[6-fluoro-3′-(trifluoromethoxy)biphenyl-3-yl]-5-oxo-4,5-dihydro-1H-imidazol-4-yl}phenyl methanesulfonate;
4-[2-Amino-4-(3′-chloro-6-fluorobiphenyl-3-yl)-5-oxo-4,5-dihydro-1H-imidazol-4-yl]phenyl methanesulfonate;
4-[2-Amino-4-(4-fluoro-3-pyridin-4-ylphenyl)-5-oxo-4,5-dihydro-1H-imidazol-4-yl]phenyl methanesulfonate;
4-[2-Amino-4-(4-fluoro-3-pyrimidin-5-ylphenyl)-5-oxo-4,5-dihydro-1H-imidazol-4-yl]phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-{2-Amino-4-[4-fluoro-3-(5-fluoropyridin-3-yl)phenyl]-5-oxo-4,5-dihydro-1H-imidazol-4-yl}phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl propane-1-sulfonate;
4-[2-Amino-4-(3′-methoxybiphenyl-3-yl)-1-methyl-5-oxo-4,5-dihydro-1H-imidazol-4-yl]phenyl propane-2-sulfonate;
4-[2-Amino-4-(3′-methoxybiphenyl-3-yl)-1-methyl-5-oxo-4,5-dihydro-1H-imidazol-4-yl]phenyl dimethylsulfamate;
4-(2-Amino-4-{3′-methoxy-5′-[(methylsulfonyl)oxy]biphenyl-3-yl}-1-methyl-5-oxo-4,5-dihydro-1H-imidazol-4-yl)phenyl propane-1-sulfonate;
3-[2-Amino-1-methyl-5-oxo-4-(3-pyridin-3-ylphenyl)-4,5-dihydro-1H-imidazol-4-yl]phenyl trifluoromethanesulfonate;
3-[2-Amino-1-methyl-5-oxo-4-(3-pyridin-3-ylphenyl)-4,5-dihydro-1H-imidazol-4-yl]phenyl dimethylsulfamate;
4-(2-Amino-4-{6-fluoro-3′-methoxy-5′-[(methylsulfonyl)oxy]biphenyl-3-yl}-5-oxo-4,5-dihydro-1H-imidazol-4-yl)phenyl methanesulfonate;
3-(2-Amino-1-methyl-5-oxo-4-phenyl-4,5-dihydro-1H-imidazol-4-yl)phenyl methanesulfonate;
3-(2-Amino-1-methyl-5-oxo-4-phenyl-4,5-dihydro-1H-imidazol-4-yl)phenyl trifluoromethanesulfonate;
3′-(2-Amino-1-methyl-5-oxo-4-pyridin-4-yl-4,5-dihydro-1H-imidazol-4-yl)-5-methoxybiphenyl-3-yl methanesulfonate;
3′-(2-Amino-1-methyl-5-oxo-4-pyridin-2-yl-4,5-dihydro-1H-imidazol-4-yl)-5-methoxybiphenyl-3-yl methanesulfonate;
3′-[2-Amino-4-(3-furyl)-1-methyl-5-oxo-4,5-dihydro-1H-imidazol-4-yl]-5-methoxybiphenyl-3-yl methanesulfonate;
3′-[2-Amino-1-methyl-5-oxo-4-(1,3-thiazol-5-yl)-4,5-dihydro-1H-imidazol-4-yl]-5-methoxybiphenyl-3-yl methanesulfonate;
3′-[2-Amino-1-methyl-5-oxo-4-(1,3-thiazol-4-yl)-4,5-dihydro-1H-imidazol-4-yl]-5-methoxybiphenyl-3-yl methanesulfonate;
4-[2-Amino-4-(5′-fluoro-2′-methylbiphenyl-3-yl)-1-methyl-5-oxo-4,5-dihydro-1H-imidazol-4-yl]phenyl methanesulfonate;
4-[2-Amino-4-(3′-cyanobiphenyl-3-yl)-1-methyl-5-oxo-4,5-dihydro-1H-imidazol-4-yl]phenyl 2-methoxyethanesulfonate;
4-[2-Amino-4-(2′,5′-dimethoxybiphenyl-3-yl)-1-methyl-5-oxo-4,5-dihydro-1H-imidazol-4-yl]phenyl 2-methoxyethanesulfonate;
4-[2-Amino-4-(2′-fluoro-3′-methoxybiphenyl-3-yl)-1-methyl-5-oxo-4,5-dihydro-1H-imidazol-4-yl]phenyl 2-methoxyethanesulfonate;
4-[2-Amino-4-(2′-fluoro-5′-methoxybiphenyl-3-yl)-1-methyl-5-oxo-4,5-dihydro-1H-imidazol-4-yl]phenyl 2-methoxyethanesulfonate;
4-[2-Amino-4-(3′-cyano-4′-fluorobiphenyl-3-yl)-1-methyl-5-oxo-4,5-dihydro-1H-imidazol-4-yl]phenyl 2-methoxyethanesulfonate;
4-[2-Amino-4-(5′-cyano-2′-fluorobiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyridin-3-ylphenyl)-4,5-dihydro-1H-imidazol-4-yl]phenyl 2-methoxyethanesulfonate;
4-{2-Amino-4-[3-(2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(6-fluoropyridin-3-yl)phenyl]-1-methyl-5-oxo-4,5-dihydro-1H-imidazol-4-yl}phenyl 2-methoxyethanesulfonate;
4-[2-Amino-1-methyl-5-oxo-4-(3-pyrimidin-5-ylphenyl)-4,5-dihydro-1H-imidazol-4-yl]phenyl 2-methoxyethanesulfonate;
4-{2-Amino-4-[3-(2-chloro-3-fluoropyridin-4-yl)phenyl]-1-methyl-5-oxo-4,5-dihydro-1H-imidazol-4-yl}phenyl 2-methoxyethanesulfonate;
4-{2-Amino-4-[3-(2-chloro-5-fluoropyridin-3-yl)phenyl]-1-methyl-5-oxo-4,5-dihydro-1H-imidazol-4-yl}phenyl 2-methoxyethanesulfonate;
4-{2-Amino-4-[3-(2,6-difluoropyridin-3-yl)phenyl]-1-methyl-5-oxo-4,5-dihydro-1H-imidazol-4-yl}phenyl 2-methoxyethanesulfonate;
4-{2-Amino-4-[3-(5-fluoropyridin-3-yl)phenyl]-1-methyl-5-oxo-4,5-dihydro-1H-imidazol-4-yl}phenyl 2-methoxyethanesulfonate;
4-[2-Amino-4-(4′-fluoro-3′-methoxybiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyrazin-2-ylphenyl)-4,5-dihydro-1H-imidazol-4-yl]phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl ethanesulfonate;
3-[2-Amino-1-methyl-5-oxo-4-(3-pyridin-3-ylphenyl)-4,5-dihydro-1H-imidazol-4-yl]phenyl 2-methoxyethanesulfonate;
3-{2-Amino-4-[3-(6-fluoropyridin-3-yl)phenyl]-1-methyl-5-oxo-4,5-dihydro-1H-imidazol-4-yl}phenyl 2-methoxyethanesulfonate;
3-{2-Amino-4-[3-(2-fluoropyridin-3-yl)phenyl]-1-methyl-5-oxo-4,5-dihydro-1H-imidazol-4-yl}phenyl 2-methoxyethanesulfonate;
3-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
3-[2-Amino-1-methyl-5-oxo-4-(3-pyrimidin-5-ylphenyl)-4,5-dihydro-1H-imidazol-4-yl]phenyl 2-methoxyethanesulfonate;
3-[2-Amino-1-methyl-5-oxo-4-(3-pyridin-3-ylphenyl)-4,5-dihydro-1H-imidazol-4-yl]phenyl cyclopropanesulfonate;
3-{2-Amino-4-[3-(6-fluoropyridin-3-yl)phenyl]-1-methyl-5-oxo-4,5-dihydro-1H-imidazol-4-yl}phenyl cyclopropanesulfonate;
3-{2-Amino-4-[3-(2-fluoropyridin-3-yl)phenyl]-1-methyl-5-oxo-4,5-dihydro-1H-imidazol-4-yl}phenyl cyclopropanesulfonate;
3-{2-Amino-4-[3-(5-chloro-2-fluoropyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-5-ylphenyl)-4,5-dihydro-1H-imidazol-4-yl]phenyl cyclopropanesulfonate;
3-{2-Amino-4-[3-(5-methoxypyridin-3-yl)phenyl]-1-methyl-5-oxo-4,5-dihydro-1H-imidazol-4-yl}phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-2-ylphenyl)-4,5-dihydro-1H-imidazol-4-yl]phenyl cyclopropanesulfonate;
3-[2-Amino-4-(4-methoxyphenyl)-1-methyl-5-oxo-4,5-dihydro-1H-imidazol-4-yl]phenyl 3-methoxypropane-1-sulfonate;
3-[2-Amino-4-(4-methoxyphenyl)-1-methyl-5-oxo-4,5-dihydro-1H-imidazol-4-yl]phenyl methanesulfonate;
3-[2-Amino-4-(4-methoxyphenyl)-1-methyl-5-oxo-4,5-dihydro-1H-imidazol-4-yl]phenyl 2-ethoxyethanesulfonate;
3-[2-Amino-4-(4-methoxyphenyl)-1-methyl-5-oxo-4,5-dihydro-1H-imidazol-4-yl]phenyl 2-methoxyethanesulfonate;
4-[2-Amino-4-(3′-methoxybiphenyl-3-yl)-1-methyl-5-oxo-4,5-dihydro-1H-imidazol-4-yl]phenyl 2-methoxyethanesulfonate; and
4-{2-Amino-4-[3-(1H-indol-5-yl)phenyl]-1-methyl-5-oxo-4,5-dihydro-1H-imidazol-4-yl}phenyl methanesulfonate;
as a free base or a pharmaceutically acceptable salt, solvate or solvate of a salt thereof.

2. A compound according to claim 1, wherein
A is selected from phenyl or pyridine, wherein said phenyl or pyridine is optionally substituted with one or more R³;
B is selected from phenyl, pyridine, pyrimidine, pyrazine, CH₂CH₂cyclopropyl and CHCHcyclopropyl, wherein said phenyl, pyridine, pyrimidine or pyrazine is optionally substituted with one or two R⁴;
C is phenyl optionally substituted with one or two R⁵;
R¹ and R² is OSO₂R⁶;
R³ is fluoro;
R⁴ is selected from fluoro, chloro, methoxy, cyano and trifluoromethoxy;
R⁵ is selected from methyl, methoxy, difluoromethoxy and trifluoromethoxy or; two R⁵ may together form a 2,3-dihydro-1,4-benzodioxine;
R⁶ is selected from methyl, trifluoromethyl, propyl, isopropyl, phenyl and N(CH₃)₂;
R⁷ is selected from methyl, ethyl or propyl;
m=0 or 1;
n=0 or 1;
wherein one of m or n is at least 1.

3. A compound according to claim 1, wherein m is 1, n is 0, and R⁶ is selected from methyl, trifluoromethyl, propyl, isopropyl, phenyl and N(CH₃)₂.

4. A compound according to claim 1, wherein A is phenyl.

5. A compound according to claim 1, wherein A is phenyl substituted with one R³ and R³ is fluoro.

6. A compound according to claim 1, wherein A is pyridine.

7. A compound according to claim 1, wherein R⁷ is methyl, ethyl or propyl.

8. A compound according to claim 1, wherein C is phenyl.

9. A compound according to claim 1, wherein C is phenyl substituted with one R⁵, said R⁵ being selected from methyl, methoxy, difluoromethoxy or trifluoromethoxy.

10. A compound according to claim 1, wherein C is phenyl substituted with two R⁵, said two R⁵ being independently selected from methyl, methoxy and trifluoromethoxy or; two R⁵ together form a 2,3-dihydro-1,4-benzodioxine.

11. A compound according to claim 1, wherein B is phenyl.

12. A compound according to claim 1, wherein B is phenyl substituted with one R⁴, said R⁴ being selected from fluoro, chloro, methoxy and cyano.

13. A compound according to claim 1, wherein B is phenyl substituted with two R⁴, said two R⁴ being independently selected from fluoro, chloro, methoxy, cyano and trifluoromethoxy.

14. A compound according to claim 1, wherein B is pyridine.

15. A compound according to claim 1, wherein B is pyridine substituted with one R⁴, said R⁴ being selected from fluoro, chloro, methoxy and cyano.

16. A compound according to claim 1, wherein B is pyridine substituted with two R⁴, said two R⁴ being independently selected from fluoro and chloro.

17. A compound according to claim 1, wherein B is pyrimidine or pyrazine.

18. A compound according to claim 1, wherein B is CH₂CH₂cyclopropyl or CHCHcyclopropyl.

19. A compound according to claim 1, wherein R⁴ is acetyl.

20. A compound according to claim 1, wherein R⁶ is selected from C₁₋₆alkyl, C₃₋₆cycloalkyl, trifluoromethyl, aryl, heteroaryl and N(CH₃)₂; said aryl being optionally fused with a 5 or 6 membered cyclyl or heterocyclyl group to form a bicyclic ring system; and said aryl, heteroaryl, or bicyclic ring system is optionally substituted with one or more halogen, OC₁₋₆alkyl, C₁₋₆alkyl or heteroaryl.

21. A compound according to claim 1, wherein
A is selected from phenyl and heteroaryl, wherein said phenyl or heteroaryl is optionally substituted with one or more R³;
B is selected from hydrogen, halo, phenyl, heteroaryl, C₃₋₆cycloalkyl, C₃₋₆cycloalkenyl, C₀₋₆alkylC₃₋₆cycloalkyl, and C₂₋₆alkenylC₃₋₆cycloalkyl, wherein said phenyl is optionally substituted with one or two R⁴;
C is selected from phenyl, heteroaryl and heterocyclyl, wherein said phenyl or heteroaryl is optionally substituted with one or two R⁵;
R¹ and R² is OSO₂R⁶;
R³ is halo;
R⁴ is selected from halo, methoxy, cyano, acetyl and trifluoromethoxy;
R⁵ is selected from halo, C₁₋₆alkyl, methoxy, trifluoromethyl, difluoromethoxy and trifluoromethoxy; or two R⁵ may together form a 2,3-dihydro-1,4-benzodioxine;
R⁶ is selected from C₁₋₆alkyl, C₃₋₆cycloalkyl, trifluoromethyl, aryl, heteroaryl and N(CH₃)₂;
said aryl being optionally fused with a 5 or 6 membered cyclyl or heterocyclyl group to form a bicyclic ring system; and said aryl, heteroaryl, or bicyclic ring system is optionally substituted with one or more halogen, OC₁₋₆alkyl, C₁₋₆alkyl or heteroaryl;
R⁷ is C₁₋₆alkyl;
m=0 or 1;
n=0 or 1;
wherein one of m or n is at least 1.

22. A compound according to claim 1, wherein C is heteroaryl.

23. A compound according to claim 1, wherein C is selected from thiazolyl, pyrimidinyl and pyridinyl.

24. A compound according to claim 1, wherein C is heterocyclyl.

25. A compound according to claim 1, wherein C is cyclopentyl.

26. A compound according to claim 1, selected from:
4-{2-Amino-4-[4-fluoro-3-(4-methoxypyrimidin-2-yl)phenyl]-1-methyl-5-oxo-4,5-dihydro-1H-imidazol-4-yl}phenyl methanesulfonate 0.25 acetate;
3-{2-Amino-4-[4-fluoro-3-(4-methoxypyrimidin-2-yl)phenyl]-1-methyl-5-oxo-4,5-dihydro-1H-imidazol-4-yl}phenyl methanesulfonate 0.5 acetate;
5-[2-Amino-4-(4-fluoro-3-pyrimidin-5-ylphenyl)-1-methyl-5-oxo-4,5-dihydro-1H-imidazol-4-yl]-2-methoxyphenyl trifluoromethanesulfonate 0.5 acetate;
4-{2-Amino-4-[5-(2-fluoro-5-methoxyphenyl)pyridin-3-yl]-1-methyl-5-oxo-4,5-dihydro-1H-imidazol-4-yl}phenyl methanesulfonate 0.5 acetate;
4-[2-amino-4-(2′-fluoro-3,3′-bipyridin-5-yl)-1-methyl-5-oxo-4,5-dihydro-1H-imidazol-4-yl]phenyl methanesulfonate 0.5 acetate;
4-[2-Amino-4-(2′-fluoro-2,3′-bipyridin-4-yl)-1-methyl-5-oxo-4,5-dihydro-1H-imidazol-4-yl]phenyl methanesulfonate 0.25 acetate;
4-{2-Amino-4-[2-(3-cyanophenyl)pyridin-4-yl]-1-methyl-5-oxo-4,5-dihydro-1H-imidazol-4-yl}phenyl methanesulfonate 0.25 acetate;
4-{2-Amino-4-[2-(3-methoxyphenyl)pyridin-4-yl]-1-methyl-5-oxo-4,5-dihydro-1H-imidazol-4-yl}phenyl methanesulfonate 0.25 acetate;
3′-[2-Amino-4-(4-methoxyphenyl)-1-methyl-5-oxo-4,5-dihydro-1H-imidazol-4-yl]-5-methoxybiphenyl-3-yl methanesulfonate;
3′-{2-Amino-1-methyl-5-oxo-4-[4-(trifluoromethoxy)phenyl]-4,5-dihydro-1H-imidazol-4-yl}-5-methoxybiphenyl-3-yl methanesulfonate;
3′-{2-Amino-1-methyl-5-oxo-4-[4-(trifluoromethoxy)phenyl]-4,5-dihydro-1H-imidazol-4-yl}-5-chlorobiphenyl-3-yl methanesulfonate;
3′-{2-Amino-4-[4-methoxy-3-(trifluoromethyl)phenyl]-1-methyl-5-oxo-4,5-dihydro-1H-imidazol-4-yl}-5-methoxybiphenyl-3-yl methanesulfonate;
3′-{2-Amino-4-[4-methoxy-3-(trifluoromethyl)phenyl]-1-methyl-5-oxo-4,5-dihydro-1H-imidazol-4-yl}-5-chlorobiphenyl-3-yl methanesulfonate;
3′-[2-Amino-4-(4-methoxy-3-methylphenyl)-1-methyl-5-oxo-4,5-dihydro-1H-imidazol-4-yl]-5-methoxybiphenyl-3-yl methanesulfonate 0.25 acetate;
3′-{2-Amino-4-[4-(difluoromethoxy)phenyl]-1-methyl-5-oxo-4,5-dihydro-1H-imidazol-4-yl}-5-methoxybiphenyl-3-yl methanesulfonate 0.25 acetate;
3′-{2-Amino-4-[4-(difluoromethoxy)phenyl]-1-methyl-5-oxo-4,5-dihydro-1H-imidazol-4-yl}-5-chlorobiphenyl-3-yl methanesulfonate;
5′-{2-Amino-4-[4-(difluoromethoxy)phenyl]-1-methyl-5-oxo-4,5-dihydro-1H-imidazol-4-yl}-2′-fluoro-5-methoxybiphenyl-3-yl methanesulfonate hydrochloride;
5′-{2-Amino-4-[4-(difluoromethoxy)phenyl]-1-methyl-5-oxo-4,5-dihydro-1H-imidazol-4-yl}-5-cyano-2′-fluorobiphenyl-3-yl methanesulfonate hydrochloride;
4-[2-Amino-4-(3′-cyano-6-fluoro-5′-methoxybiphenyl-3-yl)-1-methyl-5-oxo-4,5-dihydro-1H-imidazol-4-yl]phenyl methanesulfonate hydrochloride;
5-(5-{2-Amino-4-[4-(difluoromethoxy)phenyl]-1-methyl-5-oxo-4,5-dihydro-1H-imidazol-4-yl}-2-fluorophenyl)pyridin-3-yl methanesulfonate hydrochloride;
4-[2-Amino-4-(3-fluoro-4-pyrimidin-5-ylphenyl)-1-methyl-5-oxo-4,5-dihydro-1H-imidazol-4-yl]phenyl trifluoromethanesulfonate hydrochloride;
4-(2-Amino-4-{3-[(E)-2-cyclopropylvinyl]-4-fluorophenyl}-1-methyl-5-oxo-4,5-dihydro-1H-imidazol-4-yl)phenyl methanesulfonate;
4-{2-Amino-4-[3-(2-cyclopropylethyl)-4-fluorophenyl]-1-methyl-5-oxo-4,5-dihydro-1H-imidazol-4-yl}phenyl methanesulfonate;
5′-(2-Amino-1-methyl-5-oxo-4-phenyl-4,5-dihydro-1H-imidazol-4-yl)-2′-fluoro-5-methoxybiphenyl-3-yl methanesulfonate;
3-{2-Amino-4-[3-(5-chloro-2-fluoropyridin-3-yl)-4-fluorophenyl]-1-methyl-5-oxo-4,5-dihydro-1H-imidazol-4-yl}phenyl methanesulfonate;
3-{2-Amino-4-[4-fluoro-3-(5-fluoropyridin-3-yl)phenyl]-1-methyl-5-oxo-4,5-dihydro-1H-imidazol-4-yl}phenyl methanesulfonate;
3-{2-Amino-4-[4-fluoro-3-(2-fluoropyridin-3-yl)phenyl]-1-methyl-5-oxo-4,5-dihydro-1H-imidazol-4-yl}phenyl methanesulfonate;
3-[2-Amino-4-(3′,5′-dichloro-6-fluorobiphenyl-3-yl)-1-methyl-5-oxo-4,5-dihydro-1H-imidazol-4-yl]phenyl methanesulfonate;
3-[2-Amino-4-(4-fluoro-3-pyridin-3-ylphenyl)-1-methyl-5-oxo-4,5-dihydro-1H-imidazol-4-yl]phenyl methanesulfonate,
3-[2-Amino-4-(3′-chloro-6-fluorobiphenyl-3-yl)-1-methyl-5-oxo-4,5-dihydro-1H-imidazol-4-yl]phenyl methanesulfonate;
3-[2-Amino-4-(3′-chloro-6-fluoro-5′-methoxybiphenyl-3-yl)-1-methyl-5-oxo-4,5-dihydro-1H-imidazol-4-yl]phenyl methanesulfonate;
3-[2-Amino-4-(6-fluoro-3′-methoxybiphenyl-3-yl)-1-methyl-5-oxo-4,5-dihydro-1H-imidazol-4-yl]phenyl methanesulfonate;
3-[2-Amino-4-(4-fluoro-3-pyrimidin-5-ylphenyl)-1-methyl-5-oxo-4,5-dihydro-1H-imidazol-4-yl]phenyl methanesulfonate;
3-{2-Amino-4-[3-(5-chloro-2-fluoropyridin-3-yl)-4-fluorophenyl]-1-methyl-5-oxo-4,5-dihydro-1H-imidazol-4-yl}phenyl propane-1-sulfonate;
3-{2-Amino-4-[4-fluoro-3-(2-fluoropyridin-3-yl)phenyl]-1-methyl-5-oxo-4,5-dihydro-1H-imidazol-4-yl}phenyl propane-1-sulfonate;
3-[2-Amino-4-(4-fluoro-3-pyridin-3-ylphenyl)-1-methyl-5-oxo-4,5-dihydro-1H-imidazol-4-yl]phenyl propane-1-sulfonate;
3-[2-Amino-4-(3′-chloro-6-fluorobiphenyl-3-yl)-1-methyl-5-oxo-4,5-dihydro-1H-imidazol-4-yl]phenyl propane-1-sulfonate;
3-[2-Amino-4-(3′-chloro-6-fluoro-5′-methoxybiphenyl-3-yl)-1-methyl-5-oxo-4,5-dihydro-1H-imidazol-4-yl]phenyl propane-1-sulfonate;
3-[2-Amino-4-(3′,5′-dichloro-6-fluorobiphenyl-3-yl)-1-methyl-5-oxo-4,5-dihydro-1H-imidazol-4-yl]phenyl propane-1-sulfonate;
3-[2-Amino-4-(6-fluoro-3′-methoxybiphenyl-3-yl)-1-methyl-5-oxo-4,5-dihydro-1H-imidazol-4-yl]phenyl propane-1-sulfonate;
3-[2-Amino-4-(4-fluoro-3-pyrimidin-5-ylphenyl)-1-methyl-5-oxo-4,5-dihydro-1H-imidazol-4-yl]phenyl propane-1-sulfonate;
3-{2-Amino-4-[3-(5-chloro-2-fluoropyridin-3-yl)-4-fluorophenyl]-1-methyl-5-oxo-4,5-dihydro-1H-imidazol-4-yl}phenyl propane-2-sulfonate;
3-{2-Amino-4-[4-fluoro-3-(2-fluoropyridin-3-yl)phenyl]-1-methyl-5-oxo-4,5-dihydro-1H-imidazol-4-yl}phenyl propane-2-sulfonate;
3-[2-Amino-4-(4-fluoro-3-pyridin-3-ylphenyl)-1-methyl-5-oxo-4,5-dihydro-1H-imidazol-4-yl]phenyl propane-2-sulfonate;
3-[2-Amino-4-(3′-chloro-6-fluorobiphenyl-3-yl)-1-methyl-5-oxo-4,5-dihydro-1H-imidazol-4-yl]phenyl propane-2-sulfonate;
3-[2-Amino-4-(3′-chloro-6-fluoro-5′-methoxybiphenyl-3-yl)-1-methyl-5-oxo-4,5-dihydro-1H-imidazol-4-yl]phenyl propane-2-sulfonate;
3-[2-Amino-4-(6-fluoro-3′-methoxybiphenyl-3-yl)-1-methyl-5-oxo-4,5-dihydro-1H-imidazol-4-yl]phenyl propane-2-sulfonate;
3-[2-Amino-4-(3′,5′-dichloro-6-fluorobiphenyl-3-yl)-1-methyl-5-oxo-4,5-dihydro-1H-imidazol-4-yl]phenyl propane-2-sulfonate;
3-{2-Amino-4-[4-fluoro-3-(2-fluoropyridin-3-yl)phenyl]-1-methyl-5-oxo-4,5-dihydro-1H-imidazol-4-yl}phenyl dimethylsulfamate;
3-[2-Amino-4-(4-fluoro-3-pyridin-3-ylphenyl)-1-methyl-5-oxo-4,5-dihydro-1H-imidazol-4-yl]phenyl dimethylsulfamate;
3-[2-Amino-4-(6-fluoro-3′-methoxybiphenyl-3-yl)-1-methyl-5-oxo-4,5-dihydro-1H-imidazol-4-yl]phenyl dimethylsulfamate;
3-[2-Amino-4-(3′,5′-dichloro-6-fluorobiphenyl-3-yl)-1-methyl-5-oxo-4,5-dihydro-1H-imidazol-4-yl]phenyl dimethylsulfamate;
4-{2-Amino-4-[4-fluoro-3-(5-methoxypyridin-3-yl)phenyl]-1-methyl-5-oxo-4,5-dihydro-1H-imidazol-4-yl}phenyl propane-1-sulfonate hydrochloride;
3-{2-Amino-4-[4-fluoro-3-(5-methoxypyridin-3-yl)phenyl]-1-methyl-5-oxo-4,5-dihydro-1H-imidazol-4-yl}phenyl propane-1-sulfonate hydrochloride;
3-[2-Amino-4-(4-fluoro-3-pyrimidin-5-ylphenyl)-1-methyl-5-oxo-4,5-dihydro-1H-imidazol-4-yl]phenyl benzenesulfonate hydrochloride;
(S)-4-{2-Amino-4-[4-fluoro-3-(2-fluoropyridin-3-yl)phenyl]-5-oxo-4,5-dihydro-1H-imidazol-4-yl}phenyl methanesulfonate;
4-{2-Amino-4-[3-(5-chloropyridin-3-yl)-4-fluorophenyl]-1-methyl-5-oxo-4,5-dihydro-1H-imidazol-4-yl}phenyl trifluoromethanesulfonate 0.75 acetate;
4-[2-Amino-4-(4-fluoro-3-pyridin-3-ylphenyl)-1-methyl-5-oxo-4,5-dihydro-1H-imidazol-4-yl]phenyl trifluoromethanesulfonate 0.5 acetate;
4-{2-Amino-4-[4-fluoro-3-(2-fluoropyridin-3-yl)phenyl]-1-methyl-5-oxo-4,5-dihydro-1H-imidazol-4-yl}phenyl trifluoromethanesulfonate 0.75 acetate;
4-[2-Amino-4-(4-fluoro-3-pyrazin-2-ylphenyl)-1-methyl-5-oxo-4,5-dihydro-1H-imidazol-4-yl]phenyl trifluoromethanesulfonate 0.5 acetate;
4-{2-Amino-4-[3-(5-cyanopyridin-3-yl)-4-fluorophenyl]-1-methyl-5-oxo-4,5-dihydro-1H-imidazol-4-yl}phenyl trifluoromethanesulfonate 0.5 acetate;
4-{2-amino-4-[4-fluoro-3-(5-fluoropyridin-3-yl)phenyl]-1-methyl-5-oxo-4,5-dihydro-1H-imidazol-4-yl}phenyl trifluoromethanesulfonate 0.5 acetate;
4-{2-Amino-4-[4-fluoro-3-(5-methoxypyridin-3-yl)phenyl]-1-methyl-5-oxo-4,5-dihydro-1H-imidazol-4-yl}phenyl trifluoromethanesulfonate 0.5 acetate;
4-{2-Amino-4-[3-(4-chloropyridin-2-yl)-4-fluorophenyl]-1-methyl-5-oxo-4,5-dihydro-1H-imidazol-4-yl}phenyl trifluoromethanesulfonate 0.75 acetate;
4-[2-Amino-4-(3′,5′-dichloro-6-fluorobiphenyl-3-yl)-1-ethyl-5-oxo-4,5-dihydro-1H-imidazol-4-yl]phenyl methanesulfonate;
4-[2-Amino-4-(4-fluoro-3-pyrimidin-5-ylphenyl)-1-ethyl-5-oxo-4,5-dihydro-1H-imidazol-4-yl]phenyl methanesulfonate;
4-[2-Amino-4-(6-fluoro-3′-methoxybiphenyl-3-yl)-1-ethyl-5-oxo-4,5-dihydro-1H-imidazol-4-yl]phenyl methanesulfonate 0.25 acetate;
4-[2-Amino-4-(3′-chloro-6-fluorobiphenyl-3-yl)-1-ethyl-5-oxo-4,5-dihydro-1H-imidazol-4-yl]phenyl methanesulfonate;
4-[2-Amino-4-(4-fluoro-3-pyrimidin-5-ylphenyl)-1-propyl-5-oxo-4,5-dihydro-1H-imidazol-4-yl]phenyl methanesulfonate 0.5 acetate;
4-[2-Amino-4-(2′-cyano-6-fluoro-5′-methoxybiphenyl-3-yl)-1-methyl-5-oxo-4,5-dihydro-1H-imidazol-4-yl]phenyl methanesulfonate;
4-{2-Amino-4-[6-fluoro-5′-methoxy-2′-(trifluoromethoxy)biphenyl-3-yl]-1-methyl-5-oxo-4,5-dihydro-1H-imidazol-4-yl}phenyl methanesulfonate;
4-{2-amino-4-[3-(4-chloropyridin-3-yl)-4-fluorophenyl]-1-methyl-5-oxo-4,5-dihydro-1H-imidazol-4-yl}phenyl methanesulfonate hydrochloride;
3-{2-Amino-4-[3-(4-chloropyridin-3-yl)phenyl]-1-methyl-5-oxo-4,5-dihydro-1H-imidazol-4-yl}phenyl methanesulfonate hydrochloride;
3-[2-Amino-4-(2′-cyano-5′-methoxybiphenyl-3-yl)-1-methyl-5-oxo-4,5-dihydro-1H-imidazol-4-yl]phenyl methanesulfonate hydrochloride;
3-[2-Amino-4-(2′-cyano-6-fluoro-5′-methoxybiphenyl-3-yl)-1-methyl-5-oxo-4,5-dihydro-1H-imidazol-4-yl]phenyl methanesulfonate hydrochloride;
3′-[2-Amino-4-(2,3-dihydro-1,4-benzodioxin-6-yl)-1-methyl-5-oxo-4,5-dihydro-1H-imidazol-4-yl]-5-methoxybiphenyl-3-yl methanesulfonate;
4-[2-Amino-4-(4-fluoro-3-pyridin-3-ylphenyl)-1-methyl-5-oxo-4,5-dihydro-1H-imidazol-4-yl]phenyl 1-methyl-1H-imidazole-4-sulfonate;
4-{2-amino-4-[4-(3-methoxyphenyl)pyridin-2-yl]-1-methyl-5-oxo-4,5-dihydro-1H-imidazol-4-yl}phenyl methanesulfonate 0.25 acetate;
4-{2-amino-4-[4-(3-methoxyphenyl)pyridin-2-yl]-1-methyl-5-oxo-4,5-dihydro-1H-imidazol-4-yl}phenyl trifluoromethanesulfonate 0.5 acetate;
4-[2-amino-4-(3-cyclohex-1-en-1-yl-4-fluorophenyl)-1-methyl-5-oxo-4,5-dihydro-1H-imidazol-4-yl]phenyl methanesulfonate 0.5 acetate;
4-[2-amino-4-(3-cyclohexyl-4-fluorophenyl)-1-methyl-5-oxo-4,5-dihydro-1H-imidazol-4-yl]phenyl methanesulfonate 0.5 acetate; and
4-{2-amino-4-[4-fluoro-3-(4-methoxypyridin-2-yl)phenyl]-1-methyl-5-oxo-4,5-dihydro-1H-imidazol-4-yl}phenyl methanesulfonate 0.75 acetate;
4-[2-Amino-4-(4-fluoro-3-pyrimidin-5-ylphenyl)-1-methyl-5-oxo-4,5-dihydro-1H-imidazol-4-yl]phenyl benzenesulfonate hydrochloride;
4-{2-Amino-4-[4-fluoro-3-(1,3-thiazol-5-yl)phenyl]-1-methyl-5-oxo-4,5-dihydro-1H-imidazol-4-yl}phenyl trifluoromethanesulfonate hydrochloride;
4-{2-Amino-4-[4-fluoro-3-(1,3-thiazol-5-yl)phenyl]-1-methyl-5-oxo-4,5-dihydro-1H-imidazol-4-yl}phenyl methanesulfonate hydrochloride;
5′-[2-Amino-1-methyl-5-oxo-4-(tetrahydro-2H-pyran-4-yl)-4,5-dihydro-1H-imidazol-4-yl]-2′-fluoro-5-methoxybiphenyl-3-yl methanesulfonate hydrochloride;
5′-[2-Amino-1-methyl-5-oxo-4-(tetrahydro-2H-pyran-4-yl)-4,5-dihydro-1H-imidazol-4-yl]-5-chloro-2′-fluorobiphenyl-3-yl methanesulfonate hydrochloride;
3-[2-Amino-4-(3-cyclopent-1-en-1-yl-4-fluorophenyl)-1-methyl-5-oxo-4,5-dihydro-1H-imidazol-4-yl]phenyl methanesulfonate hydrochloride;
3-[2-Amino-4-(3-cyclopent-1-en-1-yl-4-fluorophenyl)-1-methyl-5-oxo-4,5-dihydro-1H-imidazol-4-yl]phenyl methanesulfonate hydrochloride;
3-[2-Amino-4-(3-cyclopent-1-en-1-ylphenyl)-1-methyl-5-oxo-4,5-dihydro-1H-imidazol-4-yl]phenyl trifluoromethanesulfonate hydrochloride;
5′-[2-Amino-4-(2,6-diethylpyridin-4-yl)-1-methyl-5-oxo-4,5-dihydro-1H-imidazol-4-yl]-2′-fluoro-5-methoxybiphenyl-3-yl methanesulfonate hydrochloride;
5′-[2-Amino-4-(2,6-diethylpyridin-4-yl)-1-methyl-5-oxo-4,5-dihydro-1H-imidazol-4-yl]-5-chloro-2′-fluorobiphenyl-3-yl methanesulfonate hydrochloride;
5′-[2-Amino-1-methyl-4-(2-methylpyridin-4-yl)-5-oxo-4,5-dihydro-1H-imidazol-4-yl]-2′-fluoro-5-methoxybiphenyl-3-yl methanesulfonate hydrochloride;
3-[2-Amino-4-(3-bromo-4-fluorophenyl)-1-methyl-5-oxo-4,5-dihydro-1H-imidazol-4-yl]phenyl 2,6-difluorobenzenesulfonate;
3-[2-Amino-4-(3-bromo-4-fluorophenyl)-1-methyl-5-oxo-4,5-dihydro-1H-imidazol-4-yl]phenyl pyridine-2-sulfonate;
3-[2-Amino-4-(3-bromo-4-fluorophenyl)-1-methyl-5-oxo-4,5-dihydro-1H-imidazol-4-yl]phenyl 4-(1H-pyrazol-1-yl)benzenesulfonate;
3-[2-Amino-4-(3-bromo-4-fluorophenyl)-1-methyl-5-oxo-4,5-dihydro-1H-imidazol-4-yl]phenyl indane-5-sulfonate;
3-[2-Amino-4-(3-bromo-4-fluorophenyl)-1-methyl-5-oxo-4,5-dihydro-1H-imidazol-4-yl]phenyl 3-methyl-2-oxo-2,3-dihydro-1,3-benzoxazole-6-sulfonate;
3-[2-Amino-4-(3-bromo-4-fluorophenyl)-1-methyl-5-oxo-4,5-dihydro-1H-imidazol-4-yl]phenyl 5-chloro-1,3-dimethyl-1H-pyrazole-4-sulfonate;
3-[2-Amino-4-(3-bromo-4-fluorophenyl)-1-methyl-5-oxo-4,5-dihydro-1H-imidazol-4-yl]phenyl 5-chlorothiophene-2-sulfonate;
3-[2-Amino-4-(3-bromo-4-fluorophenyl)-1-methyl-5-oxo-4,5-dihydro-1H-imidazol-4-yl]phenyl 1-methyl-1H-imidazole-4-sulfonate;
3-[2-Amino-4-(3-bromo-4-fluorophenyl)-1-methyl-5-oxo-4,5-dihydro-1H-imidazol-4-yl]phenyl 6-chloroimidazo[2,1-b][1,3]thiazole-5-sulfonate;
3-[2-Amino-4-(4-fluoro-3-pyrimidin-5-ylphenyl)-1-methyl-5-oxo-4,5-dihydro-1H-imidazol-4-yl]phenyl 4-(1,1-dimethylpropyl)benzenesulfonate;
3-[2-Amino-4-(4-fluoro-3-pyrimidin-5-ylphenyl)-1-methyl-5-oxo-4,5-dihydro-1H-imidazol-4-yl]phenyl 2,5-dimethoxybenzenesulfonate;
3-[2-Amino-4-(4-fluoro-3-pyrimidin-5-ylphenyl)-1-methyl-5-oxo-4,5-dihydro-1H-imidazol-4-yl]phenyl 2,6-difluorobenzenesulfonate;
3-[2-Amino-4-(4-fluoro-3-pyrimidin-5-ylphenyl)-1-methyl-5-oxo-4,5-dihydro-1H-imidazol-4-yl]phenyl pyridine-2-sulfonate;
3-[2-Amino-4-(4-fluoro-3-pyrimidin-5-ylphenyl)-1-methyl-5-oxo-4,5-dihydro-1H-imidazol-4-yl]phenyl 4-(1H-pyrazol-1-yl)benzenesulfonate;
3-[2-Amino-4-(4-fluoro-3-pyrimidin-5-ylphenyl)-1-methyl-5-oxo-4,5-dihydro-1H-imidazol-4-yl]phenyl indane-5-sulfonate;
3-[2-Amino-4-(4-fluoro-3-pyrimidin-5-ylphenyl)-1-methyl-5-oxo-4,5-dihydro-1H-imidazol-4-yl]phenyl 3-methyl-2-oxo-2,3-dihydro-1,3-benzoxazole-6-sulfonate;
3-[2-Amino-4-(4-fluoro-3-pyrimidin-5-ylphenyl)-1-methyl-5-oxo-4,5-dihydro-1H-imidazol-4-yl]phenyl 5-chloro-1,3-dimethyl-1H-pyrazole-4-sulfonate;
3-[2-Amino-4-(4-fluoro-3-pyrimidin-5-ylphenyl)-1-methyl-5-oxo-4,5-dihydro-1H-imidazol-4-yl]phenyl 5-chlorothiophene-2-sulfonate;
3-[2-Amino-4-(4-fluoro-3-pyrimidin-5-ylphenyl)-1-methyl-5-oxo-4,5-dihydro-1H-imidazol-4-yl]phenyl 1-methyl-1H-imidazole-4-sulfonate;
5-[2-Amino-4-(4-fluoro-3-pyrimidin-5-ylphenyl)-1-methyl-5-oxo-4,5-dihydro-1H-imidazol-4-yl]-2-ethylphenyl methanesulfonate 0.5 acetate;
4-[2-Amino-4-(4-fluoro-3-pyrimidin-5-ylphenyl)-1-methyl-5-oxo-4,5-dihydro-1H-imidazol-4-yl]phenyl 4-(1,1-dimethylpropyl)benzenesulfonate;
4-[2-Amino-4-(4-fluoro-3-pyrimidin-5-ylphenyl)-1-methyl-5-oxo-4,5-dihydro-1H-imidazol-4-yl]phenyl 2,5-dimethoxybenzenesulfonate;
4-[2-Amino-4-(4-fluoro-3-pyrimidin-5-ylphenyl)-1-methyl-5-oxo-4,5-dihydro-1H-imidazol-4-yl]phenyl 2,6-difluorobenzenesulfonate;
4-[2-amino-4-(4-fluoro-3-pyrimidin-5-ylphenyl)-1-methyl-5-oxo-4,5-dihydro-1H-imidazol-4-yl]phenyl 4-methoxybenzenesulfonate;
4-[2-amino-4-(4-fluoro-3-pyrimidin-5-ylphenyl)-1-methyl-5-oxo-4,5-dihydro-1H-imidazol-4-yl]phenyl 3,5-dimethylisoxazol-4-sulfonate;
4-[2-Amino-4-(4-fluoro-3-pyridin-3-ylphenyl)-1-methyl-5-oxo-4,5-dihydro-1H-imidazol-4-yl]phenyl 4-(1,1-dimethylpropyl)benzenesulfonate;
4-[2-Amino-4-(4-fluoro-3-pyridin-3-ylphenyl)-1-methyl-5-oxo-4,5-dihydro-1H-imidazol-4-yl]phenyl 2,5-dimethoxybenzenesulfonate 0.25 acetate;
3-[2-Amino-4-(4-fluoro-3-pyridin-3-ylphenyl)-1-methyl-5-oxo-4,5-dihydro-1H-imidazol-4-yl]phenyl indane-5-sulfonate 0.25 acetate;
4-[2-Amino-4-(4-fluoro-3-pyridin-3-ylphenyl)-1-methyl-5-oxo-4,5-dihydro-1H-imidazol-4-yl]phenyl 3-methyl-2-oxo-2,3-dihydro-1,3-benzoxazole-6-sulfonate 0.5 acetate;
4-[2-Amino-4-(4-fluoro-3-pyridin-3-ylphenyl)-1-methyl-5-oxo-4,5-dihydro-1H-imidazol-4-yl]phenyl 5-chloro-1,3-dimethyl-1H-pyrazole-4-sulfonate 0.25 acetate;
4-[2-Amino-4-(4-fluoro-3-pyridin-3-ylphenyl)-1-methyl-5-oxo-4,5-dihydro-1H-imidazol-4-yl]phenyl 6-chloroimidazo[2,1-b][1,3]thiazole-5-sulfonate 0.75 acetate;
4-[2-Amino-4-(4-fluorophenyl)-1-methyl-5-oxo-4,5-dihydro-1H-imidazol-4-yl]phenyl 3-methyl-2-oxo-2,3-dihydro-1,3-benzoxazole-6-sulfonate;
4-[4-(2′-Acetyl-6-fluoro-5′-methoxybiphenyl-3-yl)-2-amino-1-methyl-5-oxo-4,5-dihydro-1H-imidazol-4-yl]phenyl methanesulfonate;
4-[2-Amino-4-(4-fluoro-3-pyridin-3-ylphenyl)-1-methyl-5-oxo-4,5-dihydro-1H-imidazol-4-yl]phenyl 1-methyl-1H-imidazole-4-sulfonate;
4-{2-amino-4-[4-(3-methoxyphenyl)pyridin-2-yl]-1-methyl-5-oxo-4,5-dihydro-1H-imidazol-4-yl}phenyl methanesulfonate 0.25 acetate;
4-{2-amino-4-[4-(3-methoxyphenyl)pyridin-2-yl]-1-methyl-5-oxo-4,5-dihydro-1H-imidazol-4-yl}phenyl trifluoromethanesulfonate 0.5 acetate;
4-[2-amino-4-(3-cyclohex-1-en-1-yl-4-fluorophenyl)-1-methyl-5-oxo-4,5-dihydro-1H-imidazol-4-yl]phenyl methanesulfonate 0.5 acetate;
4-[2-amino-4-(3-cyclohexyl-4-fluorophenyl)-1-methyl-5-oxo-4,5-dihydro-1H-imidazol-4-yl]phenyl methanesulfonate 0.5 acetate; and
4-{2-amino-4-[4-fluoro-3-(4-methoxypyridin-2-yl)phenyl]-1-methyl-5-oxo-4,5-dihydro-1H-imidazol-4-yl}phenyl methanesulfonate 0.75 acetate;
as a free base, alternative salt or a pharmaceutically acceptable salt, solvate or solvate of a salt thereof.

27. A pharmaceutical composition comprising as active ingredient a therapeutically effective amount of a compound according to claim 1, in association with pharmaceutically acceptable excipients, carriers or diluents.

28. A compound according to claim 1, or a pharmaceutically acceptable salt thereof, for use as a medicament.

29. A method of inhibiting activity of BACE comprising contacting said BACE with a compound of claim 1.

30. A method of treating or preventing an Aβ-related pathology in a mammal, comprising administering to said patient a therapeutically effective amount of a compound of claim 1.

31. The method of claim 30, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

32. A method of treating or preventing an Aβ-related pathology in a mammal, comprising administering to said patient a therapeutically effective amount of a compound of claim 1 and at least one cognitive enhancing agent, memory enhancing agent, or choline esterase inhibitor.

33. The method of claim 32, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.
